(19) United States
(12) Patent Application Publication
(10) Pub. No.: US 2019/0269133 A1
(43) Pub. Date: Sep. 5, 2019

(54) Herbicidal Pyrimidine Compounds

(71) Applicant: BASF SE, Ludwigshafen am Rhein (DE)

(72) Inventors: Florian VOGT, Ludwigshafen (DE); Matthias WITSCHEL, Ludwigshafen (DE); Tobias SEISER, Limburgerhof (DE); Veronica LOPEZ CARRILLO, Ludwigshafen (DE); Thomas SEITZ, Ludwigshafen (DE); Gerd KRAEMER, Limburgerhof (DE); Trevor William NEWTON, Limburgerhof (DE); Doreen SCHACHTSCHABEL, Ludwigshafen (DE); Klaus KREUZ, Limburgerhof (DE); Klaus REINHARD, Limburgerhof (DE)

(21) Appl. No.: 16/320,196

(22) PCT Filed: Jul. 24, 2017
(86) PCT No.: PCT/EP2017/068636

§ 371 Date: Jan. 24, 2019

(30) Foreign Application Priority Data
Jul. 27, 2016 (EP) 16181399.3

(51) Int. Cl. A01N 43/54; C07D 405/14; C07D 409/14; C07D 405/10

(52) U.S. Cl. CPC A01N 43/54; C07D 405/10; C07D 409/14; C07D 405/14

(57) ABSTRACT

The present invention relates to the pyrimidine compounds of formula (I),

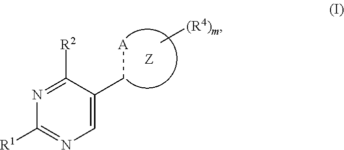

or their agriculturally acceptable salts or derivatives as herbicides, wherein the variables are defined according to the description, use of pyrimidine compounds of formula (I) as herbicide, compositions comprising them and their use as herbicides, i.e. for controlling harmful plants, and also a method for controlling unwanted vegetation which comprises allowing a herbicidal effective amount of at least one pyrimidine compounds of the formula (I) to act on plants, their seed and/or their habitat.

The present invention relates to pyrimidine compounds of the general formula (I) defined below and to their use as herbicides. Moreover, the invention relates to compositions for crop protection and to a method for controlling unwanted vegetation.

### DESCRIPTION

Compounds having a 5-phenyl pyrimidine moiety are known in the art. WO 2000/073278 describes such compounds being antagonists of the Neurokinin 1 receptor and thus having pharmaceutical properties.

In agriculture, there is a constant demand to develop novel active ingredients, which complement or outperform present methods of treatment regarding activity, selectivity and environmental safety.

These and further objects are achieved by pyrimidine compounds of formula (I), defined below, and by their agriculturally suitable salts.

Accordingly, the present invention provides the pyrimidine compounds of formula (I)

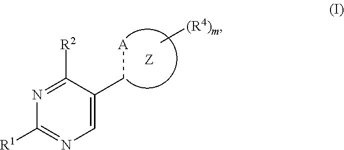

wherein

the dotted line (------) is a single bond or a double bond;
R¹ is C₁-C₆-alkyl, C₁-C₆-haloalkyl, HO—C₁-C₆-alkyl, C₂-C₆-alkenyl, C₂-C₆-haloalkenyl, C₂-C₆-alkynyl, C₃-C₆-haloalkynyl, C₁-C₆-alkoxy-C₁-C₆-alkyl, C₁-C₆-alkoxy, C₃-C₆-alkenyloxy, C₃-C₆-haloalkenyloxy, C₃-C₆-alkynyloxy, C₃-C₆-haloalkynyloxy, C₁-C₆-haloalkoxy, C₃-C₆-cycloalkoxy, C₃-C₆-halocycloalkoxy, C₃-C₆-cycloalkenyloxy, C₃-C₆-halocycloalkenyloxy, C₁-C₆-alkylthio, C₁-C₆-haloalkylthio, (C₁-C₆-alkyl)amino, di(C₁-C₆-alkyl)amino, C₁-C₆-alkylsulfinyl, C₁-C₆-alkylsulfonyl, C₃-C₆-cycloalkyl, C₃-C₆-cycloalkenyl, C₃-C₆-halocycloalkyl, C₃-C₆-halocycloalkenyl, [1-(C₁-C₆-alkyl)]-C₃-C₆-cycloalkyl, [1-(C₂-C₆-alkenyl)]-C₃-C₆-cycloalkyl, [1-(C₂-C₆-alkynyl)]-C₃-C₆-cycloalkyl, [1-(C₁-C₆-haloalkyl)]-C₃-C₆-cycloalkyl, [1-(C₂-C₆-haloalkenyl)]-C₃-C₆-cycloalkyl, [1-(C₃-C₆-haloalkynyl)]-C₃-C₆-cycloalkyl, C₃-C₆-cycloalkyl-C₁-C₆-alkyl, C₃-C₆-cycloalkyl-C₁-C₆-haloalkyl, C₃-C₆-cycloalkyl-C₁-C₆-alkoxy, C₃-C₆-cycloalkyl-C₁-C₆-haloalkoxy, phenyl, 5- or 6-membered heteroaryl, or 3- to 6-membered heterocyclyl;

wherein the cyclic groups of R¹ are unsubstituted or substituted by R^{a};
R² is C₁-C₆-alkyl, C₁-C₆-haloalkyl, C₂-C₆-alkenyl, C₂-C₆-haloalkenyl, C₁-C₆-alkoxy-C₂-C₆-alkenyl, C₁-C₆-alkoxy-C₂-C₆-haloalkenyl, C₁-C₆-haloalkoxy-C₂-C₆-alkenyl, C₁-C₆-haloalkoxy-C₂-C₆-haloalkenyl, C₂-C₆-alkynyl, C₂-C₆-haloalkynyl, C₁-C₆-alkoxy-C₂-C₆-alkynyl, C₁-C₆-alkoxy-C₃-C₆-haloalkynyl, C₁-C₆-haloalkoxy-C₂-C₆-alkynyl, C₁-C₆-haloalkoxy-C₃-C₆-haloalkynyl, C₃-C₆-cycloalkyl, C₃-C₆-halocycloalkyl, C₃-C₆-cycloalkenyl, C₃-C₆-halocycloalkenyl, C₃-C₆-cycloalkyl-C₁-C₆-alkyl, C₃-C₆-cycloalkyl-C₁-C₆-haloalkyl, C₃-C₆-halocycloalkyl-C₁-C₆-alkyl, C₃-C₆-halocycloalkyl-C₁-C₆-haloalkyl, C₃-C₆-cycloalkenyl-C₁-C₆-alkyl, C₃-C₆-cycloalkenyl-C₁-C₆-haloalkyl, C₃-C₆-halocycloalkenyl-C₁-C₆-alkyl, C₃-C₆-halocycloalkenyl-C₁-C₆-haloalkyl, C₃-C₆-cycloalkyl-C₂-C₆-alkenyl, C₃-C₆-cycloalkyl-C₂-C₆-haloalkenyl, C₃-C₆-halocycloalkyl-C₂-C₆-alkenyl, C₃-C₆-halocycloalkyl-C₂-C₆-haloalkenyl, C₃-C₆-cycloalkenyl-C₂-C₆-alkenyl, C₃-C₆-cycloalkenyl-C₂-C₆-haloalkenyl, C₃-C₆-halocycloalkenyl-C₂-C₆-alkenyl, C₃-C₆-halocycloalkenyl-C₂-C₆-haloalkenyl, C₃-C₆-cycloalkyl-C₂-C₆-alkynyl, C₃-C₆-cycloalkyl-C₃-C₆-haloalkynyl, C₃-C₆-halocycloalkyl-C₂-C₆-alkynyl, C₃-C₆-halocycloalkyl-C₃-C₆-haloalkynyl, C₃-C₆-cycloalkenyl-C₂-C₆-alkynyl, C₃-C₆-cycloalkenyl-C₃-C₆-haloalkynyl, C₃-C₆-halocycloalkenyl-C₂-C₆-alkynyl, C₃-C₆-halocycloalkenyl-C₃-C₆-haloalkynyl, C₃-C₆-cycloalkyl-C₁-C₆-alkylidenyl, C₃-C₆-cycloalkyl-C₂-C₆-haloalkylidenyl, C₃-C₆-halocycloalkyl-C₁-C₆-alkylidenyl, C₃-C₆-halocycloalkyl-C₂-C₆-haloalkylidenyl, C₃-C₆-cycloalkenyl-C₁-C₆-alkylidenyl, C₃-C₆-cycloalkenyl-C₂-C₆-haloalkylidenyl, C₃-C₆-halocycloalkenyl-C₁-C₆-alkylidenyl, C₃-C₆-halocycloalkenyl-C₂-C₆-haloalkylidenyl, heterocyclyl-C₁-C₆-alkylidenyl, heterocyclyl-C₂-C₆-haloalkylidenyl, C₃-C₆-hydroxycycloalkyl-C₁-C₆-alkyl, C₃-C₆-hydroxycycloalkyl-C₁-C₆-haloalkyl, C₃-C₆-hydroxycycloalkenyl-C₁-C₆-alkyl, C₃-C₆-hydroxycycloalkenyl-C₁-C₆-haloalkyl, C₁-C₆-hydroxyalkyl, C₂-C₆-hydroxyhaloalkyl, C₃-C₆-hydroxyalkenyl, C₃-C₆-hydroxyhaloalkenyl, C₃-C₆-hydroxyalkynyl, C₄-C₆-hydroxyhaloalkynyl, C₃-C₆-hydroxycycloalkyl, C₃-C₆-hydroxyhalocycloalkyl, C₃-C₆-hydroxycycloalkenyl, C₃-C₆-hydroxyhalocycloalkenyl, C₃-C₆-cycloalkyl-C₁-C₆-hydroxyalkyl, C₃-C₆-cycloalkyl-C₂-C₆-hydroxyhaloalkyl, C₃-C₆-halocycloalkyl-C₁-C₆-hydroxyalkyl, C₃-C₆-halocycloalkyl-C₂-C₆-hydroxyhaloalkyl, C₃-C₆-cycloalkenyl-C₁-C₆-hydroxyalkyl, C₃-C₆-cycloalkenyl-C₂-C₆-hydroxyhaloalkyl, C₃-C₆-halocycloalkenyl-C₁-C₆-hydroxyalkyl-C₃-C₆-halocycloalkenyl-C₂-C₆-hydroxyhaloalkyl, C₃-C₆-cycloalkyl-C₃-C₆-hydroxyalkenyl, C₃-C₆-cycloalkyl-C₃-C₆-hydroxyhaloalkenyl, C₃-C₆-halocycloalkyl-C₃-C₆-hydroxyalkenyl, C₃-C₆-halocycloalkyl-C₃-C₆-hydroxyhaloalkenyl, C₃-C₆-cycloalkenyl-C₃-C₆-hydroxyalkenyl, C₃-C₆-cycloalkenyl-C₃-C₆-hydroxyhaloalkenyl, C₃-C₆-halocycloalkenyl-C₃-C₆-hydroxyalkenyl, C₃-C₆-halocycloalkenyl-C₃-C₆-hydroxyhaloalkenyl, C₃-C₆-cycloalkyl-C₃-C₆-hydroxyalkynyl, C₃-C₆-halocycloalkyl-C₃-C₆-hydroxyalkynyl, C₃-C₆-cycloalkenyl-C₃-C₆-hydroxyalkynyl, C₃-C₆-halocycloalkenyl-C₃-C₆-hydroxyalkynyl, C₃-C₆-cycloalkyl-C₂-C₆-hydroxyalkylidenyl, C₃-C₆-halocycloalkyl-C₂-C₆-hydroxyalkylidenyl, C₃-C₆-cycloalkenyl-C₂-C₆-hydroxyalkylidenyl, C₃-C₆-halocycloalkyl-C₂-C₆-hydroxyalkylidenyl, heterocyclyl-C₂-C₆-hydroxyalkylidenyl, hydroxycarbonyl-C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-hydroxyalkyl, C₁-C₆-haloalkoxycarbonyl-C₁-C₆-hydroxyalkyl, C₁-C₆-alkoxycarbonyl-C₁-C₆-haloalkyl, C₁-C₆-haloalkoxycarbonyl-C₁-C₆-haloalkyl, C₁-C₆-alkoxycarbonyl-C₁-C₆-alkyl, C₁-C₆-haloalkoxycarbonyl-C₁-C₆-alkyl, C₃-C₆-hydroxycycloalkyl-C₁-C₆-hydroxyalkyl, C₃-C₆-hydroxycycloalkenyl-C₁-C₆-hydroxyalkyl, C₃-C₆-hydroxycycloalkyl-C₃-C₆-hydroxyalkenyl, C₃-C₆-hydroxycycloalkenyl-C₃-C₆-hydroxyalkenyl, C₃-C₆-hydroxycycloalkyl-C₃-C₆-hydroxyalkynyl, C₃-C₆-hydroxycycloalkenyl-C₃-C₆-hydroxyalkenyl, C₂-C₆-dihydroxyalkyl, C₃-C₆-dihydroxyhaloalkyl, C₄-C₆-dihydroxyalkenyl, C₄-C₆-dihydroxyhaloalkenyl, C₄-C₆-dihydroxyalkynyl, C₅-C₆-dihydroxyhaloalkynyl, C₄-C₆-dihydroxycycloalkyl, C₄-C₆-dihydroxyhalocycloalkyl, C₄-C₆-dihydroxycycloalkenyl, C₄-C₆-dihydroxyhalocycloalkenyl, C₃-C₆-cycloalkyl-C₂-C₆-dihydroxyalkyl, C₃-C₆-halocycloalkyl-C₂-C₆-dihydroxyalkyl, C₃-C₆-cycloalkenyl-C₂-C₆-dihydroxyalkyl, C₃-C₆-halocycloalkenyl-C₂-C₆-dihydroxyalkyl, C₃-C₆-cycloalkyl-C₃-C₆-dihydroxyalkenyl, C₃-C₆-halocycloalkyl-C₃-C₆-dihydroxyalkenyl, C₃-C₆-cycloalkenyl-C₃-C₆-dihydroxyalkenyl, C₃-C₆-halocycloalkenyl-C₃-C₆-dihydroxyalkenyl, C₃-C₆-cycloalkyl-C₄-C₆-dihydroxyalkynyl, C₃-C₆-halocycloalkyl-C₄-C₆-dihydroxyalkynyl, C₃-C₆-cycloalkenyl-C₄-C₆-dihydroxyalkynyl, C₃-C₆-halocycloalkyl-C₄-C₆-dihydroxyalkynyl, C₃-C₆-cycloalkyl-C₃-C₆-dihydroxyalkylidenyl, C₃-C₆-halocycloalkyl-C₃-C₆-dihydroxyalkylidenyl, heterocyclyl-C₃-C₆-dihydroxyalkylidenyl, hydroxycarbonyl-C₂-C₆-dihydroxyalkyl, hydroxycarbonyl-C₃-C₆-dihydroxyhaloalkyl, C₁-C₆-alkoxycarbonyl-C₂-C₆-dihydroxyalkyl, C₁-C₆-haloalkoxycarbonyl-C₂-C₆-dihydroxyalkyl, C₁-C₆-haloalkoxycarbonyl-C₃-C₆-dihydroxyhaloalkyl, C₃-C₆-dihydroxycycloalkyl-C₁-C₆-alkyl, C₁-C₆-haloalkyl-C₃-C₆-dihydroxycycloalkyl-C₂-C₆-alkenyl, C₃-C₆-dihydroxycycloalkyl-C₂-C₆-haloalkenyl, C₃-C₆-dihydroxycycloalkyl-C₂-C₆-alkynyl, C₃-C₆-dihydroxycycloalkyl-C₃-C₆-haloalkynyl, C₁-C₆-alkylcarbonyl-C₁-C₆-alkyl, C₁-C₆-haloalkylcarbonyl-C₁-C₆-alkyl, C₁-C₆-alkylcarbonyl-C₁-C₆-haloalkyl, C₁-C₆-haloalkylcarbonyl-C₁-C₆-haloalkyl, hydroxycarbonyl-C₂-C₆-alkenyl, hydroxycarbonyl-C₂-C₆-haloalkenyl, C₁-C₆-alkoxycarbonyl-C₂-C₆-alkenyl, C₁-C₆-haloalkoxycarbonyl-C₂-C₆-alkenyl, C₁-C₆-alkoxycarbonyl-C₂-C₆-haloalkenyl, C₁-C₆-haloalkoxycarbonyl-C₂-C₆-haloalkenyl, hydroxycarbonyl-C₂-C₆-alkynyl, hydroxycarbonyl-C₃-C₆-haloalkynyl, C₁-C₆-alkoxycarbonyl-C₂-C₆-alkynyl, C₁-C₆-haloalkoxycarbonyl-C₂-C₆-alkynyl, C₁-C₆-alkoxycarbonyl-C₃-C₆-haloalkynyl, C₁-C₆-haloalkoxycarbonyl-C₃-C₆-haloalkynyl, C₁-C₆-cyanoalkyl, C₂-C₆-cyanohaloalkyl, C₁-C₆-dicyanoalkyl, C₂-C₆-dicyanohaloalkyl, di(hydroxycarbonyl)-C₁-C₆-alkyl, di(hydroxycarbonyl)-C₁-C₆-haloalkyl, di(C₁-C₆-alkoxycarbonyl)-C₁-C₆-alkyl, di(C₁-C₆-haloalkoxycarbonyl)-C₁-C₆-alkyl, di(C₁-C₆-alkoxycarbonyl)-C₁-C₆-haloalkyl, di(C₁-C₆-haloalkoxycarbonyl)-C₁-C₆-haloalkyl, di(C₁-C₆-alkoxyl)phosphoryl-C₁-C₆-alkyl, di(C₁-C₆-haloalkoxyl)phosphoryl-C₁-C₆-alkyl, di(C₁-C₆-alkoxyl)phosphoryl-C₁-C₆-haloalkyl, di(C₁-C₆-haloalkoxyl)phosphoryl-C₁-C₆-haloalkyl, phosphoryl-C₁-C₆-alkyl, phosphoryl-C₁-C₆-haloalkyl, di[di(C₁-C₆-alkoxyl)phosphoryl-)]C₁-C₆-alkyl, di[di(C₁-C₆-haloalkoxyl)phosphoryl-)]C₁-C₆-alkyl, di[di(C₁-C₆-alkoxyl)phosphoryl-)]C₁-C₆-haloalkyl, di[di(C₁-C₆-haloalkoxyl)phosphoryl-)]C₁-C₆-haloalkyl, diphosphoryl-C₁-C₆-alkyl, diphosphoryl-C₁-C₆-haloalkyl, C₁-C₆-alkylthio-C₁-C₆-alkyl, C₁-C₆-haloalkylthio-C₁-C₆-alkyl, C₁-C₆-alkylthio-C₁-C₆-haloalkyl, C₁-C₆-haloalkylthio-C₁-C₆-haloalkyl, C₁-C₆-alkylsulfinly-C₁-C₆-alkyl, C₁-C₆-haloalkylsulfinly-C₁-C₆-alkyl, C₁-C₆-alkylsulfinly-C₁-C₆-haloalkyl, C₁-C₆-haloalkylsulfinly-C₁-C₆-haloalkyl, C₁-C₆-alkylsulfonyl-C₁-C₆-alkyl, C₁-C₆-haloalkylsulfonyl-C₁-C₆-alkyl, C₁-C₆-haloalkylsulfonyl-C₁-C₆-haloalkyl, phenyl, 5- or 6-membered heteroaryl, or 3- to 6-membered heterocyclyl;
wherein hydroxy groups of R² are unsubstituted or substituted by R^{b};
cyclic groups of R² are unsubstituted or substituted by R^{c}; and
acyclic aliphatic groups of R² are unsubstituted or substituted by R^{d}.
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{c} is halogen, CN, NO₂, C₁-C₆-alkyl, C₁-C₆-haloalkyl, hydroxy, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
   wherein the substituent R^{d} is unsubstituted or substituted by Re;
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylsulfonyl;
Z is a 9 or 10 membered bicyclic ring comprising A, wherein the ring comprising A is a 5- or 6-membered aromatic ring comprising 0, 1, 2, or 3 heteroatoms selected from O, N, and S, and which is fused with another 5- or 6-membered partially or fully unsaturated carbocycle comprising 0, 1, 2, or 3 heteroatoms selected from O, N, and S;
A is C*, CR³, NR^{3A}, N, O, or S;
C* is a bridge carbon of the bicyclic ring Z;
R³ is halogen, CN, CHO, NO₂, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
   wherein the cyclic groups of R³ are unsubstituted or substituted by substituents R^{a};
R^{3A} is H, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
   wherein the cyclic groups of R^{3A} are unsubstituted or substituted by R^{a};
R⁴ is halogen, CN, CHO, NO₂, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
   wherein the cyclic groups of R⁴ are unsubstituted or substituted by R^{a};
R^{a} is halogen, CN, NO₂, C₁-C₆-alkyl, C₁-C₆-haloalkyl, C₁-C₆-alkoxy, or C₁-C₆-haloalkoxy;
m is 0, 1, 2, or 3;

including agriculturally acceptable salts or derivatives of the pyrimidine compounds of formula (I) having an acidic functionality.

The present invention also provides use of the pyrimidine compounds of formula (I) as described herein including agriculturally acceptable salts or derivatives of the pyrimidine compounds of formula (I) having an acidic functionality, as herbicide.

The pyrimidine compounds of formula (I) according to the invention can be prepared by standard processes of organic chemistry, e.g. by the following processes:

Process A:

The pyrimidines of formula (II) can be obtained by reacting respective pyrimidines of formula (I) (prepared analogous to known procedures like e.g. in WO 2013186229), with R²═CH₃, with base and an electrophile, e.g. a carbonyl compound (III):

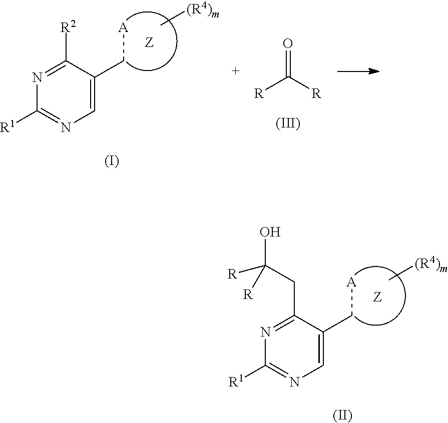

The reaction of the pyrimidine (I) with the electrophile (III), with R independent of each other equals hydrogen, alkyl, cycloalkyl, halocycloalkyl, haloalkyl, cycloalkenyl, halocycloalkenyl, alkenyl, haloalkenyl, alkynyl, phenyl, heterocyclyl, heteroaryl or both R form together a carbocycle or a heterocycle, is usually carried out at temperatures of from −100° C. to the boiling point of the reaction mixture, preferably from −80° C. to 20° C., particularly preferably from −80° C. to −20° C., in an inert organic solvent in the presence of a base.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, tert-butyl methyl ether (TBME), dioxane, anisole and tetrahydrofuran (THF), and also dimethyl sulfoxide (DMSO), dimethylformamide (DMF) and N,N-dimethylacetamide (DMAC), particularly diethyl ether, dioxane and THF.

It is also possible to use mixtures of the solvents mentioned.

Suitable bases are, in general, inorganic compounds, such as alkali metal and alkaline earth metal anhydrides, such as lithium hydride (LiH), sodium hydride (NaH), potassium hydride (KH) and calcium hydride (CaH), alkali metal amides, such as lithium hexamethyidisilazide (LHMDS) and lithium diisopropylamide (LDA), organometallic compounds, in particular alkali metal alkyls, such as methyllithium (MeLi), butyllithium (BuLi) and phenyllithium (PhLi), and also alkali metal and alkaline earth metal alkoxides, such as sodium methoxide (NaOCH₃), sodium ethoxide (NaOC₂H₅), potassium ethoxide (KOC₂H₅), potassium tert-butoxide (tBuOK), potassium tert-pentoxide and dimethoxymagnesium, moreover organic bases, e.g. tertiary amines, such as trimethylamine (TMA), triethylamine (TEA), diisopropylethylamine (DIPEA) and N-methylpiperidine, pyridine, substituted pyridines, such as collidine, lutidine and 4-dimethylaminpyridine, and also bicyclic amines. Particular preference is given to NaH, LHMDS and lithium diisopropylamide (LDA).

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine compounds (I).

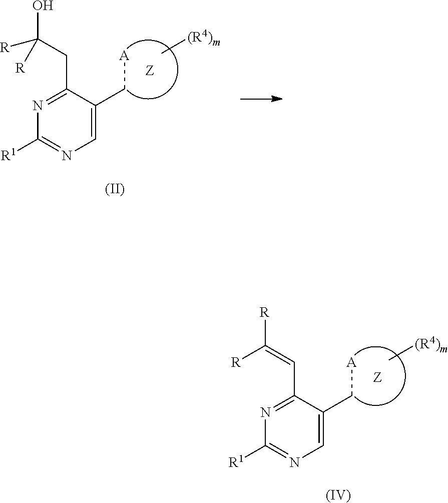

The elimination of the alcohol of the pyrimidine (II) is usually carried out at temperatures from −100° C. to the boiling point of the reaction mixture, preferably from 0° C. to 120° C., particularly preferably from 20° C. to 100° C., in an inert solvent optionally in the presence of an acid.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably toluene and o-xylene.

It is also possible to use mixtures of the solvents mentioned.

Suitable acids are inorganic acids, such as HCl, HBr, sulfuric acid; organic acids p-toluenesulfonic acid, benzene sulfonic acid, pyridinium p-toluol sulfonic acid, methanesulfonic acid, acetic acid; preferably p-toluenesulfonic acid and HCl.

The acids are generally employed in equimolar amounts; however, they can also be employed in catalytic amounts, in excess or, if appropriate, as solvents.

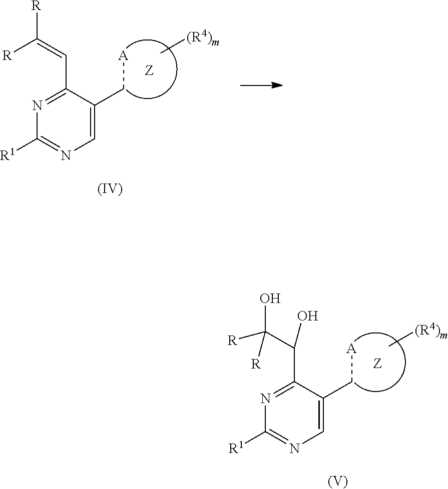

The oxidation of the olefin (IV) to the diol (V) is usually carried out at temperatures of from −100° C. to the boiling point of the reaction mixture, preferably from 0° C. to 120° C., particularly preferably from 20° C. to 100° C., in an inert solvent.

The reaction may in principle be carried out in substance. However, preference is given to reacting the pyrimidines (IV) with the oxidant in an organic solvent.

Suitable in principle are all solvents which are capable of dissolving the pyrimidines (IV) and the oxidant at least partly and preferably fully under the reaction conditions.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably TBME, THF. It is also possible to use mixtures of the solvents mentioned.

Suitable oxidants are e.g. potassium permanganate, potassium perruthenate, osmium tetroxide and other osmium salts, like potassium osmate. The oxidant can be used in equimolar amounts or in catalytic amounts together with a reoxidant like N-methylmorpholine-N-oxide or potassium hexacyanoferrate in stochiometric amounts or in excess.

Process B:

The halopyrimidines VI are known or can be prepared by known procedures (X═Cl, Br, I).

The boronic acids or esters required for the preparation of pyrimidine compounds of formula (VII) are commercially available, known from literature or can easily prepared analogously to published procedures (e.g. Kamei et al. Tetrahedron Lett. 2014, 55, 4245-4247).

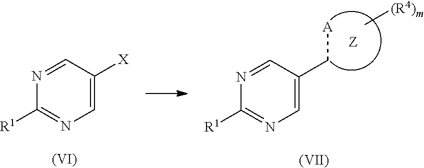

The pyrimidine compounds of formula (VII) can obtained by reacting boronic acids or esters with halides of formula (VI) in which X equals Cl, Br, or I in presence of a base and a catalyst in analogy to WO 2014202493.

The reaction may in principle be carried out in substance. However, preference is given to reacting the pyrimidines (VI) with the boronic acid or ester in an organic solvent with or without water as co-solvent.

Suitable in principle are all solvents which are capable of dissolving the pyrimidines (VI) and the boronic acid or ester at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMI, N,N′-dimethylpropylene urea (DMPU), DMSO and 1-methyl-2 pyrrolidinone (NMP).

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

The term base as used herein also includes mixtures of two or more, preferably two of the above compounds. Particular preference is given to the use of one base.

The bases are used preferably from 1 to 10 equivalents based on the pyrimidine (VI), more preferably from 1.0 to 5.0 equivalents based on the pyrimidine (VI), most preferably from 1.2 to 2.5 equivalents based on the pyrimidine (VI).

It may be advantageous to add the base offset over a period of time.

The reaction of the pyrimidines (VI) with the phenyl boronic acid or ester is carried out in the presence of a catalyst. Examples of suitable catalysts include e.g., palladium based catalysts like, e.g., Palladium(II)acetate, tetrakis(triphenylphosphine)palladium(0), bis(triphenylphosphine)palladium(II)chloride, or (1,1,-bis(diphenylphosphino)ferrocene)dichloropalladium(II), and optionally suitable additives such as, e.g., phosphines like, e.g., P(o-tolyl)₃, triphenylphosphine, or BINAP (2,2′-Bis(diphenylphospino)-1,1′-binaphthyl).

The amount of catalyst is usually 0.01 to 10 mol % (0.0001 to 0.1 equivalents) based on the pyrimidine (VI).

The pyrimidine compounds of formula (VIII) can be obtained by reacting respective pyrimidines of formula (VII) with base and an electrophile, e.g., a carbonyl compound (III):

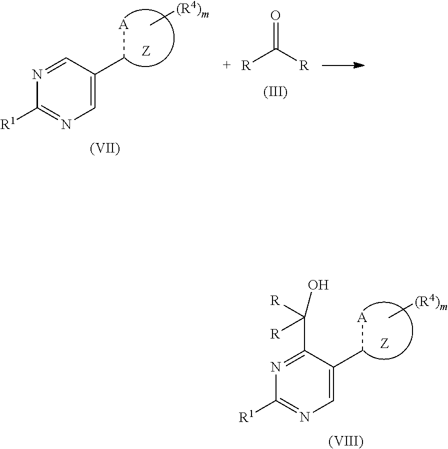

The reaction of the pyrimidine (VII) with the electrophile (III), with R independent of each other equals hydrogen, alkyl, cycloalkyl, halocycloalkyl, haloalkyl, cycloalkenyl, halocycloalkenyl, alkenyl, haloalkenyl, alkynyl, phenyl, heterocyclyl, heteroaryl or both R form together a carbocycle or a heterocycle, is usually carried out at temperatures from −100° C. to the boiling point of the reaction mixture, preferably from −80° C. to 20° C., particularly preferably from −80° C. to −20° C., in an inert organic solvent in the presence of a base.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also dimethyl sulfoxide, DMF and DMAC, particularly preferably diethyl ether, dioxane and THF.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine (VII).

Process C:

The pyrimidine compounds of formula (IX) can be obtained by reacting respective aminoketones of formula (X) with base and amidine (XI):

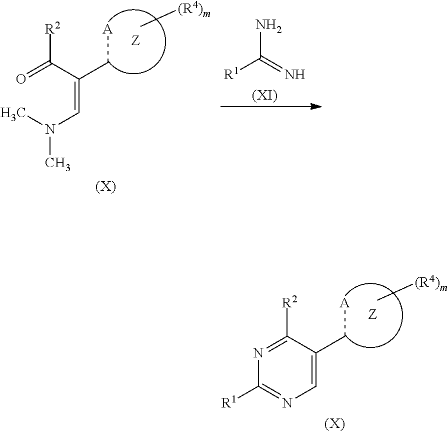

The reaction of aminoketone (X) with the amidine (XI) is usually carried out at temperatures of from −100° C. to the boiling point of the reaction mixture, preferably from 20° C. to the boiling point, particularly preferably from 40° C. to 120° C., in an inert organic solvent in the presence of a base.

The reaction may in principle be carried out in substance. However, preference is given to reacting the aminoketones (X) with the amidine (XI) in an organic solvent.

Suitable in principle are all solvents which are capable of dissolving the aminoketones (X) with the amidine (XI) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, alcohols such as methanol, ethanol, n-propanol, isopropanol, n-butanol and tert.-butanol, as well as dipolar aprotic solvents such as sulfolane, dimethylsulfoxide, DMF, DMAC, DMIDMI, DMPU, DMSO and NMP.

Preferred solvents are alcohols such as methanol and ethanol.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaOCH₃, NaOC₂H₅, KOC₂H₅, potassium tert-butoxide and potassium tert-pentoxide.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the amidine (XI), based on the aminoketone (X)

Amidines (XI) known from literature or commercial available.

Preparation of Compound (X)

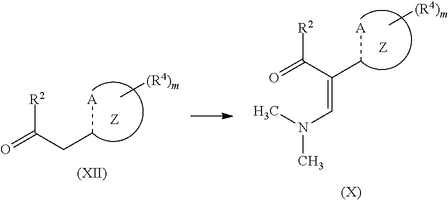

The aminoketones (X) are prepared from the corresponding ketones (XII) with N,N-dimethylformamide dimethyl acetal. The reaction is usually carried out at temperatures from −100° C. to the boiling point of the reaction mixture, preferably from 20° C. to 160° C., particularly preferably from 50° C. to 130° C. The reaction can optionally be catalyzed be an acid.

The reaction may be carried out in substance or in an organic solvent. Suitable in principle are all solvents which are capable of dissolving the ketones (XII) and N,N-dimethylformamide dimethyl acetal at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO, DMF, DMAC, DMI, DMPU, DMSO and NMP; preferably DMAC is used as solvent.

It is also possible to use mixtures of the solvents mentioned.

Suitable acids are inorganic acids, such as HCl, HBr, sulfuric acid; organic acids p-toluenesulfonic acid, benzene sulfonic acid, pyridinium p-toluol sulfonic acid, methanesulfonic acid, acetic acid; preferably p-toluenesulfonic acid and HCl. Most preferred, no acid is used.

The acids are generally employed in equimolar amounts; however, they can also be employed in catalytic amounts, in excess or, if appropriate, as solvents.

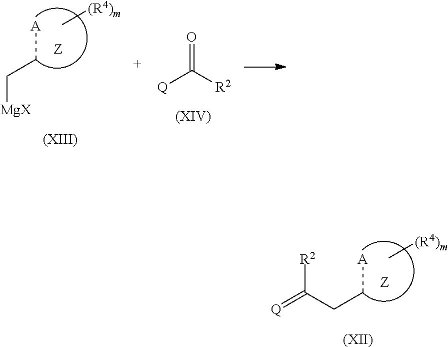

The ketones (XII) are prepared by reacting Grignard-reagent (XIII) with a carbonyl-electrophile (XIV) (e.g. an acid halide (Q=F, Cl or Br) or a Weinreb-Amide (Q=N(Me)OMe)). The reaction is usually carried out at temperatures of from −100° C. to the boiling point of the reaction mixture, preferably from −80° C. to 60° C., particularly preferably from −80° C. to 20° C., in an inert solvent.

Suitable in principle are all solvents which are capable of dissolving the Grignard-reagent (XIII) and the carbonyl-electrophile (XIV) at least partly and preferably fully under the reaction conditions.

Examples of suitable solvents are aliphatic aromatic hydrocarbons such as benzene, chlorobenzene, toluene, cresols, o-, m- and p-xylene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO,

DMF, DMAC, DMIDMI, DMPU, DMSO and NMP. Preferred solvents are ethers such as TBME or THF.

It is also possible to use mixtures of the solvents mentioned.

The Grignard-reagents (XIII) are either commercial available or can be prepared from the corresponding halides by known methods.

The carbonyl electrophiles (XIV) are either commercial available or can be prepared from the corresponding carboxylic acid or carboxylic ester by known methods.

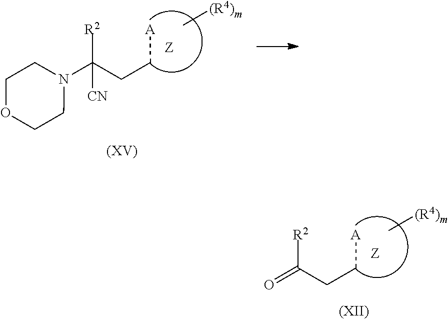

Ketones (XII) can as well be prepared from morpholinonitriles (XV) as described in the literature (European Journal of Organic Chemistry 2013, 36, 8083)

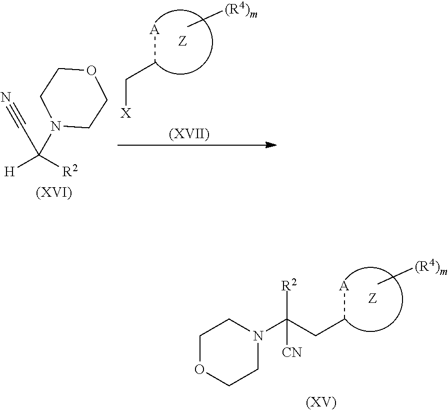

The morpholinonitriles (XV) are prepared from morpholinonitriles (XVI) and benzylhalides

(XVII) in the presence of a base. The reaction is usually carried out at temperatures of from −100° C. to the boiling point of the reaction mixture, preferably from −80° C. to 60° C., particularly preferably from −50° C. to 20° C., in an inert organic solvent in the presence of a base. Suitable in principle are all solvents which are capable of dissolving the morpholinonitriles (XVI) and the benzylhalides (XVII) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as dichloromethane, 1,2-dichloroethane, chloroform, carbon tetrachloride and chlorobenzene, ethers such as diethyl ether, diisopropyl ether, TBME, dioxane, anisole and THF, esters such as ethyl acetate and butyl acetate; nitriles such as acetonitrile and propionitrile, as well as dipolar aprotic solvents such as sulfolane, DMSO, DMF, DMAC, DMIDMI, DMPU, DMSO and NMP. Preferred solvents are dipolar aprotic solvents such as sulfolane, dimethylsulfoxide, DMF, DMAC, DMIDMI, DMPU, DMSO and NMP.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the halide (XVII), based on the morpholinonitrile (XVI).

Benzylhalides (XVII) are commercial available.

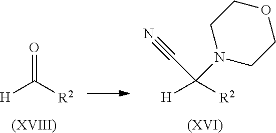

Morpholinonitriles (XVI) are prepared from the corresponding aldehydes (XVIII) as described in the literature (WO 2009/013462).

Aldehydes (XVIII) are commercial available.

Process D:

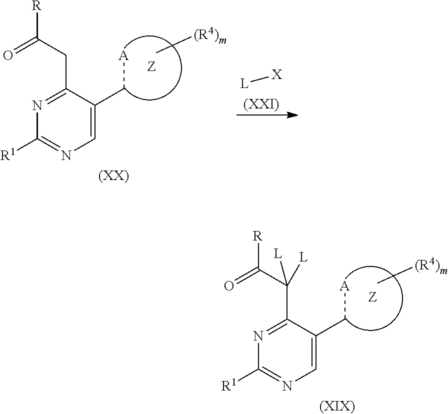

Pyrimidine compounds (XIX), with R equals alkyl, haloalkyl, alkoxy, haloalkoxy, can be obtained by reacting respective pyrimidine compounds of formula (XX) with base and an electrophile (XXI).

Electrophile (XXI) can be an alkyl-, alkenyl- or alkynyl-halide, e.g. methyl iodide, allyl bromide or propargyl bromide, or a halogenating agent, e.g. Cl₂, Br₂, I₂, NCS (N-Chlorosuccinimide), NBS (N-Bromosuccinimide), NIS (N-Iodosuccinimide), NFSI (N-Fluorobenzenesulfonimide), Selectfluor (1-Chloromethyl-4-fluoro-1,4-diazoniabicyclo[2.2.2]octane bis(tetrafluoroborate)).

The reaction of the pyrimidine (XX) with the electrophile is usually carried out at temperatures of from −100° C. to the boiling point of the reaction mixture, preferably from −80° C. to 80° C., particularly preferably from −80° C. to 30° C., in an inert organic solvent in the presence of a base.

Suitable in principle are all solvents which are capable of dissolving the pyrimidine (XX) and the electrophile (XXI) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, nitriles such as acetonitrile and propionitrile, as well as dipolar aprotic solvents such as sulfolane, dimethylsulfoxide, DMF, N,N-DMAC (DMAC), DMIDMI, DMPU, DMSO and NMP.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile (XXI), based on the pyrimidine (XX).

The pyrimidine compounds of formula (XX) can be obtained by reacting respective pyrimidines of formula (I) (prepared analogous to known procedures like e.g. in WO 2013186229), R²═CH₃, with base and an electrophile (XXII), e.g. a dialkylcarbonate (X═R=alkoxy), an alkyl chloroformiate (X=halogene, R=alkoxy) or an acid halide (X=halogene, R=alkyl or haloalkyl):

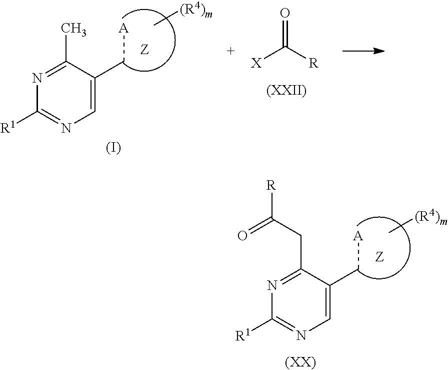

The reaction of the pyrimidine (I) with the electrophile (XXII) is usually carried out at temperatures of from −100° C. to the boiling point of the reaction mixture, preferably from −80° C. to 80° C., particularly preferably from −80° C. to 30° C., in an inert organic solvent in the presence of a base.

Suitable in principle are all solvents which are capable of dissolving the pyrimidine (I) and the electrophile (XXII) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, DMSO, DMF, DMAC, DMIDMI, DMPU, DMSO and NMP.

It is also possible to use mixtures of the solvents mentioned.

Suitable bases are, in general, inorganic compounds, such as alkali metal and alkaline earth metal anhydrides, such as LiH, NaH, KH and CaH, alkali metal amides, such as LDA, LHMDS, lithium 2,2,6,6-tetramethylpiperidide (LTMP), organometallic compounds, in particular alkali metal alkyls, such as MeLi, BuLi and PhLi, and also alkali metal and alkaline earth metal alkoxides, such as NaOCH₃, NaOC₂H₅, KOC₂H₅, tBuOK, potassium tert-pentoxide and dimethoxymagnesium, moreover organic bases, e.g. tertiary amines, such as TMA, TEA, DIPEA and N-methylpiperidine, pyridine, substituted pyridines, such as collidine, lutidine and 4-dimethylaminpyridine, and also bicyclic amines. Particular preference is given to NaH, LTMP and LDA.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile (XXII), based on the pyrimidine (I).

Process E:

The pyrimidines of formula (XXIII) can be obtained by reacting respective pyrimidines of formula (XXIV) with boronic acids/esters of formula (XXV):

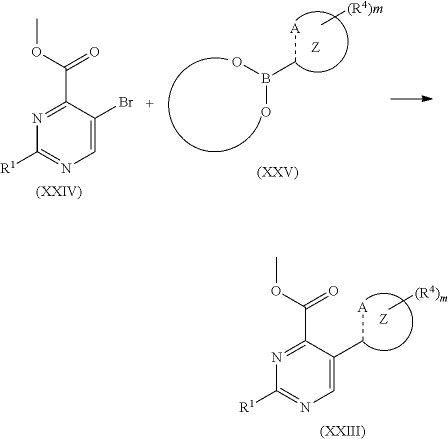

The reaction of pyrimidines (XXIV) with boronic acids/esters (XXV) is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXIV) with the boronic acids/esters (XXV) in an organic solvent with or without water as co-solvent.

Suitable in principle are all solvents which are capable of dissolving the pyrimidines (XXIV) and the boronic acids (XXV) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMIDMI, DMPU, DMSO and NMP.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

The term base as used herein also includes mixtures of two or more, preferably two of the above bases. Particular preference is given to the use of one base.

The bases are used preferably from 1 to 10 equivalents based on the pyrimidine (XXIV), more preferably from 1.0 to 5.0 equivalents based on the pyrimidine (XXIV), most preferably from 1.2 to 2.5 equivalents based on the pyrimidine (XXIV).

It may be advantageous to add the base offset over a period of time.

The reaction of the pyrimidines (XXIV) with the boronic acids/esters (XXV) is carried out in the presence of a catalyst. Examples of suitable catalysts include e.g., palladium based catalysts like, e.g., palladium(II)acetate, tetrakis(triphenylphosphine)-palladium(0), bis(triphenylphosphine)palladium(II)chloride or (1,1,-bis(diphenylphosphino)-ferrocene)-dichloropalladium(II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXIV).

The halopyrimidines (XXIV) are known from the literature (e.g. WO 2011154327), are commercially available or can be prepared by known procedures.

The boronic acids/esters (XXV) required for the preparation of pyrimidines of formula (XVII) are commercially available, known from literature or can easily prepared analogously to published procedures (e.g. Kamei et al. Tetrahedron Lett. 2014, 55, 4245-4247).

The pyrimidines of formula (XXVI) can be obtained by reacting respective pyrimidines of formula (XXIII) with a reducing agent such as LAH or DIBAIH.

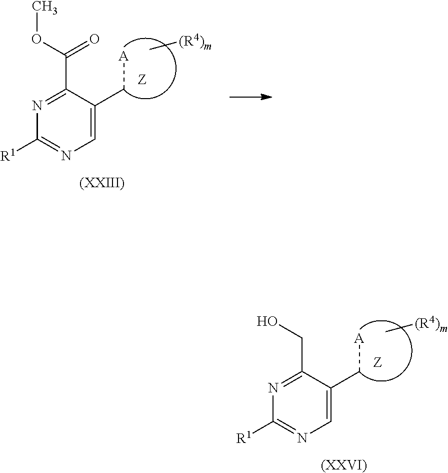

The reduction of pyrimidines (XXIII) is usually carried out from −80° C. to the boiling point of the reaction mixture, preferably from −20° C. to 60° C., particularly preferably from 0° C. to 25° C., in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably diethyl ether, dioxane and THF.

It is also possible to use mixtures of the solvents mentioned.

Examples of reducing agents for pyrimidines (XXIII) include LAH, DIBALH, LiBH₄ or Lithium triethylborohydride.

Preferred agents include LAH and DIBALH.

The hydride-source is used preferably from 1 to 10 equivalents based on the pyrimidine (XXIII), more preferably from 1.0 to 5.0 equivalents based on the pyrimidine (XXIII), most preferably from 1.2 to 2.5 equivalents based on the pyrimidine (XXIII).

Process F:

The pyrimidines of formula (XXVII) can be obtained by reacting respective pyrimidines of formula (XXIII) with a metal organic species like a Grignard reagent (R′MgX, X═Cl, Br, I; R′=alkyl, cycloalkyl, halocycloalkyl, haloalkyl, cycloalkenyl, halocycloalkenyl, alkenyl, haloalkenyl, alkynyl, phenyl, heterocyclyl or heteroaryl).

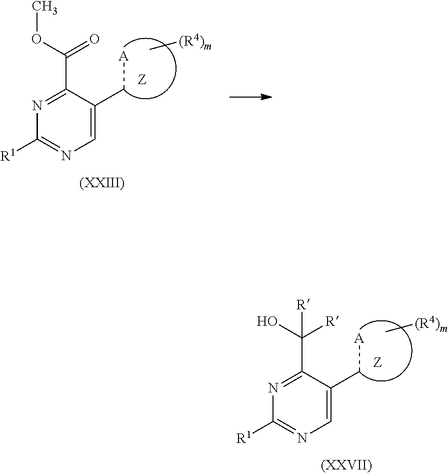

The reaction of pyrimidines (XXIII) with a metal organic species is usually carried out from −80° C. to the boiling point of the reaction mixture, preferably from −20° C. to 60° C., particularly preferably from −20° C. to 25° C., in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably diethyl ether, dioxane and THF.

It is also possible to use mixtures of the solvents mentioned.

Examples of metal organic species for the synthesis of pyrimidines (XXVII) are Grignard reagents like R′MgCl, R′MgBr or R′MgI, lithium organic species, aluminum organic species like R′₃Al, R′₂AlX and R′AlX₂, titanium organic species like R′₄Ti, R′₃TiX, R′₂TiX₂ and R′TiX₃.

Preferred agents include Grignard reagents and lithium organic species.

The metal organic species is used preferably from 2 to 10 equivalents based on the pyrimidine (XXIII), more preferably from 2.0 to 5.0 equivalents based on the pyrimidine (XXIII), most preferably from 2.0 to 3.0 equivalents based on the pyrimidine (XXIII).

Process G:

The pyrimidines of formula (XXVIII) can be obtained by reacting respective pyrimidines of formula (XXIX) with a metal organic species like a Grignard reagent (R′MgX, X═Cl, Br, I; R′=alkyl, cycloalkyl, halocycloalkyl, haloalkyl, cycloalkenyl, halocycloalkenyl, alkenyl, haloalkenyl, alkynyl, phenyl, heterocyclyl or heteroaryl).

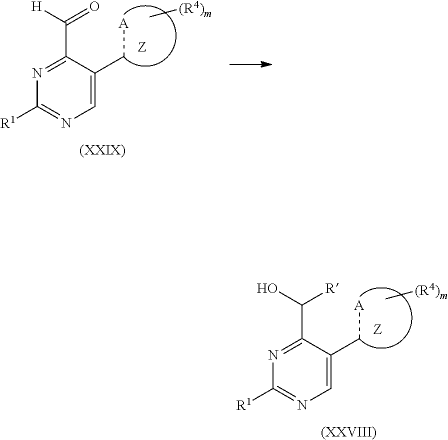

The reaction of pyrimidines (XXIX) with a metal organic species is usually carried out from −80° C. to the boiling point of the reaction mixture, preferably from −20° C. to 60° C., particularly preferably from −20° C. to 25° C., in an inert organic solvent.

Suitable solvents are aliphatic hydrocarbons, such as pentane, hexane, cyclohexane and mixtures of C₅-C₈-alkanes, aromatic hydrocarbons, such as toluene, o-, m- and p-xylene, ethers, such as diethyl ether, diisopropyl ether, TBME, dioxane, anisole and THF, and also DMSO, DMF and DMAC, particularly preferably diethyl ether, dioxane and THF.

It is also possible to use mixtures of the solvents mentioned.

Examples of metal organic species for the synthesis of pyrimidines (XXVIII) are Grignard reagents like R′MgCl, R′MgBr or R′MgI, lithium organic species, aluminum organic species like R′₃Al, R′₂AlX and R′AlX₂, titanium organic species like R′₄Ti, R′₃TiX, R′₂TiX₂ and R′TiX₃.

Preferred agents include Grignard reagents and lithium organic species.

The metal organic species is used preferably from 2 to 10 equivalents based on the pyrimidine (XXIX), more preferably from 2.0 to 5.0 equivalents based on the pyrimidine (XXIX), most preferably from 2.0 to 3.0 equivalents based on the pyrimidine (XXIX).

Process H:

The pyrimidines of formula (XXIX) can be obtained by oxidizing respective pyrimidines of formula (XXVI).

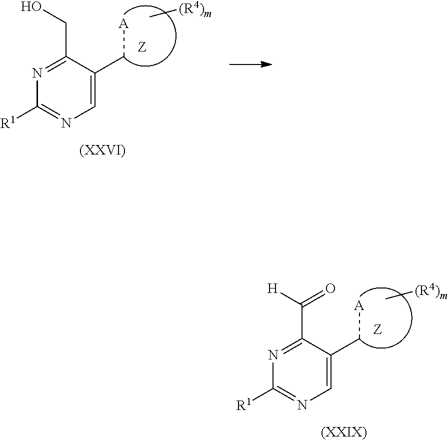

The oxidation of pyrimidines (XXVI) is usually carried out from −80° C. to the boiling point of the reaction mixture, preferably from −20° C. to 100° C., particularly preferably from 0° C. to 75° C., in an inert organic solvent.

The reaction may in principle be carried out in substance. However, preference is given to reacting the pyrimidines (XXVI) in an organic solvent.

Suitable in principle are all solvents which are capable of dissolving the pyrimidines (XXVI) at least partly and preferably fully under the reaction conditions.

Examples of suitable solvents are aromatic hydrocarbons such as benzene, chlorobenzene, toluene, cresols, o-, m- and p-xylene, halogenated hydrocarbons such as CH₂Cl₂, CHCl₃, CCH₂ClCH₂Cl or CCl₄, ethers such as diethyl ether, diisopropyl ether, TBME, dioxane, anisole and THF, as well as dipolar aprotic solvents such as sulfolane, dimethylsulfoxide, DMF, N,N-DMAC (DMAC), DMIDMI, DMPU, DMSO and NMP.

It is also possible to use mixtures of the solvents mentioned.

Examples of oxidizing agents for the synthesis of pyrimidines (XXIX) are metal oxides such as MnO₂, KMnO₄, CrO₃ or PCC, and non-metal oxides such as NaClO, NaIO₄ or pyridine/SO₃-complex. In addition methods like the Swern oxidation or the TEMPO oxidation known to a person skilled in the art can be used to obtain pyrimidines of formula (XXIX).

Preferred agents include MnO₂, KMnO₄ and PCC, more preferred MnO₂.

The oxidizing agent is used preferably from 1 to 50 equivalents based on the pyrimidine (XXVI), more preferably from 1.0 to 20.0 equivalents based on the pyrimidine (XXVI), most preferably from 1.0 to 10.0 equivalents based on the pyrimidine (XXVI).

Process I:

The pyrimidines of (XXX; Q=cycloalkyl, halocycloalkyl, alkyl, haloalkyl, alkenyl, alkynyl, phenyl, heteroaryl, heterocyclyl, alkylidenyl or halo alkylidenyl and R′=alkyl, haloalkyl, alkenyl, alkynyl, cycloalkyl, heterocyclyl, alkoxycarbonylalkyl) can be obtained reacting respective pyrimidines of formula (XXXI) with base and an electrophile.

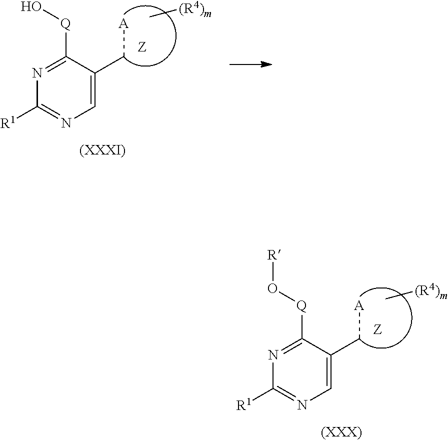

Electrophiles can be an alkyl-, alkenyl- or alkynyl-halide, e.g. methyl iodide, allyl bromide propargyl bromide, ethyl iodide, propyl bromide, or ethyl 2-bromoacetate.

The reaction of the pyrimidine (XXXI) with the electrophile is usually carried out at temperatures of from −100° C. to the boiling point of the reaction mixture, preferably from −20° C. to 100° C., particularly preferably from −0° C. to 30° C., in an inert organic solvent in the presence of a base.

Suitable in principle are all solvents which are capable of dissolving the pyrimidine (XXXI) and the electrophile at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, nitriles such as acetonitrile and propionitrile, as well as dipolar aprotic solvents such as sulfolane, dimethylsulfoxide, DMF, N,N-DMAC (DMAC), DMIDMI, DMPU, DMSO and NMP.

It is also possible to use mixtures of the solvents mentioned.

Suitable bases are, in general, inorganic compounds, such as alkali metal and alkaline earth metal anhydrides, such as lithium hydride, sodium hydride, potassium hydride and calcium hydride, alkali metal azides, such as lithium hexamethyidisilazide, organometallic compounds, in particular alkali metal alkyls, such as methyllithium, butyllithium and phenyllithium, and also alkali metal and alkaline earth metal alkoxides, such as sodium methoxide, sodium ethoxide, potassium ethoxide, potassium tert-butoxide, potassium tert-pentoxide and dimethoxymagnesium, moreover organic bases, e.g. tertiary amines, such as trimethylamine, triethylamine, diisopropylethylamine and N-methylpiperidine, pyridine, substituted pyridines, such as collidine, lutidine and 4-dimethylaminpyridine, and also bicyclic amines. Particular preference is given to sodium hydride, lithium hexamethyldisilazide and lithium diisopropylamide.

The bases are generally employed in equimolar amounts; however, they can also be employed in catalytic amounts, in excess or, if appropriate, as solvents.

The starting materials are generally reacted with one another in equimolar amounts. It may be advantageous to employ an excess of base and/or the electrophile, based on the pyrimidine (XXXI).

Process J:

The pyrimidine compounds of formula (I) can in addition be obtained by reacting respective pyrimidine boronic acid esters of formula (XXXII) with halides of formula (XXXIII) in which X equals Cl, Br, or I:

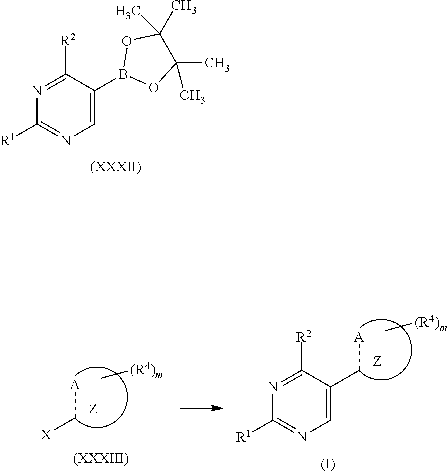

The reaction of the pyrimidine (XXXII) with halides of formula (XXXIII) is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXXII) with halides of formula (XXXIII) in an organic solvent with or without water as co-solvent.

Suitable in principle are all solvents which are capable of dissolving the pyrimidines (XXXII) with halides of formula (XXXIII) at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, N,N-DMAC (DMAC), DMIDMI, DMPU, DMSO and NMP.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂.

The term base as used herein also includes mixtures of two or more, preferably two of the above bases. Particular preference is given to the use of one base.

The bases are used preferably from 1 to 10 equivalents based on the pyrimidine (XXXII), more preferably from 1.0 to 5.0 equivalents based on the pyrimidine (XXXIII), most preferably from 1.2 to 2.5 equivalents based on the pyrimidine (XXXII).

It may be advantageous to add the base offset over a period of time.

The reaction of the pyrimidines (XXXII) with halides of formula (XXXIII) is carried out in the presence of a catalyst. Examples of suitable catalysts include e.g., palladium based catalysts like, e.g., Palladium(II)acetate, tetrakis(triphenylphosphine)palladium(0), bis(triphenylphosphine)palladium(II)chloride or (1,1,-bis(diphenylphosphino)ferrocene)dichloropalladium(II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXXII).

The halides (XXXIII) required for the preparation of pyrimidine compounds of formula (I) are known from the literature or are commercially available.

The pyrimidines of formula (XXXII) can be obtained by reacting the respective pyrimidines (XXXIV) with Bis(pinacolato)diboron XXXV.

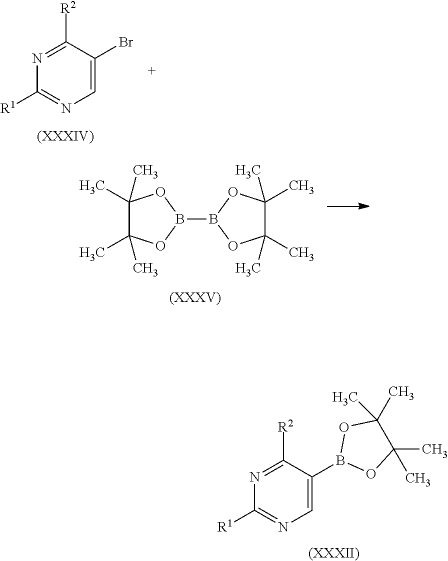

The reaction of the pyrimidine (XXXIV) with Bis(pinacolato)diboron) XXXV is usually carried out from 0° C. to the boiling point of the reaction mixture, preferably from 15° C. to 110° C., particularly preferably from 40° C. to 100° C., in an inert organic solvent in the presence of a base and a catalyst.

The reaction may in principle be carried out in substance. However, preference is given to reacting the pyrimidines (XXXIV) with bis(pinacolato)diboron) XXXV in an organic solvent with or without water as co-solvent.

Suitable in principle are all solvents which are capable of dissolving the pyrimidines (XXXIV) with bis(pinacolato)diboron XXXV at least partly and preferably fully under the reaction conditions.

Examples of suitable solvents are aromatic hydrocarbons such as benzene, chlorobenzene, toluene, cresols, o-, m- and p-xylene, ethers such as diethyl ether, diisopropyl ether, TBME, dioxane, anisole and THF, as well as dipolar aprotic solvents such as sulfolane, dimethylsulfoxide, DMF, DMAC, DMIDMI, DMPU, DMSO and NMP.

It is also possible to use mixtures of the solvents mentioned.

Examples of suitable metal-containing bases are inorganic compounds including metal-containing bases such as alkali metal and alkaline earth metal hydroxides, and other metal hydroxides, such as LiOH, NaOH, KOH, Mg(OH)₂, Ca(OH)₂ and Al(OH)₃; alkali metal and alkaline earth metal oxide, and other metal oxides, such as Li₂O, Na₂O, K₂O, MgO, and CaO, Fe₂O₃, Ag₂O; alkali metal and alkaline earth metal carbonates such as Li₂CO₃, Na₂CO₃, K₂CO₃, Cs₂CO₃, MgCO₃, and CaCO₃, as well as alkali metal hydrogen carbonates (bicarbonates) such as LiHCO₃, NaHCO₃, KHCO₃; alkali metal and alkaline earth metal phosphates such as potassium phosphate (K₃PO₄), calcium phosphate (Ca₃(PO₄)₂); alkali metal and alkaline earth metal acetates such as sodium acetate or potassium acetate.

Preferred bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide and alkali metal or alkaline earth metal carbonates such as lithium carbonate, sodium carbonate, potassium carbonate, cesium carbonate, magnesium carbonate, and calcium carbonate and alkaline earth metal phosphates such as potassium phosphate, and alkali metal acetates such as sodium acetate or potassium acetate.

Especially preferred bases are inorganic compounds such as alkali metal and alkaline earth metal hydroxides, and other metal hydroxides, such as lithium hydroxide, sodium hydroxide, potassium hydroxide, magnesium hydroxide, calcium hydroxide and aluminum hydroxide and alkaline earth metal phosphates such as potassium phosphate, and alkali metal acetates such as or potassium acetate.

The term base as used herein also includes mixtures of two or more, preferably two of the above bases. Particular preference is given to the use of one base.

The bases are used preferably from 1 to 10 equivalents based on the pyrimidine (XXXIV), more preferably from 1.0 to 5.0 equivalents based on the pyrimidine (XXXIV), most preferably from 1.2 to 2.5 equivalents based on the pyrimidine (XXXIV).

It may be advantageous to add the base offset over a period of time.

The reaction of the pyrimidines (XXXIV) with bis(pinacolato)diboron XXXV is carried out in the presence of a catalyst. Examples of suitable catalysts include e.g., palladium based catalysts like, e.g., Palladium(II)acetate, tetrakis(triphenylphosphine)palladium(0), bis(triphenylphosphine)palladium(II)chloride or (1,1,-bis(diphenylphosphino)ferrocene)dichloropalladium(II), and optionally suitable additives such as, e.g., phosphines like, e.g., P(o-tolyl)₃, triphenylphosphine or BINAP (2,2′-Bis(diphenylphospino)-1,1′-binaphthyl).

The amount of catalyst is usually 0.01 to 20 mol % (0.0001 to 0.2 equivalents) based on the pyrimidine (XXXIV).

The end of the reaction can easily be determined by the skilled worker by means of routine methods.

The reaction mixtures are worked up in a customary manner, e.g. by mixing with water, separation of the phases and, if appropriate, chromatographic purification of the crude product.

Some of the intermediates and end products are obtained in the form of viscous oils, which can be purified or freed from volatile components under reduced pressure and at moderately elevated temperature.

If the intermediates and the end products are obtained as solid, purification can also be carried out by recrystallization or digestion.

The halides (XXXIV) required for the preparation of pyrimidine compounds of formula (XXXII) are known from the literature or are commercially available.

The present invention also provides agrochemical compositions comprising at least one pyrimidine compounds of formula (I) and auxiliaries customary for formulating crop protection agents.

The present invention furthermore provides a method for controlling unwanted vegetation where a herbicidal effective amount of at least one pyrimidine compounds of formula (I) is allowed to act on plants, their seeds and/or their habitat. Application can be done before, during and/or after, preferably during and/or after, the emergence of the undesirable plants.

Further embodiments of the present invention are evident from the claims, the description and the examples. It is to be understood that the features mentioned above and still to be illustrated below of the subject matter of the invention can be applied not only in the combination given in each particular case but also in other combinations, without leaving the scope of the invention.

As used herein, the terms “controlling” and “combating” are synonyms.

As used herein, the terms “undesirable vegetation” and “harmful plants” are synonyms.

If the pyrimidine compounds of formula (I) as described herein are capable of forming geometrical isomers, e.g. E/Z isomers, it is possible to use both, the pure isomers and mixtures thereof, in the compositions according to the invention.

If the pyrimidine compounds of formula (I) as described herein have one or more centres of chirality and, as a consequence, are present as enantiomers or diastereomers, it is possible to use both, the pure enantiomers and diastereomers and their mixtures, in the compositions according to the invention.

If the pyrimidine compounds of formula (I) as described herein have ionisable functional groups, they can also be employed in the form of their agriculturally acceptable salts. Suitable are, in general, the salts of those cations and the acid addition salts of those acids whose cations and anions, respectively, have no adverse effect on the activity of the active compounds.

Preferred cations are the ions of the alkali metals, preferably of lithium, sodium and potassium, of the alkaline earth metals, preferably of calcium and magnesium, and of the transition metals, preferably of manganese, copper, zinc and iron, further ammonium and substituted ammonium in which one to four H atoms are replaced by C₁-C₄-alkyl, HO—C₁-C₄-alkyl, C₁-C₄-alkoxy-C₁-C₄-alkyl, HO—C₁-C₄-alkoxy-C₁-C₄-alkyl, phenyl or benzyl, preferably ammonium, methyl-ammonium, isopropylammonium, dimethylammonium, diisopropylammonium, trimethylammonium, heptylammonium, dodecylammonium, tetradecylammonium, tetramethylammonium, tetraethylammonium, tetrabutylammonium, 2-hydroxyethylammonium (olamine salt), 2-(2-hydroxyeth-1-oxy)eth-1-ylammonium (diglycolamine salt), di(2-hydroxyeth-1-yl)ammonium (diolamine salt), tris(2-HO-ethyl)ammonium (trolamine salt), tris(2-hydroxypropyl)ammonium, benzyltrimethylammonium, benzyltriethylammonium, N,N,N-trimethylethanolammonium (choline salt), furthermore phosphon-ium ions, sulfonium ions, preferably tri(C₁-C₄-alkyl)sulfonium, such as trimethylsulfonium, and sulfoxonium ions, preferably tri(C₁-C₄-alkyl)sulfoxonium, and finally the salts of polybasic amines such as N,N-bis-(3-aminopropyl)methylamine and diethylenetriamine.

Anions of useful acid addition salts are primarily chloride, bromide, fluoride, iodide, hydrogensulfate, methylsulfate, sulfate, dihydrogenphosphate, hydrogenphosphate, nitrate, bicarbonate, carbonate, hexafluorosilicate, hexafluorophosphate, benzoate and also the anions of C₁-C₄-alkanoic acids, preferably formate, acetate, propionate and butyrate.

Pyrimidine compounds of formula (I) as described herein having an acidic functionality can be employed, if applicable, in the form of the acid, in the form of an agriculturally suitable salt as mentioned above or else in the form of an agriculturally acceptable derivative, e.g. as amides, such as mono- and di-C₁-C₆-alkylamides or arylamides, as esters, e.g. as allyl esters, propargyl esters, C₁-C₁₀-alkyl esters, alkoxyalkyl esters, tefuryl ((tetrahydrofuran-2-yl)methyl) esters and also as thioesters, e.g. as C₁-C₁₀-alkylthio esters. Preferred mono- and di-C₁-C₆-alkylamides are the CH₃ and the dimethylamides. Preferred arylamides are, e.g., the anilides and the 2-chloroanilides. Preferred alkyl esters are, e.g., the methyl, ethyl, propyl, isopropyl, butyl, isobutyl, pentyl, mexyl (1-methylhexyl), meptyl (1-methylheptyl), heptyl, octyl or isooctyl (2-ethylhexyl) esters. Preferred C₁-C₄-alkoxy-C₁-C₄-alkyl esters are the straight-chain or branched C₁-C₄-alkoxy ethyl esters, e.g. the 2-methoxyethyl, 2-ethoxyethyl, 2-butoxyethyl (butotyl), 2-butoxypropyl or 3-butoxypropyl ester. An example of a straight-chain or branched C₁-C₁₀-alkylthio ester is the ethylthio ester.

The organic moieties mentioned in the definition of the variables R¹, R², A, Z, R³, R^{3A}, and R⁴, are—like the term halogen—collective terms for individual enumerations of the individual group members. The term halogen denotes in each case F, Cl, Br, or I. All hydrocarbon chains, e.g. all alkyl, alkenyl, alkynyl, alkoxy chains can be straight-chain or branched, the prefix Cₙ-Cₘ denoting in each case the possible number of carbon atoms in the group.

Examples of such meanings are:
C₁-C₄-alkyl: e.g. CH₃, C₂H₅, n-propyl, CH(CH₃)₂, n-butyl, CH(CH₃)—C₂H₅, CH₂—CH(CH₃)₂, and C(CH₃)₃;
C₁-C₆-alkyl: C₁-C₄-alkyl as mentioned above, and also, e.g., n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, preferably methyl, ethyl, n-propyl, 1-methylethyl, n-butyl, 1,1-dimethylethyl, n-pentyl, or n-hexyl;
C₁-C₄-haloalkyl: C₁-C₄-alkyl as mentioned above which is partially or fully substituted by fluorine, chlorine, bromine and/or iodine, e.g., chloromethyl, dichloromethyl, trichloromethyl, fluoromethyl, difluoromethyl, trifluoromethyl, chlorofluoromethyl, dichlorofluoromethyl, chlorodifluoromethyl, bromomethyl, iodomethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, pentafluoroethyl, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoro-propyl, 1-(fluoromethyl)-2-fluoroethyl, 1-(chloromethyl)-2-chloroethyl, 1-(bromomethyl)-2-bromo-ethyl, 4-fluorobutyl, 4-chlorobutyl, 4-bromobutyl, nonafluorobutyl, 1,1,2,2,-tetrafluoroethyl, and 1-trifluoromethyl-1,2,2,2-tetrafluoroethyl;
C₁-C₆-haloalkyl: C₁-C₄-haloalkyl as mentioned above, and also, e.g., 5-fluoropentyl, 5-chloropentyl, 5-bromopentyl, 5-iodopentyl, undecafluoropentyl, 6-fluorohexyl, 6-chlorohexyl, 6-bromohexyl, 6-iodohexyl, and dodecafluorohexyl;
C₃-C₆-cycloalkyl: monocyclic saturated hydrocarbons having 3 to 6 ring members, such as cyclopropyl, cyclobutyl, cyclopentyl, and cyclohexyl;
C₃-C₆-alkenyl: e.g. 1-propenyl, 2-propenyl, 1-methylethenyl, 1-butenyl, 2-butenyl, 3-butenyl, 1-methyl-1-propenyl, 2-methyl-1-propenyl, 1-methyl-2-propenyl, 2-methyl-2-propenyl, 1-pentenyl, 2-pentenyl, 3-pentenyl, 4-pentenyl, 1-methyl-1-butenyl, 2-methyl-1-butenyl, 3-methyl-1-butenyl, 1-methyl-2-butenyl, 2-methyl-2-butenyl, 3-methyl-2-butenyl, 1-methyl-3-butenyl, 2-methyl-3-butenyl, 3-methyl-3-butenyl, 1,1-dimethyl-2-propenyl, 1,2-dimethyl-1-propenyl, 1,2-dimethyl-2-propenyl, 1-ethyl-1-propenyl, 1-ethyl-2-propenyl, 1-hexenyl, 2-hexenyl, 3-hexenyl, 4-hexenyl, 5-hexenyl, 1-methyl-1-pentenyl, 2-methyl-1-pentenyl, 3-methyl-1-pentenyl, 4-methyl-1-pentenyl, 1-methyl-2-pentenyl, 2-methyl-2-pentenyl, 3-methyl-2-pentenyl, 4-methyl-2-pentenyl, 1-methyl-3-pentenyl, 2-methyl-3-pentenyl, 3-methyl-3-pentenyl, 4-methyl-3-pentenyl, 1-methyl-4-pentenyl, 2-methyl-4-pentenyl, 3-methyl-4-pentenyl, 4-methyl-4-pentenyl, 1,1-dimethyl-2-butenyl, 1,1-dimethyl-3-butenyl, 1,2-dimethyl-1-butenyl, 1,2-dimethyl-2-butenyl, 1,2-dimethyl-3-butenyl, 1,3-dimethyl-1-butenyl, 1,3-dimethyl-2-butenyl, 1,3-dimethyl-3-butenyl, 2,2-dimethyl-3-butenyl, 2,3-dimethyl-1-butenyl, 2,3-dimethyl-2-butenyl, 2,3-dimethyl-3-butenyl, 3,3-dimethyl-1-butenyl, 3,3-dimethyl-2-butenyl, 1-ethyl-1-butenyl, 1-ethyl-2-butenyl, 1-ethyl-3-butenyl, 2-ethyl-1-butenyl, 2-ethyl-2-butenyl, 2-ethyl-3-butenyl, 1,1,2-trimethyl-2-propenyl, 1-ethyl-1-methyl-2-propenyl, 1-ethyl-2-methyl-1-propenyl, and 1-ethyl-2-methyl-2-propenyl;
C₃-C₆-haloalkenyl: a C₃-C₆-alkenyl substituent as mentioned above which is partially or fully substituted by fluorine, chlorine, bromine and/or iodine, e.g. 2-chloroprop-2-en-1-yl, 3-chloroprop-2-en-1-yl, 2,3-dichloroprop-2-en-1-yl, 3,3-dichloroprop-2-en-1-yl, 2,3,3-trichloro-2-en-1-yl, 2,3-dichlorobut-2-en-1-yl, 2-bromoprop-2-en-1-yl, 3-bromoprop-2-en-1-yl, 2,3-dibromoprop-2-en-1-yl, 3,3-dibromoprop-2-en-1-yl, 2,3,3-tribromo-2-en-1-yl, or 2,3-dibromobut-2-en-1-yl;
C₃-C₆-alkynyl: e.g. 1-propynyl, 2-propynyl, 1-butynyl, 2-butynyl, 3-butynyl, 1-methyl-2-propynyl, 1-pentynyl, 2-pentynyl, 3-pentynyl, 4-pentynyl, 1-methyl-2-butynyl, 1-methyl-3-butynyl, 2-methyl-3-butynyl, 3-methyl-1-butynyl, 1,1-dimethyl-2-propynyl, 1-ethyl-2-propynyl, 1-hexynyl, 2-hexynyl, 3-hexynyl, 4-hexynyl, 5-hexynyl, 1-methyl-2-pentynyl, 1-methyl-3-pentynyl, 1-methyl-4-pentynyl, 2-methyl-3-pentynyl, 2-methyl-4-pentynyl, 3-methyl-1-pentynyl, 3-methyl-4-pentynyl, 4-methyl-1-pentynyl, 4-methyl-2-pentynyl, 1,1-dimethyl-2-butynyl, 1,1-dimethyl-3-butynyl, 1,2-dimethyl-3-butynyl, 2,2-dimethyl-3-butynyl, 3,3-dimethyl-1-butynyl, 1-ethyl-2-butynyl, 1-ethyl-3-butynyl, 2-ethyl-3-butynyl, and 1-ethyl-1-methyl-2-propynyl;
C₂-C₆-alkynyl: C₃-C₆-alkynyl as mentioned above and also ethynyl;
C₃-C₆-haloalkynyl: a C₃-C₆-alkynyl radical as mentioned above which is partially or fully substituted by F, C1, Br and/or I, e.g. 1,1-difluoroprop-2-yn-1-yl, 3-chloroprop-2-yn-1-yl, 3-bromoprop-2-yn-1-yl, 3-iodoprop-2-yn-1-yl, 4-fluorobut-2-yn-1-yl, 4-chlorobut-2-yn-1-yl, 1,1-difluorobut-2-yn-1-yl, 4-iodobut-3-yn-1-yl, 5-fluoropent-3-yn-1-yl, 5-iodopent-4-yn-1-yl, 6-fluorohex-4-yn-1-yl, or 6-iodohex-5-yn-1-yl;
C₁-C₄-alkoxy: e.g. methoxy, ethoxy, propoxy, 1-methylethoxy butoxy, 1-methylpropoxy, 2-methylpropoxy, and 1,1-dimethylethoxy;
C₁-C₆-alkoxy: C₁-C₄-alkoxy as mentioned above, and also, e.g., pentoxy, 1-methylbutoxy, 2-methylbutoxy, 3-methoxylbutoxy, 1,1-dimethylpropoxy, 1,2-dimethylpropoxy, 2,2-dimethylpropoxy, 1-ethylpropoxy, hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy, and 1-ethyl-2-methylpropoxy.
C₁-C₄-haloalkoxy: a C₁-C₄-alkoxy radical as mentioned above which is partially or fully substituted by fluorine, chlorine, bromine and/or iodine, i.e., e.g., fluoromethoxy, difluoromethoxy, trifluoromethoxy, chlorodifluoromethoxy, bromodifluoromethoxy, 2-fluoroethoxy, 2-chloroethoxy, 2-bromomethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2-chloropropoxy, 3-chloropropoxy, 2-bromopropoxy, 3-bromopropoxy, 2,2-difluoropropoxy, 2,3-difluoropropoxy, 2,3-dichloropropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy, and nonafluorobutoxy;
C₁-C₆-haloalkoxy: a C₁-C₄-haloalkoxy as mentioned above, and also, e.g., 5-fluoropentoxy, 5-chloropentoxy, 5-bromopentoxy, 5-iodopentoxy, undecafluoropentoxy, 6-fluorohexoxy, 6-chlorohexoxy, 6-bromohexoxy, 6-iodohexoxy and dodecafluorohexoxy;
C₁-C₄-alkylthio: e.g. methylthio, ethylthio, propylthio, 1-methylethylthio, butylthio, 1-methylpropylthio, 2-methylpropylthio, and 1,1-dimethylethylthio;
C₁-C₆-alkylthio: C₁-C₄-alkylthio as mentioned above, and also, e.g., pentylthio, 1-methylbutylthio, 2-methylbutylthio, 3-methylbutylthio, 2,2-dimethylpropylthio, 1-ethylpropylthio, hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio, and 1-ethyl-2-methylpropylthio;
(C₁-C₄-alkyl)amino: e.g. methylamino, ethylamino, propylamino, 1-methylethylamino, butylamino, 1-methylpropylamino, 2-methylpropylamino, or 1,1-dimethylethylamino;
(C₁-C₆-alkyl)amino: (C₁-C₄-alkylamino) as mentioned above, and also, e.g., pentylamino, 1-methylbutylamino, 2-methylbutylamino, 3-methylbutylamino, 2,2-dimethylpropylamino, 1-ethylpropylamino, hexylamino, 1,1-dimethylpropylamino, 1,2-dimethylpropylamino, 1-methylpentylamino, 2-methylpentylamino, 3-methylpentylamino, 4-methylpentylamino, 1,1-dimethylbutylamino, 1,2-dimethylbutylamino, 1,3-dimethylbutylamino, 2,2-dimethylbutylamino, 2,3-dimethylbutyl-amino 3,3-dimethylbutylamino, 1-ethylbutylamino, 2-ethylbutylamino, 1,1,2-trimethylpropylamino, 1,2,2-trimethyl-propylamino, 1-ethyl-1-methylpropylamino, or 1-ethyl-2-methylpropylamino;
di(C₁-C₄-alkyl)amino: e.g. N,N-dimethylamino, N,N-diethylamino, N,N-di(1-methylethyl)amino, N,N-dipropylamino, N,N-dibutylamino, N,N-di(1-methylpropyl)amino, N,N-di(2-methyl-propyl)amino, N,N-di(1,1-dimethylethyl)amino, N-ethyl-N-methylamino, N-methyl-N-propyl-amino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)-amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methyl-ethyl)-N-(1-methyl-propyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethyl-ethyl)-N-(1-methylpropyl)amino, or N-(1,1-dimethylethyl)N-(2-methylpropyl)amino;
di(C₁-C₆-alkyl)amino: di(C₁-C₄-alkyl)amino as mentioned above, and also, e.g., N-methyl-N-pentylamino, N-methyl-N-(1-methylbutyl)amino, N-methyl-N-(2-methylbutyl)amino, N-methyl-N-(3-methylbutyl)amino, N-methyl-N-(2,2-dimethylpropyl)amino, N-methyl-N-(1-ethylpropyl)amino, N-methyl-N-hexylamino, N-methyl-N-(1,1-dimethylpropyl)amino, N-methyl-N-(1,2-dimethylpropyl)amino, N-methyl-N-(1-methylpentyl)amino, N-methyl-N-(2-methylpentyl)amino, N-methyl-N-(3-methylpentyl)amino, N-methyl-N-(4-methylpentyl)amino, N-methyl-N-(1,1-dimethylbutyl)amino, N-methyl-N-(1,2-dimethylbutyl)amino, N-methyl-N-(1,3-dimethylbutyl)amino, N-methyl-N-(2,2-dimethylbutyl)amino, N-methyl-N-(2,3-dimethylbutyl)amino, N-methyl-N-(3,3-dimethylbutyl)amino, N-methyl-N-(1-ethylbutyl)amino, N-methyl-N-(2-ethylbutyl)amino, N-methyl-N-(1,1,2-trimethylpropyl)amino, N-methyl-N-(1,2,2-trimethylpropyl)amino, N-methyl-N-(1-ethyl-1-methylpropyl)amino, N-methyl-N-(1-ethyl-2-methylpropyl)amino, N-ethyl-N-pentylamino, N-ethyl-N-(1-methylbutyl)amino, N-ethyl-N-(2-methylbutyl)amino, N-ethyl-N-(3-methylbutyl)amino, N-ethyl-N-(2,2-dimethylpropyl)amino, N-ethyl-N-(1-ethylpropyl)amino, N-ethyl-N-hexylamino, N-ethyl-N-(1,1-dimethylpropyl)amino, N-ethyl-N-(1,2-dimethylpropyl)amino, N-ethyl-N-(1-methylpentyl)amino, N-ethyl-N-(2-methylpentyl)amino, N-ethyl-N-(3-methylpentyl)amino, N-ethyl-N-(4-methylpentyl)amino, N-ethyl-N-(1,1-dimethylbutyl)amino, N-ethyl-N-(1,2-dimethylbutyl)amino, N-ethyl-N-(1,3-dimethylbutyl)amino, N-ethyl-N-(2,2-dimethylbutyl)amino, N-ethyl-N-(2,3-dimethylbutyl)amino, N-ethyl-N-(3,3-dimethylbutyl)amino, N-ethyl-N-(1-ethylbutyl)amino, N-ethyl-N-(2-ethylbutyl)amino, N-ethyl-N-(1,1,2-trimethylpropyl)amino, N-ethyl-N-(1,2,2-trimethylpropyl)amino, N-ethyl-N-(1-ethyl-1-methylpropyl)amino, N-ethyl-N-(1-ethyl-2-methylpropyl)amino, N-propyl-N-pentylamino, N-butyl-N-pentylamino, N,N-dipentylamino, N-propyl-N-hexylamino, N-butyl-N-hexylamino, N-pentyl-N-hexylamino, or N,N-dihexylamino;
C₁-C₆-alkylsulfinyl (C₁-C₆-Alkyl-S(═O)—): e.g. methylsulfinyl, ethylsulfinyl, propylsulfinyl, 1-methylethylsulfinyl, butylsulfinyl, 1-methylpropylsulfinyl, 2-methylpropylsulfinyl, 1,1-dimethylethylsulfinyl, pentylsulfinyl, 1-methylbutylsulfinyl, 2-methylbutylsulfinyl, 3-methylbutylsulfinyl, 2,2-dimethylpropylsulfinyl, 1-ethylpropylsulfinyl, 1,1-dimethylpropylsulfinyl, 1,2-dimethylpropyl-sulfinyl, hexylsulfinyl, 1-methylpentylsulfinyl, 2-methylpentylsulfinyl, 3-methylpentylsulfinyl, 4-methylpentyl-sulfinyl, 1,1-dimethylbutylsulfinyl, 1,2-dimethylbutylsulfinyl, 1,3-dimethylbutyl-sulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropyl-sulfinyl, and 1-ethyl-2-methylpropylsulfinyl;
C₁-C₆-alkylsulfonyl (C₁-C₆-alkyl-S(O)₂—): e.g. methylsulfonyl, ethylsulfonyl, propylsulfonyl, 1-methylethylsulfonyl, butylsulfonyl, 1-methylpropylsulfonyl, 2-methyl-propylsulfonyl, 1,1-dimethylethylsulfonyl, pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutyl-sulfonyl, 1-ethylbutylsulfonyl, 2-ethylbutylsulfonyl, 1,1,2-trimethyl-propylsulfonyl, 1,2,2-trimethyl-propylsulfonyl, 1-ethyl-1-methylpropylsulfonyl, and 1-ethyl-2-methylpropylsulfonyl;
C₃-C₆-cycloalkyl: a monocyclic saturated hydrocarbon having 3 to 6 ring members, such as cyclopropyl, cyclobutyl, cyclopentyl, and cyclohexyl;
C₃-C₆-cycloalkenyl: 1-cyclopropenyl, 2-cyclopropenyl, 1-cyclobutenyl, 2-cyclobutenyl, 1-cyclopentenyl, 2-cyclopentenyl, 1,3-cyclopentadienyl, 1,4-cyclopentadienyl, 2,4-cyclopentadienyl, 1-cyclohexenyl, 2-cyclohexenyl, 3-cyclohexenyl, 1,3-cyclohexadienyl, 1,4-cyclohexadienyl, or 2,5-cyclohexadienyl;
bicyclic ring: a 9- to 10-membered bicyclic ring: a partially or fully unsaturated 9- to 10-membered carbocyclic system wherein two partially or fully unsaturated carbocyclic rings are fused with each other through 2 ring members, and which in addition to carbon atoms and independent of their position in the ring can comprise as ring members 1 to 4 nitrogen atoms, or 1 or 2 oxygen atoms, or 1 or 2 oxygen atoms and 1 to 2 nitrogen atoms, or 1 or 3 sulfur atoms, or 1 to 4 nitrogen atoms and an oxygen atom, or one to three nitrogen atoms and a sulfur atom, or one sulfur and one oxygen atom, examples of such bicyclic ring are, 2,3-dihydrobenzothiophene, benzothiophene, 2,3-dihydrobenzofuran, benzofuran, 1,3-benzodioxole, 1,3-benzodithiole, 1,3-benzoxathiole, indole, indane, [1,3]dioxolo[4,5-c]pyridine, [1,3]dioxolo[4,5-b]pyridine, 2,3-dihydrofuro[2,3-c]pyridine, furo[2,3-c]pyridine, 2,3-dihydrofuro[2,3-b]pyridine, furo[2,3-b]pyridine, 2,3-dihydrofuro[3,2-c]pyridine, furo[3,2-c]pyridine, 2,3-dihydrofuro[3,2-b]pyridine, furo[3,2-b]pyridine, furo[3,2-d]pyrimidine, furo[2,3-d]pyrimidine, 6,7-dihydrofuro[3,2-d]pyrimidine, 5,6-dihydrofuro[2,3-d]pyrimidine, thieno[3,2-d]pyrimidine, thieno[2,3-d]pyrimidine, 6,7-dihydrothieno[3,2-d]pyrimidine, 5,6-dihydrothieno[2,3-d]pyrimidine, 2,3-dihydrothieno[2,3-c]pyridine, thieno[2,3-c]pyridine, 2,3-dihydrothieno[2,3-b]pyridine, thieno[2,3-b]pyridine, 2,3-dihydrothieno[3,2-c]pyridine, thieno[3,2-c]pyridine, 2,3-dihydrothieno[3,2-b]pyridine, thieno[3,2-b]pyridine, 1H-pyrrolo[2,3-c]pyridine, 1H-pyrrolo[2,3-b]pyridine, 1H-pyrrolo[3,2-c]pyridine, 1H-pyrrolo[3,2-b]pyridine, 1H-imidazo[4,5-c]pyridine, 1H-imidazo[4,5-b]pyridine, 3H-imidazo[4,5-c]pyridine, 3H-imidazo[4,5-b]pyridine, 1H-pyrazolo[3,4-b]pyridine, 1H-pyrazolo[3,4-c]pyridine, 1H-pyrazolo[4,3-b]pyridine, 1H-pyrazolo[4,3-c]pyridine, 1H-indazole, benzimidazole, 1,2-benzoxazole, 1,3-benzoxazole, 1,3-benzothiazole, 1,2-benzothiazole, naphthalene, quinolone, isoquinoline, quinazoline, 1,3-benzoxathiole, [1,3]oxathiolo[4,5-b]pyridine, [1,3]oxathiolo[4,5-c]pyridine, [1,3]oxathiolo[5,4-c]pyridine, [1,3]oxathiolo[5,4-b]pyridine, 2,3-dihydro-1,4-benzodioxine, 2,3-dihydro-[1,4]dioxino[2,3-b]pyridine, 2,3-dihydro-[1,4]dioxino[2,3-c]pyridine;
heterocyclyl: a 3- to 6-membered heterocyclyl: a saturated or partial unsaturated cycle having three to six ring members which comprises apart from carbon atoms one to four nitrogen atoms, or one or two oxygen atoms, or one or two sulfur atoms, or one to three nitrogen atoms and an oxygen atom, or one to three nitrogen atoms and a sulfur atom, or one sulfur and one oxygen atom, e.g. 3- or 4-membered heterocycles like 2-oxiranyl, 2-aziridinyl, 2-thiiranyl, 2-oxetanyl, 3-oxetanyl, 2-thietanyl, 3-thietanyl, 1-azetidinyl, 2-azetidinyl, 1-azetinyl, or 2-azetinyl; 5-membered saturated heterocycles like 2-tetrahydrofuranyl, 3-tetrahydrofuranyl, 2-tetrahydrothienyl, 3-tetrahydrothienyl, 1-pyrrolidinyl, 2-pyrrolidinyl, 3-pyrrolidinyl, 3-isoxazolidinyl, 4-isoxazolidinyl, 5-isoxazolidinyl, 2-isothiazolidinyl, 3-isothiazolidinyl, 4-isothiazolidinyl, 5-isothiazolidinyl, 1-pyrazolidinyl, 3-pyrazolidinyl, 4-pyrazolidinyl, 5-pyrazolidinyl, 2-oxazolidinyl, 4-oxazolidinyl, 5-oxazolidinyl, 2-thiazolidinyl, 4-thiazolidinyl, 5-thiazolidinyl, 1-imidazolidinyl, 2-imidazolidinyl, 4-imidazolidinyl, 3-oxazolidinyl, 1,2,4-oxadiazolidin-3-yl, 1,2,4-oxadiazolidin-5-yl, 3-thiazolidinyl, 1,2,4-thiadiazolidin-3-yl, 1,2,4-thiadiazolidin-5-yl, 1,2,4-triazolidin-3-yl, 1,2,4-oxadiazolidin-2-yl, 1,2,4-oxadiazolidin-4-yl, 1,3,4-oxadiazolidin-2-yl, 1,2,4-thiadiazolidin-2-yl, 1,2,4-thiadiazolidin-4-yl, 1,3,4-thiadiazolidin-2-yl, 1,2,4-triazolidin-1-yl, or 1,3,4-triazolidin-2-yl; 5-membered partial unsaturated heterocycles like 2,3-dihydrofur-2-yl, 2,3-dihydrofur-3-yl, 2,4-dihydrofur-2-yl, 2,4-dihydrofur-3-yl, dioxolan-2-yl, 1,3-dioxol-2-yl, 2,3-dihydrothien-2-yl, 2,3-dihydrothien-3-yl, 2,4-dihydrothien-2-yl, 2,4-dihydrothien-3-yl, 4,5-dihydropyrrol-1-yl, 4,5-dihydropyrrol-2-yl, 4,5-dihydropyrrol-3-yl, 2,5-dihydropyrrol-1-yl, 2,5-dihydropyrrol-2-yl, 2,5-dihydropyrrol-3-yl, 2,3-dihydroisoxazol-1-yl, 2,3-dihydroisoxazol-3-yl, 2,3-dihydroisoxazol-4-yl, 2,3-dihydroisoxazol-5-yl, 2,5-dihydroisoxazol-3-yl, 2,5-dihydroisoxazol-4-yl, 2,5-dihydroisoxazol-5-yl, 4,5-dihydroisoxazol-2-yl, 4,5-dihydroisoxazol-3-yl, 4,5-dihydroisoxazol-4-yl, 4,5-dihydroisoxazol-5-yl, 2,3-dihydroisothiazol-1-yl, 2,3-dihydroisothiazol-3-yl, 2,3-dihydroisothiazol-4-yl, 2,3-dihydroisothiazol-5-yl, 2,5-dihydroisothiazol-3-yl, 2,5-dihydroisothiazol-4-yl, 2,5-dihydroisothiazol-5-yl, 4,5-dihydroisothiazol-1-yl, 4,5-dihydroisothiazol-3-yl, 4,5-dihydroisothiazol-4-yl, 4,5-dihydroisothiazol-5-yl, 2,3-dihydropyrazol-1-yl, 2,3-dihydropyrazol-2-yl, 2,3-dihydropyrazol-3-yl, 2,3-dihydropyrazol-4-yl, 2,3-dihydropyrazol-5-yl, 3,4-dihydropyrazol-1-yl, 3,4-dihydropyrazol-3-yl, 3,4-dihydropyrazol-4-yl, 3,4-dihydropyrazol-5-yl, 4,5-dihydropyrazol-1-yl, 4,5-dihydropyrazol-3-yl, 4,5-dihydropyrazol-4-yl, 4,5-dihydropyrazol-5-yl, 2,3-dihydroimidazol-1-yl, 2,3-dihydroimidazol-2-yl, 2,3-dihydroimidazol-3-yl, 2,3-dihydroimidazol-4-yl, 2,3-dihydroimidazol-5-yl, 4,5-dihydroimidazol-1-yl, 4,5-dihydroimidazol-2-yl, 4,5-dihydroimidazol-4-yl, 4,5-dihydroimidazol-5-yl, 2,5-dihydroimidazol-1-yl, 2,5-dihydroimidazol-2-yl, 2,5-dihydroimidazol-4-yl, 2,5-dihydroimidazol-5-yl, 2,3-dihydrooxazol-2-yl, 2,3-dihydrooxazol-3-yl, 2,3-dihydrooxazol-4-yl, 2,3-dihydrooxazol-5-yl, 3,4-dihydrooxazol-2-yl, 3,4-dihydrooxazol-3-yl, 3,4-dihydrooxazol-4-yl, 3,4-dihydrooxazol-5-yl, 2,3-dihydrothiazol-2-yl, 2,3-dihydrothiazol-3-yl, 2,3-dihydrothiazol-4-yl, 2,3-dihydrothiazol-5-yl, 3,4-dihydrothiazol-2-yl, 3,4-dihydrothiazol-3-yl, 3,4-dihydrothiazol-4-yl, 3,4-dihydrothiazol-5-yl, 3,4-dihydrothiazol-2-yl, 3,4-dihydrothiazol-3-yl, or 3,4-dihydrothiazol-4-yl;

6-membered saturated heterocycles like 1-piperidinyl, 2-piperidinyl, 3-piperidinyl, 4-piperidinyl, 1,3-dioxan-5-yl, 1,4-dioxanyl, 1,3-dithian-5-yl, 1,3-dithianyl, 1,3-oxathian-5-yl, 1,4-oxathianyl, 2-tetrahydropyranyl, 3-tetrahydopyranyl, 4-tetrahydropyranyl, 2-tetrahydrothiopyranyl, 3-tetrahydrothiopyranyl, 4-tetrahydrothiopyranyl, 1-hexahydropyridazinyl, 3-hexahydropyridazinyl, 4-hexahydropyridazinyl, thexahydropyrimidinyl, 2-hexahydropyrimidinyl, 4-hexahydropyrimidinyl, 5-hexahydropyrimidinyl, 1-piperazinyl, 2-piperazinyl, 1,3,5-hexahydrotriazin-1-yl, 1,3,5-hexahydrotriazin-2-yl, 1,2,4-hexahydrotriazin-1-yl, 1,2,4-hexahydrotriazin-3-yl, tetrahydro-1,3-oxazin-1-yl, tetrahydro-1,3-oxazin-2-yl, tetrahydro-1,3-oxazin-6-yl, 1-morpholinyl, or 2-morpholinyl, 3-morpholinyl;

6-membered partial unsaturated heterocycles like 2H-pyran-2-yl, 2H-pyran-3-yl, 2H-pyran-4-yl, 2H-pyran-5-yl, 2H-pyran-6-yl, 2H-thiopyran-2-yl, 2H-thiopyran-3-yl, 2H-thiopyran-4-yl, 2H-thiopyran-5-yl, 2H-thiopyran-6-yl, or 5,6-dihydro-4H-1,3-oxazin-2-yl.
heteroaryl: a 5- or 6-membered heteroaryl: monocyclic aromatic heteroaryl having 5 to 6 ring members which, in addition to carbon atoms and independent of their position in the ring, contains 1 to 4 nitrogen atoms, or 1 to 3 nitrogen atoms and an oxygen or sulfur atom, or an oxygen or a sulfur atom, e.g. 5-membered aromatic rings like furyl (e.g. 2-furyl, 3-furyl), thienyl (e.g. 2-thienyl, 3-thienyl), pyrrolyl (e.g. pyrrol-2-yl, pyrrol-3-yl), pyrazolyl (e.g. pyrazol-3-yl, pyrazol-4-yl), isoxazolyl (e.g. isoxazol-3-yl, isoxazol-4-yl, isoxazol-5-yl), isothiazolyl (e.g. isothiazol-3-yl, isothiazol-4-yl, isothiazol-5-yl), imidazolyl (e.g. imidazole-2-yl, imidazole-4-yl), oxazolyl (e.g. oxazol-2-yl, oxazol-4-yl, oxazol-5-yl), thiazolyl (e.g. thiazol-2-yl, thiazol-4-yl, thiazol-5-yl), oxadiazolyl(e.g. 1,2,3-oxadiazol-4-yl, 1,2,3-oxadiazol-5-yl, 1,2,4-oxadiazol-3-yl, 1,2,4-oxadiazol-5-yl, 1,3,4-oxadiazol-2-yl), thiadiazolyl (e.g. 1,2,3-thiadiazol-4-yl, 1,2,3-thiadiazol-5-yl, 1,2,4-thiadiazol-3-yl, 1,2,4-thiadiazol-5-yl, 1,3,4-thiadiazolyl-2-yl), triazolyl (e.g. 1,2,3-triazol-4-yl, 1,2,4-triazol-3-yl); 1-tetrazolyl; 6-membered aromatic rings like pyridyl (e.g. pyridine-2-yl, pyridine-3-yl, pyridine-4-yl), pyrazinyl (e.g. pyridazin-3-yl, pyridazin-4-yl), pyrimidinyl (e.g. pyrimidin-2-yl, pyrimidin-4-yl, pyrimidin-5-yl), pyrazin-2-yl, triazinyl (e.g. 1,3,5-triazin-2-yl, or 1,2,4-triazin-3-yl, 1,2,4-triazin-5-yl, 1,2,4-triazin-6-yl);

The term “substituted” if not specified otherwise refers to substituted by 1, 2 or maximum possible number of substituents. If substituents as defined in compounds of formula I are more than one then they are independently from each other are same or different if not mentioned otherwise.

The substitution R⁴ if present, can be present on any ring of Z.

The term “acidic functionality” if not specified otherwise refers to a functionality capable of donating a hydrogen (proton or hydrogen ion H+), such as a carboxylic group or a sulphonic group, or, alternatively, capable of forming a covalent bond with an electron pair.

The terms “compounds of formula (I)”, “pyrimidine compounds of formula (I)”, “Compounds I” and “compounds of invention” are synonyms.

The preferred embodiments of the invention mentioned herein below have to be understood as being preferred either independently from each other or in combination with one another.

In general, pyrimidine compounds of formula (I) are suitable as herbicides.

According to a preferred embodiment of the invention preference is given pyrimidine compounds of formula (I), and their use as herbicides, wherein the variables, either independently of one another or in combination with one another, have the following meanings:

Preferred R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₄-C₆-haloalkynyloxy, C₁-C₆-alkylthio, or C₃-C₆-cycloalkyl, wherein the cycloalkyl substituent is unsubstituted;

particularly preferred R¹ is C₁-C₆-alkyl, C₁-C₆-alkoxy, or C₃-C₆-cycloalkyl, wherein the cycloalkyl substituent is unsubstituted;

especially preferred R¹ is C₃-C₆-cycloalkyl, wherein the cycloalkyl substituent is unsubstituted;

also especially preferred R¹ is C₂H₅, i-C₃H₇, OCH₃, c-C₃H₅, or c-C₄H₉;

more preferred R¹ is C₂H₅, OCH₃, or c-C₃H₅;

most preferred R¹ is c-C₃H₅.

Preferred R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, hydroxycycloalkenyl-C₁-C₆-alkyl, C₁-C₆-hydroxyalkyl, C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;

wherein hydroxy groups of R² are unsubstituted or substituted by R^{b};

cyclic groups of R² are unsubstituted or substituted by R^{c}; and

acyclic aliphatic groups of R² are unsubstituted or substituted by R^{d}.

particularly preferred R² is C₂-C₆-alkenyl, C₂-C₆-alkynyl, C₃-C₆-cycloalkenyl, C₃-C₆-cycloalkyl-C₁-C₆-alkylidenyl, C₃-C₆-halocycloalkyl-C₁-C₆-alkylidenyl, C₃-C₆-hydroxycycloalkyl-C₁-C₆-alkyl, C₃-C₆-hydroxycycloalkyl-C₁-C₆-hydroxyalkyl, C₂-C₆-dihydroxyalkyl, or 5- or 6-membered heteroaryl;

also particularly preferred R² is C₂-C₆-alkenyl, C₂-C₆-alkynyl, C₃-C₆-cycloalkenyl-C₁-C₆-alkyl, C₃-C₆-cycloalkyl-C₁-C₆-alkylidenyl, C₁-C₆-hydroxyalkyl, C₁-C₆-alkoxycarbonyl-C₁-C₆-alkyl, C₂-C₆-dihydroxyalkyl, C₁-C₆-dicyanoalkyl, or 5- or 6-membered heteroaryl;

wherein hydroxy groups of R² are unsubstituted or substituted by R^{b};

cyclic groups of R² are unsubstituted or substituted by R^{c}; and

acyclic aliphatic groups of R² are unsubstituted or substituted by R^{d}.

especially preferred R² is C₂-C₆-alkenyl, C₂-C₆-alkynyl, C₃-C₆-cycloalkyl-C₁-C₆-alkylidenyl, C₃-C₆-hydroxycycloalkyl-C₁-C₆-alkyl, and 5- or 6-membered heteroaryl;

also especially preferred R² is C₂-C₆-alkenyl, C₁-C₆-hydroxyalkyl, C₃-C₆-cycloalkyl-C₁-C₆-alkylidenyl, C₂-C₆-dihydroxyalkyl, C₁-C₆-dicyanoalkyl and 5- or 6-membered heteroaryl;

also especially preferred R² is C₂-C₆-alkenyl, C₃-C₆-cycloalkyl-C₁-C₆-alkylidenyl, C₁-C₆-hydroxyalkyl, C₂-C₆-dihydroxyalkyl, or 5- or 6-membered heteroaryl;

wherein hydroxy groups of R² are unsubstituted or substituted by R^{b};

cyclic groups of R² are unsubstituted or substituted by R^{c}; and

acyclic aliphatic groups of R² are unsubstituted or substituted by R^{d}.

more preferred R² is C₂-C₆-alkenyl, C₃-C₆-cycloalkyl-C₁-C₆-alkylidenyl, or 5- or 6-membered heteroaryl;

also more preferred R² is C₁-C₆-hydroxyalkyl, C₂-C₆-dihydroxyalkyl, C₃-C₆-cycloalkyl-C₁-C₆-alkylidenyl, or 5- or 6-membered heteroaryl;

wherein hydroxy groups of R² are unsubstituted or substituted by R^{b};

cyclic groups of R² are unsubstituted or substituted by R^{c}; and

acyclic aliphatic groups of R² are unsubstituted or substituted by R^{d}.

most preferred R² is C₂-C₆-alkenyl;

also most preferred R² is C₃-C₆-cycloalkyl-C₁-C₆-alkylidenyl;

also most preferred R² is 5- or 6-membered heteroaryl;

also most preferred R² is C₁-C₆-hydroxyalkyl;

also most preferred R² is C₂-C₆-dihydroxyalkyl;

wherein hydroxy groups of R² are unsubstituted or substituted by R^{b};

cyclic groups of R² are unsubstituted or substituted by R^{c}; and

acyclic aliphatic groups of R² are unsubstituted or substituted by R^{d}.

also more preferred R² is CH═CH—CH₃, CH═C(CH₂)₃, or CH═C(CH₂)₄;

also more preferred R² is 2-furyl, 3-furyl, 2-methyl-3-furyl, 3-methyl-2-furyl, or 4-methyloxazol-5-yl;

also most preferred R² is CH═CH—CH₃, CH═C(CH₂)₃, 2-furyl, 3-furyl, or 4-methyloxazol-5-yl;

also most preferred R² is CHOH—CHOH—C₆H₅, CHOH—CHOH-2-furyl, or CHOH—CHOH—CH₃.

also most preferred R² is CHOH—CHOH—C₆H₅, CHOH—CHOH-2-furyl, CHOH—CHOH—CH₃, or 4-methyloxazol-5-yl;

also most preferred R² is selected from R²-1 to R²-16 as shown below,

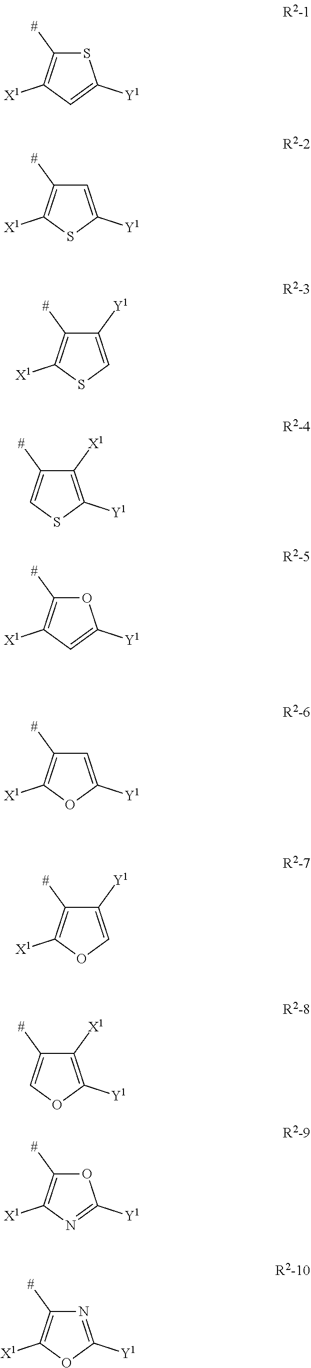

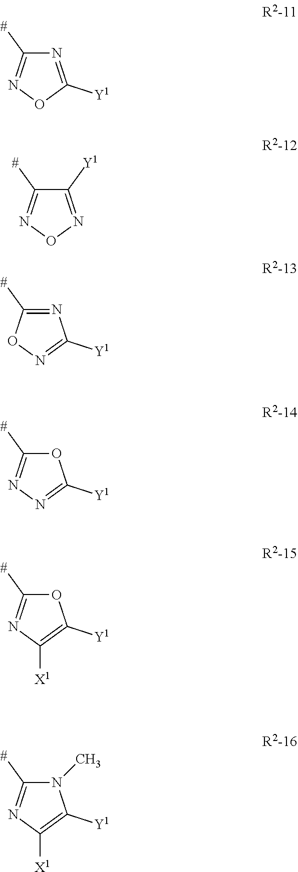

wherein # denotes attachment to the pyrimidine ring, X and Y denotes R^{c} which independently of each other are identical or different;

preferred R² is R²-1, R²-2, R²-3, R²-4, R²-5, R²-6, R²-7, or R²-8;

also preferred R² is R²-9, R²-10, R²-11, R²-13, R²-14, or R²-15;

more preferred R² is R²-9, R²-10, or R²-15;

most preferred R² is R²-9;

preferred X¹ is H, halogen, CN, C₁-C₆-alkyl, C₁-C₆-haloalkyl, OH, C₁-C₆-alkoxy, C₁-C₆-haloalkoxy, or C₁-C₆-alkylthio;

particularly preferred X¹ is H, halogen, CN, C₁-C₆-alkyl, OH, C₁-C₆-alkoxy, or C₁-C₆-alkylthio;

also particularly preferred X is H, halogen, CN, C₁-C₆-alkyl, C₁-C₆-haloalkyl, C₁-C₆-alkoxy, or C₁-C₆-haloalkoxy;

especially preferred X¹ is H, halogen, CN, C₁-C₄-alkyl, OH, C₁-C₄-alkoxy, or C₁-C₄-alkylthio;

more preferred X¹ is H, CH₃, C₂H₅, n-propyl, iso-propyl, iso-butyl, n-butyl, OH, OCH₃, SCH₃, F, Cl, Br, or I;

most preferred X¹ is H, CH₃, C₂H₅, OH, or OCH₃;

also most preferred X¹ is H, CH₃, C₂H₅, or SCH₃;

also most preferred X¹ is H, CH₃, C₂H₅, F, Cl, Br, or I.

preferred Y¹ is H, halogen, CN, C₁-C₆-alkyl, C₁-C₆-haloalkyl, OH, C₁-C₆-alkoxy, C₁-C₆-haloalkoxy, or C₁-C₆-alkylthio;

particularly preferred Y¹ is H, halogen, CN, C₁-C₆-alkyl, OH, C₁-C₆-alkoxy, or C₁-C₆-alkylthio;

also particularly preferred Y¹ is H, halogen, CN, C₁-C₆-alkyl, C₁-C₆-haloalkyl, C₁-C₆-alkoxy, or C₁-C₆-haloalkoxy;

especially preferred Y¹ is H, halogen, CN, C₁-C₄-alkyl, C₁-C₂-fluoroalkyl, OH, C₁-C₄-alkoxy, or C₁-C₄-alkylthio;

more preferred Y¹ is H, CH₃, C₂H₅, n-propyl, iso-propyl, iso-butyl, n-butyl, 2-butyl, t-butyl, OH, OCH₃, SCH₃, F, Cl, Br, or I;

most preferred Y¹ is H, CH₃, C₂H₅, n-propyl, iso-propyl, iso-butyl, n-butyl, 2-butyl, OH, or OCH₃;

also most preferred Y¹ is H, CH₃, C₂H₅, n-propyl, iso-propyl, OH, OCH₃, or SCH₃;

also most preferred Y¹ is H, CH₃, C₂H₅, n-propyl, iso-propyl, F, Cl, Br, or I.

Particularly preferred R² is 4-methyl-5-oxazolyl, 4-ethyl-5-oxazolyl, 2,4-dimethyl-5-oxazolyl, 2-ethyl-4-methyl-5-oxazolyl, 2-methyl-4-ethyl-5-oxazolyl, or 2,4-diethyl-5-oxazolyl.

Examples of more particularly preferred R² are provided in Table R²-9, Table R²-10, and Table R²-15.

Table R²-9: examples of particularly preferred R² are R²-9.1 to R²-9.676 wherein R² is R²-9 and combinations of variables X¹ and Y¹ are as defined in each row of table R2, numbering of each compound e.g. R²-9.1 means R² is R²-9 wherein X¹ and Y¹ are as defined in row 1 of table R2;

Table R²-10: examples of particularly preferred R² are R²-10.1 to R²-10.676 wherein R² is R²-10 and combinations of variables X¹ and Y¹ are as defined in each row of table R2, numbering of each compound e.g. R²-10.1 means R² is R²-10 wherein X¹ and Y¹ are as defined in row 1 of table R2;

Table R²-15: examples of particularly preferred R² are R²-15.1 to R²-15.676 wherein R² is R²-15 and combinations of variables X¹ and Y¹ are as defined in each row of table R2, numbering of each compound e.g. R²-15.1 means R² is R²-15 wherein X¹ and Y¹ are as defined in row 1 of table R2.

TABLE R2

| row | X¹ | Y¹ |
|---|---|---|
| 1 | H | H |
| 2 | H | CH₃ |
| 3 | H | C₂H₅ |
| 4 | H | n-propyl |
| 5 | H | CH(CH₃)₂ |
| 6 | H | iso-butyl |
| 7 | H | n-butyl |
| 8 | H | 2-butyl |
| 9 | H | C(CH₃)₃ |
| 10 | H | OH |
| 11 | H | OCH₃ |
| 12 | H | SCH₃ |
| 13 | H | S(O)CH₃ |
| 14 | H | S(O)₂CH₃ |
| 15 | H | CN |
| 16 | H | F |
| 17 | H | Cl |
| 18 | H | Br |
| 19 | H | I |
| 20 | H | CH₂CF₃ |
| 21 | H | CF₂CF₃ |
| 22 | H | CF₂CH₃ |
| 23 | H | CF₃ |
| 24 | H | CF₂H |
| 25 | H | OCF₂H |
| 26 | H | OCF₃ |
| 27 | CH₃ | H |
| 28 | CH₃ | CH₃ |
| 29 | CH₃ | C₂H₅ |
| 30 | CH₃ | n-propyl |
| 31 | CH₃ | CH(CH₃)₂ |
| 32 | CH₃ | iso-butyl |
| 33 | CH₃ | n-butyl |
| 34 | CH₃ | 2-butyl |
| 35 | CH₃ | C(CH₃)₃ |
| 36 | CH₃ | OH |
| 37 | CH₃ | OCH₃ |
| 38 | CH₃ | SCH₃ |
| 39 | CH₃ | S(O)CH₃ |
| 40 | CH₃ | S(O)₂CH₃ |
| 41 | CH₃ | CN |
| 42 | CH₃ | F |
| 43 | CH₃ | Cl |
| 44 | CH₃ | Br |
| 45 | CH₃ | I |
| 46 | CH₃ | CH₂CF₃ |
| 47 | CH₃ | CF₂CF₃ |
| 48 | CH₃ | CF₂CH₃ |
| 49 | CH₃ | CF₃ |
| 50 | CH₃ | CF₂H |
| 51 | CH₃ | OCF₂H |
| 52 | CH₃ | OCF₃ |
| 53 | C₂H₅ | H |
| 54 | C₂H₅ | CH₃ |
| 55 | C₂H₅ | C₂H₅ |
| 56 | C₂H₅ | n-propyl |
| 57 | C₂H₅ | CH(CH₃)₂ |
| 58 | C₂H₅ | iso-butyl |
| 59 | C₂H₅ | n-butyl |
| 60 | C₂H₅ | 2-butyl |
| 61 | C₂H₅ | C(CH₃)₃ |
| 62 | C₂H₅ | OH |
| 63 | C₂H₅ | OCH₃ |
| 64 | C₂H₅ | SCH₃ |
| 65 | C₂H₅ | S(O)CH₃ |
| 66 | C₂H₅ | S(O)₂CH₃ |
| 67 | C₂H₅ | CN |
| 68 | C₂H₅ | F |
| 69 | C₂H₅ | Cl |
| 70 | C₂H₅ | Br |
| 71 | C₂H₅ | I |
| 72 | C₂H₅ | CH₂CF₃ |
| 73 | C₂H₅ | CF₂CF₃ |
| 74 | C₂H₅ | CF₂CH₃ |
| 75 | C₂H₅ | CF₃ |
| 76 | C₂H₅ | CF₂H |
| 77 | C₂H₅ | OCF₂H |
| 78 | C₂H₅ | OCF₃ |
| 79 | n-propyl | H |
| 80 | n-propyl | CH₃ |
| 81 | n-propyl | C₂H₅ |
| 82 | n-propyl | n-propyl |
| 83 | n-propyl | CH(CH₃)₂ |
| 84 | n-propyl | iso-butyl |
| 85 | n-propyl | n-butyl |
| 86 | n-propyl | 2-butyl |
| 87 | n-propyl | C(CH₃)₃ |
| 88 | n-propyl | OH |
| 89 | n-propyl | OCH₃ |
| 90 | n-propyl | SCH₃ |
| 91 | n-propyl | S(O)CH₃ |
| 92 | n-propyl | S(O)₂CH₃ |
| 93 | n-propyl | CN |
| 94 | n-propyl | F |
| 95 | n-propyl | Cl |
| 96 | n-propyl | Br |
| 97 | n-propyl | I |
| 98 | n-propyl | CH₂CF₃ |
| 99 | n-propyl | CF₂CF₃ |
| 100 | n-propyl | CF₂CH₃ |
| 101 | n-propyl | CF₃ |
| 102 | n-propyl | CF₂H |
| 103 | n-propyl | OCF₂H |
| 104 | n-propyl | OCF₃ |
| 105 | CH(CH₃)₂ | H |
| 106 | CH(CH₃)₂ | CH₃ |
| 107 | CH(CH₃)₂ | C₂H₅ |
| 108 | CH(CH₃)₂ | n-propyl |
| 109 | CH(CH₃)₂ | CH(CH₃)₂ |
| 110 | CH(CH₃)₂ | iso-butyl |
| 111 | CH(CH₃)₂ | n-butyl |
| 112 | CH(CH₃)₂ | 2-butyl |
| 113 | CH(CH₃)₂ | C(CH₃)₃ |
| 114 | CH(CH₃)₂ | OH |
| 115 | CH(CH₃)₂ | OCH₃ |
| 116 | CH(CH₃)₂ | SCH₃ |
| 117 | CH(CH₃)₂ | S(O)CH₃ |
| 118 | CH(CH₃)₂ | S(O)₂CH₃ |
| 119 | CH(CH₃)₂ | CN |
| 120 | CH(CH₃)₂ | F |
| 121 | CH(CH₃)₂ | Cl |
| 122 | CH(CH₃)₂ | Br |
| 123 | CH(CH₃)₂ | I |
| 124 | CH(CH₃)₂ | CH₂CF₃ |
| 125 | CH(CH₃)₂ | CF₂CF₃ |
| 126 | CH(CH₃)₂ | CF₂CH₃ |
| 127 | CH(CH₃)₂ | CF₃ |
| 128 | CH(CH₃)₂ | CF₂H |
| 129 | CH(CH₃)₂ | OCF₂H |
| 130 | CH(CH₃)₂ | OCF₃ |
| 131 | iso-butyl | H |
| 132 | iso-butyl | CH₃ |
| 133 | iso-butyl | C₂H₅ |
| 134 | iso-butyl | n-propyl |
| 135 | iso-butyl | CH(CH₃)₂ |
| 136 | iso-butyl | iso-butyl |
| 137 | iso-butyl | n-butyl |
| 138 | iso-butyl | 2-butyl |
| 139 | iso-butyl | C(CH₃)₃ |
| 140 | iso-butyl | OH |
| 141 | iso-butyl | OCH₃ |
| 142 | iso-butyl | SCH₃ |
| 143 | iso-butyl | S(O)CH₃ |
| 144 | iso-butyl | S(O)₂CH₃ |
| 145 | iso-butyl | CN |
| 146 | iso-butyl | F |
| 147 | iso-butyl | Cl |
| 148 | iso-butyl | Br |
| 149 | iso-butyl | I |
| 150 | iso-butyl | CH₂CF₃ |
| 151 | iso-butyl | CF₂CF₃ |
| 152 | iso-butyl | CF₂CH₃ |
| 153 | iso-butyl | CF₃ |
| 154 | iso-butyl | CF₂H |
| 155 | iso-butyl | OCF₂H |
| 156 | iso-butyl | OCF₃ |
| 157 | n-butyl | H |
| 158 | n-butyl | CH₃ |
| 159 | n-butyl | C₂H₅ |
| 160 | n-butyl | n-propyl |
| 161 | n-butyl | CH(CH₃)₂ |
| 162 | n-butyl | iso-butyl |
| 163 | n-butyl | n-butyl |
| 164 | n-butyl | 2-butyl |
| 165 | n-butyl | C(CH₃)₃ |
| 166 | n-butyl | OH |
| 167 | n-butyl | OCH₃ |
| 168 | n-butyl | SCH₃ |
| 169 | n-butyl | S(O)CH₃ |
| 170 | n-butyl | S(O)₂CH₃ |
| 171 | n-butyl | CN |
| 172 | n-butyl | F |
| 173 | n-butyl | Cl |
| 174 | n-butyl | Br |
| 175 | n-butyl | I |
| 176 | n-butyl | CH₂CF₃ |
| 177 | n-butyl | CF₂CF₃ |
| 178 | n-butyl | CF₂CH₃ |
| 179 | n-butyl | CF₃ |
| 180 | n-butyl | CF₂H |
| 181 | n-butyl | OCF₂H |
| 182 | n-butyl | OCF₃ |
| 183 | 2-butyl | H |
| 184 | 2-butyl | CH₃ |
| 185 | 2-butyl | C₂H₅ |
| 186 | 2-butyl | n-propyl |
| 187 | 2-butyl | CH(CH₃)₂ |
| 188 | 2-butyl | iso-butyl |
| 189 | 2-butyl | n-butyl |
| 190 | 2-butyl | 2-butyl |
| 191 | 2-butyl | C(CH₃)₃ |
| 192 | 2-butyl | OH |
| 193 | 2-butyl | OCH₃ |
| 194 | 2-butyl | SCH₃ |
| 195 | 2-butyl | S(O)CH₃ |
| 196 | 2-butyl | S(O)₂CH₃ |
| 197 | 2-butyl | CN |
| 198 | 2-butyl | F |
| 199 | 2-butyl | Cl |
| 200 | 2-butyl | Br |
| 201 | 2-butyl | I |
| 202 | 2-butyl | CH₂CF₃ |
| 203 | 2-butyl | CF₂CF₃ |
| 204 | 2-butyl | CF₂CH₃ |
| 205 | 2-butyl | CF₃ |
| 206 | 2-butyl | CF₂H |
| 207 | 2-butyl | OCF₂H |
| 208 | 2-butyl | OCF₃ |
| 209 | C(CH₃)₃ | H |
| 210 | C(CH₃)₃ | CH₃ |
| 211 | C(CH₃)₃ | C₂H₅ |
| 212 | C(CH₃)₃ | n-propyl |
| 213 | C(CH₃)₃ | CH(CH₃)₂ |
| 214 | C(CH₃)₃ | iso-butyl |
| 215 | C(CH₃)₃ | n-butyl |
| 216 | C(CH₃)₃ | 2-butyl |
| 217 | C(CH₃)₃ | C(CH₃)₃ |
| 218 | C(CH₃)₃ | OH |
| 219 | C(CH₃)₃ | OCH₃ |
| 220 | C(CH₃)₃ | SCH₃ |
| 221 | C(CH₃)₃ | S(O)CH₃ |
| 222 | C(CH₃)₃ | S(O)₂CH₃ |
| 223 | C(CH₃)₃ | CN |
| 224 | C(CH₃)₃ | F |
| 225 | C(CH₃)₃ | Cl |
| 226 | C(CH₃)₃ | Br |
| 227 | C(CH₃)₃ | I |
| 228 | C(CH₃)₃ | CH₂CF₃ |
| 229 | C(CH₃)₃ | CF₂CF₃ |
| 230 | C(CH₃)₃ | CF₂CH₃ |
| 231 | C(CH₃)₃ | CF₃ |
| 232 | C(CH₃)₃ | CF₂H |
| 233 | C(CH₃)₃ | OCF₂H |
| 234 | C(CH₃)₃ | OCF₃ |
| 235 | OH | H |
| 236 | OH | CH₃ |
| 237 | OH | C₂H₅ |
| 238 | OH | n-propyl |
| 239 | OH | CH(CH₃)₂ |
| 240 | OH | iso-butyl |
| 241 | OH | n-butyl |
| 242 | OH | 2-butyl |
| 243 | OH | C(CH₃)₃ |
| 244 | OH | OH |
| 245 | OH | OCH₃ |
| 246 | OH | SCH₃ |
| 247 | OH | S(O)CH₃ |
| 248 | OH | S(O)₂CH₃ |
| 249 | OH | CN |
| 250 | OH | F |
| 251 | OH | Cl |
| 252 | OH | Br |
| 253 | OH | I |
| 254 | OH | CH₂CF₃ |
| 255 | OH | CF₂CF₃ |
| 256 | OH | CF₂CH₃ |
| 257 | OH | CF₃ |
| 258 | OH | CF₂H |
| 259 | OH | OCF₂H |
| 260 | OH | OCF₃ |
| 261 | OCH₃ | H |
| 262 | OCH₃ | CH₃ |
| 263 | OCH₃ | C₂H₅ |
| 264 | OCH₃ | n-propyl |
| 265 | OCH₃ | CH(CH₃)₂ |
| 266 | OCH₃ | iso-butyl |
| 267 | OCH₃ | n-butyl |
| 268 | OCH₃ | 2-butyl |
| 269 | OCH₃ | C(CH₃)₃ |
| 270 | OCH₃ | OH |
| 271 | OCH₃ | OCH₃ |
| 272 | OCH₃ | SCH₃ |
| 273 | OCH₃ | S(O)CH₃ |
| 274 | OCH₃ | S(O)₂CH₃ |
| 275 | OCH₃ | CN |
| 276 | OCH₃ | F |
| 277 | OCH₃ | Cl |
| 278 | OCH₃ | Br |
| 279 | OCH₃ | I |
| 280 | OCH₃ | CH₂CF₃ |
| 281 | OCH₃ | CF₂CF₃ |
| 282 | OCH₃ | CF₂CH₃ |
| 283 | OCH₃ | CF₃ |
| 284 | OCH₃ | CF₂H |
| 285 | OCH₃ | OCF₂H |
| 286 | OCH₃ | OCF₃ |
| 287 | SCH₃ | H |
| 288 | SCH₃ | CH₃ |
| 289 | SCH₃ | C₂H₅ |
| 290 | SCH₃ | n-propyl |
| 291 | SCH₃ | CH(CH₃)₂ |
| 292 | SCH₃ | iso-butyl |
| 293 | SCH₃ | n-butyl |
| 294 | SCH₃ | 2-butyl |
| 295 | SCH₃ | C(CH₃)₃ |
| 296 | SCH₃ | OH |
| 297 | SCH₃ | OCH₃ |
| 298 | SCH₃ | SCH₃ |
| 299 | SCH₃ | S(O)CH₃ |
| 300 | SCH₃ | S(O)₂CH₃ |
| 301 | SCH₃ | CN |
| 302 | SCH₃ | F |
| 303 | SCH₃ | Cl |
| 304 | SCH₃ | Br |
| 305 | SCH₃ | I |
| 306 | SCH₃ | CH₂CF₃ |
| 307 | SCH₃ | CF₂CF₃ |
| 308 | SCH₃ | CF₂CH₃ |
| 309 | SCH₃ | CF₃ |
| 310 | SCH₃ | CF₂H |
| 311 | SCH₃ | OCF₂H |
| 312 | SCH₃ | OCF₃ |
| 313 | S(O)CH₃ | H |
| 314 | S(O)CH₃ | CH₃ |
| 315 | S(O)CH₃ | C₂H₅ |
| 316 | S(O)CH₃ | n-propyl |
| 317 | S(O)CH₃ | CH(CH₃)₂ |
| 318 | S(O)CH₃ | iso-butyl |
| 319 | S(O)CH₃ | n-butyl |
| 320 | S(O)CH₃ | 2-butyl |
| 321 | S(O)CH₃ | C(CH₃)₃ |
| 322 | S(O)CH₃ | OH |
| 323 | S(O)CH₃ | OCH₃ |
| 324 | S(O)CH₃ | SCH₃ |
| 325 | S(O)CH₃ | S(O)CH₃ |
| 326 | S(O)CH₃ | S(O)₂CH₃ |
| 327 | S(O)CH₃ | CN |
| 328 | S(O)CH₃ | F |
| 329 | S(O)CH₃ | Cl |
| 330 | S(O)CH₃ | Br |
| 331 | S(O)CH₃ | I |
| 332 | S(O)CH₃ | CH₂CF₃ |
| 333 | S(O)CH₃ | CF₂CF₃ |
| 334 | S(O)CH₃ | CF₂CH₃ |
| 335 | S(O)CH₃ | CF₃ |
| 336 | S(O)CH₃ | CF₂H |
| 337 | S(O)CH₃ | OCF₂H |
| 338 | S(O)CH₃ | OCF₃ |
| 339 | S(O)₂CH₃ | H |
| 340 | S(O)₂CH₃ | CH₃ |
| 341 | S(O)₂CH₃ | C₂H₅ |
| 342 | S(O)₂CH₃ | n-propyl |
| 343 | S(O)₂CH₃ | CH(CH₃)₂ |
| 344 | S(O)₂CH₃ | iso-butyl |
| 345 | S(O)₂CH₃ | n-butyl |
| 346 | S(O)₂CH₃ | 2-butyl |
| 347 | S(O)₂CH₃ | C(CH₃)₃ |
| 348 | S(O)₂CH₃ | OH |
| 349 | S(O)₂CH₃ | OCH₃ |
| 350 | S(O)₂CH₃ | SCH₃ |
| 351 | S(O)₂CH₃ | S(O)CH₃ |
| 352 | S(O)₂CH₃ | S(O)₂CH₃ |
| 353 | S(O)₂CH₃ | CN |
| 354 | S(O)₂CH₃ | F |
| 355 | S(O)₂CH₃ | Cl |
| 356 | S(O)₂CH₃ | Br |
| 357 | S(O)₂CH₃ | I |
| 358 | S(O)₂CH₃ | CH₂CF₃ |
| 359 | S(O)₂CH₃ | CF₂CF₃ |
| 360 | S(O)₂CH₃ | CF₂CH₃ |
| 361 | S(O)₂CH₃ | CF₃ |
| 362 | S(O)₂CH₃ | CF₂H |
| 363 | S(O)₂CH₃ | OCF₂H |
| 364 | S(O)₂CH₃ | OCF₃ |
| 365 | CN | H |
| 366 | CN | CH₃ |
| 367 | CN | C₂H₅ |
| 368 | CN | n-propyl |
| 369 | CN | CH(CH₃)₂ |
| 370 | CN | iso-butyl |
| 371 | CN | n-butyl |
| 372 | CN | 2-butyl |
| 373 | CN | C(CH₃)₃ |
| 374 | CN | OH |
| 375 | CN | OCH₃ |
| 376 | CN | SCH₃ |
| 377 | CN | S(O)CH₃ |
| 378 | CN | S(O)₂CH₃ |
| 379 | CN | CN |
| 380 | CN | F |
| 381 | CN | Cl |
| 382 | CN | Br |
| 383 | CN | I |
| 384 | CN | CH₂CF₃ |
| 385 | CN | CF₂CF₃ |
| 386 | CN | CF₂CH₃ |
| 387 | CN | CF₃ |
| 388 | CN | CF₂H |
| 389 | CN | OCF₂H |
| 390 | CN | OCF₃ |
| 391 | F | H |
| 392 | F | CH₃ |
| 393 | F | C₂H₅ |
| 394 | F | n-propyl |
| 395 | F | CH(CH₃)₂ |
| 396 | F | iso-butyl |
| 397 | F | n-butyl |
| 398 | F | 2-butyl |
| 399 | F | C(CH₃)₃ |
| 400 | F | OH |
| 401 | F | OCH₃ |
| 402 | F | SCH₃ |
| 403 | F | S(O)CH₃ |
| 404 | F | S(O)₂CH₃ |
| 405 | F | CN |
| 406 | F | F |
| 407 | F | Cl |
| 408 | F | Br |
| 409 | F | I |
| 410 | F | CH₂CF₃ |
| 411 | F | CF₂CF₃ |
| 412 | F | CF₂CH₃ |
| 413 | F | CF₃ |
| 414 | F | CF₂H |
| 415 | F | OCF₂H |
| 416 | F | OCF₃ |
| 417 | Cl | H |
| 418 | Cl | CH₃ |
| 419 | Cl | C₂H₅ |
| 420 | Cl | n-propyl |
| 421 | Cl | CH(CH₃)₂ |
| 422 | Cl | iso-butyl |
| 423 | Cl | n-butyl |
| 424 | Cl | 2-butyl |
| 425 | Cl | C(CH₃)₃ |
| 426 | Cl | OH |
| 427 | Cl | OCH₃ |
| 428 | Cl | SCH₃ |
| 429 | Cl | S(O)CH₃ |
| 430 | Cl | S(O)₂CH₃ |
| 431 | Cl | CN |
| 432 | Cl | F |
| 433 | Cl | Cl |
| 434 | Cl | Br |
| 435 | Cl | I |
| 436 | Cl | CH₂CF₃ |
| 437 | Cl | CF₂CF₃ |
| 438 | Cl | CF₂CH₃ |
| 439 | Cl | CF₃ |
| 440 | Cl | CF₂H |
| 441 | Cl | OCF₂H |
| 442 | Cl | OCF₃ |
| 443 | Br | H |
| 444 | Br | CH₃ |
| 445 | Br | C₂H₅ |
| 446 | Br | n-propyl |
| 447 | Br | CH(CH₃)₂ |
| 448 | Br | iso-butyl |
| 449 | Br | n-butyl |
| 450 | Br | 2-butyl |
| 451 | Br | C(CH₃)₃ |
| 452 | Br | OH |
| 453 | Br | OCH₃ |
| 454 | Br | SCH₃ |
| 455 | Br | S(O)CH₃ |
| 456 | Br | S(O)₂CH₃ |
| 457 | Br | CN |
| 458 | Br | F |
| 459 | Br | Cl |
| 460 | Br | Br |
| 461 | Br | I |
| 462 | Br | CH₂CF₃ |
| 463 | Br | CF₂CF₃ |
| 464 | Br | CF₂CH₃ |
| 465 | Br | CF₃ |
| 466 | Br | CF₂H |
| 467 | Br | OCF₂H |
| 468 | Br | OCF₃ |
| 469 | I | H |
| 470 | I | CH₃ |
| 471 | I | C₂H₅ |
| 472 | I | n-propyl |
| 473 | I | CH(CH₃)₂ |
| 474 | I | iso-butyl |
| 475 | I | n-butyl |
| 476 | I | 2-butyl |
| 477 | I | C(CH₃)₃ |
| 478 | I | OH |
| 479 | I | OCH₃ |
| 480 | I | SCH₃ |
| 481 | I | S(O)CH₃ |
| 482 | I | S(O)₂CH₃ |
| 483 | I | CN |
| 484 | I | F |
| 485 | I | Cl |
| 486 | I | Br |
| 487 | I | I |
| 488 | I | CH₂CF₃ |
| 489 | I | CF₂CF₃ |
| 490 | I | CF₂CH₃ |
| 491 | I | CF₃ |
| 492 | I | CF₂H |
| 493 | I | OCF₂H |
| 494 | I | OCF₃ |
| 495 | CH₂CF₃ | H |
| 496 | CH₂CF₃ | CH₃ |
| 497 | CH₂CF₃ | C₂H₅ |
| 498 | CH₂CF₃ | n-propyl |
| 499 | CH₂CF₃ | CH(CH₃)₂ |
| 500 | CH₂CF₃ | iso-butyl |
| 501 | CH₂CF₃ | n-butyl |
| 502 | CH₂CF₃ | 2-butyl |
| 503 | CH₂CF₃ | C(CH₃)₃ |
| 504 | CH₂CF₃ | OH |
| 505 | CH₂CF₃ | OCH₃ |
| 506 | CH₂CF₃ | SCH₃ |
| 507 | CH₂CF₃ | S(O)CH₃ |
| 508 | CH₂CF₃ | S(O)₂CH₃ |
| 509 | CH₂CF₃ | CN |
| 510 | CH₂CF₃ | F |
| 511 | CH₂CF₃ | Cl |
| 512 | CH₂CF₃ | Br |
| 513 | CH₂CF₃ | I |
| 514 | CH₂CF₃ | CH₂CF₃ |
| 515 | CH₂CF₃ | CF₂CF₃ |
| 516 | CH₂CF₃ | CF₂CH₃ |
| 517 | CH₂CF₃ | CF₃ |
| 518 | CH₂CF₃ | CF₂H |
| 519 | CH₂CF₃ | OCF₂H |
| 520 | CH₂CF₃ | OCF₃ |
| 521 | CF₂CF₃ | H |
| 522 | CF₂CF₃ | CH₃ |
| 523 | CF₂CF₃ | C₂H₅ |
| 524 | CF₂CF₃ | n-propyl |
| 525 | CF₂CF₃ | CH(CH₃)₂ |
| 526 | CF₂CF₃ | iso-butyl |
| 527 | CF₂CF₃ | n-butyl |
| 528 | CF₂CF₃ | 2-butyl |
| 529 | CF₂CF₃ | C(CH₃)₃ |
| 530 | CF₂CF₃ | OH |
| 531 | CF₂CF₃ | OCH₃ |
| 532 | CF₂CF₃ | SCH₃ |
| 533 | CF₂CF₃ | S(O)CH₃ |
| 534 | CF₂CF₃ | S(O)₂CH₃ |
| 535 | CF₂CF₃ | CN |
| 536 | CF₂CF₃ | F |
| 537 | CF₂CF₃ | Cl |
| 538 | CF₂CF₃ | Br |
| 539 | CF₂CF₃ | I |
| 540 | CF₂CF₃ | CH₂CF₃ |
| 541 | CF₂CF₃ | CF₂CF₃ |
| 542 | CF₂CF₃ | CF₂CH₃ |
| 543 | CF₂CF₃ | CF₃ |
| 544 | CF₂CF₃ | CF₂H |
| 545 | CF₂CF₃ | OCF₂H |
| 546 | CF₂CF₃ | OCF₃ |
| 547 | CF₂CH₃ | H |
| 548 | CF₂CH₃ | CH₃ |
| 549 | CF₂CH₃ | C₂H₅ |
| 550 | CF₂CH₃ | n-propyl |
| 551 | CF₂CH₃ | CH(CH₃)₂ |
| 552 | CF₂CH₃ | iso-butyl |
| 553 | CF₂CH₃ | n-butyl |
| 554 | CF₂CH₃ | 2-butyl |
| 555 | CF₂CH₃ | C(CH₃)₃ |
| 556 | CF₂CH₃ | OH |
| 557 | CF₂CH₃ | OCH₃ |
| 558 | CF₂CH₃ | SCH₃ |
| 559 | CF₂CH₃ | S(O)CH₃ |
| 560 | CF₂CH₃ | S(O)₂CH₃ |
| 561 | CF₂CH₃ | CN |
| 562 | CF₂CH₃ | F |
| 563 | CF₂CH₃ | Cl |
| 564 | CF₂CH₃ | Br |
| 565 | CF₂CH₃ | I |
| 566 | CF₂CH₃ | CH₂CF₃ |
| 567 | CF₂CH₃ | CF₂CF₃ |
| 568 | CF₂CH₃ | CF₂CH₃ |
| 569 | CF₂CH₃ | CF₃ |
| 570 | CF₂CH₃ | CF₂H |
| 571 | CF₂CH₃ | OCF₂H |
| 572 | CF₂CH₃ | OCF₃ |
| 573 | CF₃ | H |
| 574 | CF₃ | CH₃ |
| 575 | CF₃ | C₂H₅ |
| 576 | CF₃ | n-propyl |
| 577 | CF₃ | CH(CH₃)₂ |
| 578 | CF₃ | iso-butyl |
| 579 | CF₃ | n-butyl |
| 580 | CF₃ | 2-butyl |
| 581 | CF₃ | C(CH₃)₃ |
| 582 | CF₃ | OH |
| 583 | CF₃ | OCH₃ |
| 584 | CF₃ | SCH₃ |
| 585 | CF₃ | S(O)CH₃ |
| 586 | CF₃ | S(O)₂CH₃ |
| 587 | CF₃ | CN |
| 588 | CF₃ | F |
| 589 | CF₃ | Cl |
| 590 | CF₃ | Br |
| 591 | CF₃ | I |
| 592 | CF₃ | CH₂CF₃ |
| 593 | CF₃ | CF₂CF₃ |
| 594 | CF₃ | CF₂CH₃ |
| 595 | CF₃ | CF₃ |
| 596 | CF₃ | CF₂H |
| 597 | CF₃ | OCF₂H |
| 598 | CF₃ | OCF₃ |
| 599 | CF₂H | H |
| 600 | CF₂H | CH₃ |
| 601 | CF₂H | C₂H₅ |
| 602 | CF₂H | n-propyl |
| 603 | CF₂H | CH(CH₃)₂ |
| 604 | CF₂H | iso-butyl |
| 605 | CF₂H | n-butyl |
| 606 | CF₂H | 2-butyl |
| 607 | CF₂H | C(CH₃)₃ |
| 608 | CF₂H | OH |
| 609 | CF₂H | OCH₃ |
| 610 | CF₂H | SCH₃ |
| 611 | CF₂H | S(O)CH₃ |
| 612 | CF₂H | S(O)₂CH₃ |
| 613 | CF₂H | CN |
| 614 | CF₂H | F |
| 615 | CF₂H | Cl |
| 616 | CF₂H | Br |
| 617 | CF₂H | I |
| 618 | CF₂H | CH₂CF₃ |
| 619 | CF₂H | CF₂CF₃ |
| 620 | CF₂H | CF₂CH₃ |
| 621 | CF₂H | CF₃ |
| 622 | CF₂H | CF₂H |
| 623 | CF₂H | OCF₂H |
| 624 | CF₂H | OCF₃ |
| 625 | OCF₂H | H |
| 626 | OCF₂H | CH₃ |
| 627 | OCF₂H | C₂H₅ |
| 628 | OCF₂H | n-propyl |
| 629 | OCF₂H | CH(CH₃)₂ |
| 630 | OCF₂H | iso-butyl |
| 631 | OCF₂H | n-butyl |
| 632 | OCF₂H | 2-butyl |
| 633 | OCF₂H | C(CH₃)₃ |
| 634 | OCF₂H | OH |
| 635 | OCF₂H | OCH₃ |
| 636 | OCF₂H | SCH₃ |
| 637 | OCF₂H | S(O)CH₃ |
| 638 | OCF₂H | S(O)₂CH₃ |
| 639 | OCF₂H | CN |
| 640 | OCF₂H | F |
| 641 | OCF₂H | Cl |
| 642 | OCF₂H | Br |
| 643 | OCF₂H | I |
| 644 | OCF₂H | CH₂CF₃ |
| 645 | OCF₂H | CF₂CF₃ |
| 646 | OCF₂H | CF₂CH₃ |
| 647 | OCF₂H | CF₃ |
| 648 | OCF₂H | CF₂H |
| 649 | OCF₂H | OCF₂H |
| 650 | OCF₂H | OCF₃ |
| 651 | OCF₃ | H |
| 652 | OCF₃ | CH₃ |
| 653 | OCF₃ | C₂H₅ |
| 654 | OCF₃ | n-propyl |
| 655 | OCF₃ | CH(CH₃)₂ |
| 656 | OCF₃ | iso-butyl |
| 657 | OCF₃ | n-butyl |
| 658 | OCF₃ | 2-butyl |
| 659 | OCF₃ | C(CH₃)₃ |
| 660 | OCF₃ | OH |
| 661 | OCF₃ | OCH₃ |
| 662 | OCF₃ | SCH₃ |
| 663 | OCF₃ | S(O)CH₃ |
| 664 | OCF₃ | S(O)₂CH₃ |
| 665 | OCF₃ | CN |
| 666 | OCF₃ | F |
| 667 | OCF₃ | Cl |
| 668 | OCF₃ | Br |
| 669 | OCF₃ | I |
| 670 | OCF₃ | CH₂CF₃ |
| 671 | OCF₃ | CF₂CF₃ |
| 672 | OCF₃ | CF₂CH₃ |
| 673 | OCF₃ | CF₃ |
| 674 | OCF₃ | CF₂H |
| 675 | OCF₃ | OCF₂H |
| 676 | OCF₃ | OCF₃ |

Preferred A is CR³, C*, NR^{3A}, N, S or O;

particularly preferred A is CR³, C*, NR^{3A}, or N;

also particularly preferred A is S or O;

especially preferred A is C*, CR³ or N;

most preferred A is CR³;

also most preferred A is C*;

also most preferred A is N.

Preferred Z is 9 or 10 membered bicyclic ring, wherein the ring comprising A is phenyl or pyridine ring which is fused with another 5- or 6-membered partially unsaturated 5- or 6-membered carbocycle comprising 1, 2 or 3 heteroatoms selected from O or N;

particularly preferred Z is 9 membered bicyclic ring;

more particularly preferred Z is a 9-membered bycyclic ring wherein the ring comprising A is phenyl or pyridine ring which is fused with another 5-membered partially unsaturated 5- or 6-membered carbocycle comprising 1, 2 or 3 heteroatoms selected from O or N;

also more particularly preferred Z is a 9-membered bycyclic ring wherein the ring comprising A is phenyl or pyridine ring which is fused with another 5-membered partially unsaturated 5- or 6-membered carbocycle comprising 1 or 2 oxygen atoms;

most particularly preferred Z is a 9-membered bycyclic ring wherein the ring comprising A is a phenyl ring which is fused with another partially unsaturated 5-membered carbocycle comprising 1 or 2 oxygen atoms;

also more particularly preferred Z is a 9-membered bycyclic ring wherein the ring comprising A is thiophene, furan or pyrol ring which is fused with another 5- or 6-membered partially unsaturated 5- or 6-membered carbocycle comprising 1 or 2 heteroatoms selected from O or N;

also most particularly preferred Z is a 9-membered bycyclic ring wherein the ring comprising A is thiophene, furan or pyrol ring which is fused with another 6-membered partially unsaturated 5- or 6-membered carbocycle comprising 1 or 2 oxygen atoms;

particularly preferred Z is 10 membered bicyclic ring;

more particularly preferred Z is a 10-membered bycyclic ring wherein the ring comprising A is phenyl or pyridine ring which is fused with another 5- or 6-membered partially unsaturated 5- or 6-membered carbocycle comprising 1, 2 or 3 heteroatoms selected from O or N;

also more particularly preferred Z is a 10-membered bycyclic ring wherein the ring comprising A is phenyl or pyridine ring which is fused with another 5- or 6-membered partially unsaturated 5- or 6-membered carbocycle comprising 1 or 2 oxygen atoms;

most particularly preferred Z is a 10-membered bycyclic ring wherein the ring comprising A is phenyl ring which is fused with another 6-membered partially unsaturated 5- or 6-membered carbocycle comprising 1 or 2 oxygen atoms;

more preferred Z is selected from below groups A to O,

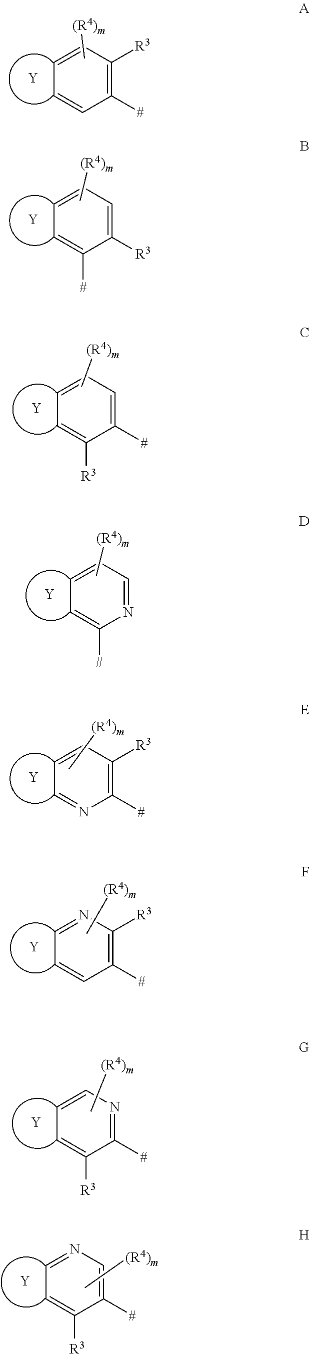

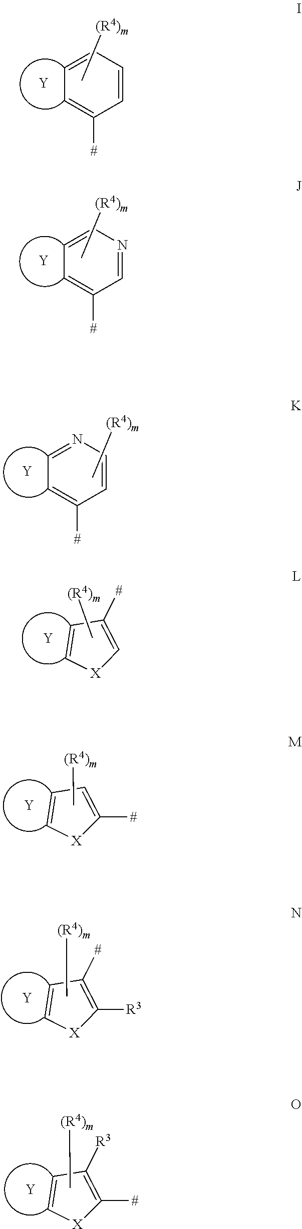

wherein

Y is 5- or 6-membered partially or fully unsaturated carbocycle comprising 0, 1, 2, or 3 heteroatoms selected from O, N, and S;

R³ is halogen, CN, C₁-C₆-alkyl, C₁-C₆-haloalkyl, or C₁-C₆-alkoxy;

m is 0, 1 or 2;

R⁴ is halogen, CN, C₁-C₆-alkyl, C₁-C₆-haloalkyl, or C₁-C₆-alkoxy;

X is O, S, or NR^{3A};

R^{3A} is H, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl; and

# denotes the point of attachment to the pyrimidine ring.

Preferred Y is phenyl;

also preferred Y is 5- or 6-membered partially or fully unsaturated carbocycle comprising 1, 2, or 3 heteroatoms selected from O, N, and S;

particularly preferred Y is phenyl;

also particularly preferred Y is 5-membered partially or fully unsaturated carbocycle comprising 1, or 2 heteroatoms selected from O, N, and S;

more preferred Y is 5-membered partially unsaturated carbocycle comprising 0, 1, or 2 heteroatoms selected from O, N, and S, e.g. 1,3-dithiolane, 1,3-oxathiolane, 1,3-dioxolane, 2,3-dihydrofuran, 2,3-dihydrothiophene, or 2,3-dihydro-1H-pyrrole; more preferably heteroatoms are selected from O and S, e.g. 1,3-oxathiolane, 1,3-dioxolane, 2,3-dihydrofuran, or 2,3-dihydrothiophene; most preferred heteroatom in Y is O, e.g. 1,3-dioxolane, or 2,3-dihydrofuran; also most preferred heteroatom in Y is S, e.g. 2,3-dihydrothiophene;

also more preferred Y is 5-membered fully unsaturated carbocycle comprising 0, 1, or 2 heteroatoms selected from O, N, and S (furan, thiophene, 1H-pyrrole, 1,2-oxazole, 1,3-oxazole, 1,2-thiazole, 1,3-thiazole, imidazole, 1H-pyrazole); more preferably heteroatoms are selected from O and S; most preferred heteroatom is 0; also most preferred heteroatom is S;

most preferred Y is 5-membered partially unsaturated carbocycle comprising 1 or 2 oxygen atoms;

also particularly preferred Y is 6-membered partially or fully unsaturated carbocycle comprising 0, 1, or 2 heteroatoms selected from O, N, and S;

more preferred Y is 6-membered partially unsaturated carbocycle comprising 0, 1, or 2 heteroatoms selected from O, N, and S; more preferably heteroatoms are selected from O and S;

also more preferably heteroatoms are selected from O and N; most preferred heteroatom is 0;

also more preferred Y is 6-membered fully unsaturated carbocycle comprising 0, 1, or 2 heteroatoms N;

Preferred R³ is halogen, CN, NO₂, C₁-C₆-alkoxy, or C₃-C₆-cycloalkyl;

also preferred R³ is halogen, CN, C₁-C₆-haloalkyl, or C₁-C₆-alkoxy,

particularly preferred R³ is halogen, CN, C₁-C₆-alkyl, or C₁-C₆-alkoxy;

especially preferred halogen, or CH₃;

also especially preferred R³ is halogen;

more preferred R³ is Cl, Br, or I;

most preferred R³ is Cl or Br.

Preferred R^{3A} is H, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl;

also preferred R^{3A} is H, C₁-C₆-haloalkyl, or C₁-C₆-alkylcarbonyl;

particularly preferred R^{3A} is H, C₁-C₆-alkyl, or C₁-C₆-alkylcarbonyl;

especially preferred R^{3A} is H, or C₁-C₆-alkyl;

most preferred R^{3A} is H, or CH₃.

Preferred R⁴ is halogen, CN, C₁-C₆-haloalkyl, or C₁-C₆-alkoxy;

particularly preferred R⁴ is halogen, C₁-C₆-haloalkyl, or C₁-C₆-alkyl;

especially preferred R⁴ is halogen;

also especially preferred R⁴ is C₁-C₆-haloalkyl, or C₁-C₆-alkyl;

more preferred R⁴ is F, Cl, CHF₂, CF₃, CH₃, or C₂H₅;

most preferred R⁴ is F;

also most preferred R⁴ is CH₃;

also most preferred R⁴ is Cl;

also most preferred R⁴ is CF₃.

Preferred m is 0, 1, or 2;

more preferred m is 0 or 1;

most preferred m is 0.

also most preferred m is 1.

Preferred Z is A, B, C, D, E, F, G, H, I, J, or K;

more preferred Z is A, E, or F;

most preferred Z is A;

Also preferred Z is L, M, N, or O;

Also preferred are the pyrimidine compounds of formula (I), and their use as herbicide, wherein

R¹ is preferably C₁-C₆-alkyl, C₁-C₆-alkoxy, or C₃-C₆-cycloalkyl, wherein the cycloalkyl substituent is unsubstituted;

particularly preferred R¹ is C₃-C₆-cycloalkyl, wherein the cycloalkyl substituent is unsubstituted;

R² is preferably C₂-C₆-alkenyl, C₃-C₆-cycloalkyl-C₁-C₆-alkylidenyl, 5- or 6-membered heteroaryl, C₁-C₆-hydroxyalkyl, or C₂-C₆-dihydroxyalkyl;

particularly preferred R² is C₂-C₆-alkenyl, 5- or 6-membered heteroaryl, or C₁-C₆-hydroxyalkyl;

also particularly preferred R² is C₃-C₆-cycloalkylC₁-C₆-alkylidenyl, 5- or 6-membered heteroaryl, or C₂-C₆-dihydroxyalkyl;

more preferred R² is CH═CH—CH₃, CH═C(CH₂)₃, or CH═C(CH₂)₄;

also more preferred R² is 2-furyl, 3-furyl, 2-methyl-3-furyl, 3-methyl-2-furyl, CHOH—CHOH—C₆H₅, or CHOH—CHOH-2-furyl, or 4-methyloxazol-5-yl;

most preferred R² is CH═CH—CH₃, CH═C(CH₂)₃, 2-furyl, 3-furyl, CHOH—CHOH—C₆H₅, CHOH—CHOH-2-furyl, or 4-methyloxazol-5-yl.

A is preferably CR³, C*, N, O, S or NR^{3A};

particularly preferred A is CR³, C*, or N;

also particularly preferred A is CR³, C*, O or S;

Preferred Z is 9 or 10 membered bicyclic ring;

particularly preferred Z is 9 membered bicyclic ring;

particularly preferred Z is 10 membered bicyclic ring;

more preferred Z is selected from radicals A to O, as defined herein; R³ is preferably halogen, CN, C₁-C₆-alkyl, C₁-C₆-haloalkyl, or C₁-C₆-alkoxy;

particularly preferred R³ is halogen or CH₃;

R^{3A} is preferably H or C₁-C₆-alkyl;

particularly preferred R^{3A} is H or CH₃;

m is preferably 0 or 1;

R⁴ is preferably halogen.

Also preferred are the pyrimidine compounds of formula (I), wherein

R¹ is c-C₃H₅;

R² is C₁-C₆-alkyl, C₁-C₆-haloalkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-alkyl, C₁-C₆-alkoxycarbonyl-C₁-C₆-haloalkyl, or 5-membered heteroaryl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d};

R^{b} is C₁-C₆-alkyl;

R^{c} is C₁-C₆-alkyl or OH;

R^{d} is phenyl or 5- or 6-membered heteroaryl;

wherein the substituent R^{d} is unsubstituted or substituted by R^{e};

R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;

Z is A, E or F;

Y is 5- or 6-membered partially unsaturated carbocycle comprising 1 or 2 oxygen atoms;

R³ is R³ is halogen, C₁-C₆-alkyl, C₁-C₆-haloalkyl, C₁-C₆-alkoxy, or C₁-C₆-haloalkoxy; preferrably C₁-C₆-alkyl, C₁-C₆-haloalkyl, or C₁-C₆-haloalkoxy; more preferrably Cl, Br, F, I, CH₃, or OCF₃; most preferably Cl;

m is 0 or 1;

R⁴ is halogen, preferably Br.

Also preferred are the pyrimidine compounds of formula (I), wherein

R¹ is c-C₃H₅;

R² is C₁-C₆-alkyl, C₁-C₆-haloalkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, C₁-C₆-alkoxycarbonyl-C₁-C₆-alkyl, or 5-membered heteroaryl;

wherein OH groups of R² are unsubstituted or substituted by R^{b},

cyclic groups of R² are unsubstituted or substituted by R^{c}, and

acyclic aliphatic groups of R² are unsubstituted or substituted by R^{d};

R^{b} is C₁-C₆-alkyl;

R^{c} is C₁-C₆-alkyl or OH;

R^{d} is 5- or 6-membered heteroaryl or 3- to 6-membered heterocyclyl; wherein the substituent R^{d} is unsubstituted or substituted by R^{e};

R^{e} is halogen, CN, NO₂, C₁-C₆-alkyl, C₁-C₆-haloalkyl, OH, C₁-C₆-alkoxy, C₁-C₆-haloalkoxy, C₁-C₆-alkylsulfonyl;

Z is A;

Y is 5- or 6-membered partially unsaturated carbocycle comprising 1 or 2 oxygen atoms;

R³ is R³ is halogen, C₁-C₆-alkyl, C₁-C₆-haloalkyl, C₁-C₆-alkoxy, or C₁-C₆-haloalkoxy; preferably C₁-C₆-alkyl, C₁-C₆-haloalkyl, or C₁-C₆-haloalkoxy; more preferably Cl, Br, F, I, CH₃, or OCF₃; most preferably Cl;

m is 0 or 1; preferably 0;

R⁴ is F, Br, Cl, CH F₂, CH₃, CF₃, or C₂H₅.

Also preferred are the pyrimidine compounds of formula (I), wherein

R¹ is c-C₃H₅;

R² is R²-9, R²-10 or R²-15, preferably R²-9;

Z is A;

Y is 5- or 6-membered partially unsaturated carbocycle comprising 1 or 2 oxygen atoms;

R³ is R³ is halogen, C₁-C₆-alkyl, C₁-C₆-haloalkyl, C₁-C₆-alkoxy, or C₁-C₆-haloalkoxy; preferably C₁-C₆-alkyl, C₁-C₆-haloalkyl, or C₁-C₆-haloalkoxy; more preferably Cl, Br, F, I, CH₃, or OCF₃; most preferably Cl;

m is 0 or 1; preferably 0;

R⁴ is F, Br, Cl, CH F₂, CH₃, CF₃, or C₂H₅.

Also preferred are the pyrimidine compounds of formula (I.1) (corresponds to pyrimidine compounds of formula (I) wherein R² is CH═CH—CH₃), and their use as herbicide,

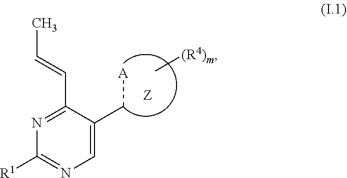

wherein the dotted line (------) is a single bond or a double bond;
   R¹ is C₃-C₆-cycloalkyl, C₁-C₆-alkyl, or C₁-C₆-alkoxy;
   A is CR³, C*, NR^{3A}, N, O or S;
   R³ is halogen, C₁-C₆-alkyl, C₁-C₆-haloalkyl, or C₁-C₆-alkoxy;
   R^{3A} is H or C₁-C₆-alkyl;
   Z is radical selected from A to O;
   m is 0 or 1;
   R⁴ is F, CHF₂, CF₃, CH₃, or C₂H₅.

Also preferred are the pyrimidine compounds of formula (1.2) (corresponds to pyrimidine compounds of formula (I) wherein R² is CH═C(CH₂)₃), and their use as herbicide,

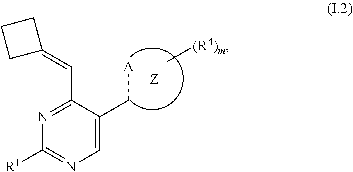

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.3) (corresponds to pyrimidine compounds of formula (I) wherein R² is CH═C(CH₂)₄), and their use as herbicide,

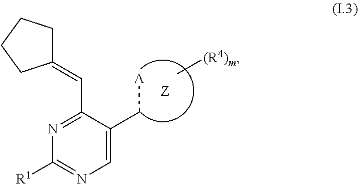

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.4) (corresponds to pyrimidine compounds of formula (I) wherein R² is 2-furyl), and their use as herbicide,

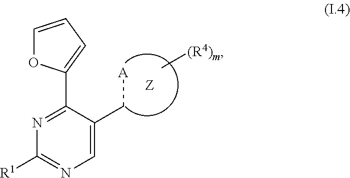

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.5) (corresponds to pyrimidine compounds of formula (I) wherein R² is 3-furyl), and their use as herbicide,

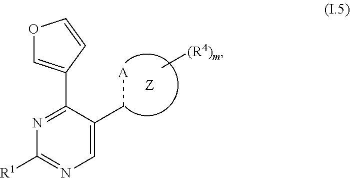

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.6) (corresponds to pyrimidine compounds of formula (I) wherein R² is 3-methyl-2-furyl), and their use as herbicide,

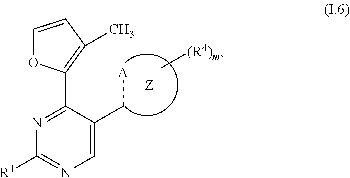

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.7) (corresponds to pyrimidine compounds of formula (I) wherein R² is 2-methyl-3-furyl), and their use as herbicide,

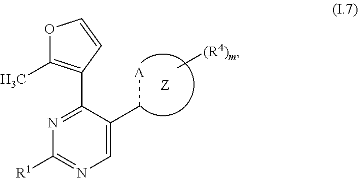

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S; R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.8) (corresponds to pyrimidine compounds of formula (I) wherein R² is CHOH—CHOH—C₆H₅), and their use as herbicide,

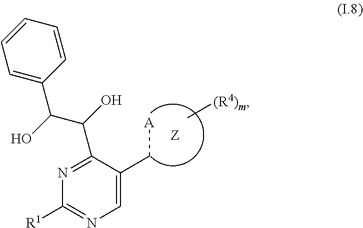

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.9) (corresponds to pyrimidine compounds of formula (I) wherein R² is CHOH—CHOH-2-furyl), and their use as herbicide,

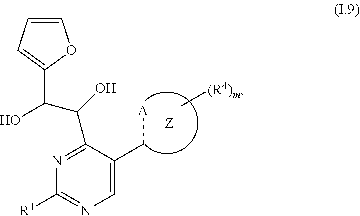

wherein the dotted line (------) is a single bond or a double bond;

R¹ is C₃-C₆-cycloalkyl, C₁-C₆-alkyl, or C₁-C₆-alkoxy;

A is CR³, C*, NR^{3A}, N, O or S;

R³ is halogen, C₁-C₆-alkyl, C₁-C₆-haloalkyl, or C₁-C₆-alkoxy;

Z is radical selected from A to O;

m is 0 or 1;

R⁴ is F, CHF₂, CH₃, CF₃, or C₂H₅.

Also preferred are the pyrimidine compounds of formula (I.10) (corresponds to pyrimidine compounds of formula (I) wherein R² is 4-methyloxazol-5-yl), and their use as herbicide,

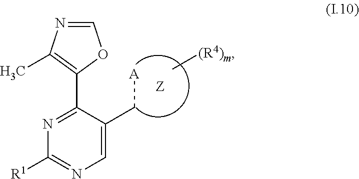

wherein the dotted line (------) is a single bond or a double bond;

R¹ is C₃-C₆-cycloalkyl, C₁-C₆-alkyl, or C₁-C₆-alkoxy;

A is CR³;

R³ is halogen, C₁-C₆-alkyl, C₁-C₆-haloalkyl, or C₁-C₆-alkoxy;

Z is radical selected from A to O;

m is 0 or 1;

R⁴ is F, Cl, CHF₂, CH₃, CF₃, or C₂H₅.

Also preferred are the pyrimidine compounds of formula (I.11) (corresponds to pyrimidine compounds of formula (I) wherein R² is R²-9), and their use as herbicide,

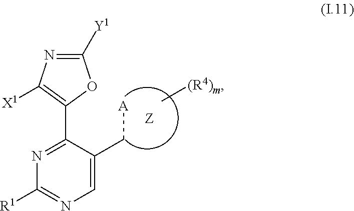

wherein the dotted line (------) is a single bond or a double bond;

X¹ and Y¹ independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, Cl, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

A is CR³;

R³ is halogen, C₁-C₆-alkyl, C₁-C₆-haloalkyl, or C₁-C₆-alkoxy;

Z is radical selected from A to O;

m is 0 or 1;

R⁴ is F, Br, Cl, CHF₂, CH₃, CF₃, or C₂H₅; preferably F, Cl, CHF₂, CH₃, CF₃, or C₂H₅; more preferably Cl.

Also preferred are the pyrimidine compounds of formula (I.11.A) (corresponds to pyrimidine compounds of formula (I), wherein wherein R² is R²-9 and Z is A) and their use as herbicide,

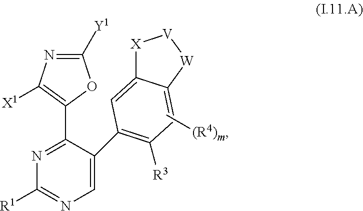

wherein

X¹ and Y¹ independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, Cl, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

R³ is halogen, C₁-C₆-alkyl, C₁-C₆-haloalkyl, or C₁-C₆-alkoxy;

m is 0 or 1;

R⁴ is F, Br, Cl, CHF₂, CH₃, CF₃, or C₂H₅; preferably F, Cl, CHF₂, CH₃, CF₃, or C₂H₅; more preferably Cl.

Also preferred are the pyrimidine compounds of formula (I.11.C) (corresponds to pyrimidine compounds of formula (I), wherein wherein R² is R²-9 and Z is C) and their use as herbicide,

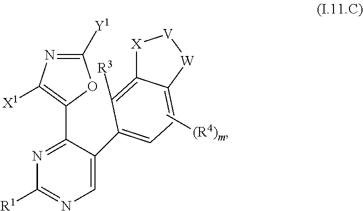

wherein

X¹ and Y¹ independently are selected from H, CH₃, C₂H₅, n-propyl, iso-propyl, iso-butyl, n-butyl, 2-butyl, t-butyl, OH, OCH₃, SCH₃, S(O)CH₃, S(O)₂CH₃, CN, F, Cl, Br, I, CH₂CF₃, CF₂CF₃, CF₂CH₃, CF₃, CF₂H, OCF₂H, and OCF₃;

R¹ is C₃-C₆-cycloalkyl, C₁-C₆-alkyl, or C₁-C₆-alkoxy; preferably c-C₃H₅;

R³ is halogen, C₁-C₆-alkyl, C₁-C₆-haloalkyl, or C₁-C₆-alkoxy;

m is 0 or 1;

R⁴ is F, Br, Cl, CHF₂, CH₃, CF₃, or C₂H₅; preferably F, Cl, CHF₂, CH₃, CF₃, or C₂H₅; more preferably Cl.

Particular preference is given to the pyrimidine compounds of formula I which corresponds to compounds of formulae I.a to I.1, and their use as herbicide, wherein X, V and W independently are CH₂, CF₂, O, NR^{3A}, N or S.

Also particular preference is given to the pyrimidine compounds of formula I which corresponds to compounds of formulae I.m and I.n, and their use as herbicide, wherein X, and V independently are CH₂, CF₂, O, NR^{3A}, N or S, W independently is CH or N.

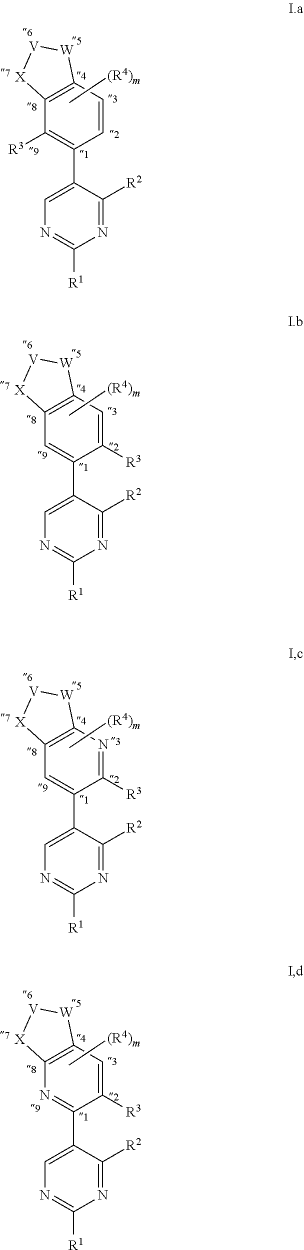

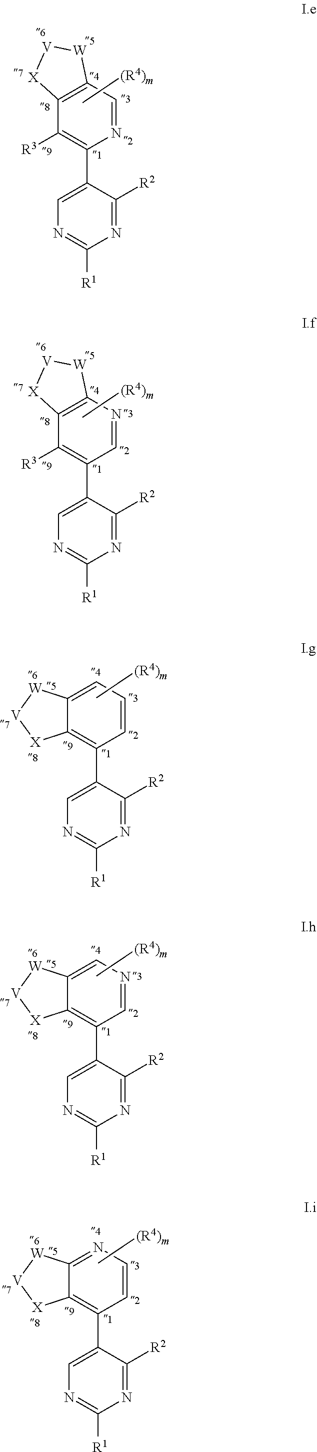

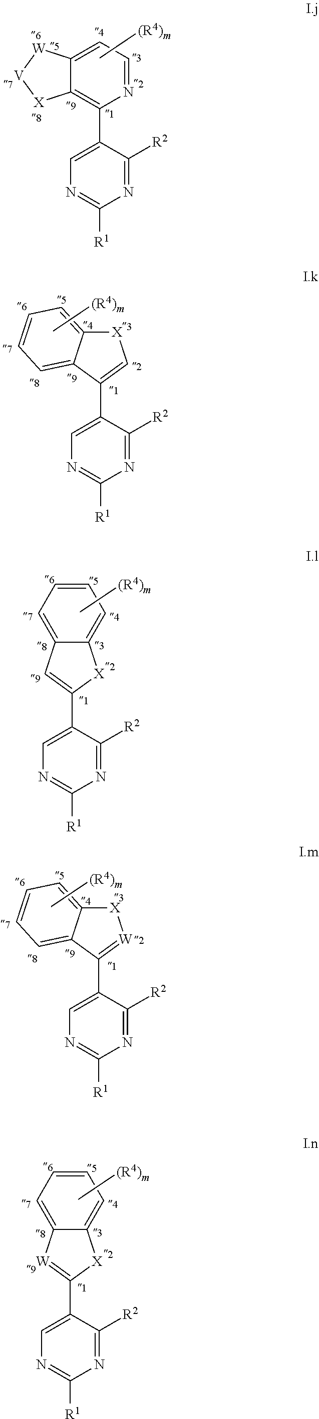

Most preferred compounds of formula I, and their use as herbicide, are the compounds of the formulae I.a to I.1 wherein

R¹ is C₂H₅, c-C₃H₅, c-C₄H₇, or OCH₃;

R² is CH═CH—CH₃, CH═C(CH₂)₃, CH═C(CH₂)₄, 2-furyl, 3-furyl, 3-methyl-2-furyl, 2-methyl-3-furyl, CHOH—CHOH—C₆H₅, CHOH—CHOH-2-furyl, or 4-methyloxazol-5-yl;

R³ is CH₃, OCH₃, Cl, Br, CHF₂, CF₃, F, or I;

X is CH₂O, S, or NCH₃;

V is CH₂ or CF₂;

W is CH₂, O, N, or S;

m is 0 or 1;

R⁴ is F, Cl or CF₃.

Also most preferred compounds of formula I, and their use as herbicide, are the compounds of the formulae I.m and I.n wherein

R¹ is C₂H₅, c-C₃H₅, c-C₄H₇, or OCH₃;

R² is CH═CH—CH₃, CH═C(CH₂)₃, CH═C(CH₂)₄, 2-furyl, 3-furyl, 3-methyl-2-furyl, 2-methyl-3-furyl, CHOH—CHOH—C₆H₅, CHOH—CHOH-2-furyl, or 4-methyloxazol-5-yl;

R³ is CH₃, OCH₃, Cl, Br, CHF₂, CF₃, F, or I;

X is CH₂O, S, or NCH₃;

V is CH₂ or CF₂;

W is CH or N;

m is 0 or 1;

R⁴ is F, Cl or CF₃.

Each of the groups mentioned for a substituent in the tables is furthermore per se, independently of the combination in which it is mentioned, a particularly preferred aspect of the substituent in question.

Particularly preferred compounds of formula I, and their use as herbicide, compounds of the invention are the compounds of the formulae that are compiled in the following Tables 1 to 52, and wherein the meaning for the combination of variables R¹, R², and R³ for each individual compound of tables 1 to 52 corresponds to each line of Table A.

Table 1. Compounds of formula I.a, wherein m is 0, V is CH₂, X and W are O, (=compounds of formula I.1).

Table 2. Compounds of formula I.a, wherein m is 1, R⁴ is 2″-Cl, V is CH₂, X and W are O, (=compounds of formula I.2).

Table 3. Compounds of formula I.b, wherein m is 0, V is CH₂, X and W are O, (=compounds of formula I.3).

Table 4. Compounds of formula I.b, wherein m is 1, R⁴ is 9″-Cl, V is CH₂, X and W are O, (=compounds of formula I.4).

Table 5. Compounds of formula I.c, wherein m is 0, V is CH₂, X and W are O, (=compounds of formula I.5).

Table 6. Compounds of formula I.c, wherein m is 1, R⁴ is 9″-Cl, V is CH₂, X and W are O, (=compounds of formula I.6).

Table 7. Compounds of formula I.d, wherein m is 0, V is CH₂, X and W are O, (=compounds of formula I.7).

Table 8. Compounds of formula I.d, wherein m is 1, R⁴ is 3″-Cl, V is CH₂, X and W are O, (=compounds of formula I.8).

Table 9. Compounds of formula I.e, wherein m is 0, V is CH₂, X and W are O, (=compounds of formula I.9).

Table 10. Compounds of formula I.e, wherein m is 1, R⁴ is 3″-Cl, V is CH₂, X and W are O, (=compounds of formula I.10).

Table 11. Compounds of formula I.f, wherein m is 0, V is CH₂, X and W are O, (=compounds of formula I.11).

Table 12. Compounds of formula I.f, wherein m is 1, R⁴ is 2″-Cl, V is CH₂, X and W are O, (=compounds of formula I.12).

Table 13. Compounds of formula I.g, wherein m is 0, V is CF₂, V is CH₂, X and W are O, (=compounds of formula I.13).

Table 14. Compounds of formula I.g, wherein m is 1, R⁴ is 2″-Cl, V is CF₂, X and W are O, (=compounds of formula I.14).

Table 15. Compounds of formula I.g, wherein m is 0, V is CH₂, X and W are O (=compounds of formula I.15).

Table 16. Compounds of formula I.g, wherein m is 1, R⁴ is 2″-Cl, V is CH₂, X and W are O, (=compounds of formula I.16).

Table 17. Compounds of formula I.g, wherein m is 0, V is CH₂, X is CH₂ and W is O, (=compounds of formula I.17).

Table 18. Compounds of formula I.g, wherein m is 1, R⁴ is 2″-Cl, V is CH₂, X is CH₂ and W is O (=compounds of formula I.18).

Table 19. Compounds of formula I.g, wherein m is 0, V, X, and W are CH₂, (=compounds of formula I.19).

Table 20. Compounds of formula I.g, wherein m is 1, R⁴ is 2″-Cl, V, X, and W are CH₂, (=compounds of formula I.20).

Table 21. Compounds of formula I.h, wherein m is 0, V is CF₂, X and W are O, (=compounds of formula I.21).

Table 22. Compounds of formula I.h, wherein m is 1, R⁴ is 2″-Cl, V is CF₂, X and W are O, (=compounds of formula I.22).

Table 23. Compounds of formula I.h, wherein m is 0, V is CH₂, X and W are O (=compounds of formula I.23).

Table 24. Compounds of formula I.h, wherein m is 1, R⁴ is 2″-Cl, V is CH₂, X and W are O, (=compounds of formula I.24).

Table 25. Compounds of formula I.h, wherein m is 0, V is CH₂, X is CH₂ and W is O, (=compounds of formula I.25).

Table 26. Compounds of formula I.h, wherein m is 1, R⁴ is 2″-Cl, V is CH₂, X is CH₂ (=compounds of formula I.26), and W is O. Table 27. Compounds of formula I.h, wherein m is 0, V, X, and W are CH₂, (=compounds of formula I.27).

Table 28. Compounds of formula I.h, wherein m is 1, R⁴ is 2″-Cl, V, X, and W are CH₂, (=compounds of formula I.28).

Table 29. Compounds of formula I.i, wherein m is 0, X and W are O (=compounds of formula I.29).

Table 30. Compounds of formula I.i, wherein m is 1, R⁴ is 2″-Cl, X and W are O (=compounds of formula I.30.

Table 31. Compounds of formula I.j, wherein m is 0, X and W are O (=compounds of formula I.31).

Table 32. Compounds of formula I.j, wherein m is 1, R⁴ is 3″-Cl, X and W are O (=compounds of formula I.32).

Table 33. Compounds of formula I.k, wherein m is 0, X is O (=compounds of formula I.33).

Table 34. Compounds of formula I.k, wherein m is 1, X is O, R⁴ is 2″-Cl (=compounds of formula I.34).

Table 35. Compounds of formula I.l, wherein m is 0, X is O (=compounds of formula I.35).

Table 36. Compounds of formula I.l, wherein m is 1, X is O, R⁴ is 9″-Cl (=compounds of formula I.36).

Table 37. Compounds of formula I.l, wherein m is 0, X is S (=compounds of formula I.37).

Table 38. Compounds of formula I.l, wherein m is 1, X is S, R⁴ is 9″-Cl (=compounds of formula I.38).

Table 39. Compounds of formula I.l, wherein m is 0, X is NCH₃ (=compounds of formula I.39).

Table 40. Compounds of formula I.l, wherein m is 1, X is NCH₃, R⁴ is 9″-Cl (=compounds of formula I.40).

Table 41. Compounds of formula I.m, wherein m is 0, X is O, W is N (=compounds of formula I.41).

Table 42. Compounds of formula I.m, wherein m is 1, X is O, W is N, R⁴ is 8″-Cl (=compounds of formula I.42).

Table 43. Compounds of formula I.m, wherein m is 0, X is S, W is N (=compounds of formula I.43).

Table 44. Compounds of formula I.m, wherein m is 1, X is S, W is N, R⁴ is 8″-Cl (=compounds of formula I.44).

Table 45. Compounds of formula I.m, wherein m is 0, X is NCH₃, W is N (=compounds of formula I.45).

Table 46. Compounds of formula I.m, wherein m is 1, X is NCH₃, W is N, R⁴ is 8″-Cl (=compounds of formula I.46).

Table 47. Compounds of formula I.n, wherein m is 0, X is O, W is N (=compounds of formula I.47).

Table 48. Compounds of formula I.n, wherein m is 1, X is O, W is N, R⁴ is 7″-Cl (=compounds of formula I.48).

Table 49. Compounds of formula I.n, wherein m is 0, X is S, W is N (=compounds of formula I.49).

Table 50. Compounds of formula I.n, wherein m is 1, X is S, W is N, R⁴ is 7″-Cl (=compounds of formula I.50).

Table 51. Compounds of formula I.n, wherein m is 0, X is NCH₃, W is N (=compounds of formula I.51).

Table 52. Compounds of formula I.n, wherein m is 1, X is NCH₃, W is N, R⁴ is 7″-Cl (=compounds of formula I.52).

TABLE A

| Line | R¹ | R² | R³ |
|---|---|---|---|
| I-1 | c-C₃H₅ | CH═CH—CH₃ | CH₃ |
| I-2 | c-C₃H₅ | CH═CH—CH₃ | OCH₃ |
| I-3 | c-C₃H₅ | CH═CH—CH₃ | Cl |
| I-4 | c-C₃H₅ | CH═CH—CH₃ | Br |
| I-5 | c-C₃H₅ | CH═CH—CH₃ | CHF₂ |
| I-6 | c-C₃H₅ | CH═CH—CH₃ | F |
| I-7 | c-C₃H₅ | CH═CH—CH₃ | I |
| I-8 | c-C₃H₅ | CH═C(CH₂)₃ | CH₃ |
| I-9 | c-C₃H₅ | CH═C(CH₂)₃ | OCH₃ |
| I-10 | c-C₃H₅ | CH═C(CH₂)₃ | Cl |
| I-11 | c-C₃H₅ | CH═C(CH₂)₃ | Br |
| I-12 | c-C₃H₅ | CH═C(CH₂)₃ | CHF₂ |
| I-13 | c-C₃H₅ | CH═C(CH₂)₃ | F |
| I-14 | c-C₃H₅ | CH═C(CH₂)₃ | I |
| I-15 | c-C₃H₅ | CH═C(CH₂)₄ | CH₃ |
| I-16 | c-C₃H₅ | CH═C(CH₂)₄ | OCH₃ |
| I-17 | c-C₃H₅ | CH═C(CH₂)₄ | Cl |
| I-18 | c-C₃H₅ | CH═C(CH₂)₄ | Br |
| I-19 | c-C₃H₅ | CH═C(CH₂)₄ | CHF₂ |
| I-20 | c-C₃H₅ | CH═C(CH₂)₄ | F |
| I-21 | c-C₃H₅ | CH═C(CH₂)₄ | I |
| I-22 | c-C₃H₅ | 2-furyl | CH₃ |
| I-23 | c-C₃H₅ | 2-furyl | OCH₃ |
| I-24 | c-C₃H₅ | 2-furyl | Cl |
| I-25 | c-C₃H₅ | 2-furyl | Br |
| I-26 | c-C₃H₅ | 2-furyl | CHF₂ |
| I-27 | c-C₃H₅ | 2-furyl | F |
| I-28 | c-C₃H₅ | 2-furyl | I |
| I-29 | c-C₃H₅ | 3-furyl | CH₃ |
| I-30 | c-C₃H₅ | 3-furyl | OCH₃ |
| I-31 | c-C₃H₅ | 3-furyl | Cl |
| I-32 | c-C₃H₅ | 3-furyl | Br |
| I-33 | c-C₃H₅ | 3-furyl | CHF₂ |
| I-34 | c-C₃H₅ | 3-furyl | F |
| I-35 | c-C₃H₅ | 3-furyl | I |
| I-36 | c-C₃H₅ | 3-methyl-2-furyl | CH₃ |
| I-37 | c-C₃H₅ | 3-methyl-2-furyl | OCH₃ |
| I-38 | c-C₃H₅ | 3-methyl-2-furyl | Cl |
| I-39 | c-C₃H₅ | 3-methyl-2-furyl | Br |
| I-40 | c-C₃H₅ | 3-methyl-2-furyl | CHF₂ |
| I-41 | c-C₃H₅ | 3-methyl-2-furyl | F |
| I-42 | c-C₃H₅ | 3-methyl-2-furyl | I |
| I-43 | c-C₃H₅ | 2-methyl-3-furyl | CH₃ |
| I-44 | c-C₃H₅ | 2-methyl-3-furyl | OCH₃ |
| I-45 | c-C₃H₅ | 2-methyl-3-furyl | Cl |
| I-46 | c-C₃H₅ | 2-methyl-3-furyl | Br |
| I-47 | c-C₃H₅ | 2-methyl-3-furyl | CHF₂ |
| I-48 | c-C₃H₅ | 2-methyl-3-furyl | F |
| I-49 | c-C₃H₅ | 2-methyl-3-furyl | I |
| I-50 | c-C₃H₅ | CHOH—CHOH—C₆H₅ | CH₃ |
| I-51 | c-C₃H₅ | CHOH—CHOH—C₆H₅ | OCH₃ |
| I-52 | c-C₃H₅ | CHOH—CHOH—C₆H₅ | Cl |
| I-53 | c-C₃H₅ | CHOH—CHOH—C₆H₅ | Br |
| I-54 | c-C₃H₅ | CHOH—CHOH—C₆H₅ | CHF₂ |
| I-55 | c-C₃H₅ | CHOH—CHOH—C₆H₅ | F |
| I-56 | c-C₃H₅ | CHOH—CHOH—C₆H₅ | I |
| I-57 | c-C₃H₅ | CHOH—CHOH-2-furyl | CH₃ |
| I-58 | c-C₃H₅ | CHOH—CHOH-2-furyl | OCH₃ |
| I-59 | c-C₃H₅ | CHOH—CHOH-2-furyl | Cl |
| I-60 | c-C₃H₅ | CHOH—CHOH-2-furyl | Br |
| I-61 | c-C₃H₅ | CHOH—CHOH-2-furyl | CHF₂ |
| I-62 | c-C₃H₅ | CHOH—CHOH-2-furyl | F |
| I-63 | c-C₃H₅ | CHOH—CHOH-2-furyl | I |
| I-64 | c-C₃H₅ | 4-methyl-5-oxazolyl | CH₃ |
| I-65 | c-C₃H₅ | 4-methyl-5-oxazolyl | OCH₃ |
| I-66 | c-C₃H₅ | 4-methyl-5-oxazolyl | Cl |
| I-67 | c-C₃H₅ | 4-methyl-5-oxazolyl | Br |
| I-68 | c-C₃H₅ | 4-methyl-5-oxazolyl | CHF₂ |
| I-69 | c-C₃H₅ | 4-methyl-5-oxazolyl | F |
| I-70 | c-C₃H₅ | 4-methyl-5-oxazolyl | I |
| I-71 | c-C₄H₇ | CH═CH—CH₃ | CH₃ |
| I-72 | c-C₄H₇ | CH═CH—CH₃ | OCH₃ |
| I-73 | c-C₄H₇ | CH═CH—CH₃ | Cl |
| I-74 | c-C₄H₇ | CH═CH—CH₃ | Br |
| I-75 | c-C₄H₇ | CH═CH—CH₃ | CHF₂ |
| I-76 | c-C₄H₇ | CH═CH—CH₃ | F |
| I-77 | c-C₄H₇ | CH═CH—CH₃ | I |
| I-78 | c-C₄H₇ | CH═C(CH₂)₃ | CH₃ |
| I-79 | c-C₄H₇ | CH═C(CH₂)₃ | OCH₃ |
| I-80 | c-C₄H₇ | CH═C(CH₂)₃ | Cl |
| I-81 | c-C₄H₇ | CH═C(CH₂)₃ | Br |
| I-82 | c-C₄H₇ | CH═C(CH₂)₃ | CHF₂ |
| I-83 | c-C₄H₇ | CH═C(CH₂)₃ | F |
| I-84 | c-C₄H₇ | CH═C(CH₂)₃ | I |
| I-85 | c-C₄H₇ | CH═C(CH₂)₄ | CH₃ |
| I-86 | c-C₄H₇ | CH═C(CH₂)₄ | OCH₃ |
| I-87 | c-C₄H₇ | CH═C(CH₂)₄ | Cl |
| I-88 | c-C₄H₇ | CH═C(CH₂)₄ | Br |
| I-89 | c-C₄H₇ | CH═C(CH₂)₄ | CHF₂ |
| I-90 | c-C₄H₇ | CH═C(CH₂)₄ | F |
| I-91 | c-C₄H₇ | CH═C(CH₂)₄ | I |
| I-92 | c-C₄H₇ | 2-furyl | CH₃ |
| I-93 | c-C₄H₇ | 2-furyl | OCH₃ |
| I-94 | c-C₄H₇ | 2-furyl | Cl |
| I-95 | c-C₄H₇ | 2-furyl | Br |
| I-96 | c-C₄H₇ | 2-furyl | CHF₂ |
| I-97 | c-C₄H₇ | 2-furyl | F |
| I-98 | c-C₄H₇ | 2-furyl | I |
| I-99 | c-C₄H₇ | 3-furyl | CH₃ |
| I-100 | c-C₄H₇ | 3-furyl | OCH₃ |
| I-101 | c-C₄H₇ | 3-furyl | Cl |
| I-102 | c-C₄H₇ | 3-furyl | Br |
| I-103 | c-C₄H₇ | 3-furyl | CHF₂ |
| I-104 | c-C₄H₇ | 3-furyl | F |
| I-105 | c-C₄H₇ | 3-furyl | I |
| I-106 | c-C₄H₇ | 3-methyl-2-furyl | CH₃ |
| I-107 | c-C₄H₇ | 3-methyl-2-furyl | OCH₃ |
| I-108 | c-C₄H₇ | 3-methyl-2-furyl | Cl |
| I-109 | c-C₄H₇ | 3-methyl-2-furyl | Br |
| I-110 | c-C₄H₇ | 3-methyl-2-furyl | CHF₂ |
| I-111 | c-C₄H₇ | 3-methyl-2-furyl | F |
| I-112 | c-C₄H₇ | 3-methyl-2-furyl | I |
| I-113 | c-C₄H₇ | 2-methyl-3-furyl | CH₃ |
| I-114 | c-C₄H₇ | 2-methyl-3-furyl | OCH₃ |
| I-115 | c-C₄H₇ | 2-methyl-3-furyl | Cl |
| I-116 | c-C₄H₇ | 2-methyl-3-furyl | Br |
| I-117 | c-C₄H₇ | 2-methyl-3-furyl | CHF₂ |
| I-118 | c-C₄H₇ | 2-methyl-3-furyl | F |
| I-119 | c-C₄H₇ | 2-methyl-3-furyl | I |
| I-120 | c-C₄H₇ | CHOH—CHOH—C₆H₅ | CH₃ |
| I-121 | c-C₄H₇ | CHOH—CHOH—C₆H₅ | OCH₃ |
| I-122 | c-C₄H₇ | CHOH—CHOH—C₆H₅ | Cl |
| I-123 | c-C₄H₇ | CHOH—CHOH—C₆H₅ | Br |
| I-124 | c-C₄H₇ | CHOH—CHOH—C₆H₅ | CHF₂ |
| I-125 | c-C₄H₇ | CHOH—CHOH—C₆H₅ | F |
| I-126 | c-C₄H₇ | CHOH—CHOH—C₆H₅ | I |
| I-127 | c-C₄H₇ | CHOH—CHOH-2-furyl | CH₃ |
| I-128 | c-C₄H₇ | CHOH—CHOH-2-furyl | OCH₃ |
| I-129 | c-C₄H₇ | CHOH—CHOH-2-furyl | Cl |
| I-130 | c-C₄H₇ | CHOH—CHOH-2-furyl | Br |
| I-131 | c-C₄H₇ | CHOH—CHOH-2-furyl | CHF₂ |
| I-132 | c-C₄H₇ | CHOH—CHOH-2-furyl | F |
| I-133 | c-C₄H₇ | CHOH—CHOH-2-furyl | I |
| I-134 | c-C₄H₇ | 4-methyl-5-oxazolyl | CH₃ |
| I-135 | c-C₄H₇ | 4-methyl-5-oxazolyl | OCH₃ |
| I-136 | c-C₄H₇ | 4-methyl-5-oxazolyl | Cl |
| I-137 | c-C₄H₇ | 4-methyl-5-oxazolyl | Br |
| I-138 | c-C₄H₇ | 4-methyl-5-oxazolyl | CHF₂ |
| I-139 | c-C₄H₇ | 4-methyl-5-oxazolyl | F |
| I-140 | c-C₄H₇ | 4-methyl-5-oxazolyl | I |
| I-141 | C₂H₅ | CH═CH—CH₃ | CH₃ |
| I-142 | C₂H₅ | CH═CH—CH₃ | OCH₃ |
| I-143 | C₂H₅ | CH═CH—CH₃ | Cl |
| I-144 | C₂H₅ | CH═CH—CH₃ | Br |
| I-145 | C₂H₅ | CH═CH—CH₃ | CHF₂ |
| I-146 | C₂H₅ | CH═CH—CH₃ | F |
| I-147 | C₂H₅ | CH═CH—CH₃ | I |
| I-148 | C₂H₅ | CH═C(CH₂)₃ | CH₃ |
| I-149 | C₂H₅ | CH═C(CH₂)₃ | OCH₃ |
| I-150 | C₂H₅ | CH═C(CH₂)₃ | Cl |
| I-151 | C₂H₅ | CH═C(CH₂)₃ | Br |
| I-152 | C₂H₅ | CH═C(CH₂)₃ | CHF₂ |
| I-153 | C₂H₅ | CH═C(CH₂)₃ | F |
| I-154 | C₂H₅ | CH═C(CH₂)₃ | I |
| I-155 | C₂H₅ | CH═C(CH₂)₄ | CH₃ |
| I-156 | C₂H₅ | CH═C(CH₂)₄ | OCH₃ |
| I-157 | C₂H₅ | CH═C(CH₂)₄ | Cl |
| I-158 | C₂H₅ | CH═C(CH₂)₄ | Br |
| I-159 | C₂H₅ | CH═C(CH₂)₄ | CHF₂ |
| I-160 | C₂H₅ | CH═C(CH₂)₄ | F |
| I-161 | C₂H₅ | CH═C(CH₂)₄ | I |
| I-162 | C₂H₅ | 2-furyl | CH₃ |
| I-163 | C₂H₅ | 2-furyl | OCH₃ |
| I-164 | C₂H₅ | 2-furyl | Cl |
| I-165 | C₂H₅ | 2-furyl | Br |
| I-166 | C₂H₅ | 2-furyl | CHF₂ |
| I-167 | C₂H₅ | 2-furyl | F |
| I-168 | C₂H₅ | 2-furyl | I |
| I-169 | C₂H₅ | 3-furyl | CH₃ |
| I-170 | C₂H₅ | 3-furyl | OCH₃ |
| I-171 | C₂H₅ | 3-furyl | Cl |
| I-172 | C₂H₅ | 3-furyl | Br |
| I-173 | C₂H₅ | 3-furyl | CHF₂ |
| I-174 | C₂H₅ | 3-furyl | F |
| I-175 | C₂H₅ | 3-furyl | I |
| I-176 | C₂H₅ | 3-methyl-2-furyl | CH₃ |
| I-177 | C₂H₅ | 3-methyl-2-furyl | OCH₃ |
| I-178 | C₂H₅ | 3-methyl-2-furyl | Cl |
| I-179 | C₂H₅ | 3-methyl-2-furyl | Br |
| I-180 | C₂H₅ | 3-methyl-2-furyl | CHF₂ |
| I-181 | C₂H₅ | 3-methyl-2-furyl | F |
| I-182 | C₂H₅ | 3-methyl-2-furyl | I |
| I-183 | C₂H₅ | 2-methyl-3-furyl | CH₃ |
| I-184 | C₂H₅ | 2-methyl-3-furyl | OCH₃ |
| I-185 | C₂H₅ | 2-methyl-3-furyl | Cl |
| I-186 | C₂H₅ | 2-methyl-3-furyl | Br |
| I-187 | C₂H₅ | 2-methyl-3-furyl | CHF₂ |
| I-188 | C₂H₅ | 2-methyl-3-furyl | F |
| I-189 | C₂H₅ | 2-methyl-3-furyl | I |
| I-190 | C₂H₅ | CHOH—CHOH—C₆H₅ | CH₃ |
| I-191 | C₂H₅ | CHOH—CHOH—C₆H₅ | OCH₃ |
| I-192 | C₂H₅ | CHOH—CHOH—C₆H₅ | Cl |
| I-193 | C₂H₅ | CHOH—CHOH—C₆H₅ | Br |
| I-194 | C₂H₅ | CHOH—CHOH—C₆H₅ | CHF₂ |
| I-195 | C₂H₅ | CHOH—CHOH—C₆H₅ | F |
| I-196 | C₂H₅ | CHOH—CHOH—C₆H₅ | I |
| I-197 | C₂H₅ | CHOH—CHOH-2-furyl | CH₃ |
| I-198 | C₂H₅ | CHOH—CHOH-2-furyl | OCH₃ |
| I-199 | C₂H₅ | CHOH—CHOH-2-furyl | Cl |
| I-200 | C₂H₅ | CHOH—CHOH-2-furyl | Br |
| I-201 | C₂H₅ | CHOH—CHOH-2-furyl | CHF₂ |
| I-202 | C₂H₅ | CHOH—CHOH-2-furyl | F |
| I-203 | C₂H₅ | CHOH—CHOH-2-furyl | I |
| I-204 | C₂H₅ | 4-methyl-5-oxazolyl | CH₃ |
| I-205 | C₂H₅ | 4-methyl-5-oxazolyl | OCH₃ |
| I-206 | C₂H₅ | 4-methyl-5-oxazolyl | Cl |
| I-207 | C₂H₅ | 4-methyl-5-oxazolyl | Br |
| I-208 | C₂H₅ | 4-methyl-5-oxazolyl | CHF₂ |
| I-209 | C₂H₅ | 4-methyl-5-oxazolyl | F |
| I-210 | C₂H₅ | 4-methyl-5-oxazolyl | I |
| I-211 | OCH₃ | CH═CH—CH₃ | CH₃ |
| I-212 | OCH₃ | CH═CH—CH₃ | OCH₃ |
| I-213 | OCH₃ | CH═CH—CH₃ | Cl |
| I-214 | OCH₃ | CH═CH—CH₃ | Br |
| I-215 | OCH₃ | CH═CH—CH₃ | CHF₂ |
| I-216 | OCH₃ | CH═CH—CH₃ | F |
| I-217 | OCH₃ | CH═CH—CH₃ | I |
| I-218 | OCH₃ | CH═C(CH₂)₃ | CH₃ |
| I-219 | OCH₃ | CH═C(CH₂)₃ | OCH₃ |
| I-220 | OCH₃ | CH═C(CH₂)₃ | Cl |
| I-221 | OCH₃ | CH═C(CH₂)₃ | Br |
| I-222 | OCH₃ | CH═C(CH₂)₃ | CHF₂ |
| I-223 | OCH₃ | CH═C(CH₂)₃ | F |
| I-224 | OCH₃ | CH═C(CH₂)₃ | I |
| I-225 | OCH₃ | CH═C(CH₂)₄ | CH₃ |
| I-226 | OCH₃ | CH═C(CH₂)₄ | OCH₃ |
| I-227 | OCH₃ | CH═C(CH₂)₄ | Cl |
| I-228 | OCH₃ | CH═C(CH₂)₄ | Br |
| I-229 | OCH₃ | CH═C(CH₂)₄ | CHF₂ |
| I-230 | OCH₃ | CH═C(CH₂)₄ | F |
| I-231 | OCH₃ | CH═C(CH₂)₄ | I |
| I-232 | OCH₃ | 2-furyl | CH₃ |
| I-233 | OCH₃ | 2-furyl | OCH₃ |
| I-234 | OCH₃ | 2-furyl | Cl |
| I-235 | OCH₃ | 2-furyl | Br |
| I-236 | OCH₃ | 2-furyl | CHF₂ |
| I-237 | OCH₃ | 2-furyl | F |
| I-238 | OCH₃ | 2-furyl | I |
| I-239 | OCH₃ | 3-furyl | CH₃ |
| I-240 | OCH₃ | 3-furyl | OCH₃ |
| I-241 | OCH₃ | 3-furyl | Cl |
| I-242 | OCH₃ | 3-furyl | Br |
| I-243 | OCH₃ | 3-furyl | CHF₂ |
| I-244 | OCH₃ | 3-furyl | F |
| I-245 | OCH₃ | 3-furyl | I |
| I-246 | OCH₃ | 3-methyl-2-furyl | CH₃ |
| I-247 | OCH₃ | 3-methyl-2-furyl | OCH₃ |
| I-248 | OCH₃ | 3-methyl-2-furyl | Cl |
| I-249 | OCH₃ | 3-methyl-2-furyl | Br |
| I-250 | OCH₃ | 3-methyl-2-furyl | CHF₂ |
| I-251 | OCH₃ | 3-methyl-2-furyl | F |
| I-252 | OCH₃ | 3-methyl-2-furyl | I |
| I-253 | OCH₃ | 2-methyl-3-furyl | CH₃ |
| I-254 | OCH₃ | 2-methyl-3-furyl | OCH₃ |
| I-255 | OCH₃ | 2-methyl-3-furyl | Cl |
| I-256 | OCH₃ | 2-methyl-3-furyl | Br |
| I-257 | OCH₃ | 2-methyl-3-furyl | CHF₂ |
| I-258 | OCH₃ | 2-methyl-3-furyl | F |
| I-259 | OCH₃ | 2-methyl-3-furyl | I |
| I-260 | OCH₃ | CHOH—CHOH—C₆H₅ | CH₃ |
| I-261 | OCH₃ | CHOH—CHOH—C₆H₅ | OCH₃ |
| I-262 | OCH₃ | CHOH—CHOH—C₆H₅ | Cl |
| I-263 | OCH₃ | CHOH—CHOH—C₆H₅ | Br |
| I-264 | OCH₃ | CHOH—CHOH—C₆H₅ | CHF₂ |
| I-265 | OCH₃ | CHOH—CHOH—C₆H₅ | F |
| I-266 | OCH₃ | CHOH—CHOH—C₆H₅ | I |
| I-267 | OCH₃ | CHOH—CHOH-2-furyl | CH₃ |
| I-268 | OCH₃ | CHOH—CHOH-2-furyl | OCH₃ |
| I-269 | OCH₃ | CHOH—CHOH-2-furyl | Cl |
| I-270 | OCH₃ | CHOH—CHOH-2-furyl | Br |
| I-271 | OCH₃ | CHOH—CHOH-2-furyl | CHF₂ |
| I-272 | OCH₃ | CHOH—CHOH-2-furyl | F |
| I-273 | OCH₃ | CHOH—CHOH-2-furyl | I |
| I-274 | OCH₃ | 4-methyl-5-oxazolyl | CH₃ |
| I-275 | OCH₃ | 4-methyl-5-oxazolyl | OCH₃ |
| I-276 | OCH₃ | 4-methyl-5-oxazolyl | Cl |
| I-277 | OCH₃ | 4-methyl-5-oxazolyl | Br |
| I-278 | OCH₃ | 4-methyl-5-oxazolyl | CHF₂ |
| I-279 | OCH₃ | 4-methyl-5-oxazolyl | F |
| I-280 | OCH₃ | 4-methyl-5-oxazolyl | I |

The specific number for each single compound is deductible as follows:

Compound 1.1.1-3 e.g. comprises the compound of formula I.1 from Table 1 and line I-3 from Table A;

According to another particularly preferred embodiment of the compounds of formula 1 and their use as herbicide, compounds of the invention are the compounds of formulae I.b, wherein

V is CH₂;

X and W are O;

R¹ is C₂H₅, c-C₃H₅, c-C₄H₇, or OCH₃;

R² is selected from R²-9.1 to R²-9.676 from Table R²-9, R²-10.1 to R²-10.676 from Table R²-10, and R²-15.1 to R²-15.676 from Table R²-15; preferably R²-9.1 to R²-9.676 from Table R²-9,

R³ is CH₃, OCH₃, Cl, Br, CHF₂, F, or I;

m is 0 or 1;

R⁴ is C1.

According to another particularly preferred embodiment of the compounds of formula I and their use as herbicide, compounds of the invention are the compounds of formulae I.b that are compiled in tables 53 to 2080, wherein V is CH₂, X and W are O, and the meaning for the combination of variables R¹, R³, m, and R⁴ for each individual compound of tables 53 to 2080 corresponds to each line of Table A1. Each of the groups mentioned for a substituent in the tables is furthermore per se, independently of the combination in which it is mentioned, a particularly preferred aspect of the substituent in question.

The term “formula” used in below tables 53 to 2080 denotes “compounds of formula”.

Table 53. Compounds of formula I.b, wherein R² is R²-9.43 (=formula 1.53).

Table 54. Compounds of formula I.b, wherein R² is R²-9.44 (=formula 1.54).

Table 55. Compounds of formula I.b, wherein R² is R²-9.45 (=formula 1.55).

Table 56. Compounds of formula I.b, wherein R² is R²-9.46 (=formula 1.56).

Table 57. Compounds of formula I.b, wherein R² is R²-9.47 (=formula 1.57).

Table 58. Compounds of formula I.b, wherein R² is R²-9.48 (=formula 1.58).

Table 59. Compounds of formula I.b, wherein R² is R²-9.49 (=formula 1.59).

Table 60. Compounds of formula I.b, wherein R² is R²-9.50 (=formula 1.60).

Table 61. Compounds of formula I.b, wherein R² is R²-9.51 (=formula 1.61).

Table 62. Compounds of formula I.b, wherein R² is R²-9.52 (=formula 1.62).

Table 63. Compounds of formula I.b, wherein R² is R²-9.53 (=formula 1.63).

Table 64. Compounds of formula I.b, wherein R² is R²-9.54 (=formula 1.64).

Table 65. Compounds of formula I.b, wherein R² is R²-9.55 (=formula 1.65).

Table 66. Compounds of formula I.b, wherein R² is R²-9.56 (=formula 1.66).

Table 67. Compounds of formula I.b, wherein R² is R²-9.57 (=formula 1.67).

Table 68. Compounds of formula I.b, wherein R² is R²-9.58 (=formula 1.68).

Table 69. Compounds of formula I.b, wherein R² is R²-9.59 (=formula 1.69).

Table 70. Compounds of formula I.b, wherein R² is R²-9.60 (=formula 1.70).

Table 71. Compounds of formula I.b, wherein R² is R²-9.61 (=formula 1.71).

Table 72. Compounds of formula I.b, wherein R² is R²-9.62 (=formula 1.72).

Table 73. Compounds of formula I.b, wherein R² is R²-9.63 (=formula 1.73).

Table 74. Compounds of formula I.b, wherein R² is R²-9.64 (=formula 1.74).

Table 75. Compounds of formula I.b, wherein R² is R²-9.65 (=formula 1.75).

Table 76. Compounds of formula I.b, wherein R² is R²-9.66 (=formula 1.76).

Table 77. Compounds of formula I.b, wherein R² is R²-9.67 (=formula 1.77).

Table 78. Compounds of formula I.b, wherein R² is R²-9.68 (=formula 1.78).

Table 79. Compounds of formula I.b, wherein R² is R²-9.69 (=formula 1.79).

Table 80. Compounds of formula I.b, wherein R² is R²-9.70 (=formula 1.80).

Table 81. Compounds of formula I.b, wherein R² is R²-9.71 (=formula 1.81).

Table 82. Compounds of formula I.b, wherein R² is R²-9.72 (=formula 1.82).

Table 83. Compounds of formula I.b, wherein R² is R²-9.73 (=formula 1.83).

Table 84. Compounds of formula I.b, wherein R² is R²-9.74 (=formula 1.84).

Table 85. Compounds of formula I.b, wherein R² is R²-9.75 (=formula 1.85).

Table 86. Compounds of formula I.b, wherein R² is R²-9.76 (=formula 1.86).

Table 87. Compounds of formula I.b, wherein R² is R²-9.77 (=formula 1.87).

Table 88. Compounds of formula I.b, wherein R² is R²-9.78 (=formula 1.88).

Table 89. Compounds of formula I.b, wherein R² is R²-9.79 (=formula 1.89).

Table 90. Compounds of formula I.b, wherein R² is R²-9.80 (=formula 1.90).

Table 91. Compounds of formula I.b, wherein R² is R²-9.81 (=formula 1.91).

Table 92. Compounds of formula I.b, wherein R² is R²-9.82 (=formula 1.92).

Table 93. Compounds of formula I.b, wherein R² is R²-9.83 (=formula 1.93).

Table 94. Compounds of formula I.b, wherein R² is R²-9.84 (=formula 1.94).

Table 95. Compounds of formula I.b, wherein R² is R²-9.85 (=formula 1.95).

Table 96. Compounds of formula I.b, wherein R² is R²-9.86 (=formula 1.96).

Table 97. Compounds of formula I.b, wherein R² is R²-9.87 (=formula 1.97).

Table 98. Compounds of formula I.b, wherein R² is R²-9.88 (=formula 1.98).

Table 99. Compounds of formula I.b, wherein R² is R²-9.89 (=formula 1.99).

Table 100. Compounds of formula I.b, wherein R² is R²-9.90 (=formula 1.100).

Table 101. Compounds of formula I.b, wherein R² is R²-9.91 (=formula 1.101).

Table 102. Compounds of formula I.b, wherein R² is R²-9.92 (=formula 1.102).

Table 103. Compounds of formula I.b, wherein R² is R²-9.93 (=formula 1.103).

Table 104. Compounds of formula I.b, wherein R² is R²-9.94 (=formula 1.104).

Table 105. Compounds of formula I.b, wherein R² is R²-9.95 (=formula 1.105).

Table 106. Compounds of formula I.b, wherein R² is R²-9.96 (=formula 1.106).

Table 107. Compounds of formula I.b, wherein R² is R²-9.97 (=formula 1.107).

Table 108. Compounds of formula I.b, wherein R² is R²-9.98 (=formula 1.108).

Table 109. Compounds of formula I.b, wherein R² is R²-9.99 (=formula 1.109).

Table 110. Compounds of formula I.b, wherein R² is R²-9.100 (=formula 1.110).

Table 111. Compounds of formula I.b, wherein R² is R²-9.101 (=formula 1.111).

Table 112. Compounds of formula I.b, wherein R² is R²-9.102 (=formula 1.112).

Table 113. Compounds of formula I.b, wherein R² is R²-9.103 (=formula 1.113).

Table 114. Compounds of formula I.b, wherein R² is R²-9.104 (=formula 1.114).

Table 115. Compounds of formula I.b, wherein R² is R²-9.105 (=formula 1.115).

Table 116. Compounds of formula I.b, wherein R² is R²-9.106 (=formula 1.116).

Table 117. Compounds of formula I.b, wherein R² is R²-9.107 (=formula 1.117).

Table 118. Compounds of formula I.b, wherein R² is R²-9.108 (=formula 1.118).

Table 119. Compounds of formula I.b, wherein R² is R²-9.109 (=formula 1.119).

Table 120. Compounds of formula I.b, wherein R² is R²-9.110 (=formula 1.120).

Table 121. Compounds of formula I.b, wherein R² is R²-9.111 (=formula 1.121).

Table 122. Compounds of formula I.b, wherein R² is R²-9.112 (=formula 1.122).

Table 123. Compounds of formula I.b, wherein R² is R²-9.113 (=formula 1.123).

Table 124. Compounds of formula I.b, wherein R² is R²-9.114 (=formula 1.124).

Table 125. Compounds of formula I.b, wherein R² is R²-9.115 (=formula 1.125).

Table 126. Compounds of formula I.b, wherein R² is R²-9.116 (=formula 1.126).

Table 127. Compounds of formula I.b, wherein R² is R²-9.117 (=formula 1.127).

Table 128. Compounds of formula I.b, wherein R² is R²-9.118 (=formula 1.128).

Table 129. Compounds of formula I.b, wherein R² is R²-9.119 (=formula 1.129).

Table 130. Compounds of formula I.b, wherein R² is R²-9.120 (=formula 1.130).

Table 131. Compounds of formula I.b, wherein R² is R²-9.121 (=formula 1.131).

Table 132. Compounds of formula I.b, wherein R² is R²-9.122 (=formula 1.132).

Table 133. Compounds of formula I.b, wherein R² is R²-9.123 (=formula 1.133).

Table 134. Compounds of formula I.b, wherein R² is R²-9.124 (=formula 1.134).

Table 135. Compounds of formula I.b, wherein R² is R²-9.125 (=formula 1.135).

Table 136. Compounds of formula I.b, wherein R² is R²-9.126 (=formula 1.136).

Table 137. Compounds of formula I.b, wherein R² is R²-9.127 (=formula 1.137).

Table 138. Compounds of formula I.b, wherein R² is R²-9.128 (=formula 1.138).

Table 139. Compounds of formula I.b, wherein R² is R²-9.129 (=formula 1.139).

Table 140. Compounds of formula I.b, wherein R² is R²-9.130 (=formula 1.140).

Table 141. Compounds of formula I.b, wherein R² is R²-9.131 (=formula 1.141).

Table 142. Compounds of formula I.b, wherein R² is R²-9.132 (=formula 1.142).

Table 143. Compounds of formula I.b, wherein R² is R²-9.133 (=formula 1.143).

Table 144. Compounds of formula I.b, wherein R² is R²-9.134 (=formula 1.144).

Table 145. Compounds of formula I.b, wherein R² is R²-9.135 (=formula 1.145).

Table 146. Compounds of formula I.b, wherein R² is R²-9.136 (=formula 1.146).

Table 147. Compounds of formula I.b, wherein R² is R²-9.137 (=formula 1.147).

Table 148. Compounds of formula I.b, wherein R² is R²-9.138 (=formula 1.148).

Table 149. Compounds of formula I.b, wherein R² is R²-9.139 (=formula 1.149).

Table 150. Compounds of formula I.b, wherein R² is R²-9.140 (=formula 1.150).

Table 151. Compounds of formula I.b, wherein R² is R²-9.141 (=formula 1.151).

Table 152. Compounds of formula I.b, wherein R² is R²-9.142 (=formula 1.152).

Table 153. Compounds of formula I.b, wherein R² is R²-9.143 (=formula 1.153).

Table 154. Compounds of formula I.b, wherein R² is R²-9.144 (=formula 1.154).

Table 155. Compounds of formula I.b, wherein R² is R²-9.145 (=formula 1.155).

Table 156. Compounds of formula I.b, wherein R² is R²-9.146 (=formula 1.156).

Table 157. Compounds of formula I.b, wherein R² is R²-9.147 (=formula 1.157).

Table 158. Compounds of formula I.b, wherein R² is R²-9.148 (=formula 1.158).

Table 159. Compounds of formula I.b, wherein R² is R²-9.149 (=formula 1.159).

Table 160. Compounds of formula I.b, wherein R² is R²-9.150 (=formula 1.160).

Table 161. Compounds of formula I.b, wherein R² is R²-9.151 (=formula 1.161).

Table 162. Compounds of formula I.b, wherein R² is R²-9.152 (=formula 1.162).

Table 163. Compounds of formula I.b, wherein R² is R²-9.153 (=formula 1.163).

Table 164. Compounds of formula I.b, wherein R² is R²-9.154 (=formula 1.164).

Table 165. Compounds of formula I.b, wherein R² is R²-9.155 (=formula 1.165).

Table 166. Compounds of formula I.b, wherein R² is R²-9.156 (=formula 1.166).

Table 167. Compounds of formula I.b, wherein R² is R²-9.157 (=formula 1.167).

Table 168. Compounds of formula I.b, wherein R² is R²-9.158 (=formula 1.168).

Table 169. Compounds of formula I.b, wherein R² is R²-9.159 (=formula 1.169).

Table 170. Compounds of formula I.b, wherein R² is R²-9.160 (=formula 1.170).

Table 171. Compounds of formula I.b, wherein R² is R²-9.161 (=formula 1.171).

Table 172. Compounds of formula I.b, wherein R² is R²-9.162 (=formula 1.172).

Table 173. Compounds of formula I.b, wherein R² is R²-9.163 (=formula 1.173).

Table 174. Compounds of formula I.b, wherein R² is R²-9.164 (=formula 1.174).

Table 175. Compounds of formula I.b, wherein R² is R²-9.165 (=formula 1.175).

Table 176. Compounds of formula I.b, wherein R² is R²-9.166 (=formula 1.176).

Table 177. Compounds of formula I.b, wherein R² is R²-9.167 (=formula 1.177).

Table 178. Compounds of formula I.b, wherein R² is R²-9.168 (=formula 1.178).

Table 179. Compounds of formula I.b, wherein R² is R²-9.169 (=formula 1.179).

Table 180. Compounds of formula I.b, wherein R² is R²-9.170 (=formula 1.180).

Table 181. Compounds of formula I.b, wherein R² is R²-9.171 (=formula 1.181).

Table 182. Compounds of formula I.b, wherein R² is R²-9.172 (=formula 1.182).

Table 183. Compounds of formula I.b, wherein R² is R²-9.173 (=formula 1.183).

Table 184. Compounds of formula I.b, wherein R² is R²-9.174 (=formula 1.184).

Table 185. Compounds of formula I.b, wherein R² is R²-9.175 (=formula 1.185).

Table 186. Compounds of formula I.b, wherein R² is R²-9.176 (=formula 1.186).

Table 187. Compounds of formula I.b, wherein R² is R²-9.177 (=formula 1.187).

Table 188. Compounds of formula I.b, wherein R² is R²-9.178 (=formula 1.188).

Table 189. Compounds of formula I.b, wherein R² is R²-9.179 (=formula 1.189).

Table 190. Compounds of formula I.b, wherein R² is R²-9.180 (=formula 1.190).

Table 191. Compounds of formula I.b, wherein R² is R²-9.181 (=formula 1.191).

Table 192. Compounds of formula I.b, wherein R² is R²-9.182 (=formula 1.192).

Table 193. Compounds of formula I.b, wherein R² is R²-9.183 (=formula 1.193).

Table 194. Compounds of formula I.b, wherein R² is R²-9.184 (=formula 1.194).

Table 195. Compounds of formula I.b, wherein R² is R²-9.185 (=formula 1.195).

Table 196. Compounds of formula I.b, wherein R² is R²-9.186 (=formula 1.196).

Table 197. Compounds of formula I.b, wherein R² is R²-9.187 (=formula 1.197).

Table 198. Compounds of formula I.b, wherein R² is R²-9.188 (=formula 1.198).

Table 199. Compounds of formula I.b, wherein R² is R²-9.189 (=formula 1.199).

Table 200. Compounds of formula I.b, wherein R² is R²-9.190 (=formula 1.200).

Table 201. Compounds of formula I.b, wherein R² is R²-9.191 (=formula 1.201).

Table 202. Compounds of formula I.b, wherein R² is R²-9.192 (=formula 1.202).

Table 203. Compounds of formula I.b, wherein R² is R²-9.193 (=formula 1.203).

Table 204. Compounds of formula I.b, wherein R² is R²-9.194 (=formula 1.204).

Table 205. Compounds of formula I.b, wherein R² is R²-9.195 (=formula 1.205).

Table 206. Compounds of formula I.b, wherein R² is R²-9.196 (=formula 1.206).

Table 207. Compounds of formula I.b, wherein R² is R²-9.197 (=formula 1.207).

Table 208. Compounds of formula I.b, wherein R² is R²-9.198 (=formula 1.208).

Table 209. Compounds of formula I.b, wherein R² is R²-9.199 (=formula 1.209).

Table 210. Compounds of formula I.b, wherein R² is R²-9.200 (=formula 1.210).

Table 211. Compounds of formula I.b, wherein R² is R²-9.201 (=formula 1.211).

Table 212. Compounds of formula I.b, wherein R² is R²-9.202 (=formula 1.212).

Table 213. Compounds of formula I.b, wherein R² is R²-9.203 (=formula 1.213).

Table 214. Compounds of formula I.b, wherein R² is R²-9.204 (=formula 1.214).

Table 215. Compounds of formula I.b, wherein R² is R²-9.205 (=formula 1.215).

Table 216. Compounds of formula I.b, wherein R² is R²-9.206 (=formula 1.216).

Table 217. Compounds of formula I.b, wherein R² is R²-9.207 (=formula 1.217).

Table 218. Compounds of formula I.b, wherein R² is R²-9.208 (=formula 1.218).

Table 219. Compounds of formula I.b, wherein R² is R²-9.209 (=formula 1.219).

Table 220. Compounds of formula I.b, wherein R² is R²-9.210 (=formula 1.220).

Table 221. Compounds of formula I.b, wherein R² is R²-9.211 (=formula 1.221).

Table 222. Compounds of formula I.b, wherein R² is R²-9.212 (=formula 1.222).

Table 223. Compounds of formula I.b, wherein R² is R²-9.213 (=formula 1.223).

Table 224. Compounds of formula I.b, wherein R² is R²-9.214 (=formula 1.224).

Table 225. Compounds of formula I.b, wherein R² is R²-9.215 (=formula 1.225).

Table 226. Compounds of formula I.b, wherein R² is R²-9.216 (=formula 1.226).

Table 227. Compounds of formula I.b, wherein R² is R²-9.217 (=formula 1.227).

Table 228. Compounds of formula I.b, wherein R² is R²-9.218 (=formula 1.228).

Table 229. Compounds of formula I.b, wherein R² is R²-9.219 (=formula 1.229).

Table 230. Compounds of formula I.b, wherein R² is R²-9.220 (=formula 1.230).

Table 231. Compounds of formula I.b, wherein R² is R²-9.221 (=formula 1.231).

Table 232. Compounds of formula I.b, wherein R² is R²-9.222 (=formula 1.232).

Table 233. Compounds of formula I.b, wherein R² is R²-9.223 (=formula 1.233).

Table 234. Compounds of formula I.b, wherein R² is R²-9.224 (=formula 1.234).

Table 235. Compounds of formula I.b, wherein R² is R²-9.225 (=formula 1.235).

Table 236. Compounds of formula I.b, wherein R² is R²-9.226 (=formula 1.236).

Table 237. Compounds of formula I.b, wherein R² is R²-9.227 (=formula 1.237).

Table 238. Compounds of formula I.b, wherein R² is R²-9.228 (=formula 1.238).

Table 239. Compounds of formula I.b, wherein R² is R²-9.229 (=formula 1.239).

Table 240. Compounds of formula I.b, wherein R² is R²-9.230 (=formula 1.240).

Table 241. Compounds of formula I.b, wherein R² is R²-9.231 (=formula 1.241).

Table 242. Compounds of formula I.b, wherein R² is R²-9.232 (=formula 1.242).

Table 243. Compounds of formula I.b, wherein R² is R²-9.233 (=formula 1.243).

Table 244. Compounds of formula I.b, wherein R² is R²-9.234 (=formula 1.244).

Table 245. Compounds of formula I.b, wherein R² is R²-9.235 (=formula 1.245).

Table 246. Compounds of formula I.b, wherein R² is R²-9.236 (=formula 1.246).

Table 247. Compounds of formula I.b, wherein R² is R²-9.237 (=formula 1.247).

Table 248. Compounds of formula I.b, wherein R² is R²-9.238 (=formula 1.248).

Table 249. Compounds of formula I.b, wherein R² is R²-9.239 (=formula 1.249).

Table 250. Compounds of formula I.b, wherein R² is R²-9.240 (=formula 1.250).

Table 251. Compounds of formula I.b, wherein R² is R²-9.241 (=formula 1.251).

Table 252. Compounds of formula I.b, wherein R² is R²-9.242 (=formula 1.252).

Table 253. Compounds of formula I.b, wherein R² is R²-9.243 (=formula 1.253).

Table 254. Compounds of formula I.b, wherein R² is R²-9.244 (=formula 1.254).

Table 255. Compounds of formula I.b, wherein R² is R²-9.245 (=formula 1.255).

Table 256. Compounds of formula I.b, wherein R² is R²-9.246 (=formula 1.256).

Table 257. Compounds of formula I.b, wherein R² is R²-9.247 (=formula 1.257).

Table 258. Compounds of formula I.b, wherein R² is R²-9.248 (=formula 1.258).

Table 259. Compounds of formula I.b, wherein R² is R²-9.249 (=formula 1.259).

Table 260. Compounds of formula I.b, wherein R² is R²-9.250 (=formula 1.260).

Table 261. Compounds of formula I.b, wherein R² is R²-9.251 (=formula 1.261).

Table 262. Compounds of formula I.b, wherein R² is R²-9.252 (=formula 1.262).

Table 263. Compounds of formula I.b, wherein R² is R²-9.253 (=formula 1.263).

Table 264. Compounds of formula I.b, wherein R² is R²-9.254 (=formula 1.264).

Table 265. Compounds of formula I.b, wherein R² is R²-9.255 (=formula 1.265).

Table 266. Compounds of formula I.b, wherein R² is R²-9.256 (=formula 1.266).

Table 267. Compounds of formula I.b, wherein R² is R²-9.257 (=formula 1.267).

Table 268. Compounds of formula I.b, wherein R² is R²-9.258 (=formula 1.268).

Table 269. Compounds of formula I.b, wherein R² is R²-9.259 (=formula 1.269).

Table 270. Compounds of formula I.b, wherein R² is R²-9.260 (=formula 1.270).

Table 271. Compounds of formula I.b, wherein R² is R²-9.261 (=formula 1.271).

Table 272. Compounds of formula I.b, wherein R² is R²-9.262 (=formula 1.272).

Table 273. Compounds of formula I.b, wherein R² is R²-9.263 (=formula 1.273).

Table 274. Compounds of formula I.b, wherein R² is R²-9.264 (=formula 1.274).

Table 275. Compounds of formula I.b, wherein R² is R²-9.265 (=formula 1.275).

Table 276. Compounds of formula I.b, wherein R² is R²-9.266 (=formula 1.276).

Table 277. Compounds of formula I.b, wherein R² is R²-9.267 (=formula 1.277).

Table 278. Compounds of formula I.b, wherein R² is R²-9.268 (=formula 1.278).

Table 279. Compounds of formula I.b, wherein R² is R²-9.269 (=formula 1.279).

Table 280. Compounds of formula I.b, wherein R² is R²-9.270 (=formula 1.280).

Table 281. Compounds of formula I.b, wherein R² is R²-9.271 (=formula 1.281).

Table 282. Compounds of formula I.b, wherein R² is R²-9.272 (=formula 1.282).

Table 283. Compounds of formula I.b, wherein R² is R²-9.273 (=formula 1.283).

Table 284. Compounds of formula I.b, wherein R² is R²-9.274 (=formula 1.284).

Table 285. Compounds of formula I.b, wherein R² is R²-9.275 (=formula 1.285).

Table 286. Compounds of formula I.b, wherein R² is R²-9.276 (=formula 1.286).

Table 287. Compounds of formula I.b, wherein R² is R²-9.277 (=formula 1.287).

Table 288. Compounds of formula I.b, wherein R² is R²-9.278 (=formula 1.288).

Table 289. Compounds of formula I.b, wherein R² is R²-9.279 (=formula 1.289).

Table 290. Compounds of formula I.b, wherein R² is R²-9.280 (=formula 1.290).

Table 291. Compounds of formula I.b, wherein R² is R²-9.281 (=formula 1.291).

Table 292. Compounds of formula I.b, wherein R² is R²-9.282 (=formula 1.292).

Table 293. Compounds of formula I.b, wherein R² is R²-9.283 (=formula 1.293).

Table 294. Compounds of formula I.b, wherein R² is R²-9.284 (=formula 1.294).

Table 295. Compounds of formula I.b, wherein R² is R²-9.285 (=formula 1.295).

Table 296. Compounds of formula I.b, wherein R² is R²-9.286 (=formula 1.296).

Table 297. Compounds of formula I.b, wherein R² is R²-9.287 (=formula 1.297).

Table 298. Compounds of formula I.b, wherein R² is R²-9.288 (=formula 1.298).

Table 299. Compounds of formula I.b, wherein R² is R²-9.289 (=formula 1.299).

Table 300. Compounds of formula I.b, wherein R² is R²-9.290 (=formula 1.300).

Table 301. Compounds of formula I.b, wherein R² is R²-9.291 (=formula 1.301).

Table 302. Compounds of formula I.b, wherein R² is R²-9.292 (=formula 1.302).

Table 303. Compounds of formula I.b, wherein R² is R²-9.293 (=formula 1.303).

Table 304. Compounds of formula I.b, wherein R² is R²-9.294 (=formula 1.304).

Table 305. Compounds of formula I.b, wherein R² is R²-9.295 (=formula 1.305).

Table 306. Compounds of formula I.b, wherein R² is R²-9.296 (=formula 1.306).

Table 307. Compounds of formula I.b, wherein R² is R²-9.297 (=formula 1.307).

Table 308. Compounds of formula I.b, wherein R² is R²-9.298 (=formula 1.308).

Table 309. Compounds of formula I.b, wherein R² is R²-9.299 (=formula 1.309).

Table 310. Compounds of formula I.b, wherein R² is R²-9.300 (=formula 1.310).

Table 311. Compounds of formula I.b, wherein R² is R²-9.301 (=formula 1.311).

Table 312. Compounds of formula I.b, wherein R² is R²-9.302 (=formula 1.312).

Table 313. Compounds of formula I.b, wherein R² is R²-9.303 (=formula 1.313).

Table 314. Compounds of formula I.b, wherein R² is R²-9.304 (=formula 1.314).

Table 315. Compounds of formula I.b, wherein R² is R²-9.305 (=formula 1.315).

Table 316. Compounds of formula I.b, wherein R² is R²-9.306 (=formula 1.316).

Table 317. Compounds of formula I.b, wherein R² is R²-9.307 (=formula 1.317).

Table 318. Compounds of formula I.b, wherein R² is R²-9.308 (=formula 1.318).

Table 319. Compounds of formula I.b, wherein R² is R²-9.309 (=formula 1.319).

Table 320. Compounds of formula I.b, wherein R² is R²-9.310 (=formula 1.320).

Table 321. Compounds of formula I.b, wherein R² is R²-9.311 (=formula 1.321).

Table 322. Compounds of formula I.b, wherein R² is R²-9.312 (=formula 1.322).

Table 323. Compounds of formula I.b, wherein R² is R²-9.313 (=formula 1.323).

Table 324. Compounds of formula I.b, wherein R² is R²-9.314 (=formula 1.324).

Table 325. Compounds of formula I.b, wherein R² is R²-9.315 (=formula 1.325).

Table 326. Compounds of formula I.b, wherein R² is R²-9.316 (=formula 1.326).

Table 327. Compounds of formula I.b, wherein R² is R²-9.317 (=formula 1.327).

Table 328. Compounds of formula I.b, wherein R² is R²-9.318 (=formula 1.328).

Table 329. Compounds of formula I.b, wherein R² is R²-9.319 (=formula 1.329).

Table 330. Compounds of formula I.b, wherein R² is R²-9.320 (=formula 1.330).

Table 331. Compounds of formula I.b, wherein R² is R²-9.321 (=formula 1.331).

Table 332. Compounds of formula I.b, wherein R² is R²-9.322 (=formula 1.332).

Table 333. Compounds of formula I.b, wherein R² is R²-9.323 (=formula 1.333).

Table 334. Compounds of formula I.b, wherein R² is R²-9.324 (=formula 1.334).

Table 335. Compounds of formula I.b, wherein R² is R²-9.325 (=formula 1.335).

Table 336. Compounds of formula I.b, wherein R² is R²-9.326 (=formula 1.336).

Table 337. Compounds of formula I.b, wherein R² is R²-9.327 (=formula 1.337).

Table 338. Compounds of formula I.b, wherein R² is R²-9.328 (=formula 1.338).

Table 339. Compounds of formula I.b, wherein R² is R²-9.329 (=formula 1.339).

Table 340. Compounds of formula I.b, wherein R² is R²-9.330 (=formula 1.340).

Table 341. Compounds of formula I.b, wherein R² is R²-9.331 (=formula 1.341).

Table 342. Compounds of formula I.b, wherein R² is R²-9.332 (=formula 1.342).

Table 343. Compounds of formula I.b, wherein R² is R²-9.333 (=formula 1.343).

Table 344. Compounds of formula I.b, wherein R² is R²-9.334 (=formula 1.344).

Table 345. Compounds of formula I.b, wherein R² is R²-9.335 (=formula 1.345).

Table 346. Compounds of formula I.b, wherein R² is R²-9.336 (=formula 1.346).

Table 347. Compounds of formula I.b, wherein R² is R²-9.334 (=formula 1.347).

Table 348. Compounds of formula I.b, wherein R² is R²-9.338 (=formula 1.348).

Table 349. Compounds of formula I.b, wherein R² is R²-9.339 (=formula 1.349).

Table 350. Compounds of formula I.b, wherein R² is R²-9.340 (=formula 1.350).

Table 351. Compounds of formula I.b, wherein R² is R²-9.341 (=formula 1.351).

Table 352. Compounds of formula I.b, wherein R² is R²-9.342 (=formula 1.352).

Table 353. Compounds of formula I.b, wherein R² is R²-9.343 (=formula 1.353).

Table 354. Compounds of formula I.b, wherein R² is R²-9.344 (=formula 1.354).

Table 355. Compounds of formula I.b, wherein R² is R²-9.345 (=formula 1.355).

Table 356. Compounds of formula I.b, wherein R² is R²-9.346 (=formula 1.356).

Table 357. Compounds of formula I.b, wherein R² is R²-9.347 (=formula 1.357).

Table 358. Compounds of formula I.b, wherein R² is R²-9.348 (=formula 1.358).

Table 359. Compounds of formula I.b, wherein R² is R²-9.349 (=formula 1.359).

Table 360. Compounds of formula I.b, wherein R² is R²-9.350 (=formula 1.360).

Table 361. Compounds of formula I.b, wherein R² is R²-9.351 (=formula 1.361).

Table 362. Compounds of formula I.b, wherein R² is R²-9.352 (=formula 1.362).

Table 363. Compounds of formula I.b, wherein R² is R²-9.353 (=formula 1.363).

Table 364. Compounds of formula I.b, wherein R² is R²-9.354 (=formula 1.364).

Table 365. Compounds of formula I.b, wherein R² is R²-9.355 (=formula 1.365).

Table 366. Compounds of formula I.b, wherein R² is R²-9.356 (=formula 1.366).

Table 367. Compounds of formula I.b, wherein R² is R²-9.357 (=formula 1.367).

Table 368. Compounds of formula I.b, wherein R² is R²-9.358 (=formula 1.368).

Table 369. Compounds of formula I.b, wherein R² is R²-9.359 (=formula 1.369).

Table 370. Compounds of formula I.b, wherein R² is R²-9.360 (=formula 1.370).

Table 371. Compounds of formula I.b, wherein R² is R²-9.361 (=formula 1.371).

Table 372. Compounds of formula I.b, wherein R² is R²-9.362 (=formula 1.372).

Table 373. Compounds of formula I.b, wherein R² is R²-9.363 (=formula 1.373).

Table 374. Compounds of formula I.b, wherein R² is R²-9.364 (=formula 1.374).

Table 375. Compounds of formula I.b, wherein R² is R²-9.365 (=formula 1.375).

Table 376. Compounds of formula I.b, wherein R² is R²-9.366 (=formula 1.376).

Table 377. Compounds of formula I.b, wherein R² is R²-9.367 (=formula 1.377).

Table 378. Compounds of formula I.b, wherein R² is R²-9.368 (=formula 1.378).

Table 379. Compounds of formula I.b, wherein R² is R²-9.369 (=formula 1.379).

Table 380. Compounds of formula I.b, wherein R² is R²-9.370 (=formula 1.380).

Table 381. Compounds of formula I.b, wherein R² is R²-9.371 (=formula 1.381).

Table 382. Compounds of formula I.b, wherein R² is R²-9.372 (=formula 1.382).

Table 383. Compounds of formula I.b, wherein R² is R²-9.373 (=formula 1.383).

Table 384. Compounds of formula I.b, wherein R² is R²-9.374 (=formula 1.384).

Table 385. Compounds of formula I.b, wherein R² is R²-9.375 (=formula 1.385).

Table 386. Compounds of formula I.b, wherein R² is R²-9.376 (=formula 1.386).

Table 387. Compounds of formula I.b, wherein R² is R²-9.377 (=formula 1.387).

Table 388. Compounds of formula I.b, wherein R² is R²-9.378 (=formula 1.388).

Table 389. Compounds of formula I.b, wherein R² is R²-9.379 (=formula 1.389).

Table 390. Compounds of formula I.b, wherein R² is R²-9.380 (=formula 1.390).

Table 391. Compounds of formula I.b, wherein R² is R²-9.381 (=formula 1.391).

Table 392. Compounds of formula I.b, wherein R² is R²-9.382 (=formula 1.392).

Table 393. Compounds of formula I.b, wherein R² is R²-9.383 (=formula 1.393).

Table 394. Compounds of formula I.b, wherein R² is R²-9.384 (=formula 1.394).

Table 395. Compounds of formula I.b, wherein R² is R²-9.385 (=formula 1.395).

Table 396. Compounds of formula I.b, wherein R² is R²-9.386 (=formula 1.396).

Table 397. Compounds of formula I.b, wherein R² is R²-9.387 (=formula 1.397).

Table 398. Compounds of formula I.b, wherein R² is R²-9.388 (=formula 1.398).

Table 399. Compounds of formula I.b, wherein R² is R²-9.389 (=formula 1.399).

Table 400. Compounds of formula I.b, wherein R² is R²-9.390 (=formula 1.400).

Table 401. Compounds of formula I.b, wherein R² is R²-9.391 (=formula 1.401).

Table 402. Compounds of formula I.b, wherein R² is R²-9.392 (=formula 1.402).

Table 403. Compounds of formula I.b, wherein R² is R²-9.393 (=formula 1.403).

Table 404. Compounds of formula I.b, wherein R² is R²-9.394 (=formula 1.404).

Table 405. Compounds of formula I.b, wherein R² is R²-9.395 (=formula 1.405).

Table 406. Compounds of formula I.b, wherein R² is R²-9.396 (=formula 1.406).

Table 407. Compounds of formula I.b, wherein R² is R²-9.397 (=formula 1.407).

Table 408. Compounds of formula I.b, wherein R² is R²-9.398 (=formula 1.408).

Table 409. Compounds of formula I.b, wherein R² is R²-9.399 (=formula 1.409).

Table 410. Compounds of formula I.b, wherein R² is R²-9.400 (=formula 1.410).

Table 411. Compounds of formula I.b, wherein R² is R²-9.401 (=formula 1.411).

Table 412. Compounds of formula I.b, wherein R² is R²-9.402 (=formula 1.412).

Table 413. Compounds of formula I.b, wherein R² is R²-9.403 (=formula 1.413).

Table 414. Compounds of formula I.b, wherein R² is R²-9.404 (=formula 1.414).

Table 415. Compounds of formula I.b, wherein R² is R²-9.405 (=formula 1.415).

Table 416. Compounds of formula I.b, wherein R² is R²-9.406 (=formula 1.416).

Table 417. Compounds of formula I.b, wherein R² is R²-9.407 (=formula 1.417).

Table 418. Compounds of formula I.b, wherein R² is R²-9.408 (=formula 1.418).

Table 419. Compounds of formula I.b, wherein R² is R²-9.409 (=formula 1.419).

Table 420. Compounds of formula I.b, wherein R² is R²-9.410 (=formula 1.420).

Table 421. Compounds of formula I.b, wherein R² is R²-9.411 (=formula 1.421).

Table 422. Compounds of formula I.b, wherein R² is R²-9.412 (=formula 1.422).

Table 423. Compounds of formula I.b, wherein R² is R²-9.413 (=formula 1.423).

Table 424. Compounds of formula I.b, wherein R² is R²-9.414 (=formula 1.424).

Table 425. Compounds of formula I.b, wherein R² is R²-9.415 (=formula 1.425).

Table 426. Compounds of formula I.b, wherein R² is R²-9.416 (=formula 1.426).

Table 427. Compounds of formula I.b, wherein R² is R²-9.417 (=formula 1.427).

Table 428. Compounds of formula I.b, wherein R² is R²-9.418 (=formula 1.428).

Table 429. Compounds of formula I.b, wherein R² is R²-9.419 (=formula 1.429).

Table 430. Compounds of formula I.b, wherein R² is R²-9.420 (=formula 1.430).

Table 431. Compounds of formula I.b, wherein R² is R²-9.421 (=formula 1.431).

Table 432. Compounds of formula I.b, wherein R² is R²-9.422 (=formula 1.432).

Table 433. Compounds of formula I.b, wherein R² is R²-9.423 (=formula 1.433).

Table 434. Compounds of formula I.b, wherein R² is R²-9.424 (=formula 1.434).

Table 435. Compounds of formula I.b, wherein R² is R²-9.425 (=formula 1.435).

Table 436. Compounds of formula I.b, wherein R² is R²-9.426 (=formula 1.436).

Table 437. Compounds of formula I.b, wherein R² is R²-9.427 (=formula 1.437).

Table 438. Compounds of formula I.b, wherein R² is R²-9.428 (=formula 1.438).

Table 439. Compounds of formula I.b, wherein R² is R²-9.429 (=formula 1.439).

Table 440. Compounds of formula I.b, wherein R² is R²-9.430 (=formula 1.440).

Table 441. Compounds of formula I.b, wherein R² is R²-9.431 (=formula 1.441).

Table 442. Compounds of formula I.b, wherein R² is R²-9.432 (=formula 1.442).

Table 443. Compounds of formula I.b, wherein R² is R²-9.433 (=formula 1.443).

Table 444. Compounds of formula I.b, wherein R² is R²-9.434 (=formula 1.444).

Table 445. Compounds of formula I.b, wherein R² is R²-9.435 (=formula 1.445).

Table 446. Compounds of formula I.b, wherein R² is R²-9.436 (=formula 1.446).

Table 447. Compounds of formula I.b, wherein R² is R²-9.437 (=formula 1.447).

Table 448. Compounds of formula I.b, wherein R² is R²-9.438 (=formula 1.448).

Table 449. Compounds of formula I.b, wherein R² is R²-9.439 (=formula 1.449).

Table 450. Compounds of formula I.b, wherein R² is R²-9.440 (=formula 1.450).

Table 451. Compounds of formula I.b, wherein R² is R²-9.441 (=formula 1.451).

Table 452. Compounds of formula I.b, wherein R² is R²-9.442 (=formula 1.452).

Table 453. Compounds of formula I.b, wherein R² is R²-9.443 (=formula 1.453).

Table 454. Compounds of formula I.b, wherein R² is R²-9.444 (=formula 1.454).

Table 455. Compounds of formula I.b, wherein R² is R²-9.445 (=formula 1.455).

Table 456. Compounds of formula I.b, wherein R² is R²-9.446 (=formula 1.456).

Table 457. Compounds of formula I.b, wherein R² is R²-9.447 (=formula 1.457).

Table 458. Compounds of formula I.b, wherein R² is R²-9.448 (=formula 1.458).

Table 459. Compounds of formula I.b, wherein R² is R²-9.449 (=formula 1.459).

Table 460. Compounds of formula I.b, wherein R² is R²-9.450 (=formula 1.460).

Table 461. Compounds of formula I.b, wherein R² is R²-9.451 (=formula 1.461).

Table 462. Compounds of formula I.b, wherein R² is R²-9.452 (=formula 1.462).

Table 463. Compounds of formula I.b, wherein R² is R²-9.453 (=formula 1.463).

Table 464. Compounds of formula I.b, wherein R² is R²-9.454 (=formula 1.464).

Table 465. Compounds of formula I.b, wherein R² is R²-9.455 (=formula 1.465).

Table 466. Compounds of formula I.b, wherein R² is R²-9.456 (=formula 1.466).

Table 467. Compounds of formula I.b, wherein R² is R²-9.457 (=formula 1.467).

Table 468. Compounds of formula I.b, wherein R² is R²-9.458 (=formula 1.468).

Table 469. Compounds of formula I.b, wherein R² is R²-9.459 (=formula 1.469).

Table 470. Compounds of formula I.b, wherein R² is R²-9.460 (=formula 1.470).

Table 471. Compounds of formula I.b, wherein R² is R²-9.461 (=formula 1.471).

Table 472. Compounds of formula I.b, wherein R² is R²-9.462 (=formula 1.472).

Table 473. Compounds of formula I.b, wherein R² is R²-9.463 (=formula 1.473).

Table 474. Compounds of formula I.b, wherein R² is R²-9.464 (=formula 1.474).

Table 475. Compounds of formula I.b, wherein R² is R²-9.465 (=formula 1.475).

Table 476. Compounds of formula I.b, wherein R² is R²-9.466 (=formula 1.476).

Table 477. Compounds of formula I.b, wherein R² is R²-9.467 (=formula 1.477).

Table 478. Compounds of formula I.b, wherein R² is R²-9.468 (=formula 1.478).

Table 479. Compounds of formula I.b, wherein R² is R²-9.469 (=formula 1.479).

Table 480. Compounds of formula I.b, wherein R² is R²-9.470 (=formula 1.480).

Table 481. Compounds of formula I.b, wherein R² is R²-9.471 (=formula 1.481).

Table 482. Compounds of formula I.b, wherein R² is R²-9.472 (=formula 1.482).

Table 483. Compounds of formula I.b, wherein R² is R²-9.473 (=formula 1.483).

Table 484. Compounds of formula I.b, wherein R² is R²-9.474 (=formula 1.484).

Table 485. Compounds of formula I.b, wherein R² is R²-9.475 (=formula 1.485).

Table 486. Compounds of formula I.b, wherein R² is R²-9.476 (=formula 1.486).

Table 487. Compounds of formula I.b, wherein R² is R²-9.477 (=formula 1.487).

Table 488. Compounds of formula I.b, wherein R² is R²-9.478 (=formula 1.488).

Table 489. Compounds of formula I.b, wherein R² is R²-9.479 (=formula 1.489).

Table 490. Compounds of formula I.b, wherein R² is R²-9.480 (=formula 1.490).

Table 491. Compounds of formula I.b, wherein R² is R²-9.481 (=formula 1.491).

Table 492. Compounds of formula I.b, wherein R² is R²-9.482 (=formula 1.492).

Table 493. Compounds of formula I.b, wherein R² is R²-9.483 (=formula 1.493).

Table 494. Compounds of formula I.b, wherein R² is R²-9.484 (=formula 1.494).

Table 495. Compounds of formula I.b, wherein R² is R²-9.485 (=formula 1.495).

Table 496. Compounds of formula I.b, wherein R² is R²-9.486 (=formula 1.496).

Table 497. Compounds of formula I.b, wherein R² is R²-9.487 (=formula 1.497).

Table 498. Compounds of formula I.b, wherein R² is R²-9.488 (=formula 1.498).

Table 499. Compounds of formula I.b, wherein R² is R²-9.489 (=formula 1.499).

Table 500. Compounds of formula I.b, wherein R² is R²-9.490 (=formula 1.500).

Table 501. Compounds of formula I.b, wherein R² is R²-9.491 (=formula 1.501).

Table 502. Compounds of formula I.b, wherein R² is R²-9.492 (=formula 1.502).

Table 503. Compounds of formula I.b, wherein R² is R²-9.493 (=formula 1.503).

Table 504. Compounds of formula I.b, wherein R² is R²-9.494 (=formula 1.504).

Table 505. Compounds of formula I.b, wherein R² is R²-9.495 (=formula 1.505).

Table 506. Compounds of formula I.b, wherein R² is R²-9.496 (=formula 1.506).

Table 507. Compounds of formula I.b, wherein R² is R²-9.497 (=formula 1.507).

Table 508. Compounds of formula I.b, wherein R² is R²-9.498 (=formula 1.508).

Table 509. Compounds of formula I.b, wherein R² is R²-9.499 (=formula 1.509).

Table 510. Compounds of formula I.b, wherein R² is R²-9.500 (=formula 1.510).

Table 511. Compounds of formula I.b, wherein R² is R²-9.501 (=formula 1.511).

Table 512. Compounds of formula I.b, wherein R² is R²-9.502 (=formula 1.512).

Table 513. Compounds of formula I.b, wherein R² is R²-9.503 (=formula 1.513).

Table 514. Compounds of formula I.b, wherein R² is R²-9.504 (=formula 1.514).

Table 515. Compounds of formula I.b, wherein R² is R²-9.505 (=formula 1.515).

Table 516. Compounds of formula I.b, wherein R² is R²-9.506 (=formula 1.516).

Table 517. Compounds of formula I.b, wherein R² is R²-9.507 (=formula 1.517).

Table 518. Compounds of formula I.b, wherein R² is R²-9.508 (=formula 1.518).

Table 519. Compounds of formula I.b, wherein R² is R²-9.509 (=formula 1.519).

Table 520. Compounds of formula I.b, wherein R² is R²-9.510 (=formula 1.520).

Table 521. Compounds of formula I.b, wherein R² is R²-9.511 (=formula 1.521).

Table 522. Compounds of formula I.b, wherein R² is R²-9.512 (=formula 1.522).

Table 523. Compounds of formula I.b, wherein R² is R²-9.513 (=formula 1.523).

Table 524. Compounds of formula I.b, wherein R² is R²-9.514 (=formula 1.524).

Table 525. Compounds of formula I.b, wherein R² is R²-9.515 (=formula 1.525).

Table 526. Compounds of formula I.b, wherein R² is R²-9.516 (=formula 1.526).

Table 527. Compounds of formula I.b, wherein R² is R²-9.517 (=formula 1.527).

Table 528. Compounds of formula I.b, wherein R² is R²-9.518 (=formula 1.528).

Table 529. Compounds of formula I.b, wherein R² is R²-9.519 (=formula 1.529).

Table 530. Compounds of formula I.b, wherein R² is R²-9.520 (=formula 1.530).

Table 531. Compounds of formula I.b, wherein R² is R²-9.521 (=formula 1.531).

Table 532. Compounds of formula I.b, wherein R² is R²-9.522 (=formula 1.532).

Table 533. Compounds of formula I.b, wherein R² is R²-9.523 (=formula 1.533).

Table 534. Compounds of formula I.b, wherein R² is R²-9.524 (=formula 1.534).

Table 535. Compounds of formula I.b, wherein R² is R²-9.525 (=formula 1.535).

Table 536. Compounds of formula I.b, wherein R² is R²-9.526 (=formula 1.536).

Table 537. Compounds of formula I.b, wherein R² is R²-9.527 (=formula 1.537).

Table 538. Compounds of formula I.b, wherein R² is R²-9.528 (=formula 1.538).

Table 539. Compounds of formula I.b, wherein R² is R²-9.529 (=formula 1.539).

Table 540. Compounds of formula I.b, wherein R² is R²-9.530 (=formula 1.540).

Table 541. Compounds of formula I.b, wherein R² is R²-9.531 (=formula 1.541).

Table 542. Compounds of formula I.b, wherein R² is R²-9.532 (=formula 1.542).

Table 543. Compounds of formula I.b, wherein R² is R²-9.533 (=formula 1.543).

Table 544. Compounds of formula I.b, wherein R² is R²-9.534 (=formula 1.544).

Table 545. Compounds of formula I.b, wherein R² is R²-9.535 (=formula 1.545).

Table 546. Compounds of formula I.b, wherein R² is R²-9.536 (=formula 1.546).

Table 547. Compounds of formula I.b, wherein R² is R²-9.537 (=formula 1.547).

Table 548. Compounds of formula I.b, wherein R² is R²-9.538 (=formula 1.548).

Table 549. Compounds of formula I.b, wherein R² is R²-9.539 (=formula 1.549).

Table 550. Compounds of formula I.b, wherein R² is R²-9.540 (=formula 1.550).

Table 551. Compounds of formula I.b, wherein R² is R²-9.541 (=formula 1.551).

Table 552. Compounds of formula I.b, wherein R² is R²-9.542 (=formula 1.552).

Table 553. Compounds of formula I.b, wherein R² is R²-9.543 (=formula 1.553).

Table 554. Compounds of formula I.b, wherein R² is R²-9.544 (=formula 1.554).

Table 555. Compounds of formula I.b, wherein R² is R²-9.545 (=formula 1.555).

Table 556. Compounds of formula I.b, wherein R² is R²-9.546 (=formula 1.556).

Table 557. Compounds of formula I.b, wherein R² is R²-9.547 (=formula 1.557).

Table 558. Compounds of formula I.b, wherein R² is R²-9.548 (=formula 1.558).

Table 559. Compounds of formula I.b, wherein R² is R²-9.549 (=formula 1.559).

Table 560. Compounds of formula I.b, wherein R² is R²-9.550 (=formula 1.560).

Table 561. Compounds of formula I.b, wherein R² is R²-9.551 (=formula 1.561).

Table 562. Compounds of formula I.b, wherein R² is R²-9.552 (=formula 1.562).

Table 563. Compounds of formula I.b, wherein R² is R²-9.553 (=formula 1.563).

Table 564. Compounds of formula I.b, wherein R² is R²-9.554 (=formula 1.564).

Table 565. Compounds of formula I.b, wherein R² is R²-9.555 (=formula 1.565).

Table 566. Compounds of formula I.b, wherein R² is R²-9.556 (=formula 1.566).

Table 567. Compounds of formula I.b, wherein R² is R²-9.557 (=formula 1.567).

Table 568. Compounds of formula I.b, wherein R² is R²-9.558 (=formula 1.568).

Table 569. Compounds of formula I.b, wherein R² is R²-9.559 (=formula 1.569).

Table 570. Compounds of formula I.b, wherein R² is R²-9.560 (=formula 1.570).

Table 571. Compounds of formula I.b, wherein R² is R²-9.561 (=formula 1.571).

Table 572. Compounds of formula I.b, wherein R² is R²-9.562 (=formula 1.572).

Table 573. Compounds of formula I.b, wherein R² is R²-9.563 (=formula 1.573).

Table 574. Compounds of formula I.b, wherein R² is R²-9.564 (=formula 1.574).

Table 575. Compounds of formula I.b, wherein R² is R²-9.565 (=formula 1.575).

Table 576. Compounds of formula I.b, wherein R² is R²-9.566 (=formula 1.576).

Table 577. Compounds of formula I.b, wherein R² is R²-9.567 (=formula 1.577).

Table 578. Compounds of formula I.b, wherein R² is R²-9.568 (=formula 1.578).

Table 579. Compounds of formula I.b, wherein R² is R²-9.569 (=formula 1.579).

Table 580. Compounds of formula I.b, wherein R² is R²-9.570 (=formula 1.580).

Table 581. Compounds of formula I.b, wherein R² is R²-9.571 (=formula 1.581).

Table 582. Compounds of formula I.b, wherein R² is R²-9.572 (=formula 1.582).

Table 583. Compounds of formula I.b, wherein R² is R²-9.573 (=formula 1.583).

Table 584. Compounds of formula I.b, wherein R² is R²-9.574 (=formula 1.584).

Table 585. Compounds of formula I.b, wherein R² is R²-9.575 (=formula 1.585).

Table 586. Compounds of formula I.b, wherein R² is R²-9.576 (=formula 1.586).

Table 587. Compounds of formula I.b, wherein R² is R²-9.577 (=formula 1.587).

Table 588. Compounds of formula I.b, wherein R² is R²-9.578 (=formula 1.588).

Table 589. Compounds of formula I.b, wherein R² is R²-9.579 (=formula 1.589).

Table 590. Compounds of formula I.b, wherein R² is R²-9.580 (=formula 1.590).

Table 591. Compounds of formula I.b, wherein R² is R²-9.581 (=formula 1.591).

Table 592. Compounds of formula I.b, wherein R² is R²-9.582 (=formula 1.592).

Table 593. Compounds of formula I.b, wherein R² is R²-9.583 (=formula 1.593).

Table 594. Compounds of formula I.b, wherein R² is R²-9.584 (=formula 1.594).

Table 595. Compounds of formula I.b, wherein R² is R²-9.585 (=formula 1.595).

Table 596. Compounds of formula I.b, wherein R² is R²-9.586 (=formula 1.596).

Table 597. Compounds of formula I.b, wherein R² is R²-9.587 (=formula 1.597).

Table 598. Compounds of formula I.b, wherein R² is R²-9.588 (=formula 1.598).

Table 599. Compounds of formula I.b, wherein R² is R²-9.589 (=formula 1.599).

Table 600. Compounds of formula I.b, wherein R² is R²-9.590 (=formula 1.600).

Table 601. Compounds of formula I.b, wherein R² is R²-9.591 (=formula 1.601).

Table 602. Compounds of formula I.b, wherein R² is R²-9.592 (=formula 1.602).

Table 603. Compounds of formula I.b, wherein R² is R²-9.593 (=formula 1.603).

Table 604. Compounds of formula I.b, wherein R² is R²-9.594 (=formula 1.604).

Table 605. Compounds of formula I.b, wherein R² is R²-9.595 (=formula 1.605).

Table 606. Compounds of formula I.b, wherein R² is R²-9.596 (=formula 1.606).

Table 607. Compounds of formula I.b, wherein R² is R²-9.597 (=formula 1.607).

Table 608. Compounds of formula I.b, wherein R² is R²-9.598 (=formula 1.608).

Table 609. Compounds of formula I.b, wherein R² is R²-9.599 (=formula 1.609).

Table 610. Compounds of formula I.b, wherein R² is R²-9.600 (=formula 1.610).

Table 611. Compounds of formula I.b, wherein R² is R²-9.601 (=formula 1.611).

Table 612. Compounds of formula I.b, wherein R² is R²-9.602 (=formula 1.612).

Table 613. Compounds of formula I.b, wherein R² is R²-9.603 (=formula 1.613).

Table 614. Compounds of formula I.b, wherein R² is R²-9.604 (=formula 1.614).

Table 615. Compounds of formula I.b, wherein R² is R²-9.605 (=formula 1.615).

Table 616. Compounds of formula I.b, wherein R² is R²-9.606 (=formula 1.616).

Table 617. Compounds of formula I.b, wherein R² is R²-9.607 (=formula 1.617).

Table 618. Compounds of formula I.b, wherein R² is R²-9.608 (=formula 1.618).

Table 619. Compounds of formula I.b, wherein R² is R²-9.609 (=formula 1.619).

Table 620. Compounds of formula I.b, wherein R² is R²-9.610 (=formula 1.620).

Table 621. Compounds of formula I.b, wherein R² is R²-9.611 (=formula 1.621).

Table 622. Compounds of formula I.b, wherein R² is R²-9.612 (=formula 1.622).

Table 623. Compounds of formula I.b, wherein R² is R²-9.613 (=formula 1.623).

Table 624. Compounds of formula I.b, wherein R² is R²-9.614 (=formula 1.624).

Table 625. Compounds of formula I.b, wherein R² is R²-9.615 (=formula 1.625).

Table 626. Compounds of formula I.b, wherein R² is R²-9.616 (=formula 1.626).

Table 627. Compounds of formula I.b, wherein R² is R²-9.617 (=formula 1.627).

Table 628. Compounds of formula I.b, wherein R² is R²-9.618 (=formula 1.628).

Table 629. Compounds of formula I.b, wherein R² is R²-9.619 (=formula 1.629).

Table 630. Compounds of formula I.b, wherein R² is R²-9.620 (=formula 1.630).

Table 631. Compounds of formula I.b, wherein R² is R²-9.621 (=formula 1.631).

Table 632. Compounds of formula I.b, wherein R² is R²-9.622 (=formula 1.632).

Table 633. Compounds of formula I.b, wherein R² is R²-9.623 (=formula 1.633).

Table 634. Compounds of formula I.b, wherein R² is R²-9.624 (=formula 1.634).

Table 635. Compounds of formula I.b, wherein R² is R²-9.625 (=formula 1.635).

Table 636. Compounds of formula I.b, wherein R² is R²-9.626 (=formula 1.636).

Table 637. Compounds of formula I.b, wherein R² is R²-9.627 (=formula 1.637).

Table 638. Compounds of formula I.b, wherein R² is R²-9.628 (=formula 1.638).

Table 639. Compounds of formula I.b, wherein R² is R²-9.629 (=formula 1.639).

Table 640. Compounds of formula I.b, wherein R² is R²-9.630 (=formula 1.640).

Table 641. Compounds of formula I.b, wherein R² is R²-9.631 (=formula 1.641).

Table 642. Compounds of formula I.b, wherein R² is R²-9.632 (=formula 1.642).

Table 643. Compounds of formula I.b, wherein R² is R²-9.633 (=formula 1.643).

Table 644. Compounds of formula I.b, wherein R² is R²-9.634 (=formula 1.644).

Table 645. Compounds of formula I.b, wherein R² is R²-9.635 (=formula 1.645).

Table 646. Compounds of formula I.b, wherein R² is R²-9.636 (=formula 1.646).

Table 647. Compounds of formula I.b, wherein R² is R²-9.637 (=formula 1.647).

Table 648. Compounds of formula I.b, wherein R² is R²-9.638 (=formula 1.648).

Table 649. Compounds of formula I.b, wherein R² is R²-9.639 (=formula 1.649).

Table 650. Compounds of formula I.b, wherein R² is R²-9.640 (=formula 1.650).

Table 651. Compounds of formula I.b, wherein R² is R²-9.641 (=formula 1.651).

Table 652. Compounds of formula I.b, wherein R² is R²-9.642 (=formula 1.652).

Table 653. Compounds of formula I.b, wherein R² is R²-9.643 (=formula 1.653).

Table 654. Compounds of formula I.b, wherein R² is R²-9.644 (=formula 1.654).

Table 655. Compounds of formula I.b, wherein R² is R²-9.645 (=formula 1.655).

Table 656. Compounds of formula I.b, wherein R² is R²-9.646 (=formula 1.656).

Table 657. Compounds of formula I.b, wherein R² is R²-9.647 (=formula 1.657).

Table 658. Compounds of formula I.b, wherein R² is R²-9.648 (=formula 1.658).

Table 659. Compounds of formula I.b, wherein R² is R²-9.649 (=formula 1.659).

Table 660. Compounds of formula I.b, wherein R² is R²-9.650 (=formula 1.660).

Table 661. Compounds of formula I.b, wherein R² is R²-9.651 (=formula 1.661).

Table 662. Compounds of formula I.b, wherein R² is R²-9.652 (=formula 1.662).

Table 663. Compounds of formula I.b, wherein R² is R²-9.653 (=formula 1.663).

Table 664. Compounds of formula I.b, wherein R² is R²-9.654 (=formula 1.664).

Table 665. Compounds of formula I.b, wherein R² is R²-9.655 (=formula 1.665).

Table 666. Compounds of formula I.b, wherein R² is R²-9.656 (=formula 1.666).

Table 667. Compounds of formula I.b, wherein R² is R²-9.657 (=formula 1.667).

Table 668. Compounds of formula I.b, wherein R² is R²-9.658 (=formula 1.668).

Table 669. Compounds of formula I.b, wherein R² is R²-9.659 (=formula 1.669).

Table 670. Compounds of formula I.b, wherein R² is R²-9.660 (=formula 1.670).

Table 671. Compounds of formula I.b, wherein R² is R²-9.661 (=formula 1.671).

Table 672. Compounds of formula I.b, wherein R² is R²-9.662 (=formula 1.672).

Table 673. Compounds of formula I.b, wherein R² is R²-9.663 (=formula 1.673).

Table 674. Compounds of formula I.b, wherein R² is R²-9.664 (=formula 1.674).

Table 675. Compounds of formula I.b, wherein R² is R²-9.665 (=formula 1.675).

Table 676. Compounds of formula I.b, wherein R² is R²-9.666 (=formula 1.676).

Table 677. Compounds of formula I.b, wherein R² is R²-9.667 (=formula 1.677).

Table 678. Compounds of formula I.b, wherein R² is R²-9.668 (=formula 1.678).

Table 679. Compounds of formula I.b, wherein R² is R²-9.669 (=formula 1.679).

Table 680. Compounds of formula I.b, wherein R² is R²-9.670 (=formula 1.680).

Table 681. Compounds of formula I.b, wherein R² is R²-9.671 (=formula 1.681).

Table 682. Compounds of formula I.b, wherein R² is R²-9.672 (=formula 1.682).

Table 683. Compounds of formula I.b, wherein R² is R²-9.673 (=formula 1.683).

Table 684. Compounds of formula I.b, wherein R² is R²-9.674 (=formula 1.684).

Table 685. Compounds of formula I.b, wherein R² is R²-9.675 (=formula 1.685).

Table 686. Compounds of formula I.b, wherein R² is R²-9.676 (=formula 1.686).

Table 687. Compounds of formula I.b, wherein R² is R²-10.1 (=formula 1.687).

Table 688. Compounds of formula I.b, wherein R² is R²-10.2 (=formula 1.688).

Table 689. Compounds of formula I.b, wherein R² is R²-10.3 (=formula 1.689).

Table 690. Compounds of formula I.b, wherein R² is R²-10.4 (=formula 1.690).

Table 691. Compounds of formula I.b, wherein R² is R²-10.5 (=formula 1.691).

Table 692. Compounds of formula I.b, wherein R² is R²-10.6 (=formula 1.692).

Table 693. Compounds of formula I.b, wherein R² is R²-10.7 (=formula 1.693).

Table 694. Compounds of formula I.b, wherein R² is R²-10.8 (=formula 1.694).

Table 695. Compounds of formula I.b, wherein R² is R²-10.9 (=formula 1.695).

Table 696. Compounds of formula I.b, wherein R² is R²-10.10 (=formula 1.696).

Table 697. Compounds of formula I.b, wherein R² is R²-10.11 (=formula 1.697).

Table 698. Compounds of formula I.b, wherein R² is R²-10.12 (=formula 1.698).

Table 699. Compounds of formula I.b, wherein R² is R²-10.13 (=formula 1.699).

Table 700. Compounds of formula I.b, wherein R² is R²-10.14 (=formula 1.700).

Table 701. Compounds of formula I.b, wherein R² is R²-10.15 (=formula 1.701).

Table 702. Compounds of formula I.b, wherein R² is R²-10.16 (=formula 1.702).

Table 703. Compounds of formula I.b, wherein R² is R²-10.17 (=formula 1.703).

Table 704. Compounds of formula I.b, wherein R² is R²-10.18 (=formula 1.704).

Table 705. Compounds of formula I.b, wherein R² is R²-10.19 (=formula 1.705).

Table 706. Compounds of formula I.b, wherein R² is R²-10.20 (=formula 1.706).

Table 707. Compounds of formula I.b, wherein R² is R²-10.21 (=formula 1.707).

Table 708. Compounds of formula I.b, wherein R² is R²-10.22 (=formula 1.708).

Table 709. Compounds of formula I.b, wherein R² is R²-10.23 (=formula 1.709).

Table 710. Compounds of formula I.b, wherein R² is R²-10.24 (=formula 1.710).

Table 711. Compounds of formula I.b, wherein R² is R²-10.25 (=formula 1.711).

Table 712. Compounds of formula I.b, wherein R² is R²-10.26 (=formula 1.712).

Table 713. Compounds of formula I.b, wherein R² is R²-10.27 (=formula 1.713).

Table 714. Compounds of formula I.b, wherein R² is R²-10.28 (=formula 1.714).

Table 715. Compounds of formula I.b, wherein R² is R²-10.29 (=formula 1.715).

Table 716. Compounds of formula I.b, wherein R² is R²-10.30 (=formula 1.716).

Table 717. Compounds of formula I.b, wherein R² is R²-10.31 (=formula 1.717).

Table 718. Compounds of formula I.b, wherein R² is R²-10.32 (=formula 1.718).

Table 719. Compounds of formula I.b, wherein R² is R²-10.33 (=formula 1.719).

Table 720. Compounds of formula I.b, wherein R² is R²-10.34 (=formula 1.720).

Table 721. Compounds of formula I.b, wherein R² is R²-10.35 (=formula 1.721).

Table 722. Compounds of formula I.b, wherein R² is R²-10.36 (=formula 1.722).

Table 723. Compounds of formula I.b, wherein R² is R²-10.37 (=formula 1.723).

Table 724. Compounds of formula I.b, wherein R² is R²-10.38 (=formula 1.724).

Table 725. Compounds of formula I.b, wherein R² is R²-10.39 (=formula 1.725).

Table 726. Compounds of formula I.b, wherein R² is R²-10.40 (=formula 1.726).

Table 727. Compounds of formula I.b, wherein R² is R²-10.41 (=formula 1.727).

Table 728. Compounds of formula I.b, wherein R² is R²-10.42 (=formula 1.728).

Table 729. Compounds of formula I.b, wherein R² is R²-10.43 (=formula 1.729).

Table 730. Compounds of formula I.b, wherein R² is R²-10.44 (=formula 1.730).

Table 731. Compounds of formula I.b, wherein R² is R²-10.45 (=formula 1.731).

Table 732. Compounds of formula I.b, wherein R² is R²-10.46 (=formula 1.732).

Table 733. Compounds of formula I.b, wherein R² is R²-10.47 (=formula 1.733).

Table 734. Compounds of formula I.b, wherein R² is R²-10.48 (=formula 1.734).

Table 735. Compounds of formula I.b, wherein R² is R²-10.49 (=formula 1.735).

Table 736. Compounds of formula I.b, wherein R² is R²-10.50 (=formula 1.736).

Table 737. Compounds of formula I.b, wherein R² is R²-10.51 (=formula 1.737).

Table 738. Compounds of formula I.b, wherein R² is R²-10.52 (=formula 1.738).

Table 739. Compounds of formula I.b, wherein R² is R²-10.53 (=formula 1.739).

Table 740. Compounds of formula I.b, wherein R² is R²-10.54 (=formula 1.740).

Table 741. Compounds of formula I.b, wherein R² is R²-10.55 (=formula 1.741).

Table 742. Compounds of formula I.b, wherein R² is R²-10.56 (=formula 1.742).

Table 743. Compounds of formula I.b, wherein R² is R²-10.57 (=formula 1.743).

Table 744. Compounds of formula I.b, wherein R² is R²-10.58 (=formula 1.744).

Table 745. Compounds of formula I.b, wherein R² is R²-10.59 (=formula 1.745).

Table 746. Compounds of formula I.b, wherein R² is R²-10.60 (=formula 1.746).

Table 747. Compounds of formula I.b, wherein R² is R²-10.61 (=formula 1.747).

Table 748. Compounds of formula I.b, wherein R² is R²-10.62 (=formula 1.748).

Table 749. Compounds of formula I.b, wherein R² is R²-10.63 (=formula 1.749).

Table 750. Compounds of formula I.b, wherein R² is R²-10.64 (=formula 1.750).

Table 751. Compounds of formula I.b, wherein R² is R²-10.65 (=formula 1.751).

Table 752. Compounds of formula I.b, wherein R² is R²-10.66 (=formula 1.752).

Table 753. Compounds of formula I.b, wherein R² is R²-10.67 (=formula 1.753).

Table 754. Compounds of formula I.b, wherein R² is R²-10.68 (=formula 1.754).

Table 755. Compounds of formula I.b, wherein R² is R²-10.69 (=formula 1.755).

Table 756. Compounds of formula I.b, wherein R² is R²-10.70 (=formula 1.756).

Table 757. Compounds of formula I.b, wherein R² is R²-10.71 (=formula 1.757).

Table 758. Compounds of formula I.b, wherein R² is R²-10.72 (=formula 1.758).

Table 759. Compounds of formula I.b, wherein R² is R²-10.73 (=formula 1.759).

Table 760. Compounds of formula I.b, wherein R² is R²-10.74 (=formula 1.760).

Table 761. Compounds of formula I.b, wherein R² is R²-10.75 (=formula 1.761).

Table 762. Compounds of formula I.b, wherein R² is R²-10.76 (=formula 1.762).

Table 763. Compounds of formula I.b, wherein R² is R²-10.77 (=formula 1.763).

Table 764. Compounds of formula I.b, wherein R² is R²-10.78 (=formula 1.764).

Table 765. Compounds of formula I.b, wherein R² is R²-10.79 (=formula 1.765).

Table 766. Compounds of formula I.b, wherein R² is R²-10.80 (=formula 1.766).

Table 767. Compounds of formula I.b, wherein R² is R²-10.81 (=formula 1.767).

Table 768. Compounds of formula I.b, wherein R² is R²-10.82 (=formula 1.768).

Table 769. Compounds of formula I.b, wherein R² is R²-10.83 (=formula 1.769).

Table 770. Compounds of formula I.b, wherein R² is R²-10.84 (=formula 1.770).

Table 771. Compounds of formula I.b, wherein R² is R²-10.85 (=formula 1.771).

Table 772. Compounds of formula I.b, wherein R² is R²-10.86 (=formula 1.772).

Table 773. Compounds of formula I.b, wherein R² is R²-10.87 (=formula 1.773).

Table 774. Compounds of formula I.b, wherein R² is R²-10.88 (=formula 1.774).

Table 775. Compounds of formula I.b, wherein R² is R²-10.89 (=formula 1.775).

Table 776. Compounds of formula I.b, wherein R² is R²-10.90 (=formula 1.776).

Table 777. Compounds of formula I.b, wherein R² is R²-10.91 (=formula 1.777).

Table 778. Compounds of formula I.b, wherein R² is R²-10.92 (=formula 1.778).

Table 779. Compounds of formula I.b, wherein R² is R²-10.93 (=formula 1.779).

Table 780. Compounds of formula I.b, wherein R² is R²-10.94 (=formula 1.780).

Table 781. Compounds of formula I.b, wherein R² is R²-10.95 (=formula 1.781).

Table 782. Compounds of formula I.b, wherein R² is R²-10.96 (=formula 1.782).

Table 783. Compounds of formula I.b, wherein R² is R²-10.97 (=formula 1.783).

Table 784. Compounds of formula I.b, wherein R² is R²-10.98 (=formula 1.784).

Table 785. Compounds of formula I.b, wherein R² is R²-10.99 (=formula 1.785).

Table 786. Compounds of formula I.b, wherein R² is R²-10.100 (=formula 1.786).

Table 787. Compounds of formula I.b, wherein R² is R²-10.101 (=formula 1.787).

Table 788. Compounds of formula I.b, wherein R² is R²-10.102 (=formula 1.788).

Table 789. Compounds of formula I.b, wherein R² is R²-10.103 (=formula 1.789).

Table 790. Compounds of formula I.b, wherein R² is R²-10.104 (=formula 1.790).

Table 791. Compounds of formula I.b, wherein R² is R²-10.105 (=formula 1.791).

Table 792. Compounds of formula I.b, wherein R² is R²-10.106 (=formula 1.792).

Table 793. Compounds of formula I.b, wherein R² is R²-10.107 (=formula 1.793).

Table 794. Compounds of formula I.b, wherein R² is R²-10.108 (=formula 1.794).

Table 795. Compounds of formula I.b, wherein R² is R²-10.109 (=formula 1.795).

Table 796. Compounds of formula I.b, wherein R² is R²-10.110 (=formula 1.796).

Table 797. Compounds of formula I.b, wherein R² is R²-10.111 (=formula 1.797).

Table 798. Compounds of formula I.b, wherein R² is R²-10.112 (=formula 1.798).

Table 799. Compounds of formula I.b, wherein R² is R²-10.113 (=formula 1.799).

Table 800. Compounds of formula I.b, wherein R² is R²-10.114 (=formula 1.800).

Table 801. Compounds of formula I.b, wherein R² is R²-10.115 (=formula 1.801).

Table 802. Compounds of formula I.b, wherein R² is R²-10.116 (=formula 1.802).

Table 803. Compounds of formula I.b, wherein R² is R²-10.117 (=formula 1.803).

Table 804. Compounds of formula I.b, wherein R² is R²-10.118 (=formula 1.804).

Table 805. Compounds of formula I.b, wherein R² is R²-10.119 (=formula 1.805).

Table 806. Compounds of formula I.b, wherein R² is R²-10.120 (=formula 1.806).

Table 807. Compounds of formula I.b, wherein R² is R²-10.121 (=formula 1.807).

Table 808. Compounds of formula I.b, wherein R² is R²-10.122 (=formula 1.808).

Table 809. Compounds of formula I.b, wherein R² is R²-10.123 (=formula 1.809).

Table 810. Compounds of formula I.b, wherein R² is R²-10.124 (=formula 1.810).

Table 811. Compounds of formula I.b, wherein R² is R²-10.125 (=formula 1.811).

Table 812. Compounds of formula I.b, wherein R² is R²-10.126 (=formula 1.812).

Table 813. Compounds of formula I.b, wherein R² is R²-10.127 (=formula 1.813).

Table 814. Compounds of formula I.b, wherein R² is R²-10.128 (=formula 1.814).

Table 815. Compounds of formula I.b, wherein R² is R²-10.129 (=formula 1.815).

Table 816. Compounds of formula I.b, wherein R² is R²-10.130 (=formula 1.816).

Table 817. Compounds of formula I.b, wherein R² is R²-10.131 (=formula 1.817).

Table 818. Compounds of formula I.b, wherein R² is R²-10.132 (=formula 1.818).

Table 819. Compounds of formula I.b, wherein R² is R²-10.133 (=formula 1.819).

Table 820. Compounds of formula I.b, wherein R² is R²-10.134 (=formula 1.820).

Table 821. Compounds of formula I.b, wherein R² is R²-10.135 (=formula 1.821).

Table 822. Compounds of formula I.b, wherein R² is R²-10.136 (=formula 1.822).

Table 823. Compounds of formula I.b, wherein R² is R²-10.137 (=formula 1.823).

Table 824. Compounds of formula I.b, wherein R² is R²-10.138 (=formula 1.824).

Table 825. Compounds of formula I.b, wherein R² is R²-10.139 (=formula 1.825).

Table 826. Compounds of formula I.b, wherein R² is R²-10.140 (=formula 1.826).

Table 827. Compounds of formula I.b, wherein R² is R²-10.141 (=formula 1.827).

Table 828. Compounds of formula I.b, wherein R² is R²-10.142 (=formula 1.828).

Table 829. Compounds of formula I.b, wherein R² is R²-10.143 (=formula 1.829).

Table 830. Compounds of formula I.b, wherein R² is R²-10.144 (=formula 1.830).

Table 831. Compounds of formula I.b, wherein R² is R²-10.145 (=formula 1.831).

Table 832. Compounds of formula I.b, wherein R² is R²-10.146 (=formula 1.832).

Table 833. Compounds of formula I.b, wherein R² is R²-10.147 (=formula 1.833).

Table 834. Compounds of formula I.b, wherein R² is R²-10.148 (=formula 1.834).

Table 835. Compounds of formula I.b, wherein R² is R²-10.149 (=formula 1.835).

Table 836. Compounds of formula I.b, wherein R² is R²-10.150 (=formula 1.836).

Table 837. Compounds of formula I.b, wherein R² is R²-10.151 (=formula 1.837).

Table 838. Compounds of formula I.b, wherein R² is R²-10.152 (=formula 1.838).

Table 839. Compounds of formula I.b, wherein R² is R²-10.153 (=formula 1.839).

Table 840. Compounds of formula I.b, wherein R² is R²-10.154 (=formula 1.840).

Table 841. Compounds of formula I.b, wherein R² is R²-10.155 (=formula 1.841).

Table 842. Compounds of formula I.b, wherein R² is R²-10.156 (=formula 1.842).

Table 843. Compounds of formula I.b, wherein R² is R²-10.157 (=formula 1.843).

Table 844. Compounds of formula I.b, wherein R² is R²-10.158 (=formula 1.844).

Table 845. Compounds of formula I.b, wherein R² is R²-10.159 (=formula 1.845).

Table 846. Compounds of formula I.b, wherein R² is R²-10.160 (=formula 1.846).

Table 847. Compounds of formula I.b, wherein R² is R²-10.161 (=formula 1.847).

Table 848. Compounds of formula I.b, wherein R² is R²-10.162 (=formula 1.848).

Table 849. Compounds of formula I.b, wherein R² is R²-10.163 (=formula 1.849).

Table 850. Compounds of formula I.b, wherein R² is R²-10.164 (=formula 1.850).

Table 851. Compounds of formula I.b, wherein R² is R²-10.165 (=formula 1.851).

Table 852. Compounds of formula I.b, wherein R² is R²-10.166 (=formula 1.852).

Table 853. Compounds of formula I.b, wherein R² is R²-10.167 (=formula 1.853).

Table 854. Compounds of formula I.b, wherein R² is R²-10.168 (=formula 1.854).

Table 855. Compounds of formula I.b, wherein R² is R²-10.169 (=formula 1.855).

Table 856. Compounds of formula I.b, wherein R² is R²-10.170 (=formula 1.856).

Table 857. Compounds of formula I.b, wherein R² is R²-10.171 (=formula 1.857).

Table 858. Compounds of formula I.b, wherein R² is R²-10.172 (=formula 1.858).

Table 859. Compounds of formula I.b, wherein R² is R²-10.173 (=formula 1.859).

Table 860. Compounds of formula I.b, wherein R² is R²-10.174 (=formula 1.860).

Table 861. Compounds of formula I.b, wherein R² is R²-10.175 (=formula 1.861).

Table 862. Compounds of formula I.b, wherein R² is R²-10.176 (=formula 1.862).

Table 863. Compounds of formula I.b, wherein R² is R²-10.177 (=formula 1.863).

Table 864. Compounds of formula I.b, wherein R² is R²-10.178 (=formula 1.864).

Table 865. Compounds of formula I.b, wherein R² is R²-10.179 (=formula 1.865).

Table 866. Compounds of formula I.b, wherein R² is R²-10.180 (=formula 1.866).

Table 867. Compounds of formula I.b, wherein R² is R²-10.181 (=formula 1.867).

Table 868. Compounds of formula I.b, wherein R² is R²-10.182 (=formula 1.868).

Table 869. Compounds of formula I.b, wherein R² is R²-10.183 (=formula 1.869).

Table 870. Compounds of formula I.b, wherein R² is R²-10.184 (=formula 1.870).

Table 871. Compounds of formula I.b, wherein R² is R²-10.185 (=formula 1.871).

Table 872. Compounds of formula I.b, wherein R² is R²-10.186 (=formula 1.872).

Table 873. Compounds of formula I.b, wherein R² is R²-10.187 (=formula 1.873).

Table 874. Compounds of formula I.b, wherein R² is R²-10.188 (=formula 1.874).

Table 875. Compounds of formula I.b, wherein R² is R²-10.189 (=formula 1.875).

Table 876. Compounds of formula I.b, wherein R² is R²-10.190 (=formula 1.876).

Table 877. Compounds of formula I.b, wherein R² is R²-10.191 (=formula 1.877).

Table 878. Compounds of formula I.b, wherein R² is R²-10.192 (=formula 1.878).

Table 879. Compounds of formula I.b, wherein R² is R²-10.193 (=formula 1.879).

Table 880. Compounds of formula I.b, wherein R² is R²-10.194 (=formula 1.880).

Table 881. Compounds of formula I.b, wherein R² is R²-10.195 (=formula 1.881).

Table 882. Compounds of formula I.b, wherein R² is R²-10.196 (=formula 1.882).

Table 883. Compounds of formula I.b, wherein R² is R²-10.197 (=formula 1.883).

Table 884. Compounds of formula I.b, wherein R² is R²-10.198 (=formula 1.884).

Table 885. Compounds of formula I.b, wherein R² is R²-10.199 (=formula 1.885).

Table 886. Compounds of formula I.b, wherein R² is R²-10.200 (=formula 1.886).

Table 887. Compounds of formula I.b, wherein R² is R²-10.201 (=formula 1.887).

Table 888. Compounds of formula I.b, wherein R² is R²-10.202 (=formula 1.888).

Table 889. Compounds of formula I.b, wherein R² is R²-10.203 (=formula 1.889).

Table 890. Compounds of formula I.b, wherein R² is R²-10.204 (=formula 1.890).

Table 891. Compounds of formula I.b, wherein R² is R²-10.205 (=formula 1.891).

Table 892. Compounds of formula I.b, wherein R² is R²-10.206 (=formula 1.892).

Table 893. Compounds of formula I.b, wherein R² is R²-10.207 (=formula 1.893).

Table 894. Compounds of formula I.b, wherein R² is R²-10.208 (=formula 1.894).

Table 895. Compounds of formula I.b, wherein R² is R²-10.209 (=formula 1.895).

Table 896. Compounds of formula I.b, wherein R² is R²-10.210 (=formula 1.896).

Table 897. Compounds of formula I.b, wherein R² is R²-10.211 (=formula 1.897).

Table 898. Compounds of formula I.b, wherein R² is R²-10.212 (=formula 1.898).

Table 899. Compounds of formula I.b, wherein R² is R²-10.213 (=formula 1.899).

Table 900. Compounds of formula I.b, wherein R² is R²-10.214 (=formula 1.900).

Table 901. Compounds of formula I.b, wherein R² is R²-10.215 (=formula 1.901).

Table 902. Compounds of formula I.b, wherein R² is R²-10.216 (=formula 1.902).

Table 903. Compounds of formula I.b, wherein R² is R²-10.217 (=formula 1.903).

Table 904. Compounds of formula I.b, wherein R² is R²-10.218 (=formula 1.904).

Table 905. Compounds of formula I.b, wherein R² is R²-10.219 (=formula 1.905).

Table 906. Compounds of formula I.b, wherein R² is R²-10.220 (=formula 1.906).

Table 907. Compounds of formula I.b, wherein R² is R²-10.221 (=formula 1.907).

Table 908. Compounds of formula I.b, wherein R² is R²-10.222 (=formula 1.908).

Table 909. Compounds of formula I.b, wherein R² is R²-10.223 (=formula 1.909).

Table 910. Compounds of formula I.b, wherein R² is R²-10.224 (=formula 1.910).

Table 911. Compounds of formula I.b, wherein R² is R²-10.225 (=formula 1.911).

Table 912. Compounds of formula I.b, wherein R² is R²-10.226 (=formula 1.912).

Table 913. Compounds of formula I.b, wherein R² is R²-10.227 (=formula 1.913).

Table 914. Compounds of formula I.b, wherein R² is R²-10.228 (=formula 1.914).

Table 915. Compounds of formula I.b, wherein R² is R²-10.229 (=formula 1.915).

Table 916. Compounds of formula I.b, wherein R² is R²-10.230 (=formula 1.916).

Table 917. Compounds of formula I.b, wherein R² is R²-10.231 (=formula 1.917).

Table 918. Compounds of formula I.b, wherein R² is R²-10.232 (=formula 1.918).

Table 919. Compounds of formula I.b, wherein R² is R²-10.233 (=formula 1.919).

Table 920. Compounds of formula I.b, wherein R² is R²-10.234 (=formula 1.920).

Table 921. Compounds of formula I.b, wherein R² is R²-10.235 (=formula 1.921).

Table 922. Compounds of formula I.b, wherein R² is R²-10.236 (=formula 1.922).

Table 923. Compounds of formula I.b, wherein R² is R²-10.237 (=formula 1.923).

Table 924. Compounds of formula I.b, wherein R² is R²-10.238 (=formula 1.924).

Table 925. Compounds of formula I.b, wherein R² is R²-10.239 (=formula 1.925).

Table 926. Compounds of formula I.b, wherein R² is R²-10.240 (=formula 1.926).

Table 927. Compounds of formula I.b, wherein R² is R²-10.241 (=formula 1.927).

Table 928. Compounds of formula I.b, wherein R² is R²-10.242 (=formula 1.928).

Table 929. Compounds of formula I.b, wherein R² is R²-10.243 (=formula 1.929).

Table 930. Compounds of formula I.b, wherein R² is R²-10.244 (=formula 1.930).

Table 931. Compounds of formula I.b, wherein R² is R²-10.245 (=formula 1.931).

Table 932. Compounds of formula I.b, wherein R² is R²-10.246 (=formula 1.932).

Table 933. Compounds of formula I.b, wherein R² is R²-10.247 (=formula 1.933).

Table 934. Compounds of formula I.b, wherein R² is R²-10.248 (=formula 1.934).

Table 935. Compounds of formula I.b, wherein R² is R²-10.249 (=formula 1.935).

Table 936. Compounds of formula I.b, wherein R² is R²-10.250 (=formula 1.936).

Table 937. Compounds of formula I.b, wherein R² is R²-10.251 (=formula 1.937).

Table 938. Compounds of formula I.b, wherein R² is R²-10.252 (=formula 1.938).

Table 939. Compounds of formula I.b, wherein R² is R²-10.253 (=formula 1.939).

Table 940. Compounds of formula I.b, wherein R² is R²-10.254 (=formula 1.940).

Table 941. Compounds of formula I.b, wherein R² is R²-10.255 (=formula 1.941).

Table 942. Compounds of formula I.b, wherein R² is R²-10.256 (=formula 1.942).

Table 943. Compounds of formula I.b, wherein R² is R²-10.257 (=formula 1.943).

Table 944. Compounds of formula I.b, wherein R² is R²-10.258 (=formula 1.944).

Table 945. Compounds of formula I.b, wherein R² is R²-10.259 (=formula 1.945).

Table 946. Compounds of formula I.b, wherein R² is R²-10.260 (=formula 1.946).

Table 947. Compounds of formula I.b, wherein R² is R²-10.261 (=formula 1.947).

Table 948. Compounds of formula I.b, wherein R² is R²-10.262 (=formula 1.948).

Table 949. Compounds of formula I.b, wherein R² is R²-10.263 (=formula 1.949).

Table 950. Compounds of formula I.b, wherein R² is R²-10.264 (=formula 1.950).

Table 951. Compounds of formula I.b, wherein R² is R²-10.265 (=formula 1.951).

Table 952. Compounds of formula I.b, wherein R² is R²-10.266 (=formula 1.952).

Table 953. Compounds of formula I.b, wherein R² is R²-10.267 (=formula 1.953).

Table 954. Compounds of formula I.b, wherein R² is R²-10.268 (=formula 1.954).

Table 955. Compounds of formula I.b, wherein R² is R²-10.269 (=formula 1.955).

Table 956. Compounds of formula I.b, wherein R² is R²-10.270 (=formula 1.956).

Table 957. Compounds of formula I.b, wherein R² is R²-10.271 (=formula 1.957).

Table 958. Compounds of formula I.b, wherein R² is R²-10.272 (=formula 1.958).

Table 959. Compounds of formula I.b, wherein R² is R²-10.273 (=formula 1.959).

Table 960. Compounds of formula I.b, wherein R² is R²-10.274 (=formula 1.960).

Table 961. Compounds of formula I.b, wherein R² is R²-10.275 (=formula 1.961).

Table 962. Compounds of formula I.b, wherein R² is R²-10.276 (=formula 1.962).

Table 963. Compounds of formula I.b, wherein R² is R²-10.277 (=formula 1.963).

Table 964. Compounds of formula I.b, wherein R² is R²-10.278 (=formula 1.964).

Table 965. Compounds of formula I.b, wherein R² is R²-10.279 (=formula 1.965).

Table 966. Compounds of formula I.b, wherein R² is R²-10.280 (=formula 1.966).

Table 967. Compounds of formula I.b, wherein R² is R²-10.281 (=formula 1.967).

Table 968. Compounds of formula I.b, wherein R² is R²-10.282 (=formula 1.968).

Table 969. Compounds of formula I.b, wherein R² is R²-10.283 (=formula 1.969).

Table 970. Compounds of formula I.b, wherein R² is R²-10.284 (=formula 1.970).

Table 971. Compounds of formula I.b, wherein R² is R²-10.285 (=formula 1.971).

Table 972. Compounds of formula I.b, wherein R² is R²-10.286 (=formula 1.972).

Table 973. Compounds of formula I.b, wherein R² is R²-10.287 (=formula 1.973).

Table 974. Compounds of formula I.b, wherein R² is R²-10.288 (=formula 1.974).

Table 975. Compounds of formula I.b, wherein R² is R²-10.289 (=formula 1.975).

Table 976. Compounds of formula I.b, wherein R² is R²-10.290 (=formula 1.976).

Table 977. Compounds of formula I.b, wherein R² is R²-10.291 (=formula 1.977).

Table 978. Compounds of formula I.b, wherein R² is R²-10.292 (=formula 1.978).

Table 979. Compounds of formula I.b, wherein R² is R²-10.293 (=formula 1.979).

Table 980. Compounds of formula I.b, wherein R² is R²-10.294 (=formula 1.980).

Table 981. Compounds of formula I.b, wherein R² is R²-10.295 (=formula 1.981).

Table 982. Compounds of formula I.b, wherein R² is R²-10.296 (=formula 1.982).

Table 983. Compounds of formula I.b, wherein R² is R²-10.297 (=formula 1.983).

Table 984. Compounds of formula I.b, wherein R² is R²-10.298 (=formula 1.984).

Table 985. Compounds of formula I.b, wherein R² is R²-10.299 (=formula 1.985).

Table 986. Compounds of formula I.b, wherein R² is R²-10.300 (=formula 1.986).

Table 987. Compounds of formula I.b, wherein R² is R²-10.301 (=formula 1.987).

Table 988. Compounds of formula I.b, wherein R² is R²-10.302 (=formula 1.988).

Table 989. Compounds of formula I.b, wherein R² is R²-10.303 (=formula 1.989).

Table 990. Compounds of formula I.b, wherein R² is R²-10.304 (=formula 1.990).

Table 991. Compounds of formula I.b, wherein R² is R²-10.305 (=formula 1.991).

Table 992. Compounds of formula I.b, wherein R² is R²-10.306 (=formula 1.992).

Table 993. Compounds of formula I.b, wherein R² is R²-10.307 (=formula 1.993).

Table 994. Compounds of formula I.b, wherein R² is R²-10.308 (=formula 1.994).

Table 995. Compounds of formula I.b, wherein R² is R²-10.309 (=formula 1.995).

Table 996. Compounds of formula I.b, wherein R² is R²-10.310 (=formula 1.996).

Table 997. Compounds of formula I.b, wherein R² is R²-10.311 (=formula 1.997).

Table 998. Compounds of formula I.b, wherein R² is R²-10.312 (=formula 1.998).

Table 999. Compounds of formula I.b, wherein R² is R²-10.313 (=formula 1.999).

Table 1000. Compounds of formula I.b, wherein R² is R²-10.314 (=formula 1.1000).

Table 1001. Compounds of formula I.b, wherein R² is R²-10.315 (=formula 1.1001).

Table 1002. Compounds of formula I.b, wherein R² is R²-10.316 (=formula 1.1002).

Table 1003. Compounds of formula I.b, wherein R² is R²-10.317 (=formula 1.1003).

Table 1004. Compounds of formula I.b, wherein R² is R²-10.318 (=formula 1.1004).

Table 1005. Compounds of formula I.b, wherein R² is R²-10.319 (=formula 1.1005).

Table 1006. Compounds of formula I.b, wherein R² is R²-10.320 (=formula 1.1006).

Table 1007. Compounds of formula I.b, wherein R² is R²-10.321 (=formula 1.1007).

Table 1008. Compounds of formula I.b, wherein R² is R²-10.322 (=formula 1.1008).

Table 1009. Compounds of formula I.b, wherein R² is R²-10.323 (=formula 1.1009).

Table 1010. Compounds of formula I.b, wherein R² is R²-10.324 (=formula 1.1010).

Table 1011. Compounds of formula I.b, wherein R² is R²-10.325 (=formula 1.1011).

Table 1012. Compounds of formula I.b, wherein R² is R²-10.326 (=formula 1.1012).

Table 1013. Compounds of formula I.b, wherein R² is R²-10.327 (=formula 1.1013).

Table 1014. Compounds of formula I.b, wherein R² is R²-10.328 (=formula 1.1014).

Table 1015. Compounds of formula I.b, wherein R² is R²-10.329 (=formula 1.1015).

Table 1016. Compounds of formula I.b, wherein R² is R²-10.330 (=formula 1.1016).

Table 1017. Compounds of formula I.b, wherein R² is R²-10.331 (=formula 1.1017).

Table 1018. Compounds of formula I.b, wherein R² is R²-10.332 (=formula 1.1018).

Table 1019. Compounds of formula I.b, wherein R² is R²-10.333 (=formula 1.1019).

Table 1020. Compounds of formula I.b, wherein R² is R²-10.334 (=formula 1.1020).

Table 1021. Compounds of formula I.b, wherein R² is R²-10.335 (=formula 1.1021).

Table 1022. Compounds of formula I.b, wherein R² is R²-10.336 (=formula 1.1022).

Table 1023. Compounds of formula I.b, wherein R² is R²-10.334 (=formula 1.1023).

Table 1024. Compounds of formula I.b, wherein R² is R²-10.338 (=formula 1.1024).

Table 1025. Compounds of formula I.b, wherein R² is R²-10.339 (=formula 1.1025).

Table 1026. Compounds of formula I.b, wherein R² is R²-10.340 (=formula 1.1026).

Table 1027. Compounds of formula I.b, wherein R² is R²-10.341 (=formula 1.1027).

Table 1028. Compounds of formula I.b, wherein R² is R²-10.342 (=formula 1.1028).

Table 1029. Compounds of formula I.b, wherein R² is R²-10.343 (=formula 1.1029).

Table 1030. Compounds of formula I.b, wherein R² is R²-10.344 (=formula 1.1030).

Table 1031. Compounds of formula I.b, wherein R² is R²-10.345 (=formula 1.1031).

Table 1032. Compounds of formula I.b, wherein R² is R²-10.346 (=formula 1.1032).

Table 1033. Compounds of formula I.b, wherein R² is R²-10.347 (=formula 1.1033).

Table 1034. Compounds of formula I.b, wherein R² is R²-10.348 (=formula 1.1034).

Table 1035. Compounds of formula I.b, wherein R² is R²-10.349 (=formula 1.1035).

Table 1036. Compounds of formula I.b, wherein R² is R²-10.350 (=formula 1.1036).

Table 1037. Compounds of formula I.b, wherein R² is R²-10.351 (=formula 1.1037).

Table 1038. Compounds of formula I.b, wherein R² is R²-10.352 (=formula 1.1038).

Table 1039. Compounds of formula I.b, wherein R² is R²-10.353 (=formula 1.1039).

Table 1040. Compounds of formula I.b, wherein R² is R²-10.354 (=formula 1.1040).

Table 1041. Compounds of formula I.b, wherein R² is R²-10.355 (=formula 1.1041).

Table 1042. Compounds of formula I.b, wherein R² is R²-10.356 (=formula 1.1042).

Table 1043. Compounds of formula I.b, wherein R² is R²-10.357 (=formula 1.1043).

Table 1044. Compounds of formula I.b, wherein R² is R²-10.358 (=formula 1.1044).

Table 1045. Compounds of formula I.b, wherein R² is R²-10.359 (=formula 1.1045).

Table 1046. Compounds of formula I.b, wherein R² is R²-10.360 (=formula 1.1046).

Table 1047. Compounds of formula I.b, wherein R² is R²-10.361 (=formula 1.1047).

Table 1048. Compounds of formula I.b, wherein R² is R²-10.362 (=formula 1.1048).

Table 1049. Compounds of formula I.b, wherein R² is R²-10.363 (=formula 1.1049).

Table 1050. Compounds of formula I.b, wherein R² is R²-10.364 (=formula 1.1050).

Table 1051. Compounds of formula I.b, wherein R² is R²-10.365 (=formula 1.1051).

Table 1052. Compounds of formula I.b, wherein R² is R²-10.366 (=formula 1.1052).

Table 1053. Compounds of formula I.b, wherein R² is R²-10.367 (=formula 1.1053).

Table 1054. Compounds of formula I.b, wherein R² is R²-10.368 (=formula 1.1054).

Table 1055. Compounds of formula I.b, wherein R² is R²-10.369 (=formula 1.1055).

Table 1056. Compounds of formula I.b, wherein R² is R²-10.370 (=formula 1.1056).

Table 1057. Compounds of formula I.b, wherein R² is R²-10.371 (=formula 1.1057).

Table 1058. Compounds of formula I.b, wherein R² is R²-10.372 (=formula 1.1058).

Table 1059. Compounds of formula I.b, wherein R² is R²-10.373 (=formula 1.1059).

Table 1060. Compounds of formula I.b, wherein R² is R²-10.374 (=formula 1.1060).

Table 1061. Compounds of formula I.b, wherein R² is R²-10.375 (=formula 1.1061).

Table 1062. Compounds of formula I.b, wherein R² is R²-10.376 (=formula 1.1062).

Table 1063. Compounds of formula I.b, wherein R² is R²-10.377 (=formula 1.1063).

Table 1064. Compounds of formula I.b, wherein R² is R²-10.378 (=formula 1.1064).

Table 1065. Compounds of formula I.b, wherein R² is R²-10.379 (=formula 1.1065).

Table 1066. Compounds of formula I.b, wherein R² is R²-10.380 (=formula 1.1066).

Table 1067. Compounds of formula I.b, wherein R² is R²-10.381 (=formula 1.1067).

Table 1068. Compounds of formula I.b, wherein R² is R²-10.382 (=formula 1.1068).

Table 1069. Compounds of formula I.b, wherein R² is R²-10.383 (=formula 1.1069).

Table 1070. Compounds of formula I.b, wherein R² is R²-10.384 (=formula 1.1070).

Table 1071. Compounds of formula I.b, wherein R² is R²-10.385 (=formula 1.1071).

Table 1072. Compounds of formula I.b, wherein R² is R²-10.386 (=formula 1.1072).

Table 1073. Compounds of formula I.b, wherein R² is R²-10.387 (=formula 1.1073).

Table 1074. Compounds of formula I.b, wherein R² is R²-10.388 (=formula 1.1074).

Table 1075. Compounds of formula I.b, wherein R² is R²-10.389 (=formula 1.1075).

Table 1076. Compounds of formula I.b, wherein R² is R²-10.390 (=formula 1.7076).

Table 1077. Compounds of formula I.b, wherein R² is R²-10.391 (=formula 1.1077).

Table 1078. Compounds of formula I.b, wherein R² is R²-10.392 (=formula 1.1078).

Table 1079. Compounds of formula I.b, wherein R² is R²-10.393 (=formula 1.1079).

Table 1080. Compounds of formula I.b, wherein R² is R²-10.394 (=formula 1.1080).

Table 1081. Compounds of formula I.b, wherein R² is R²-10.395 (=formula 1.1081).

Table 1082. Compounds of formula I.b, wherein R² is R²-10.396 (=formula 1.1082).

Table 1083. Compounds of formula I.b, wherein R² is R²-10.397 (=formula 1.1083).

Table 1084. Compounds of formula I.b, wherein R² is R²-10.398 (=formula 1.1084).

Table 1085. Compounds of formula I.b, wherein R² is R²-10.399 (=formula 1.1085).

Table 1086. Compounds of formula I.b, wherein R² is R²-10.400 (=formula 1.1086).

Table 1087. Compounds of formula I.b, wherein R² is R²-10.401 (=formula 1.1087).

Table 1088. Compounds of formula I.b, wherein R² is R²-10.402 (=formula 1.1088).

Table 1089. Compounds of formula I.b, wherein R² is R²-10.403 (=formula 1.1089).

Table 1090. Compounds of formula I.b, wherein R² is R²-10.404 (=formula 1.1090).

Table 1091. Compounds of formula I.b, wherein R² is R²-10.405 (=formula 1.1091).

Table 1092. Compounds of formula I.b, wherein R² is R²-10.406 (=formula 1.1092).

Table 1093. Compounds of formula I.b, wherein R² is R²-10.407 (=formula 1.1093).

Table 1094. Compounds of formula I.b, wherein R² is R²-10.408 (=formula 1.1094).

Table 1095. Compounds of formula I.b, wherein R² is R²-10.409 (=formula 1.1095).

Table 1096. Compounds of formula I.b, wherein R² is R²-10.410 (=formula 1.1096).

Table 1097. Compounds of formula I.b, wherein R² is R²-10.411 (=formula 1.1097).

Table 1098. Compounds of formula I.b, wherein R² is R²-10.412 (=formula 1.1098).

Table 1099. Compounds of formula I.b, wherein R² is R²-10.413 (=formula 1.1099).

Table 1100. Compounds of formula I.b, wherein R² is R²-10.414 (=formula 1.1100).

Table 1101. Compounds of formula I.b, wherein R² is R²-10.415 (=formula 1.1101).

Table 1102. Compounds of formula I.b, wherein R² is R²-10.416 (=formula 1.1102).

Table 1103. Compounds of formula I.b, wherein R² is R²-10.417 (=formula 1.1103).

Table 1104. Compounds of formula I.b, wherein R² is R²-10.418 (=formula 1.1104).

Table 1105. Compounds of formula I.b, wherein R² is R²-10.419 (=formula 1.1105).

Table 1106. Compounds of formula I.b, wherein R² is R²-10.420 (=formula 1.1106).

Table 1107. Compounds of formula I.b, wherein R² is R²-10.421 (=formula 1.1107).

Table 1108. Compounds of formula I.b, wherein R² is R²-10.422 (=formula 1.1108).

Table 1109. Compounds of formula I.b, wherein R² is R²-10.423 (=formula 1.1109).

Table 1110. Compounds of formula I.b, wherein R² is R²-10.424 (=formula 1.1110).

Table 1111. Compounds of formula I.b, wherein R² is R²-10.425 (=formula 1.1111).

Table 1112. Compounds of formula I.b, wherein R² is R²-10.426 (=formula 1.1112).

Table 1113. Compounds of formula I.b, wherein R² is R²-10.427 (=formula 1.1113).

Table 1114. Compounds of formula I.b, wherein R² is R²-10.428 (=formula 1.1114).

Table 1115. Compounds of formula I.b, wherein R² is R²-10.429 (=formula 1.1115).

Table 1116. Compounds of formula I.b, wherein R² is R²-10.430 (=formula 1.1116).

Table 1117. Compounds of formula I.b, wherein R² is R²-10.431 (=formula 1.1117).

Table 1118. Compounds of formula I.b, wherein R² is R²-10.432 (=formula 1.1118).

Table 1119. Compounds of formula I.b, wherein R² is R²-10.433 (=formula 1.1119).

Table 1120. Compounds of formula I.b, wherein R² is R²-10.434 (=formula 1.1120).

Table 1121. Compounds of formula I.b, wherein R² is R²-10.435 (=formula 1.1121).

Table 1122. Compounds of formula I.b, wherein R² is R²-10.436 (=formula 1.1122).

Table 1123. Compounds of formula I.b, wherein R² is R²-10.437 (=formula 1.1123).

Table 1124. Compounds of formula I.b, wherein R² is R²-10.438 (=formula 1.1124).

Table 1125. Compounds of formula I.b, wherein R² is R²-10.439 (=formula 1.1125).

Table 1126. Compounds of formula I.b, wherein R² is R²-10.440 (=formula 1.1126).

Table 1127. Compounds of formula I.b, wherein R² is R²-10.441 (=formula 1.1127).

Table 1128. Compounds of formula I.b, wherein R² is R²-10.442 (=formula 1.1128).

Table 1129. Compounds of formula I.b, wherein R² is R²-10.443 (=formula 1.1129).

Table 1130. Compounds of formula I.b, wherein R² is R²-10.444 (=formula 1.1130).

Table 1131. Compounds of formula I.b, wherein R² is R²-10.445 (=formula 1.1131).

Table 1132. Compounds of formula I.b, wherein R² is R²-10.446 (=formula 1.1132).

Table 1133. Compounds of formula I.b, wherein R² is R²-10.447 (=formula 1.1133).

Table 1134. Compounds of formula I.b, wherein R² is R²-10.448 (=formula 1.1134).

Table 1135. Compounds of formula I.b, wherein R² is R²-10.449 (=formula 1.1135).

Table 1136. Compounds of formula I.b, wherein R² is R²-10.450 (=formula 1.1136).

Table 1137. Compounds of formula I.b, wherein R² is R²-10.451 (=formula 1.1137).

Table 1138. Compounds of formula I.b, wherein R² is R²-10.452 (=formula 1.1138).

Table 1139. Compounds of formula I.b, wherein R² is R²-10.453 (=formula 1.1139).

Table 1140. Compounds of formula I.b, wherein R² is R²-10.454 (=formula 1.1140).

Table 1141. Compounds of formula I.b, wherein R² is R²-10.455 (=formula 1.1141).

Table 1142. Compounds of formula I.b, wherein R² is R²-10.456 (=formula 1.1142).

Table 1143. Compounds of formula I.b, wherein R² is R²-10.457 (=formula 1.1143).

Table 1144. Compounds of formula I.b, wherein R² is R²-10.458 (=formula 1.1144).

Table 1145. Compounds of formula I.b, wherein R² is R²-10.459 (=formula 1.1145).

Table 1146. Compounds of formula I.b, wherein R² is R²-10.460 (=formula 1.1146).

Table 1147. Compounds of formula I.b, wherein R² is R²-10.461 (=formula 1.1147).

Table 1148. Compounds of formula I.b, wherein R² is R²-10.462 (=formula 1.1148).

Table 1149. Compounds of formula I.b, wherein R² is R²-10.463 (=formula 1.1149).

Table 1150. Compounds of formula I.b, wherein R² is R²-10.464 (=formula 1.1150).

Table 1151. Compounds of formula I.b, wherein R² is R²-10.465 (=formula 1.1151).

Table 1152. Compounds of formula I.b, wherein R² is R²-10.466 (=formula 1.1152).

Table 1153. Compounds of formula I.b, wherein R² is R²-10.467 (=formula 1.1153).

Table 1154. Compounds of formula I.b, wherein R² is R²-10.468 (=formula 1.1154).

Table 1155. Compounds of formula I.b, wherein R² is R²-10.469 (=formula 1.1154).

Table 1156. Compounds of formula I.b, wherein R² is R²-10.470 (=formula 1.1156).

Table 1157. Compounds of formula I.b, wherein R² is R²-10.471 (=formula 1.1157).

Table 1158. Compounds of formula I.b, wherein R² is R²-10.472 (=formula 1.1158).

Table 1159. Compounds of formula I.b, wherein R² is R²-10.473 (=formula 1.1159).

Table 1160. Compounds of formula I.b, wherein R² is R²-10.474 (=formula 1.1160).

Table 1161. Compounds of formula I.b, wherein R² is R²-10.475 (=formula 1.1161).

Table 1162. Compounds of formula I.b, wherein R² is R²-10.476 (=formula 1.1162).

Table 1163. Compounds of formula I.b, wherein R² is R²-10.477 (=formula 1.1163).

Table 1164. Compounds of formula I.b, wherein R² is R²-10.478 (=formula 1.1164).

Table 1165. Compounds of formula I.b, wherein R² is R²-10.479 (=formula 1.1165).

Table 1166. Compounds of formula I.b, wherein R² is R²-10.480 (=formula 1.1166).

Table 1167. Compounds of formula I.b, wherein R² is R²-10.481 (=formula 1.1167).

Table 1168. Compounds of formula I.b, wherein R² is R²-10.482 (=formula 1.1168).

Table 1169. Compounds of formula I.b, wherein R² is R²-10.483 (=formula 1.1169).

Table 1170. Compounds of formula I.b, wherein R² is R²-10.484 (=formula 1.1170).

Table 1171. Compounds of formula I.b, wherein R² is R²-10.485 (=formula 1.1171).

Table 1172. Compounds of formula I.b, wherein R² is R²-10.486 (=formula 1.1172).

Table 1173. Compounds of formula I.b, wherein R² is R²-10.487 (=formula 1.1173).

Table 1174. Compounds of formula I.b, wherein R² is R²-10.488 (=formula 1.1174).

Table 1175. Compounds of formula I.b, wherein R² is R²-10.489 (=formula 1.1175).

Table 1176. Compounds of formula I.b, wherein R² is R²-10.490 (=formula 1.1176).

Table 1177. Compounds of formula I.b, wherein R² is R²-10.491 (=formula 1.1177).

Table 1178. Compounds of formula I.b, wherein R² is R²-10.492 (=formula 1.1178).

Table 1179. Compounds of formula I.b, wherein R² is R²-10.493 (=formula 1.1179).

Table 1180. Compounds of formula I.b, wherein R² is R²-10.494 (=formula 1.1180).

Table 1181. Compounds of formula I.b, wherein R² is R²-10.495 (=formula 1.1181).

Table 1182. Compounds of formula I.b, wherein R² is R²-10.496 (=formula 1.1182).

Table 1183. Compounds of formula I.b, wherein R² is R²-10.497 (=formula 1.1183).

Table 1184. Compounds of formula I.b, wherein R² is R²-10.498 (=formula 1.1184).

Table 1185. Compounds of formula I.b, wherein R² is R²-10.499 (=formula 1.1185).

Table 1186. Compounds of formula I.b, wherein R² is R²-10.500 (=formula 1.1186).

Table 1187. Compounds of formula I.b, wherein R² is R²-10.501 (=formula 1.1187).

Table 1188. Compounds of formula I.b, wherein R² is R²-10.502 (=formula 1.1188).

Table 1189. Compounds of formula I.b, wherein R² is R²-10.503 (=formula 1.1189).

Table 1190. Compounds of formula I.b, wherein R² is R²-10.504 (=formula 1.1190).

Table 1191. Compounds of formula I.b, wherein R² is R²-10.505 (=formula 1.1191).

Table 1192. Compounds of formula I.b, wherein R² is R²-10.506 (=formula 1.1192).

Table 1193. Compounds of formula I.b, wherein R² is R²-10.507 (=formula 1.1193).

Table 1194. Compounds of formula I.b, wherein R² is R²-10.508 (=formula 1.1194).

Table 1195. Compounds of formula I.b, wherein R² is R²-10.509 (=formula 1.1195).

Table 1196. Compounds of formula I.b, wherein R² is R²-10.510 (=formula 1.1196).

Table 1197. Compounds of formula I.b, wherein R² is R²-10.511 (=formula 1.1197).

Table 1198. Compounds of formula I.b, wherein R² is R²-10.512 (=formula 1.1198).

Table 1199. Compounds of formula I.b, wherein R² is R²-10.513 (=formula 1.1199).

Table 1200. Compounds of formula I.b, wherein R² is R²-10.514 (=formula 1.1200).

Table 1201. Compounds of formula I.b, wherein R² is R²-10.515 (=formula 1.1201).

Table 1202. Compounds of formula I.b, wherein R² is R²-10.516 (=formula 1.1202).

Table 1203. Compounds of formula I.b, wherein R² is R²-10.517 (=formula 1.1203).

Table 1204. Compounds of formula I.b, wherein R² is R²-10.518 (=formula 1.1204).

Table 1205. Compounds of formula I.b, wherein R² is R²-10.519 (=formula 1.1205).

Table 1206. Compounds of formula I.b, wherein R² is R²-10.520 (=formula 1.1206).

Table 1207. Compounds of formula I.b, wherein R² is R²-10.521 (=formula 1.1207).

Table 1208. Compounds of formula I.b, wherein R² is R²-10.522 (=formula 1.1208).

Table 1209. Compounds of formula I.b, wherein R² is R²-10.523 (=formula 1.1209).

Table 1210. Compounds of formula I.b, wherein R² is R²-10.524 (=formula 1.1210).

Table 1211. Compounds of formula I.b, wherein R² is R²-10.525 (=formula 1.1211).

Table 1212. Compounds of formula I.b, wherein R² is R²-10.526 (=formula 1.1212).

Table 1213. Compounds of formula I.b, wherein R² is R²-10.527 (=formula 1.1213).

Table 1214. Compounds of formula I.b, wherein R² is R²-10.528 (=formula 1.1212).

Table 1215. Compounds of formula I.b, wherein R² is R²-10.529 (=formula 1.1215).

Table 1216. Compounds of formula I.b, wherein R² is R²-10.530 (=formula 1.1216).

Table 1217. Compounds of formula I.b, wherein R² is R²-10.531 (=formula 1.1217).

Table 1218. Compounds of formula I.b, wherein R² is R²-10.532 (=formula 1.1218).

Table 1219. Compounds of formula I.b, wherein R² is R²-10.533 (=formula 1.1219).

Table 1220. Compounds of formula I.b, wherein R² is R²-10.534 (=formula 1.1220).

Table 1221. Compounds of formula I.b, wherein R² is R²-10.535 (=formula 1.1221).

Table 1222. Compounds of formula I.b, wherein R² is R²-10.536 (=formula 1.1222).

Table 1223. Compounds of formula I.b, wherein R² is R²-10.537 (=formula 1.1223).

Table 1224. Compounds of formula I.b, wherein R² is R²-10.538 (=formula 1.1224).

Table 1225. Compounds of formula I.b, wherein R² is R²-10.539 (=formula 1.1225).

Table 1226. Compounds of formula I.b, wherein R² is R²-10.540 (=formula 1.1226).

Table 1227. Compounds of formula I.b, wherein R² is R²-10.541 (=formula 1.1227).

Table 1228. Compounds of formula I.b, wherein R² is R²-10.542 (=formula 1.1228).

Table 1229. Compounds of formula I.b, wherein R² is R²-10.543 (=formula 1.1229).

Table 1230. Compounds of formula I.b, wherein R² is R²-10.544 (=formula 1.1230).

Table 1231. Compounds of formula I.b, wherein R² is R²-10.545 (=formula 1.1231).

Table 1232. Compounds of formula I.b, wherein R² is R²-10.546 (=formula 1.1232).

Table 1233. Compounds of formula I.b, wherein R² is R²-10.547 (=formula 1.1233).

Table 1234. Compounds of formula I.b, wherein R² is R²-10.548 (=formula 1.1234).

Table 1235. Compounds of formula I.b, wherein R² is R²-10.549 (=formula 1.1235).

Table 1236. Compounds of formula I.b, wherein R² is R²-10.550 (=formula 1.1236).

Table 1237. Compounds of formula I.b, wherein R² is R²-10.551 (=formula 1.1237).

Table 1238. Compounds of formula I.b, wherein R² is R²-10.552 (=formula 1.1238).

Table 1239. Compounds of formula I.b, wherein R² is R²-10.553 (=formula 1.1239).

Table 1240. Compounds of formula I.b, wherein R² is R²-10.554 (=formula 1.1240).

Table 1241. Compounds of formula I.b, wherein R² is R²-10.555 (=formula 1.1241).

Table 1242. Compounds of formula I.b, wherein R² is R²-10.556 (=formula 1.1242).

Table 1243. Compounds of formula I.b, wherein R² is R²-10.557 (=formula 1.1243).

Table 1244. Compounds of formula I.b, wherein R² is R²-10.558 (=formula 1.1244).

Table 1245. Compounds of formula I.b, wherein R² is R²-10.559 (=formula 1.1245).

Table 1246. Compounds of formula I.b, wherein R² is R²-10.560 (=formula 1.1246).

Table 1247. Compounds of formula I.b, wherein R² is R²-10.561 (=formula 1.1247).

Table 1248. Compounds of formula I.b, wherein R² is R²-10.562 (=formula 1.1248).

Table 1249. Compounds of formula I.b, wherein R² is R²-10.563 (=formula 1.1249).

Table 1250. Compounds of formula I.b, wherein R² is R²-10.564 (=formula 1.1350).

Table 1251. Compounds of formula I.b, wherein R² is R²-10.565 (=formula 1.1251).

Table 1252. Compounds of formula I.b, wherein R² is R²-10.566 (=formula 1.1252).

Table 1253. Compounds of formula I.b, wherein R² is R²-10.567 (=formula 1.1253).

Table 1254. Compounds of formula I.b, wherein R² is R²-10.568 (=formula 1.1254).

Table 1255. Compounds of formula I.b, wherein R² is R²-10.569 (=formula 1.1255).

Table 1256. Compounds of formula I.b, wherein R² is R²-10.570 (=formula 1.1256).

Table 1257. Compounds of formula I.b, wherein R² is R²-10.571 (=formula 1.1257).

Table 1258. Compounds of formula I.b, wherein R² is R²-10.572 (=formula 1.1258).

Table 1259. Compounds of formula I.b, wherein R² is R²-10.573 (=formula 1.1259).

Table 1260. Compounds of formula I.b, wherein R² is R²-10.574 (=formula 1.1260).

Table 1261. Compounds of formula I.b, wherein R² is R²-10.575 (=formula 1.1261).

Table 1262. Compounds of formula I.b, wherein R² is R²-10.576 (=formula 1.1262).

Table 1263. Compounds of formula I.b, wherein R² is R²-10.577 (=formula 1.1263).

Table 1264. Compounds of formula I.b, wherein R² is R²-10.578 (=formula 1.1264).

Table 1265. Compounds of formula I.b, wherein R² is R²-10.579 (=formula 1.1265).

Table 1266. Compounds of formula I.b, wherein R² is R²-10.580 (=formula 1.1266).

Table 1267. Compounds of formula I.b, wherein R² is R²-10.581 (=formula 1.1267).

Table 1268. Compounds of formula I.b, wherein R² is R²-10.582 (=formula 1.1268).

Table 1269. Compounds of formula I.b, wherein R² is R²-10.583 (=formula 1.1269).

Table 1270. Compounds of formula I.b, wherein R² is R²-10.584 (=formula 1.1270).

Table 1271. Compounds of formula I.b, wherein R² is R²-10.585 (=formula 1.1271).

Table 1272. Compounds of formula I.b, wherein R² is R²-10.586 (=formula 1.1272).

Table 1273. Compounds of formula I.b, wherein R² is R²-10.587 (=formula 1.1273).

Table 1274. Compounds of formula I.b, wherein R² is R²-10.588 (=formula 1.1274).

Table 1275. Compounds of formula I.b, wherein R² is R²-10.589 (=formula 1.1275).

Table 1276. Compounds of formula I.b, wherein R² is R²-10.590 (=formula 1.1276).

Table 1277. Compounds of formula I.b, wherein R² is R²-10.591 (=formula 1.1277).

Table 1278. Compounds of formula I.b, wherein R² is R²-10.592 (=formula 1.1278).

Table 1279. Compounds of formula I.b, wherein R² is R²-10.593 (=formula 1.1279).

Table 1280. Compounds of formula I.b, wherein R² is R²-10.594 (=formula 1.1280).

Table 1281. Compounds of formula I.b, wherein R² is R²-10.595 (=formula 1.1281).

Table 1282. Compounds of formula I.b, wherein R² is R²-10.596 (=formula 1.1282).

Table 1283. Compounds of formula I.b, wherein R² is R²-10.597 (=formula 1.1283).

Table 1284. Compounds of formula I.b, wherein R² is R²-10.598 (=formula 1.1284).

Table 1285. Compounds of formula I.b, wherein R² is R²-10.599 (=formula 1.1285).

Table 1286. Compounds of formula I.b, wherein R² is R²-10.600 (=formula 1.1286).

Table 1287. Compounds of formula I.b, wherein R² is R²-10.601 (=formula 1.1287).

Table 1288. Compounds of formula I.b, wherein R² is R²-10.602 (=formula 1.1288).

Table 1289. Compounds of formula I.b, wherein R² is R²-10.603 (=formula 1.1289).

Table 1290. Compounds of formula I.b, wherein R² is R²-10.604 (=formula 1.1290).

Table 1291. Compounds of formula I.b, wherein R² is R²-10.605 (=formula 1.1291).

Table 1292. Compounds of formula I.b, wherein R² is R²-10.606 (=formula 1.1292).

Table 1293. Compounds of formula I.b, wherein R² is R²-10.607 (=formula 1.1293).

Table 1294. Compounds of formula I.b, wherein R² is R²-10.608 (=formula 1.1294).

Table 1295. Compounds of formula I.b, wherein R² is R²-10.609 (=formula 1.1295).

Table 1296. Compounds of formula I.b, wherein R² is R²-10.610 (=formula 1.1296).

Table 1297. Compounds of formula I.b, wherein R² is R²-10.611 (=formula 1.1297).

Table 1298. Compounds of formula I.b, wherein R² is R²-10.612 (=formula 1.1298).

Table 1299. Compounds of formula I.b, wherein R² is R²-10.613 (=formula 1.1299).

Table 1300. Compounds of formula I.b, wherein R² is R²-10.614 (=formula 1.1300).

Table 1301. Compounds of formula I.b, wherein R² is R²-10.615 (=formula 1.1301).

Table 1302. Compounds of formula I.b, wherein R² is R²-10.616 (=formula 1.1302).

Table 1303. Compounds of formula I.b, wherein R² is R²-10.617 (=formula 1.1303).

Table 1304. Compounds of formula I.b, wherein R² is R²-10.618 (=formula 1.1304).

Table 1305. Compounds of formula I.b, wherein R² is R²-10.619 (=formula 1.1305).

Table 1306. Compounds of formula I.b, wherein R² is R²-10.620 (=formula 1.1306).

Table 1307. Compounds of formula I.b, wherein R² is R²-10.621 (=formula 1.1307).

Table 1308. Compounds of formula I.b, wherein R² is R²-10.622 (=formula 1.1308).

Table 1309. Compounds of formula I.b, wherein R² is R²-10.623 (=formula 1.1309).

Table 1310. Compounds of formula I.b, wherein R² is R²-10.624 (=formula 1.1310).

Table 1311. Compounds of formula I.b, wherein R² is R²-10.625 (=formula 1.1311).

Table 1312. Compounds of formula I.b, wherein R² is R²-10.626 (=formula 1.1312).

Table 1313. Compounds of formula I.b, wherein R² is R²-10.627 (=formula 1.1313).

Table 1314. Compounds of formula I.b, wherein R² is R²-10.628 (=formula 1.1314).

Table 1315. Compounds of formula I.b, wherein R² is R²-10.629 (=formula 1.1315).

Table 1316. Compounds of formula I.b, wherein R² is R²-10.630 (=formula 1.1316).

Table 1317. Compounds of formula I.b, wherein R² is R²-10.631 (=formula 1.1317).

Table 1318. Compounds of formula I.b, wherein R² is R²-10.632 (=formula 1.1318).

Table 1319. Compounds of formula I.b, wherein R² is R²-10.633 (=formula 1.1319).

Table 1320. Compounds of formula I.b, wherein R² is R²-10.634 (=formula 1.1320).

Table 1321. Compounds of formula I.b, wherein R² is R²-10.635 (=formula 1.1321).

Table 1322. Compounds of formula I.b, wherein R² is R²-10.636 (=formula 1.1322).

Table 1323. Compounds of formula I.b, wherein R² is R²-10.637 (=formula 1.1323).

Table 1324. Compounds of formula I.b, wherein R² is R²-10.638 (=formula 1.1324).

Table 1325. Compounds of formula I.b, wherein R² is R²-10.639 (=formula 1.1325).

Table 1326. Compounds of formula I.b, wherein R² is R²-10.640 (=formula 1.1326).

Table 1327. Compounds of formula I.b, wherein R² is R²-10.641 (=formula 1.1327).

Table 1328. Compounds of formula I.b, wherein R² is R²-10.642 (=formula 1.1328).

Table 1329. Compounds of formula I.b, wherein R² is R²-10.643 (=formula 1.1329).

Table 1330. Compounds of formula I.b, wherein R² is R²-10.644 (=formula 1.1330).

Table 1331. Compounds of formula I.b, wherein R² is R²-10.645 (=formula 1.1331).

Table 1332. Compounds of formula I.b, wherein R² is R²-10.646 (=formula 1.1332).

Table 1333. Compounds of formula I.b, wherein R² is R²-10.647 (=formula 1.1333).

Table 1334. Compounds of formula I.b, wherein R² is R²-10.648 (=formula 1.1334).

Table 1335. Compounds of formula I.b, wherein R² is R²-10.649 (=formula 1.1335).

Table 1336. Compounds of formula I.b, wherein R² is R²-10.650 (=formula 1.1336).

Table 1337. Compounds of formula I.b, wherein R² is R²-10.651 (=formula 1.1337).

Table 1338. Compounds of formula I.b, wherein R² is R²-10.652 (=formula 1.1338).

Table 1339. Compounds of formula I.b, wherein R² is R²-10.653 (=formula 1.1339).

Table 1340. Compounds of formula I.b, wherein R² is R²-10.654 (=formula 1.1340).

Table 1341. Compounds of formula I.b, wherein R² is R²-10.655 (=formula 1.1341).

Table 1342. Compounds of formula I.b, wherein R² is R²-10.656 (=formula 1.1342).

Table 1343. Compounds of formula I.b, wherein R² is R²-10.657 (=formula 1.1343).

Table 1344. Compounds of formula I.b, wherein R² is R²-10.658 (=formula 1.1344).

Table 1345. Compounds of formula I.b, wherein R² is R²-10.659 (=formula 1.1345).

Table 1346. Compounds of formula I.b, wherein R² is R²-10.660 (=formula 1.1346).

Table 1347. Compounds of formula I.b, wherein R² is R²-10.661 (=formula 1.1347).

Table 1348. Compounds of formula I.b, wherein R² is R²-10.662 (=formula 1.1348).

Table 1349. Compounds of formula I.b, wherein R² is R²-10.663 (=formula 1.1349).

Table 1350. Compounds of formula I.b, wherein R² is R²-10.664 (=formula 1.1350).

Table 1351. Compounds of formula I.b, wherein R² is R²-10.665 (=formula 1.1351).

Table 1352. Compounds of formula I.b, wherein R² is R²-10.666 (=formula 1.1352).

Table 1353. Compounds of formula I.b, wherein R² is R²-10.667 (=formula 1.1353).

Table 1354. Compounds of formula I.b, wherein R² is R²-10.668 (=formula 1.1354).

Table 1355. Compounds of formula I.b, wherein R² is R²-10.669 (=formula 1.1355).

Table 1356. Compounds of formula I.b, wherein R² is R²-10.670 (=formula 1.1356).

Table 1357. Compounds of formula I.b, wherein R² is R²-10.671 (=formula 1.1357).

Table 1358. Compounds of formula I.b, wherein R² is R²-10.672 (=formula 1.1358).

Table 1359. Compounds of formula I.b, wherein R² is R²-10.673 (=formula 1.1359).

Table 1360. Compounds of formula I.b, wherein R² is R²-10.674 (=formula 1.1360).

Table 1361. Compounds of formula I.b, wherein R² is R²-10.675 (=formula 1.1361).

Table 1362. Compounds of formula I.b, wherein R² is R²-10.676 (=formula 1.1362).

Table 1363. Compounds of formula I.b, wherein R² is R²-15.1 (=formula 1.1363).

Table 1364. Compounds of formula I.b, wherein R² is R²-15.2 (=formula 1.1364).

Table 1365. Compounds of formula I.b, wherein R² is R²-15.3 (=formula 1.1365).

Table 1366. Compounds of formula I.b, wherein R² is R²-15.4 (=formula 1.1366).

Table 1367. Compounds of formula I.b, wherein R² is R²-15.5 (=formula 1.1367).

Table 1368. Compounds of formula I.b, wherein R² is R²-15.6 (=formula 1.1368).

Table 1369. Compounds of formula I.b, wherein R² is R²-15.7 (=formula 1.1369).

Table 1370. Compounds of formula I.b, wherein R² is R²-15.8 (=formula 1.1370).

Table 1371. Compounds of formula I.b, wherein R² is R²-15.9 (=formula 1.1371).

Table 1372. Compounds of formula I.b, wherein R² is R²-15.10 (=formula 1.1372).

Table 1373. Compounds of formula I.b, wherein R² is R²-15.11 (=formula 1.1373).

Table 1374. Compounds of formula I.b, wherein R² is R²-15.12 (=formula 1.1374).

Table 1375. Compounds of formula I.b, wherein R² is R²-15.13 (=formula 1.1375).

Table 1376. Compounds of formula I.b, wherein R² is R²-15.14 (=formula 1.1376).

Table 1377. Compounds of formula I.b, wherein R² is R²-15.15 (=formula 1.1377).

Table 1378. Compounds of formula I.b, wherein R² is R²-15.16 (=formula 1.1378).

Table 1379. Compounds of formula I.b, wherein R² is R²-15.17 (=formula 1.1379).

Table 1380. Compounds of formula I.b, wherein R² is R²-15.18 (=formula 1.1380).

Table 1381. Compounds of formula I.b, wherein R² is R²-15.19 (=formula 1.1381).

Table 1382. Compounds of formula I.b, wherein R² is R²-15.20 (=formula 1.1382).

Table 1383. Compounds of formula I.b, wherein R² is R²-15.21 (=formula 1.1383).

Table 1384. Compounds of formula I.b, wherein R² is R²-15.22 (=formula 1.1384).

Table 1385. Compounds of formula I.b, wherein R² is R²-15.23 (=formula 1.1385).

Table 1386. Compounds of formula I.b, wherein R² is R²-15.24 (=formula 1.1386).

Table 1387. Compounds of formula I.b, wherein R² is R²-15.25 (=formula 1.1387).

Table 1388. Compounds of formula I.b, wherein R² is R²-15.26 (=formula 1.1388).

Table 1389. Compounds of formula I.b, wherein R² is R²-15.27 (=formula 1.1389).

Table 1390. Compounds of formula I.b, wherein R² is R²-15.28 (=formula 1.1390).

Table 1391. Compounds of formula I.b, wherein R² is R²-15.29 (=formula 1.1391).

Table 1392. Compounds of formula I.b, wherein R² is R²-15.30 (=formula 1.1392).

Table 1393. Compounds of formula I.b, wherein R² is R²-15.31 (=formula 1.1393).

Table 1394. Compounds of formula I.b, wherein R² is R²-15.32 (=formula 1.1394).

Table 1395. Compounds of formula I.b, wherein R² is R²-15.33 (=formula 1.1395).

Table 1396. Compounds of formula I.b, wherein R² is R²-15.34 (=formula 1.1396).

Table 1397. Compounds of formula I.b, wherein R² is R²-15.35 (=formula 1.1397).

Table 1398. Compounds of formula I.b, wherein R² is R²-15.36 (=formula 1.1398).

Table 1399. Compounds of formula I.b, wherein R² is R²-15.37 (=formula 1.1399).

Table 1400. Compounds of formula I.b, wherein R² is R²-15.38 (=formula 1.1400).

Table 1401. Compounds of formula I.b, wherein R² is R²-15.39 (=formula 1.1401).

Table 1402. Compounds of formula I.b, wherein R² is R²-15.40 (=formula 1.1402).

Table 1403. Compounds of formula I.b, wherein R² is R²-15.41 (=formula 1.1403).

Table 1404. Compounds of formula I.b, wherein R² is R²-15.42 (=formula 1.1404).

Table 1405. Compounds of formula I.b, wherein R² is R²-15.43 (=formula 1.1405).

Table 1406. Compounds of formula I.b, wherein R² is R²-15.44 (=formula 1.1406).

Table 1407. Compounds of formula I.b, wherein R² is R²-15.45 (=formula 1.1407).

Table 1408. Compounds of formula I.b, wherein R² is R²-15.46 (=formula 1.1408).

Table 1409. Compounds of formula I.b, wherein R² is R²-15.47 (=formula 1.1409).

Table 1410. Compounds of formula I.b, wherein R² is R²-15.48 (=formula 1.1410).

Table 1411. Compounds of formula I.b, wherein R² is R²-15.49 (=formula 1.1411).

Table 1412. Compounds of formula I.b, wherein R² is R²-15.50 (=formula 1.1412).

Table 1413. Compounds of formula I.b, wherein R² is R²-15.51 (=formula 1.1413).

Table 1414. Compounds of formula I.b, wherein R² is R²-15.52 (=formula 1.1414).

Table 1415. Compounds of formula I.b, wherein R² is R²-15.53 (=formula 1.1415).

Table 1416. Compounds of formula I.b, wherein R² is R²-15.54 (=formula 1.1416).

Table 1417. Compounds of formula I.b, wherein R² is R²-15.55 (=formula 1.1417).

Table 1418. Compounds of formula I.b, wherein R² is R²-15.56 (=formula 1.1418).

Table 1419. Compounds of formula I.b, wherein R² is R²-15.57 (=formula 1.1419).

Table 1420. Compounds of formula I.b, wherein R² is R²-15.58 (=formula 1.1420).

Table 1421. Compounds of formula I.b, wherein R² is R²-15.59 (=formula 1.1421).

Table 1422. Compounds of formula I.b, wherein R² is R²-15.60 (=formula 1.1422).

Table 1423. Compounds of formula I.b, wherein R² is R²-15.61 (=formula 1.1423).

Table 1424. Compounds of formula I.b, wherein R² is R²-15.62 (=formula 1.1424).

Table 1425. Compounds of formula I.b, wherein R² is R²-15.63 (=formula 1.1425).

Table 1426. Compounds of formula I.b, wherein R² is R²-15.64 (=formula 1.1426).

Table 1427. Compounds of formula I.b, wherein R² is R²-15.65 (=formula 1.1427).

Table 1428. Compounds of formula I.b, wherein R² is R²-15.66 (=formula 1.1428).

Table 1429. Compounds of formula I.b, wherein R² is R²-15.67 (=formula 1.1429).

Table 1430. Compounds of formula I.b, wherein R² is R²-15.68 (=formula 1.1430).

Table 1431. Compounds of formula I.b, wherein R² is R²-15.69 (=formula 1.1431).

Table 1432. Compounds of formula I.b, wherein R² is R²-15.70 (=formula 1.1432).

Table 1433. Compounds of formula I.b, wherein R² is R²-15.71 (=formula 1.1433).

Table 1434. Compounds of formula I.b, wherein R² is R²-15.72 (=formula 1.1434).

Table 1435. Compounds of formula I.b, wherein R² is R²-15.73 (=formula 1.1435).

Table 1436. Compounds of formula I.b, wherein R² is R²-15.74 (=formula 1.1436).

Table 1437. Compounds of formula I.b, wherein R² is R²-15.75 (=formula 1.1437).

Table 1438. Compounds of formula I.b, wherein R² is R²-15.76 (=formula 1.1438).

Table 1439. Compounds of formula I.b, wherein R² is R²-15.77 (=formula 1.1439).

Table 1440. Compounds of formula I.b, wherein R² is R²-15.78 (=formula 1.1440).

Table 1441. Compounds of formula I.b, wherein R² is R²-15.79 (=formula 1.1441).

Table 1442. Compounds of formula I.b, wherein R² is R²-15.80 (=formula 1.1442).

Table 1443. Compounds of formula I.b, wherein R² is R²-15.81 (=formula 1.1443).

Table 1444. Compounds of formula I.b, wherein R² is R²-15.82 (=formula 1.1444).

Table 1445. Compounds of formula I.b, wherein R² is R²-15.83 (=formula 1.1445).

Table 1446. Compounds of formula I.b, wherein R² is R²-15.84 (=formula 1.1446).

Table 1447. Compounds of formula I.b, wherein R² is R²-15.85 (=formula 1.1447).

Table 1448. Compounds of formula I.b, wherein R² is R²-15.86 (=formula 1.1448).

Table 1449. Compounds of formula I.b, wherein R² is R²-15.87 (=formula 1.1449).

Table 1450. Compounds of formula I.b, wherein R² is R²-15.88 (=formula 1.1450).

Table 1451. Compounds of formula I.b, wherein R² is R²-15.89 (=formula 1.1451).

Table 1452. Compounds of formula I.b, wherein R² is R²-15.90 (=formula 1.1452).

Table 1453. Compounds of formula I.b, wherein R² is R²-15.91 (=formula 1.1453).

Table 1454. Compounds of formula I.b, wherein R² is R²-15.92 (=formula 1.1454).

Table 1455. Compounds of formula I.b, wherein R² is R²-15.93 (=formula 1.1455).

Table 1456. Compounds of formula I.b, wherein R² is R²-15.94 (=formula 1.1456).

Table 1457. Compounds of formula I.b, wherein R² is R²-15.95 (=formula 1.1457).

Table 1458. Compounds of formula I.b, wherein R² is R²-15.96 (=formula 1.1458).

Table 1459. Compounds of formula I.b, wherein R² is R²-15.97 (=formula 1.1459).

Table 1460. Compounds of formula I.b, wherein R² is R²-15.98 (=formula 1.1460).

Table 1461. Compounds of formula I.b, wherein R² is R²-15.99 (=formula 1.1461).

Table 1462. Compounds of formula I.b, wherein R² is R²-15.100 (=formula 1.1462).

Table 1463. Compounds of formula I.b, wherein R² is R²-15.101 (=formula 1.1463).

Table 1464. Compounds of formula I.b, wherein R² is R²-15.102 (=formula 1.1464).

Table 1465. Compounds of formula I.b, wherein R² is R²-15.103 (=formula 1.1465).

Table 1466. Compounds of formula I.b, wherein R² is R²-15.104 (=formula 1.1466).

Table 1467. Compounds of formula I.b, wherein R² is R²-15.105 (=formula 1.1467).

Table 1468. Compounds of formula I.b, wherein R² is R²-15.106 (=formula 1.1468).

Table 1469. Compounds of formula I.b, wherein R² is R²-15.107 (=formula 1.1469).

Table 1470. Compounds of formula I.b, wherein R² is R²-15.108 (=formula 1.1470).

Table 1471. Compounds of formula I.b, wherein R² is R²-15.109 (=formula 1.1471).

Table 1472. Compounds of formula I.b, wherein R² is R²-15.110 (=formula 1.1472).

Table 1473. Compounds of formula I.b, wherein R² is R²-15.111 (=formula 1.1473).

Table 1474. Compounds of formula I.b, wherein R² is R²-15.112 (=formula 1.1475).

Table 1475. Compounds of formula I.b, wherein R² is R²-15.113 (=formula 1.1475).

Table 1476. Compounds of formula I.b, wherein R² is R²-15.114 (=formula 1.1476).

Table 1477. Compounds of formula I.b, wherein R² is R²-15.115 (=formula 1.1477).

Table 1478. Compounds of formula I.b, wherein R² is R²-15.116 (=formula 1.1478).

Table 1479. Compounds of formula I.b, wherein R² is R²-15.117 (=formula 1.1479).

Table 1480. Compounds of formula I.b, wherein R² is R²-15.118 (=formula 1.1480).

Table 1481. Compounds of formula I.b, wherein R² is R²-15.119 (=formula 1.1481).

Table 1482. Compounds of formula I.b, wherein R² is R²-15.120 (=formula 1.1482).

Table 1483. Compounds of formula I.b, wherein R² is R²-15.121 (=formula 1.1483).

Table 1484. Compounds of formula I.b, wherein R² is R²-15.122 (=formula 1.1484).

Table 1485. Compounds of formula I.b, wherein R² is R²-15.123 (=formula 1.1485).

Table 1486. Compounds of formula I.b, wherein R² is R²-15.124 (=formula 1.1486).

Table 1487. Compounds of formula I.b, wherein R² is R²-15.125 (=formula 1.1487).

Table 1488. Compounds of formula I.b, wherein R² is R²-15.126 (=formula 1.1488).

Table 1489. Compounds of formula I.b, wherein R² is R²-15.127 (=formula 1.1489).

Table 1490. Compounds of formula I.b, wherein R² is R²-15.128 (=formula 1.1490).

Table 1491. Compounds of formula I.b, wherein R² is R²-15.129 (=formula 1.1491).

Table 1492. Compounds of formula I.b, wherein R² is R²-15.130 (=formula 1.1492).

Table 1493. Compounds of formula I.b, wherein R² is R²-15.131 (=formula 1.1493).

Table 1494. Compounds of formula I.b, wherein R² is R²-15.132 (=formula 1.1494).

Table 1495. Compounds of formula I.b, wherein R² is R²-15.133 (=formula 1.1495).

Table 1496. Compounds of formula I.b, wherein R² is R²-15.134 (=formula 1.1496).

Table 1497. Compounds of formula I.b, wherein R² is R²-15.135 (=formula 1.1497).

Table 1498. Compounds of formula I.b, wherein R² is R²-15.136 (=formula 1.1498).

Table 1499. Compounds of formula I.b, wherein R² is R²-15.137 (=formula 1.1499).

Table 1500. Compounds of formula I.b, wherein R² is R²-15.138 (=formula 1.1500).

Table 1501. Compounds of formula I.b, wherein R² is R²-15.139 (=formula 1.1501).

Table 1502. Compounds of formula I.b, wherein R² is R²-15.140 (=formula 1.1502).

Table 1503. Compounds of formula I.b, wherein R² is R²-15.141 (=formula 1.1503).

Table 1504. Compounds of formula I.b, wherein R² is R²-15.142 (=formula 1.1504).

Table 1505. Compounds of formula I.b, wherein R² is R²-15.143 (=formula 1.1505).

Table 1506. Compounds of formula I.b, wherein R² is R²-15.144 (=formula 1.1506).

Table 1507. Compounds of formula I.b, wherein R² is R²-15.145 (=formula 1.1507).

Table 1508. Compounds of formula I.b, wherein R² is R²-15.146 (=formula 1.1508).

Table 1509. Compounds of formula I.b, wherein R² is R²-15.147 (=formula 1.1509).

Table 1510. Compounds of formula I.b, wherein R² is R²-15.148 (=formula 1.1510).

Table 1511. Compounds of formula I.b, wherein R² is R²-15.149 (=formula 1.1511).

Table 1512. Compounds of formula I.b, wherein R² is R²-15.150 (=formula 1.1512).

Table 1513. Compounds of formula I.b, wherein R² is R²-15.151 (=formula 1.1513).

Table 1514. Compounds of formula I.b, wherein R² is R²-15.152 (=formula 1.1514).

Table 1515. Compounds of formula I.b, wherein R² is R²-15.153 (=formula 1.1515).

Table 1516. Compounds of formula I.b, wherein R² is R²-15.154 (=formula 1.1516).

Table 1517. Compounds of formula I.b, wherein R² is R²-15.155 (=formula 1.1517).

Table 1518. Compounds of formula I.b, wherein R² is R²-15.156 (=formula 1.1518).

Table 1519. Compounds of formula I.b, wherein R² is R²-15.157 (=formula 1.1519).

Table 1520. Compounds of formula I.b, wherein R² is R²-15.158 (=formula 1.1520).

Table 1521. Compounds of formula I.b, wherein R² is R²-15.159 (=formula 1.1521).

Table 1522. Compounds of formula I.b, wherein R² is R²-15.160 (=formula 1.1522).

Table 1523. Compounds of formula I.b, wherein R² is R²-15.161 (=formula 1.1523).

Table 1524. Compounds of formula I.b, wherein R² is R²-15.162 (=formula 1.1524).

Table 1525. Compounds of formula I.b, wherein R² is R²-15.163 (=formula 1.1525).

Table 1526. Compounds of formula I.b, wherein R² is R²-15.164 (=formula 1.1526).

Table 1527. Compounds of formula I.b, wherein R² is R²-15.165 (=formula 1.1527).

Table 1528. Compounds of formula I.b, wherein R² is R²-15.166 (=formula 1.1528).

Table 1529. Compounds of formula I.b, wherein R² is R²-15.167 (=formula 1.1529).

Table 1530. Compounds of formula I.b, wherein R² is R²-15.168 (=formula 1.1530).

Table 1531. Compounds of formula I.b, wherein R² is R²-15.169 (=formula 1.1531).

Table 1532. Compounds of formula I.b, wherein R² is R²-15.170 (=formula 1.1532).

Table 1533. Compounds of formula I.b, wherein R² is R²-15.171 (=formula 1.1533).

Table 1534. Compounds of formula I.b, wherein R² is R²-15.172 (=formula 1.1534).

Table 1535. Compounds of formula I.b, wherein R² is R²-15.173 (=formula 1.1535).

Table 1536. Compounds of formula I.b, wherein R² is R²-15.174 (=formula 1.1536).

Table 1537. Compounds of formula I.b, wherein R² is R²-15.175 (=formula 1.1537).

Table 1538. Compounds of formula I.b, wherein R² is R²-15.176 (=formula 1.1538).

Table 1539. Compounds of formula I.b, wherein R² is R²-15.177 (=formula 1.1539).

Table 1540. Compounds of formula I.b, wherein R² is R²-15.178 (=formula 1.1540).

Table 1541. Compounds of formula I.b, wherein R² is R²-15.179 (=formula 1.1541).

Table 1542. Compounds of formula I.b, wherein R² is R²-15.180 (=formula 1.1542).

Table 1543. Compounds of formula I.b, wherein R² is R²-15.181 (=formula 1.1543).

Table 1544. Compounds of formula I.b, wherein R² is R²-15.182 (=formula 1.1544).

Table 1545. Compounds of formula I.b, wherein R² is R²-15.183 (=formula 1.1545).

Table 1546. Compounds of formula I.b, wherein R² is R²-15.184 (=formula 1.1546).

Table 1547. Compounds of formula I.b, wherein R² is R²-15.185 (=formula 1.1547).

Table 1548. Compounds of formula I.b, wherein R² is R²-15.186 (=formula 1.1548).

Table 1549. Compounds of formula I.b, wherein R² is R²-15.187 (=formula 1.1549).

Table 1550. Compounds of formula I.b, wherein R² is R²-15.188 (=formula 1.1550).

Table 1551. Compounds of formula I.b, wherein R² is R²-15.189 (=formula 1.1551).

Table 1552. Compounds of formula I.b, wherein R² is R²-15.190 (=formula 1.1552).

Table 1553. Compounds of formula I.b, wherein R² is R²-15.191 (=formula 1.1553).

Table 1554. Compounds of formula I.b, wherein R² is R²-15.192 (=formula 1.1554).

Table 1555. Compounds of formula I.b, wherein R² is R²-15.193 (=formula 1.1555).

Table 1556. Compounds of formula I.b, wherein R² is R²-15.194 (=formula 1.1556).

Table 1557. Compounds of formula I.b, wherein R² is R²-15.195 (=formula 1.1557).

Table 1558. Compounds of formula I.b, wherein R² is R²-15.196 (=formula 1.1558).

Table 1559. Compounds of formula I.b, wherein R² is R²-15.197 (=formula 1.1559).

Table 1560. Compounds of formula I.b, wherein R² is R²-15.198 (=formula 1.1560).

Table 1561. Compounds of formula I.b, wherein R² is R²-15.199 (=formula 1.1561).

Table 1562. Compounds of formula I.b, wherein R² is R²-15.200 (=formula 1.1562).

Table 1563. Compounds of formula I.b, wherein R² is R²-15.201 (=formula 1.1563).

Table 1564. Compounds of formula I.b, wherein R² is R²-15.202 (=formula 1.1564).

Table 1565. Compounds of formula I.b, wherein R² is R²-15.203 (=formula 1.1565).

Table 1566. Compounds of formula I.b, wherein R² is R²-15.204 (=formula 1.1566).

Table 1567. Compounds of formula I.b, wherein R² is R²-15.205 (=formula 1.1567).

Table 1568. Compounds of formula I.b, wherein R² is R²-15.206 (=formula 1.1568).

Table 1569. Compounds of formula I.b, wherein R² is R²-15.207 (=formula 1.1569).

Table 1570. Compounds of formula I.b, wherein R² is R²-15.208 (=formula 1.1570).

Table 1571. Compounds of formula I.b, wherein R² is R²-15.209 (=formula 1.1571).

Table 1572. Compounds of formula I.b, wherein R² is R²-15.210 (=formula 1.1572).

Table 1573. Compounds of formula I.b, wherein R² is R²-15.211 (=formula 1.1573).

Table 1574. Compounds of formula I.b, wherein R² is R²-15.212 (=formula 1.1574).

Table 1575. Compounds of formula I.b, wherein R² is R²-15.213 (=formula 1.1575).

Table 1576. Compounds of formula I.b, wherein R² is R²-15.214 (=formula 1.1576).

Table 1577. Compounds of formula I.b, wherein R² is R²-15.215 (=formula 1.1577).

Table 1578. Compounds of formula I.b, wherein R² is R²-15.216 (=formula 1.1578).

Table 1579. Compounds of formula I.b, wherein R² is R²-15.217 (=formula 1.1579).

Table 1580. Compounds of formula I.b, wherein R² is R²-15.218 (=formula 1.1580).

Table 1581. Compounds of formula I.b, wherein R² is R²-15.219 (=formula 1.1581).

Table 1582. Compounds of formula I.b, wherein R² is R²-15.220 (=formula 1.1582).

Table 1583. Compounds of formula I.b, wherein R² is R²-15.221 (=formula 1.1583).

Table 1584. Compounds of formula I.b, wherein R² is R²-15.222 (=formula 1.1584).

Table 1585. Compounds of formula I.b, wherein R² is R²-15.223 (=formula 1.1585).

Table 1586. Compounds of formula I.b, wherein R² is R²-15.224 (=formula 1.1586).

Table 1587. Compounds of formula I.b, wherein R² is R²-15.225 (=formula 1.1587).

Table 1588. Compounds of formula I.b, wherein R² is R²-15.226 (=formula 1.1588).

Table 1589. Compounds of formula I.b, wherein R² is R²-15.227 (=formula 1.1589).

Table 1590. Compounds of formula I.b, wherein R² is R²-15.228 (=formula 1.1590).

Table 1591. Compounds of formula I.b, wherein R² is R²-15.229 (=formula 1.1591).

Table 1592. Compounds of formula I.b, wherein R² is R²-15.230 (=formula 1.1592).

Table 1593. Compounds of formula I.b, wherein R² is R²-15.231 (=formula 1.1593).

Table 1594. Compounds of formula I.b, wherein R² is R²-15.232 (=formula 1.1594).

Table 1595. Compounds of formula I.b, wherein R² is R²-15.233 (=formula 1.1595).

Table 1596. Compounds of formula I.b, wherein R² is R²-15.234 (=formula 1.1596).

Table 1597. Compounds of formula I.b, wherein R² is R²-15.235 (=formula 1.1597).

Table 1598. Compounds of formula I.b, wherein R² is R²-15.236 (=formula 1.1598).

Table 1599. Compounds of formula I.b, wherein R² is R²-15.237 (=formula 1.1599).

Table 1600. Compounds of formula I.b, wherein R² is R²-15.238 (=formula 1.1600).

Table 1601. Compounds of formula I.b, wherein R² is R²-15.239 (=formula 1.1601).

Table 1602. Compounds of formula I.b, wherein R² is R²-15.240 (=formula 1.1602).

Table 1603. Compounds of formula I.b, wherein R² is R²-15.241 (=formula 1.1603).

Table 1604. Compounds of formula I.b, wherein R² is R²-15.242 (=formula 1.1604).

Table 1605. Compounds of formula I.b, wherein R² is R²-15.243 (=formula 1.1605).

Table 1606. Compounds of formula I.b, wherein R² is R²-15.244 (=formula 1.1606).

Table 1607. Compounds of formula I.b, wherein R² is R²-15.245 (=formula 1.1607).

Table 1608. Compounds of formula I.b, wherein R² is R²-15.246 (=formula 1.1608).

Table 1609. Compounds of formula I.b, wherein R² is R²-15.247 (=formula 1.1609).

Table 1610. Compounds of formula I.b, wherein R² is R²-15.248 (=formula 1.1610).

Table 1611. Compounds of formula I.b, wherein R² is R²-15.249 (=formula 1.1611).

Table 1612. Compounds of formula I.b, wherein R² is R²-15.250 (=formula 1.1612).

Table 1613. Compounds of formula I.b, wherein R² is R²-15.251 (=formula 1.1613).

Table 1614. Compounds of formula I.b, wherein R² is R²-15.252 (=formula 1.1614).

Table 1615. Compounds of formula I.b, wherein R² is R²-15.253 (=formula 1.1615).

Table 1616. Compounds of formula I.b, wherein R² is R²-15.254 (=formula 1.1616).

Table 1617. Compounds of formula I.b, wherein R² is R²-15.255 (=formula 1.1617).

Table 1618. Compounds of formula I.b, wherein R² is R²-15.256 (=formula 1.1618).

Table 1619. Compounds of formula I.b, wherein R² is R²-15.257 (=formula 1.1619).

Table 1620. Compounds of formula I.b, wherein R² is R²-15.258 (=formula 1.1620).

Table 1621. Compounds of formula I.b, wherein R² is R²-15.259 (=formula 1.1621).

Table 1622. Compounds of formula I.b, wherein R² is R²-15.260 (=formula 1.1622).

Table 1623. Compounds of formula I.b, wherein R² is R²-15.261 (=formula 1.1623).

Table 1624. Compounds of formula I.b, wherein R² is R²-15.262 (=formula 1.1624).

Table 1625. Compounds of formula I.b, wherein R² is R²-15.263 (=formula 1.1625).

Table 1626. Compounds of formula I.b, wherein R² is R²-15.264 (=formula 1.1626).

Table 1627. Compounds of formula I.b, wherein R² is R²-15.265 (=formula 1.1627).

Table 1628. Compounds of formula I.b, wherein R² is R²-15.266 (=formula 1.1628).

Table 1629. Compounds of formula I.b, wherein R² is R²-15.267 (=formula 1.1629).

Table 1630. Compounds of formula I.b, wherein R² is R²-15.268 (=formula 1.1630).

Table 1631. Compounds of formula I.b, wherein R² is R²-15.269 (=formula 1.1631).

Table 1632. Compounds of formula I.b, wherein R² is R²-15.270 (=formula 1.1632).

Table 1633. Compounds of formula I.b, wherein R² is R²-15.271 (=formula 1.1633).

Table 1634. Compounds of formula I.b, wherein R² is R²-15.272 (=formula 1.1634).

Table 1635. Compounds of formula I.b, wherein R² is R²-15.273 (=formula 1.1635).

Table 1636. Compounds of formula I.b, wherein R² is R²-15.274 (=formula 1.1636).

Table 1637. Compounds of formula I.b, wherein R² is R²-15.275 (=formula 1.1637).

Table 1638. Compounds of formula I.b, wherein R² is R²-15.276 (=formula 1.1638).

Table 1639. Compounds of formula I.b, wherein R² is R²-15.277 (=formula 1.1639).

Table 1640. Compounds of formula I.b, wherein R² is R²-15.278 (=formula 1.1640).

Table 1641. Compounds of formula I.b, wherein R² is R²-15.279 (=formula 1.1641).

Table 1642. Compounds of formula I.b, wherein R² is R²-15.280 (=formula 1.1642).

Table 1643. Compounds of formula I.b, wherein R² is R²-15.281 (=formula 1.1643).

Table 1644. Compounds of formula I.b, wherein R² is R²-15.282 (=formula 1.1644).

Table 1645. Compounds of formula I.b, wherein R² is R²-15.283 (=formula 1.1645).

Table 1646. Compounds of formula I.b, wherein R² is R²-15.284 (=formula 1.1646).

Table 1647. Compounds of formula I.b, wherein R² is R²-15.285 (=formula 1.1647).

Table 1648. Compounds of formula I.b, wherein R² is R²-15.286 (=formula 1.1648).

Table 1649. Compounds of formula I.b, wherein R² is R²-15.287 (=formula 1.1649).

Table 1650. Compounds of formula I.b, wherein R² is R²-15.288 (=formula 1.1650).

Table 1651. Compounds of formula I.b, wherein R² is R²-15.289 (=formula 1.1651).

Table 1652. Compounds of formula I.b, wherein R² is R²-15.290 (=formula 1.1652).

Table 1653. Compounds of formula I.b, wherein R² is R²-15.291 (=formula 1.1653).

Table 1654. Compounds of formula I.b, wherein R² is R²-15.292 (=formula 1.1654).

Table 1655. Compounds of formula I.b, wherein R² is R²-15.293 (=formula 1.1655).

Table 1656. Compounds of formula I.b, wherein R² is R²-15.294 (=formula 1.1656).

Table 1657. Compounds of formula I.b, wherein R² is R²-15.295 (=formula 1.1657).

Table 1658. Compounds of formula I.b, wherein R² is R²-15.296 (=formula 1.1658).

Table 1659. Compounds of formula I.b, wherein R² is R²-15.297 (=formula 1.1659).

Table 1660. Compounds of formula I.b, wherein R² is R²-15.298 (=formula 1.1660).

Table 1661. Compounds of formula I.b, wherein R² is R²-15.299 (=formula 1.1661).

Table 1662. Compounds of formula I.b, wherein R² is R²-15.300 (=formula 1.1662).

Table 1663. Compounds of formula I.b, wherein R² is R²-15.301 (=formula 1.1663).

Table 1664. Compounds of formula I.b, wherein R² is R²-15.302 (=formula 1.1664).

Table 1665. Compounds of formula I.b, wherein R² is R²-15.303 (=formula 1.1665).

Table 1666. Compounds of formula I.b, wherein R² is R²-15.304 (=formula 1.1666).

Table 1667. Compounds of formula I.b, wherein R² is R²-15.305 (=formula 1.1667).

Table 1668. Compounds of formula I.b, wherein R² is R²-15.306 (=formula 1.1668).

Table 1669. Compounds of formula I.b, wherein R² is R²-15.307 (=formula 1.1669).

Table 1670. Compounds of formula I.b, wherein R² is R²-15.308 (=formula 1.1670).

Table 1671. Compounds of formula I.b, wherein R² is R²-15.309 (=formula 1.1671).

Table 1672. Compounds of formula I.b, wherein R² is R²-15.310 (=formula 1.1672).

Table 1673. Compounds of formula I.b, wherein R² is R²-15.311 (=formula 1.1673).

Table 1674. Compounds of formula I.b, wherein R² is R²-15.312 (=formula 1.1674).

Table 1675. Compounds of formula I.b, wherein R² is R²-15.313 (=formula 1.1675).

Table 1676. Compounds of formula I.b, wherein R² is R²-15.314 (=formula 1.1676).

Table 1677. Compounds of formula I.b, wherein R² is R²-15.315 (=formula 1.1676).

Table 1678. Compounds of formula I.b, wherein R² is R²-15.316 (=formula 1.1678).

Table 1679. Compounds of formula I.b, wherein R² is R²-15.317 (=formula 1.1679).

Table 1680. Compounds of formula I.b, wherein R² is R²-15.318 (=formula 1.1680).

Table 1681. Compounds of formula I.b, wherein R² is R²-15.319 (=formula 1.1681).

Table 1682. Compounds of formula I.b, wherein R² is R²-15.320 (=formula 1.1682).

Table 1683. Compounds of formula I.b, wherein R² is R²-15.321 (=formula 1.1683).

Table 1684. Compounds of formula I.b, wherein R² is R²-15.322 (=formula 1.1684).

Table 1685. Compounds of formula I.b, wherein R² is R²-15.323 (=formula 1.1685).

Table 1686. Compounds of formula I.b, wherein R² is R²-15.324 (=formula 1.1686).

Table 1687. Compounds of formula I.b, wherein R² is R²-15.325 (=formula 1.1687).

Table 1688. Compounds of formula I.b, wherein R² is R²-15.326 (=formula 1.1688).

Table 1689. Compounds of formula I.b, wherein R² is R²-15.327 (=formula 1.1689).

Table 1690. Compounds of formula I.b, wherein R² is R²-15.328 (=formula 1.1690).

Table 1691. Compounds of formula I.b, wherein R² is R²-15.329 (=formula 1.1691).

Table 1692. Compounds of formula I.b, wherein R² is R²-15.330 (=formula 1.1692).

Table 1693. Compounds of formula I.b, wherein R² is R²-15.331 (=formula 1.1693).

Table 1694. Compounds of formula I.b, wherein R² is R²-15.332 (=formula 1.1694).

Table 1695. Compounds of formula I.b, wherein R² is R²-15.333 (=formula 1.1695).

Table 1696. Compounds of formula I.b, wherein R² is R²-15.334 (=formula 1.1696).

Table 1697. Compounds of formula I.b, wherein R² is R²-15.335 (=formula 1.1697).

Table 1698. Compounds of formula I.b, wherein R² is R²-15.336 (=formula 1.1698).

Table 1699. Compounds of formula I.b, wherein R² is R²-15.334 (=formula 1.1699).

Table 1700. Compounds of formula I.b, wherein R² is R²-15.338 (=formula 1.1700).

Table 1701. Compounds of formula I.b, wherein R² is R²-15.339 (=formula 1.1701).

Table 1702. Compounds of formula I.b, wherein R² is R²-15.340 (=formula 1.1702).

Table 1703. Compounds of formula I.b, wherein R² is R²-15.341 (=formula 1.1703).

Table 1704. Compounds of formula I.b, wherein R² is R²-15.342 (=formula 1.1704).

Table 1705. Compounds of formula I.b, wherein R² is R²-15.343 (=formula 1.1705).

Table 1706. Compounds of formula I.b, wherein R² is R²-15.344 (=formula 1.1706).

Table 1707. Compounds of formula I.b, wherein R² is R²-15.345 (=formula 1.1707).

Table 1708. Compounds of formula I.b, wherein R² is R²-15.346 (=formula 1.1708).

Table 1709. Compounds of formula I.b, wherein R² is R²-15.347 (=formula 1.1709).

Table 1710. Compounds of formula I.b, wherein R² is R²-15.348 (=formula 1.1710).

Table 1711. Compounds of formula I.b, wherein R² is R²-15.349 (=formula 1.1711).

Table 1712. Compounds of formula I.b, wherein R² is R²-15.350 (=formula 1.1712).

Table 1713. Compounds of formula I.b, wherein R² is R²-15.351 (=formula 1.1713).

Table 1714. Compounds of formula I.b, wherein R² is R²-15.352 (=formula 1.1714).

Table 1715. Compounds of formula I.b, wherein R² is R²-15.353 (=formula 1.1715).

Table 1716. Compounds of formula I.b, wherein R² is R²-15.354 (=formula 1.1716).

Table 1717. Compounds of formula I.b, wherein R² is R²-15.355 (=formula 1.1717).

Table 1718. Compounds of formula I.b, wherein R² is R²-15.356 (=formula 1.1718).

Table 1719. Compounds of formula I.b, wherein R² is R²-15.357 (=formula 1.1719).

Table 1720. Compounds of formula I.b, wherein R² is R²-15.358 (=formula 1.1720).

Table 1721. Compounds of formula I.b, wherein R² is R²-15.359 (=formula 1.1721).

Table 1722. Compounds of formula I.b, wherein R² is R²-15.360 (=formula 1.1722).

Table 1723. Compounds of formula I.b, wherein R² is R²-15.361 (=formula 1.1723).

Table 1724. Compounds of formula I.b, wherein R² is R²-15.362 (=formula 1.1724).

Table 1725. Compounds of formula I.b, wherein R² is R²-15.363 (=formula 1.1725).

Table 1726. Compounds of formula I.b, wherein R² is R²-15.364 (=formula 1.1726).

Table 1727. Compounds of formula I.b, wherein R² is R²-15.365 (=formula 1.1727).

Table 1728. Compounds of formula I.b, wherein R² is R²-15.366 (=formula 1.1728).

Table 1729. Compounds of formula I.b, wherein R² is R²-15.367 (=formula 1.1729).

Table 1730. Compounds of formula I.b, wherein R² is R²-15.368 (=formula 1.1730).

Table 1731. Compounds of formula I.b, wherein R² is R²-15.369 (=formula 1.1731).

Table 1732. Compounds of formula I.b, wherein R² is R²-15.370 (=formula 1.1732).

Table 1733. Compounds of formula I.b, wherein R² is R²-15.371 (=formula 1.1733).

Table 1734. Compounds of formula I.b, wherein R² is R²-15.372 (=formula 1.1734).

Table 1735. Compounds of formula I.b, wherein R² is R²-15.373 (=formula 1.1735).

Table 1736. Compounds of formula I.b, wherein R² is R²-15.374 (=formula 1.1736).

Table 1737. Compounds of formula I.b, wherein R² is R²-15.375 (=formula 1.1737).

Table 1738. Compounds of formula I.b, wherein R² is R²-15.376 (=formula 1.1738).

Table 1739. Compounds of formula I.b, wherein R² is R²-15.377 (=formula 1.1739).

Table 1740. Compounds of formula I.b, wherein R² is R²-15.378 (=formula 1.1740).

Table 1741. Compounds of formula I.b, wherein R² is R²-15.379 (=formula 1.1741).

Table 1742. Compounds of formula I.b, wherein R² is R²-15.380 (=formula 1.1742).

Table 1743. Compounds of formula I.b, wherein R² is R²-15.381 (=formula 1.1743).

Table 1744. Compounds of formula I.b, wherein R² is R²-15.382 (=formula 1.1744).

Table 1745. Compounds of formula I.b, wherein R² is R²-15.383 (=formula 1.1745).

Table 1746. Compounds of formula I.b, wherein R² is R²-15.384 (=formula 1.1746).

Table 1747. Compounds of formula I.b, wherein R² is R²-15.385 (=formula 1.1747).

Table 1748. Compounds of formula I.b, wherein R² is R²-15.386 (=formula 1.1748).

Table 1749. Compounds of formula I.b, wherein R² is R²-15.387 (=formula 1.1749).

Table 1750. Compounds of formula I.b, wherein R² is R²-15.388 (=formula 1.1750).

Table 1751. Compounds of formula I.b, wherein R² is R²-15.389 (=formula 1.1751).

Table 1752. Compounds of formula I.b, wherein R² is R²-15.390 (=formula 1.1752).

Table 1753. Compounds of formula I.b, wherein R² is R²-15.391 (=formula 1.1753).

Table 1754. Compounds of formula I.b, wherein R² is R²-15.392 (=formula 1.1754).

Table 1755. Compounds of formula I.b, wherein R² is R²-15.393 (=formula 1.1755).

Table 1756. Compounds of formula I.b, wherein R² is R²-15.394 (=formula 1.1756).

Table 1757. Compounds of formula I.b, wherein R² is R²-15.395 (=formula 1.1757).

Table 1758. Compounds of formula I.b, wherein R² is R²-15.396 (=formula 1.1758).

Table 1759. Compounds of formula I.b, wherein R² is R²-15.397 (=formula 1.1759).

Table 1760. Compounds of formula I.b, wherein R² is R²-15.398 (=formula 1.1760).

Table 1761. Compounds of formula I.b, wherein R² is R²-15.399 (=formula 1.1761).

Table 1762. Compounds of formula I.b, wherein R² is R²-15.400 (=formula 1.1762).

Table 1763. Compounds of formula I.b, wherein R² is R²-15.401 (=formula 1.1763).

Table 1764. Compounds of formula I.b, wherein R² is R²-15.402 (=formula 1.1764).

Table 1765. Compounds of formula I.b, wherein R² is R²-15.403 (=formula 1.1765).

Table 1766. Compounds of formula I.b, wherein R² is R²-15.404 (=formula 1.1766).

Table 1767. Compounds of formula I.b, wherein R² is R²-15.405 (=formula 1.1767).

Table 1768. Compounds of formula I.b, wherein R² is R²-15.406 (=formula 1.1768).

Table 1769. Compounds of formula I.b, wherein R² is R²-15.407 (=formula 1.1769).

Table 1770. Compounds of formula I.b, wherein R² is R²-15.408 (=formula 1.1770).

Table 1771. Compounds of formula I.b, wherein R² is R²-15.409 (=formula 1.1771).

Table 1772. Compounds of formula I.b, wherein R² is R²-15.410 (=formula 1.1772).

Table 1773. Compounds of formula I.b, wherein R² is R²-15.411 (=formula 1.1773).

Table 1774. Compounds of formula I.b, wherein R² is R²-15.412 (=formula 1.1774).

Table 1775. Compounds of formula I.b, wherein R² is R²-15.413 (=formula 1.1775).

Table 1776. Compounds of formula I.b, wherein R² is R²-15.414 (=formula 1.1776).

Table 1777. Compounds of formula I.b, wherein R² is R²-15.415 (=formula 1.1777).

Table 1778. Compounds of formula I.b, wherein R² is R²-15.416 (=formula 1.1778).

Table 1779. Compounds of formula I.b, wherein R² is R²-15.417 (=formula 1.1779).

Table 1780. Compounds of formula I.b, wherein R² is R²-15.418 (=formula 1.1780).

Table 1781. Compounds of formula I.b, wherein R² is R²-15.419 (=formula 1.1781).

Table 1782. Compounds of formula I.b, wherein R² is R²-15.420 (=formula 1.1782).

Table 1783. Compounds of formula I.b, wherein R² is R²-15.421 (=formula 1.1783).

Table 1784. Compounds of formula I.b, wherein R² is R²-15.422 (=formula 1.1784).

Table 1785. Compounds of formula I.b, wherein R² is R²-15.423 (=formula 1.1785).

Table 1786. Compounds of formula I.b, wherein R² is R²-15.424 (=formula 1.1786).

Table 1787. Compounds of formula I.b, wherein R² is R²-15.425 (=formula 1.1787).

Table 1788. Compounds of formula I.b, wherein R² is R²-15.426 (=formula 1.1788).

Table 1789. Compounds of formula I.b, wherein R² is R²-15.427 (=formula 1.1789).

Table 1790. Compounds of formula I.b, wherein R² is R²-15.428 (=formula 1.1790).

Table 1791. Compounds of formula I.b, wherein R² is R²-15.429 (=formula 1.1791).

Table 1792. Compounds of formula I.b, wherein R² is R²-15.430 (=formula 1.1792).

Table 1793. Compounds of formula I.b, wherein R² is R²-15.431 (=formula 1.1793).

Table 1794. Compounds of formula I.b, wherein R² is R²-15.432 (=formula 1.1794).

Table 1795. Compounds of formula I.b, wherein R² is R²-15.433 (=formula 1.1795).

Table 1796. Compounds of formula I.b, wherein R² is R²-15.434 (=formula 1.1796).

Table 1797. Compounds of formula I.b, wherein R² is R²-15.435 (=formula 1.1797).

Table 1798. Compounds of formula I.b, wherein R² is R²-15.436 (=formula 1.1798).

Table 1799. Compounds of formula I.b, wherein R² is R²-15.437 (=formula 1.1799).

Table 1800. Compounds of formula I.b, wherein R² is R²-15.438 (=formula 1.1800).

Table 1801. Compounds of formula I.b, wherein R² is R²-15.439 (=formula 1.1801).

Table 1802. Compounds of formula I.b, wherein R² is R²-15.440 (=formula 1.1802).

Table 1803. Compounds of formula I.b, wherein R² is R²-15.441 (=formula 1.1803).

Table 1804. Compounds of formula I.b, wherein R² is R²-15.442 (=formula 1.1804).

Table 1805. Compounds of formula I.b, wherein R² is R²-15.443 (=formula 1.1805).

Table 1806. Compounds of formula I.b, wherein R² is R²-15.444 (=formula 1.1806).

Table 1807. Compounds of formula I.b, wherein R² is R²-15.445 (=formula 1.1807).

Table 1808. Compounds of formula I.b, wherein R² is R²-15.446 (=formula 1.1808).

Table 1809. Compounds of formula I.b, wherein R² is R²-15.447 (=formula 1.1809).

Table 1810. Compounds of formula I.b, wherein R² is R²-15.448 (=formula 1.1810).

Table 1811. Compounds of formula I.b, wherein R² is R²-15.449 (=formula 1.1811).

Table 1812. Compounds of formula I.b, wherein R² is R²-15.450 (=formula 1.1812).

Table 1813. Compounds of formula I.b, wherein R² is R²-15.451 (=formula 1.1813).

Table 1814. Compounds of formula I.b, wherein R² is R²-15.452 (=formula 1.1814).

Table 1815. Compounds of formula I.b, wherein R² is R²-15.453 (=formula 1.1815).

Table 1816. Compounds of formula I.b, wherein R² is R²-15.454 (=formula 1.1816).

Table 1817. Compounds of formula I.b, wherein R² is R²-15.455 (=formula 1.1817).

Table 1818. Compounds of formula I.b, wherein R² is R²-15.456 (=formula 1.1818).

Table 1819. Compounds of formula I.b, wherein R² is R²-15.457 (=formula 1.1819).

Table 1820. Compounds of formula I.b, wherein R² is R²-15.458 (=formula 1.1820).

Table 1821. Compounds of formula I.b, wherein R² is R²-15.459 (=formula 1.1821).

Table 1822. Compounds of formula I.b, wherein R² is R²-15.460 (=formula 1.1822).

Table 1823. Compounds of formula I.b, wherein R² is R²-15.461 (=formula 1.1823).

Table 1824. Compounds of formula I.b, wherein R² is R²-15.462 (=formula 1.1824).

Table 1825. Compounds of formula I.b, wherein R² is R²-15.463 (=formula 1.1825).

Table 1826. Compounds of formula I.b, wherein R² is R²-15.464 (=formula 1.1826).

Table 1827. Compounds of formula I.b, wherein R² is R²-15.465 (=formula 1.1827).

Table 1828. Compounds of formula I.b, wherein R² is R²-15.466 (=formula 1.1828).

Table 1829. Compounds of formula I.b, wherein R² is R²-15.467 (=formula 1.1829).

Table 1830. Compounds of formula I.b, wherein R² is R²-15.468 (=formula 1.1830).

Table 1831. Compounds of formula I.b, wherein R² is R²-15.469 (=formula 1.1831).

Table 1832. Compounds of formula I.b, wherein R² is R²-15.470 (=formula 1.1832).

Table 1833. Compounds of formula I.b, wherein R² is R²-15.471 (=formula 1.1833).

Table 1834. Compounds of formula I.b, wherein R² is R²-15.472 (=formula 1.1834).

Table 1835. Compounds of formula I.b, wherein R² is R²-15.473 (=formula 1.1835).

Table 1836. Compounds of formula I.b, wherein R² is R²-15.474 (=formula 1.1836).

Table 1837. Compounds of formula I.b, wherein R² is R²-15.475 (=formula 1.1837).

Table 1838. Compounds of formula I.b, wherein R² is R²-15.476 (=formula 1.1838).

Table 1839. Compounds of formula I.b, wherein R² is R²-15.477 (=formula 1.1839).

Table 1840. Compounds of formula I.b, wherein R² is R²-15.478 (=formula 1.1840).

Table 1841. Compounds of formula I.b, wherein R² is R²-15.479 (=formula 1.1841).

Table 1842. Compounds of formula I.b, wherein R² is R²-15.480 (=formula 1.1842).

Table 1843. Compounds of formula I.b, wherein R² is R²-15.481 (=formula 1.1843).

Table 1844. Compounds of formula I.b, wherein R² is R²-15.482 (=formula 1.1844).

Table 1845. Compounds of formula I.b, wherein R² is R²-15.483 (=formula 1.1845).

Table 1846. Compounds of formula I.b, wherein R² is R²-15.484 (=formula 1.1846).

Table 1847. Compounds of formula I.b, wherein R² is R²-15.485 (=formula 1.1847).

Table 1848. Compounds of formula I.b, wherein R² is R²-15.486 (=formula 1.1848).

Table 1849. Compounds of formula I.b, wherein R² is R²-15.487 (=formula 1.1849).

Table 1850. Compounds of formula I.b, wherein R² is R²-15.488 (=formula 1.1850).

Table 1851. Compounds of formula I.b, wherein R² is R²-15.489 (=formula 1.1851).

Table 1852. Compounds of formula I.b, wherein R² is R²-15.490 (=formula 1.1852).

Table 1853. Compounds of formula I.b, wherein R² is R²-15.491 (=formula 1.1853).

Table 1854. Compounds of formula I.b, wherein R² is R²-15.492 (=formula 1.1854).

Table 1855. Compounds of formula I.b, wherein R² is R²-15.493 (=formula 1.1855).

Table 1856. Compounds of formula I.b, wherein R² is R²-15.494 (=formula 1.1856).

Table 1857. Compounds of formula I.b, wherein R² is R²-15.495 (=formula 1.1857).

Table 1858. Compounds of formula I.b, wherein R² is R²-15.496 (=formula 1.1858).

Table 1859. Compounds of formula I.b, wherein R² is R²-15.497 (=formula 1.1859).

Table 1860. Compounds of formula I.b, wherein R² is R²-15.498 (=formula 1.1860).

Table 1861. Compounds of formula I.b, wherein R² is R²-15.499 (=formula 1.1861).

Table 1862. Compounds of formula I.b, wherein R² is R²-15.500 (=formula 1.1862).

Table 1863. Compounds of formula I.b, wherein R² is R²-15.501 (=formula 1.1863).

Table 1864. Compounds of formula I.b, wherein R² is R²-15.502 (=formula 1.1864).

Table 1865. Compounds of formula I.b, wherein R² is R²-15.503 (=formula 1.1865).

Table 1866. Compounds of formula I.b, wherein R² is R²-15.504 (=formula 1.1866).

Table 1867. Compounds of formula I.b, wherein R² is R²-15.505 (=formula 1.1867).

Table 1868. Compounds of formula I.b, wherein R² is R²-15.506 (=formula 1.1868).

Table 1869. Compounds of formula I.b, wherein R² is R²-15.507 (=formula 1.1869).

Table 1870. Compounds of formula I.b, wherein R² is R²-15.508 (=formula 1.1870).

Table 1871. Compounds of formula I.b, wherein R² is R²-15.509 (=formula 1.1871).

Table 1872. Compounds of formula I.b, wherein R² is R²-15.510 (=formula 1.1872).

Table 1873. Compounds of formula I.b, wherein R² is R²-15.511 (=formula 1.1873).

Table 1874. Compounds of formula I.b, wherein R² is R²-15.512 (=formula 1.1874).

Table 1875. Compounds of formula I.b, wherein R² is R²-15.513 (=formula 1.1875).

Table 1876. Compounds of formula I.b, wherein R² is R²-15.514 (=formula 1.1876).

Table 1877. Compounds of formula I.b, wherein R² is R²-15.515 (=formula 1.1877).

Table 1878. Compounds of formula I.b, wherein R² is R²-15.516 (=formula 1.1878).

Table 1879. Compounds of formula I.b, wherein R² is R²-15.517 (=formula 1.1879).

Table 1880. Compounds of formula I.b, wherein R² is R²-15.518 (=formula 1.1880).

Table 1881. Compounds of formula I.b, wherein R² is R²-15.519 (=formula 1.1881).

Table 1882. Compounds of formula I.b, wherein R² is R²-15.520 (=formula 1.1882).

Table 1883. Compounds of formula I.b, wherein R² is R²-15.521 (=formula 1.1883).

Table 1884. Compounds of formula I.b, wherein R² is R²-15.522 (=formula 1.1884).

Table 1885. Compounds of formula I.b, wherein R² is R²-15.523 (=formula 1.1885).

Table 1886. Compounds of formula I.b, wherein R² is R²-15.524 (=formula 1.1886).

Table 1887. Compounds of formula I.b, wherein R² is R²-15.525 (=formula 1.1887).

Table 1888. Compounds of formula I.b, wherein R² is R²-15.526 (=formula 1.1888).

Table 1889. Compounds of formula I.b, wherein R² is R²-15.527 (=formula 1.1889).

Table 1890. Compounds of formula I.b, wherein R² is R²-15.528 (=formula 1.1890).

Table 1891. Compounds of formula I.b, wherein R² is R²-15.529 (=formula 1.1891).

Table 1892. Compounds of formula I.b, wherein R² is R²-15.530 (=formula 1.1892).

Table 1893. Compounds of formula I.b, wherein R² is R²-15.531 (=formula 1.1893).

Table 1894. Compounds of formula I.b, wherein R² is R²-15.532 (=formula 1.1894).

Table 1895. Compounds of formula I.b, wherein R² is R²-15.533 (=formula 1.1895).

Table 1896. Compounds of formula I.b, wherein R² is R²-15.534 (=formula 1.1896).

Table 1897. Compounds of formula I.b, wherein R² is R²-15.535 (=formula 1.1897).

Table 1898. Compounds of formula I.b, wherein R² is R²-15.536 (=formula 1.1898).

Table 1899. Compounds of formula I.b, wherein R² is R²-15.537 (=formula 1.1899).

Table 1900. Compounds of formula I.b, wherein R² is R²-15.538 (=formula 1.1900).

Table 1901. Compounds of formula I.b, wherein R² is R²-15.539 (=formula 1.1901).

Table 1902. Compounds of formula I.b, wherein R² is R²-15.540 (=formula 1.1902).

Table 1903. Compounds of formula I.b, wherein R² is R²-15.541 (=formula 1.1903).

Table 1904. Compounds of formula I.b, wherein R² is R²-15.542 (=formula 1.1904).

Table 1905. Compounds of formula I.b, wherein R² is R²-15.543 (=formula 1.1905).

Table 1906. Compounds of formula I.b, wherein R² is R²-15.544 (=formula 1.1906).

Table 1907. Compounds of formula I.b, wherein R² is R²-15.545 (=formula 1.1907).

Table 1908. Compounds of formula I.b, wherein R² is R²-15.546 (=formula 1.1908).

Table 1909. Compounds of formula I.b, wherein R² is R²-15.547 (=formula 1.1909).

Table 1910. Compounds of formula I.b, wherein R² is R²-15.548 (=formula 1.1910).

Table 1911. Compounds of formula I.b, wherein R² is R²-15.549 (=formula 1.1911).

Table 1912. Compounds of formula I.b, wherein R² is R²-15.550 (=formula 1.1912).

Table 1913. Compounds of formula I.b, wherein R² is R²-15.551 (=formula 1.1913).

Table 1914. Compounds of formula I.b, wherein R² is R²-15.552 (=formula 1.1914).

Table 1915. Compounds of formula I.b, wherein R² is R²-15.553 (=formula 1.1915).

Table 1916. Compounds of formula I.b, wherein R² is R²-15.554 (=formula 1.1916).

Table 1917. Compounds of formula I.b, wherein R² is R²-15.555 (=formula 1.1917).

Table 1918. Compounds of formula I.b, wherein R² is R²-15.556 (=formula 1.1918).

Table 1919. Compounds of formula I.b, wherein R² is R²-15.557 (=formula 1.1919).

Table 1920. Compounds of formula I.b, wherein R² is R²-15.558 (=formula 1.1920).

Table 1921. Compounds of formula I.b, wherein R² is R²-15.559 (=formula 1.1921).

Table 1922. Compounds of formula I.b, wherein R² is R²-15.560 (=formula 1.1922).

Table 1923. Compounds of formula I.b, wherein R² is R²-15.561 (=formula 1.1923).

Table 1924. Compounds of formula I.b, wherein R² is R²-15.562 (=formula 1.1924).

Table 1925. Compounds of formula I.b, wherein R² is R²-15.563 (=formula 1.1925).

Table 1926. Compounds of formula I.b, wherein R² is R²-15.564 (=formula 1.1926).

Table 1927. Compounds of formula I.b, wherein R² is R²-15.565 (=formula 1.1927).

Table 1928. Compounds of formula I.b, wherein R² is R²-15.566 (=formula 1.1928).

Table 1929. Compounds of formula I.b, wherein R² is R²-15.567 (=formula 1.1929).

Table 1930. Compounds of formula I.b, wherein R² is R²-15.568 (=formula 1.1930).

Table 1931. Compounds of formula I.b, wherein R² is R²-15.569 (=formula 1.1931).

Table 1932. Compounds of formula I.b, wherein R² is R²-15.570 (=formula 1.1932).

Table 1933. Compounds of formula I.b, wherein R² is R²-15.571 (=formula 1.1933).

Table 1934. Compounds of formula I.b, wherein R² is R²-15.572 (=formula 1.1934).

Table 1935. Compounds of formula I.b, wherein R² is R²-15.573 (=formula 1.1935).

Table 1936. Compounds of formula I.b, wherein R² is R²-15.574 (=formula 1.1936).

Table 1937. Compounds of formula I.b, wherein R² is R²-15.575 (=formula 1.1937).

Table 1938. Compounds of formula I.b, wherein R² is R²-15.576 (=formula 1.1938).

Table 1939. Compounds of formula I.b, wherein R² is R²-15.577 (=formula 1.1939).

Table 1940. Compounds of formula I.b, wherein R² is R²-15.578 (=formula 1.1940).

Table 1941. Compounds of formula I.b, wherein R² is R²-15.579 (=formula 1.1941).

Table 1942. Compounds of formula I.b, wherein R² is R²-15.580 (=formula 1.1942).

Table 1943. Compounds of formula I.b, wherein R² is R²-15.581 (=formula 1.1943).

Table 1944. Compounds of formula I.b, wherein R² is R²-15.582 (=formula 1.1944).

Table 1945. Compounds of formula I.b, wherein R² is R²-15.583 (=formula 1.1945).

Table 1946. Compounds of formula I.b, wherein R² is R²-15.584 (=formula 1.1946).

Table 1947. Compounds of formula I.b, wherein R² is R²-15.585 (=formula 1.1947).

Table 1948. Compounds of formula I.b, wherein R² is R²-15.586 (=formula 1.1948).

Table 1949. Compounds of formula I.b, wherein R² is R²-15.587 (=formula 1.1949).

Table 1950. Compounds of formula I.b, wherein R² is R²-15.588 (=formula 1.1950).

Table 1951. Compounds of formula I.b, wherein R² is R²-15.589 (=formula 1.1951).

Table 1952. Compounds of formula I.b, wherein R² is R²-15.590 (=formula 1.1952).

Table 1953. Compounds of formula I.b, wherein R² is R²-15.591 (=formula 1.1953).

Table 1954. Compounds of formula I.b, wherein R² is R²-15.592 (=formula 1.1954).

Table 1955. Compounds of formula I.b, wherein R² is R²-15.593 (=formula 1.1955).

Table 1956. Compounds of formula I.b, wherein R² is R²-15.594 (=formula 1.1956).

Table 1957. Compounds of formula I.b, wherein R² is R²-15.595 (=formula 1.1957).

Table 1958. Compounds of formula I.b, wherein R² is R²-15.596 (=formula 1.1958).

Table 1959. Compounds of formula I.b, wherein R² is R²-15.597 (=formula 1.1959).

Table 1960. Compounds of formula I.b, wherein R² is R²-15.598 (=formula 1.1960).

Table 1961. Compounds of formula I.b, wherein R² is R²-15.599 (=formula 1.1961).

Table 1962. Compounds of formula I.b, wherein R² is R²-15.600 (=formula 1.1962).

Table 1963. Compounds of formula I.b, wherein R² is R²-15.601 (=formula 1.1963).

Table 1964. Compounds of formula I.b, wherein R² is R²-15.602 (=formula 1.1964).

Table 1965. Compounds of formula I.b, wherein R² is R²-15.603 (=formula 1.1965).

Table 1966. Compounds of formula I.b, wherein R² is R²-15.604 (=formula 1.1966).

Table 1967. Compounds of formula I.b, wherein R² is R²-15.605 (=formula 1.1967).

Table 1968. Compounds of formula I.b, wherein R² is R²-15.606 (=formula 1.1968).

Table 1969. Compounds of formula I.b, wherein R² is R²-15.607 (=formula 1.1969).

Table 1970. Compounds of formula I.b, wherein R² is R²-15.608 (=formula 1.1970).

Table 1971. Compounds of formula I.b, wherein R² is R²-15.609 (=formula 1.1971).

Table 1972. Compounds of formula I.b, wherein R² is R²-15.610 (=formula 1.1972).

Table 1973. Compounds of formula I.b, wherein R² is R²-15.611 (=formula 1.1973).

Table 1974. Compounds of formula I.b, wherein R² is R²-15.612 (=formula 1.1974).

Table 1975. Compounds of formula I.b, wherein R² is R²-15.613 (=formula 1.1975).

Table 1976. Compounds of formula I.b, wherein R² is R²-15.614 (=formula 1.1976).

Table 1977. Compounds of formula I.b, wherein R² is R²-15.615 (=formula 1.1977).

Table 1978. Compounds of formula I.b, wherein R² is R²-15.616 (=formula 1.1978).

Table 1979. Compounds of formula I.b, wherein R² is R²-15.617 (=formula 1.1979).

Table 1980. Compounds of formula I.b, wherein R² is R²-15.618 (=formula 1.1980).

Table 1981. Compounds of formula I.b, wherein R² is R²-15.619 (=formula 1.1981).

Table 1982. Compounds of formula I.b, wherein R² is R²-15.620 (=formula 1.1982).

Table 1983. Compounds of formula I.b, wherein R² is R²-15.621 (=formula 1.1983).

Table 1984. Compounds of formula I.b, wherein R² is R²-15.622 (=formula 1.1984).

Table 1985. Compounds of formula I.b, wherein R² is R²-15.623 (=formula 1.1985).

Table 1986. Compounds of formula I.b, wherein R² is R²-15.624 (=formula 1.1986).

Table 1987. Compounds of formula I.b, wherein R² is R²-15.625 (=formula 1.1987).

Table 1988. Compounds of formula I.b, wherein R² is R²-15.626 (=formula 1.1988).

Table 1989. Compounds of formula I.b, wherein R² is R²-15.627 (=formula 1.1989).

Table 1990. Compounds of formula I.b, wherein R² is R²-15.628 (=formula 1.1990).

Table 1991. Compounds of formula I.b, wherein R² is R²-15.629 (=formula 1.1991).

Table 1992. Compounds of formula I.b, wherein R² is R²-15.630 (=formula 1.1992).

Table 1993. Compounds of formula I.b, wherein R² is R²-15.631 (=formula 1.1993).

Table 1994. Compounds of formula I.b, wherein R² is R²-15.632 (=formula 1.1994).

Table 1995. Compounds of formula I.b, wherein R² is R²-15.633 (=formula 1.1995).

Table 1996. Compounds of formula I.b, wherein R² is R²-15.634 (=formula 1.1996).

Table 1997. Compounds of formula I.b, wherein R² is R²-15.635 (=formula 1.1997).

Table 1998. Compounds of formula I.b, wherein R² is R²-15.636 (=formula 1.1998).

Table 1999. Compounds of formula I.b, wherein R² is R²-15.637 (=formula 1.1999).

Table 2000. Compounds of formula I.b, wherein R² is R²-15.638 (=formula 1.2000).

Table 2001. Compounds of formula I.b, wherein R² is R²-15.639 (=formula 1.2001).

Table 2002. Compounds of formula I.b, wherein R² is R²-15.640 (=formula 1.2002).

Table 2003. Compounds of formula I.b, wherein R² is R²-15.641 (=formula 1.2003).

Table 2004. Compounds of formula I.b, wherein R² is R²-15.642 (=formula 1.2004).

Table 2005. Compounds of formula I.b, wherein R² is R²-15.643 (=formula 1.2005).

Table 2006. Compounds of formula I.b, wherein R² is R²-15.644 (=formula 1.2006).

Table 2007. Compounds of formula I.b, wherein R² is R²-15.645 (=formula 1.2007).

Table 2008. Compounds of formula I.b, wherein R² is R²-15.646 (=formula 1.2008).

Table 2009. Compounds of formula I.b, wherein R² is R²-15.647 (=formula 1.2009).

Table 2010. Compounds of formula I.b, wherein R² is R²-15.648 (=formula 1.2010).

Table 2011. Compounds of formula I.b, wherein R² is R²-15.649 (=formula 1.2011).

Table 2012. Compounds of formula I.b, wherein R² is R²-15.650 (=formula 1.2012).

Table 2013. Compounds of formula I.b, wherein R² is R²-15.651 (=formula 1.2013).

Table 2014. Compounds of formula I.b, wherein R² is R²-15.652 (=formula 1.2014).

Table 2015. Compounds of formula I.b, wherein R² is R²-15.653 (=formula 1.2015).

Table 2016. Compounds of formula I.b, wherein R² is R²-15.654 (=formula 1.2016).

Table 2017. Compounds of formula I.b, wherein R² is R²-15.655 (=formula 1.2017).

Table 2018. Compounds of formula I.b, wherein R² is R²-15.656 (=formula 1.2018).

Table 2019. Compounds of formula I.b, wherein R² is R²-15.657 (=formula 1.2019).

Table 2020. Compounds of formula I.b, wherein R² is R²-15.658 (=formula 1.2020).

Table 2021. Compounds of formula I.b, wherein R² is R²-15.659 (=formula 1.2021).

Table 2022. Compounds of formula I.b, wherein R² is R²-15.660 (=formula 1.2022).

Table 2023. Compounds of formula I.b, wherein R² is R²-15.661 (=formula 1.2023).

Table 2024. Compounds of formula I.b, wherein R² is R²-15.662 (=formula 1.2024).

Table 2025. Compounds of formula I.b, wherein R² is R²-15.663 (=formula 1.2025).

Table 2026. Compounds of formula I.b, wherein R² is R²-15.664 (=formula 1.2026).

Table 2027. Compounds of formula I.b, wherein R² is R²-15.665 (=formula 1.2027).

Table 2028. Compounds of formula I.b, wherein R² is R²-15.666 (=formula 1.2028).

Table 2029. Compounds of formula I.b, wherein R² is R²-15.667 (=formula 1.2029).

Table 2030. Compounds of formula I.b, wherein R² is R²-15.668 (=formula 1.2030).

Table 2031. Compounds of formula I.b, wherein R² is R²-15.669 (=formula 1.2031).

Table 2032. Compounds of formula I.b, wherein R² is R²-15.670 (=formula 1.2032).

Table 2033. Compounds of formula I.b, wherein R² is R²-15.671 (=formula 1.2033).

Table 2034. Compounds of formula I.b, wherein R² is R²-15.672 (=formula 1.2034).

Table 2035. Compounds of formula I.b, wherein R² is R²-15.673 (=formula 1.2035).

Table 2036. Compounds of formula I.b, wherein R² is R²-15.674 (=formula 1.2036).

Table 2037. Compounds of formula I.b, wherein R² is R²-15.675 (=formula 1.2037).

Table 2038. Compounds of formula I.b, wherein R² is R²-15.676 (=formula 1.2038).

Table 2039. Compounds of formula I.b, wherein R² is R²-9.1 (=formula 1.2039).

Table 2040. Compounds of formula I.b, wherein R² is R²-9.2 (=formula 1.2040).

Table 2041. Compounds of formula I.b, wherein R² is R²-9.3 (=formula 1.2041).

Table 2042. Compounds of formula I.b, wherein R² is R²-9.4 (=formula 1.2042).

Table 2043. Compounds of formula I.b, wherein R² is R²-9.5 (=formula 1.2043).

Table 2044. Compounds of formula I.b, wherein R² is R²-9.6 (=formula 1.2044).

Table 2045. Compounds of formula I.b, wherein R² is R²-9.7 (=formula 1.2045).

Table 2046. Compounds of formula I.b, wherein R² is R²-9.8 (=formula 1.2046).

Table 2047. Compounds of formula I.b, wherein R² is R²-9.9 (=formula 1.2047).

Table 2048. Compounds of formula I.b, wherein R² is R²-9.10 (=formula 1.2048).

Table 2049. Compounds of formula I.b, wherein R² is R²-9.11 (=formula 1.2049).

Table 2050. Compounds of formula I.b, wherein R² is R²-9.12 (=formula 1.2050).

Table 2051. Compounds of formula I.b, wherein R² is R²-9.13 (=formula 1.2051).

Table 2052. Compounds of formula I.b, wherein R² is R²-9.14 (=formula 1.2052).

Table 2053. Compounds of formula I.b, wherein R² is R²-9.15 (=formula 1.2053).

Table 2054. Compounds of formula I.b, wherein R² is R²-9.16 (=formula 1.2054).

Table 2055. Compounds of formula I.b, wherein R² is R²-9.17 (=formula 1.2055).

Table 2056. Compounds of formula I.b, wherein R² is R²-9.18 (=formula 1.2056).

Table 2057. Compounds of formula I.b, wherein R² is R²-9.19 (=formula 1.2057).

Table 2058. Compounds of formula I.b, wherein R² is R²-9.20 (=formula 1.2058).

Table 2059. Compounds of formula I.b, wherein R² is R²-9.21 (=formula 1.2059).

Table 2060. Compounds of formula I.b, wherein R² is R²-9.22 (=formula 1.2060).

Table 2061. Compounds of formula I.b, wherein R² is R²-9.23 (=formula 1.2061).

Table 2062. Compounds of formula I.b, wherein R² is R²-9.24 (=formula 1.2062).

Table 2063. Compounds of formula I.b, wherein R² is R²-9.25 (=formula 1.2063).

Table 2064. Compounds of formula I.b, wherein R² is R²-9.26 (=formula 1.2064).

Table 2065. Compounds of formula I.b, wherein R² is R²-9.27 (=formula 1.2065).

Table 2066. Compounds of formula I.b, wherein R² is R²-9.28 (=formula 1.2066).

Table 2067. Compounds of formula I.b, wherein R² is R²-9.29 (=formula 1.2067).

Table 2068. Compounds of formula I.b, wherein R² is R²-9.30 (=formula 1.2068).

Table 2069. Compounds of formula I.b, wherein R² is R²-9.31 (=formula 1.2069).

Table 2070. Compounds of formula I.b, wherein R² is R²-9.32 (=formula 1.2070).

Table 2071. Compounds of formula I.b, wherein R² is R²-9.33 (=formula 1.2071).

Table 2072. Compounds of formula I.b, wherein R² is R²-9.34 (=formula 1.2072).

Table 2073. Compounds of formula I.b, wherein R² is R²-9.35 (=formula 1.2073).

Table 2074. Compounds of formula I.b, wherein R² is R²-9.36 (=formula 1.2074).

Table 2075. Compounds of formula I.b, wherein R² is R²-9.37 (=formula 1.2075).

Table 2076. Compounds of formula I.b, wherein R² is R²-9.38 (=formula 1.2076).

Table 2077. Compounds of formula I.b, wherein R² is R²-9.39 (=formula 1.2077).

Table 2078. Compounds of formula I.b, wherein R² is R²-9.40 (=formula 1.2078).

Table 2079. Compounds of formula I.b, wherein R² is R²-9.41 (=formula 1.2079).

Table 2080. Compounds of formula I.b, wherein R² is R²-9.42 (=formula 1.2080).

TABLE A1

| Line | R¹ | R³ | m | R⁴ |
|---|---|---|---|---|
| I-281 | c-C₃H₅ | CH₃ | 0 | — |
| I-282 | c-C₃H₅ | OCH₃ | 0 | — |
| I-283 | c-C₃H₅ | Cl | 0 | — |
| I-284 | c-C₃H₅ | Br | 0 | — |
| I-285 | c-C₃H₅ | CHF₂ | 0 | — |
| I-286 | c-C₃H₅ | F | 0 | — |
| I-287 | c-C₃H₅ | I | 0 | — |
| I-288 | c-C₃H₅ | CH₃ | 1 | 9″-Cl |
| I-289 | c-C₃H₅ | OCH₃ | 1 | 9″-Cl |
| I-290 | c-C₃H₅ | Cl | 1 | 9″-Cl |
| I-291 | c-C₃H₅ | Br | 1 | 9″-Cl |
| I-292 | c-C₃H₅ | CHF₂ | 1 | 9″-Cl |
| I-293 | c-C₃H₅ | F | 1 | 9″-Cl |
| I-294 | c-C₃H₅ | I | 1 | 9″-Cl |
| I-295 | c-C₄H₇ | CH₃ | 0 | — |
| I-296 | c-C₄H₇ | OCH₃ | 0 | — |
| I-297 | c-C₄H₇ | Cl | 0 | — |
| I-298 | c-C₄H₇ | Br | 0 | — |
| I-299 | c-C₄H₇ | CHF₂ | 0 | — |
| I-300 | c-C₄H₇ | F | 0 | — |
| I-301 | c-C₄H₇ | I | 0 | — |
| I-302 | c-C₄H₇ | CH₃ | 1 | 9″-Cl |
| I-303 | c-C₄H₇ | OCH₃ | 1 | 9″-Cl |
| I-304 | c-C₄H₇ | Cl | 1 | 9″-Cl |
| I-305 | c-C₄H₇ | Br | 1 | 9″-Cl |
| I-306 | c-C₄H₇ | CHF₂ | 1 | 9″-Cl |
| I-307 | c-C₄H₇ | F | 1 | 9″-Cl |
| I-308 | c-C₄H₇ | I | 1 | 9″-Cl |
| I-309 | C₂H₅ | CH₃ | 0 | — |
| I-310 | C₂H₅ | OCH₃ | 0 | — |
| I-311 | C₂H₅ | Cl | 0 | — |
| I-312 | C₂H₅ | Br | 0 | — |
| I-313 | C₂H₅ | CHF₂ | 0 | — |
| I-314 | C₂H₅ | F | 0 | — |
| I-315 | C₂H₅ | I | 0 | — |
| I-316 | C₂H₅ | CH₃ | 1 | 9″-Cl |
| I-317 | C₂H₅ | OCH₃ | 1 | 9″-Cl |
| I-318 | C₂H₅ | Cl | 1 | 9″-Cl |
| I-319 | C₂H₅ | Br | 1 | 9″-Cl |
| I-320 | C₂H₅ | CHF₂ | 1 | 9″-Cl |
| I-321 | C₂H₅ | F | 1 | 9″-Cl |
| I-322 | C₂H₅ | I | 1 | 9″-Cl |
| I-323 | OCH₃ | CH₃ | 0 | — |
| I-324 | OCH₃ | OCH₃ | 0 | — |
| I-325 | OCH₃ | Cl | 0 | — |
| I-326 | OCH₃ | Br | 0 | — |
| I-327 | OCH₃ | CHF₂ | 0 | — |
| I-328 | OCH₃ | F | 0 | — |
| I-329 | OCH₃ | I | 0 | — |
| I-330 | OCH₃ | CH₃ | 1 | 9″-Cl |
| I-331 | OCH₃ | OCH₃ | 1 | 9″-Cl |
| I-332 | OCH₃ | Cl | 1 | 9″-Cl |
| I-333 | OCH₃ | Br | 1 | 9″-Cl |
| I-334 | OCH₃ | CHF₂ | 1 | 9″-Cl |
| I-335 | OCH₃ | F | 1 | 9″-Cl |
| I-336 | OCH₃ | I | 1 | 9″-Cl |

The specific number for each single compound is deductible as follows: Compound 1.57.1-317 e.g. comprises the compound of formula I.57 from Table 57 and line I-317 from Table A1;

To widen the spectrum of action and to achieve synergistic effects, the pyrimidine compounds of formula (I) may be mixed with a large number of representatives of other herbicidal or growth-regulating active ingredient groups and then applied concomitantly. Suitable components for mixtures are, e.g., herbicides from the classes of the acetamides, amides, aryloxyphenoxypropionates, benzamides, benzofuran, benzoic acids, benzothiadiazinones, bipyridylium, carbamates, chloroacetamides, chlorocarboxylic acids, cyclohexanediones, dinitroanilines, dinitrophenol, diphenyl ether, glycines, imidazolinones, isoxazoles, isoxazolidinones, nitriles, N-phenylphthalimides, oxadiazoles, oxazolidinediones, oxyacetamides, phenoxycarboxylic acids, phenylcarbamates, phenylpyrazoles, phenylpyrazolines, phenylpyridazines, phosphinic acids, phosphoroamidates, phosphorodithioates, phthalamates, pyrazoles, pyridazinones, pyridines, pyridinecarboxylic acids, pyridinecarboxamides, pyrimidinediones, pyrimidinyl(thio)benzoates, quinolinecarboxylic acids, semicarbazones, sulfonylaminocarbonyltriazolinones, sulfonylureas, tetrazolinones, thiadiazoles, thiocarbamates, triazines, triazinones, triazoles, triazolinones, triazolocarboxamides, triazolopyrimidines, triketones, uracils, or ureas.

It may furthermore be beneficial to apply the pyrimidine compounds of formula (I) alone or in combination with other herbicides, or else in the form of a mixture with other crop protection agents, e.g. together with agents for controlling pests or phytopathogenic fungi or bacteria. Also of interest is the miscibility with mineral salt solutions, which are employed for treating nutritional and trace element deficiencies. Other additives such as non-phytotoxic oils and oil concentrates may also be added.

In one embodiment of the present invention the compositions according to the present invention comprise at least one pyrimidine compound of formula (I) (compound A) and at least one further active compound selected from herbicides B, preferably herbicides B of class b1) to b15), and safeners C (compound C).

In a preferred embodiment of the invention, the composition comprises as active compound A or component A at least one, preferably exactly one, pyrimidine compound of formula (I.1) (corresponds to pyrimidine compound of formula (I)), as defined herein;

In another preferred embodiment of the invention, the composition comprises as active compound A or component A at least one, preferably exactly one, pyrimidine compound of formula (I.2) (corresponds to pyrimidine compound of formula (I)), as defined herein;

In another preferred embodiment of the invention, the composition comprises as active compound A or component A at least one, preferably exactly one, pyrimidine compound of formula (I.3) (corresponds to pyrimidine compound of formula (I)), as defined herein;

In another preferred embodiment of the invention, the composition comprises as active compound A or component A at least one, preferably exactly one, pyrimidine compound of formula (I.4) (corresponds to pyrimidine compound of formula (I)), as defined herein; In another preferred embodiment of the invention, the composition comprises as active compound A or component A at least one, preferably exactly one, pyrimidine compound of formula (I.5) (corresponds to pyrimidine compound of formula (I)), as defined herein;

In another preferred embodiment of the invention, the composition comprises as active compound A or component A at least one, preferably exactly one, pyrimidine compound of formula (I.6) (corresponds to pyrimidine compound of formula (I)), as defined herein;

In another preferred embodiment of the invention, the composition comprises as active compound A or component A at least one, preferably exactly one, pyrimidine compound of formula (I.7) (corresponds to pyrimidine compound of formula (I)), as defined herein;

In another preferred embodiment of the invention, the composition comprises as active compound A or component A at least one, preferably exactly one, pyrimidine compound of formula (I.8) (corresponds to pyrimidine compound of formula (I)), as defined herein;

In another preferred embodiment of the invention, the composition comprises as active compound A or component A at least one, preferably exactly one, pyrimidine compound of formula (I.9) (corresponds to pyrimidine compound of formula (I)), as defined herein;

In another preferred embodiment of the invention, the composition comprises as active compound A or component A at least one, preferably exactly one, pyrimidine compound of formula (I.10) (corresponds to pyrimidine compound of formula (I)), as defined herein;

In another preferred embodiment of the invention, the composition comprises as active compound A or component A at least one, preferably exactly one, pyrimidine compound of formula (I.11) (corresponds to pyrimidine compound of formula (I)), as defined herein;

In another preferred embodiment of the invention, the composition comprises as active compound A or component A at least one, preferably exactly one, pyrimidine compound of formula (I.11.A) (corresponds to pyrimidine compound of formula (I)), as defined herein;

In another preferred embodiment of the invention, the composition comprises as active compound A or component A at least one, preferably exactly one, pyrimidine compound of formula (I.11.C) (corresponds to pyrimidine compound of formula (1)), as defined herein;

Preferred compounds of the formula (I) which, as component A, are constituent of the composition according to the invention are the compounds I.1 to I.11 and I.11.A and I.11.C., as defined above;

In another embodiment of the present invention the compositions according to the present invention comprise at least one pyrimidine compound of formula (1) and at least one further active compound B (herbicide B).

The further herbicidal compound B (component B) is preferably selected from the herbicides of class b1) to b15):

The further herbicidal compound B (component B) is preferably selected from the herbicides of class b1) to b15):

Mixing partners for the composition can be selected from below herbicides B as defined below:

B) herbicides of class b1) to b15):

b1) lipid biosynthesis inhibitors;

b2) acetolactate synthase inhibitors (ALS inhibitors);

b3) photosynthesis inhibitors;

b4) protoporphyrinogen-IX oxidase inhibitors (PPO inhibitors);

b5) bleacher herbicides;

b6) enolpyruvyl shikimate 3-phosphate synthase inhibitors (EPSP inhibitors);

b7) glutamine synthetase inhibitors;

b8) 7,8-dihydropteroate synthase inhibitors (DHP inhibitors);

b9) mitosis inhibitors;

b10) inhibitors of the synthesis of very long chain fatty acids (VLCFA inhibitors);

b11) cellulose biosynthesis inhibitors;

b12) decoupler herbicides;

b13) auxinic herbicides;

b14) auxin transport inhibitors; and

b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol (CAS 499223-49-3) and its salts and esters;

including their agriculturally acceptable salts or derivatives;

In one embodiment of the invention, the compositions contain at least one inhibitor of the lipid biosynthesis (herbicide b1). These compounds inhibit lipid biosynthesis. Inhibition of the lipid biosynthesis can be affected either through inhibition of acetylCoA carboxylase (hereinafter-termed ACCase herbicides) or through a different mode of action (hereinafter termed non-ACCase herbicides). The ACCase herbicides belong to the group A of the HRAC classification system whereas the non-ACCase herbicides belong to the group N of the HRAC classification. In another embodiment of the invention, the compositions contain at least one ALS inhibitor (herbicide b2). The herbicidal activity of these compounds is based on the inhibition of acetolactate synthase and thus on the inhibition of the branched chain amino acid biosynthesis. These inhibitors belong to the group B of the HRAC classification system.

In another embodiment of the invention, the compositions contain at least one inhibitor of photosynthesis (herbicide b3). The herbicidal activity of these compounds is based either on the inhibition of the photosystem II in plants (so-called PSII inhibitors, groups C1, C2 and C3 of HRAC classification) or on diverting the electron transfer in photosystem I in plants (so-called PSI inhibitors, group D of HRAC classification) and thus on an inhibition of photosynthesis. Amongst these, PSII inhibitors are preferred.

In another embodiment of the invention, the compositions contain at least one inhibitor of protoporphyrinogen-IX-oxidase (herbicide b4). The herbicidal activity of these compounds is based on the inhibition of the protoporphyrinogen-IX-oxidase. These inhibitors belong to the group E of the HRAC classification system.

In another embodiment of the invention, the compositions contain at least one bleacher-herbicide (herbicide b5). The herbicidal activity of these compounds is based on the inhibition of the carotenoid biosynthesis. These include compounds which inhibit carotenoid biosynthesis by inhibition of phytoene desaturase (so-called PDS inhibitors, group F1 of HRAC classification), compounds that inhibit the 4-hydroxyphenylpyruvate-dioxygenase (HPPD inhibitors, group F2 of HRAC classification), compounds that inhibit DOXsynthase (group F4 of HRAC class) and compounds which inhibit carotenoid biosynthesis by an unknown mode of action (bleacher—unknown target, group F3 of HRAC classification).

In another embodiment of the invention, the compositions contain at least one EPSP synthase inhibitor (herbicide b6). The herbicidal activity of these compounds is based on the inhibition of enolpyruvyl shikimate 3-phosphate synthase, and thus on the inhibition of the amino acid biosynthesis in plants. These inhibitors belong to the group G of the HRAC classification system.

In another embodiment of the invention, the compositions contain at least one glutamine synthetase inhibitor (herbicide b7). The herbicidal activity of these compounds is based on the inhibition of glutamine synthetase, and thus on the inhibition of the aminoacid biosynthesis in plants. These inhibitors belong to the group H of the HRAC classification system.

In another embodiment of the invention, the compositions contain at least one DHP synthase inhibitor (herbicide b8). The herbicidal activity of these compounds is based on the inhibition of 7,8-dihydropteroate synthase. These inhibitors belong to the group I of the HRAC classification system.

In another embodiment of the invention, the compositions contain at least one mitosis inhibitor (herbicide b9). The herbicidal activity of these compounds is based on the disturbance or inhibition of microtubule formation or organization, and thus on the inhibition of mitosis. These inhibitors belong to the groups K1 and K2 of the HRAC classification system. Among these, compounds of the group K1, in particular dinitroanilines, are preferred.

In another embodiment of the invention, the compositions contain at least one VLCFA inhibitor (herbicide b10). The herbicidal activity of these compounds is based on the inhibition of the synthesis of very long chain fatty acids and thus on the disturbance or inhibition of cell division in plants. These inhibitors belong to the group K3 of the HRAC classification system.

In another embodiment of the invention, the compositions contain at least one cellulose biosynthesis inhibitor (herbicide b11). The herbicidal activity of these compounds is based on the inhibition of the biosynthesis of cellulose and thus on the inhibition of the synthesis of cell walls in plants. These inhibitors belong to the group L of the HRAC classification system.

In another embodiment of the invention, the compositions contain at least one decoupler herbicide (herbicide b12). The herbicidal activity of these compounds is based on the disruption of the cell membrane. These inhibitors belong to the group M of the HRAC classification system.

In another embodiment of the invention, the compositions contain at least one auxinic herbicide (herbicide b13). These include compounds that mimic auxins, i.e. plant hormones, and affect the growth of the plants. These compounds belong to the group O of the HRAC classification system.

In another embodiment of the invention, the compositions contain at least one auxin transport inhibitor (herbicide b14). The herbicidal activity of these compounds is based on the inhibition of the auxin transport in plants. These compounds belong to the group P of the HRAC classification system. As to the given mechanisms of action and classification of the active substances, see e.g. “HRAC, Classification of Herbicides According to Mode of Action”, http://www.plantprotection.org/hrac/MOA.html).

Preference is given to those compositions according to the present invention comprising at least one herbicide B selected from herbicides of class b1, b2, b3, b4, b5, b6, b9, b10, b13, and b14.

Specific preference is given to those compositions according to the present invention which comprise at least one herbicide B selected from the herbicides of class b1, b2, b4, b5, b9, b10, b13, and b14.

Particular preference is given to those compositions according to the present invention which comprise at least one herbicide B selected from the herbicides of class b1, b2, b4, b5, b9, b10, and b13

Examples of herbicides B which can be used in combination with the compound of formula (I) according to the present invention are:

b1) from the group of the lipid biosynthesis inhibitors:

ACC-herbicides such as alloxydim, alloxydim-sodium, butroxydim, clethodim, clodinafop, clodinafop-propargyl, cycloxydim, cyhalofop, cyhalofop-butyl, diclofop, diclofop-methyl, fenoxaprop, fenoxaprop-ethyl, fenoxaprop-P, fenoxaprop-P-ethyl, fluazifop, fluazifop-butyl, fluazifop-P, fluazifop-P-butyl, haloxyfop, haloxyfop-methyl, haloxyfop-P, haloxyfop-P-methyl, metamifop, pinoxaden, profoxydim, propaquizafop, quizalofop, quizalofop-ethyl, quizalofop-tefuryl, quizalofop-P, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2″,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2″,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); and non ACC herbicides such as benfuresate, butylate, cycloate, dalapon, dimepiperate, EPTC, esprocarb, ethofumesate, flupropanate, molinate, orbencarb, pebulate, prosulfocarb, TCA, thiobencarb, tiocarbazil, triallate and vernolate;

b2) from the group of the ALS inhibitors:

sulfonylureas such as amidosulfuron, azimsulfuron, bensulfuron, bensulfuron-methyl, chlorimuron, chlorimuron-ethyl, chlorsulfuron, cinosulfuron, cyclosulfamuron, ethametsulfuron, ethametsulfuron-methyl, ethoxysulfuron, flazasulfuron, flucetosulfuron, flupyrsulfuron, flupyrsulfuron-methyl-sodium, foramsulfuron, halosulfuron, halosulfuron-methyl, imazosulfuron, iodosulfuron, iodosulfuron-methyl-sodium, iofensulfuron, iofensulfuron-sodium, mesosulfuron, metazosulfuron, metsulfuron, metsulfuron-methyl, nicosulfuron, orthosulfamuron, oxasulfuron, primisulfuron, primisulfuron-methyl, propyrisulfuron, prosulfuron, pyrazosulfuron, pyrazosulfuron-ethyl, rimsulfuron, sulfometuron, sulfometuron-methyl, sulfosulfuron, thifensulfuron, thifensulfuron-methyl, triasulfuron, tribenuron, tribenuron-methyl, trifloxysulfuron, triflusulfuron, triflusulfuron-methyl and tritosulfuron,

imidazolinones such as imazamethabenz, imazamethabenz-methyl, imazamox, imazapic, imazapyr, imazaquin and imazethapyr, triazolopyrimidine herbicides and sulfonanilides such as cloransulam, cloransulam-methyl, diclosulam, flumetsulam, florasulam, metosulam, penoxsulam, pyrimisulfan and pyroxsulam,

pyrimidinylbenzoates such as bispyribac, bispyribac-sodium, pyribenzoxim, pyriftalid, pyriminobac, pyriminobac-methyl, pyrithiobac, pyrithiobac-sodium, 4-[[[2-[(4,6-dimethoxy-2-pyrimidinyl)oxy]phenyl]methyl]amino]-benzoic acid-1-methylethyl ester (CAS 420138-41-6), 4-[[[2-[(4,6-dimethoxy-2-pyrimidinyl)oxy]phenyl]methyl]amino]-benzoic acid propyl ester (CAS 420138-40-5), N-(4-bromophenyl)-2-[(4,6-dimethoxy-2-pyrimidinyl)oxy]benzenemethanamine (CAS 420138-01-8),

sulfonylaminocarbonyl-triazolinone herbicides such as flucarbazone, flucarbazone-sodium, propoxycarbazone, propoxycarbazone-sodium, thiencarbazone and thiencarbazone-methyl; and triafamone;

among these, a preferred embodiment of the invention relates to those compositions comprising at least one imidazolinone herbicide;

b3) from the group of the photosynthesis inhibitors:

amicarbazone, inhibitors of the photosystem II, e.g. 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methyl-pyrazol-3-yl)-4-chloro-2-hydroxy-3-methyl-2H-pyrrol-5-one (CAS 1654057-29-0), 1-(5-tert-butyl-1-methylpyrazol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8), 1-(5-tert-butylisoxazol-3-yl)-4-ethoxy-5-hydroxy-3-methyl-imidazolidin-2-one (CAS 1844836-64-1), triazine herbicides, including of chlorotriazine, triazinones, triazindiones, methylthiotriazines and pyridazinones such as ametryn, atrazine, chloridazone, cyanazine, desmetryn, dimethametryn, hexazinone, metribuzin, prometon, prometryn, propazine, simazine, simetryn, terbumeton, terbuthylazin, terbutryn and trietazin, aryl urea such as chlorobromuron, chlorotoluron, chloroxuron, dimefuron, diuron, fluometuron, isoproturon, isouron, linuron, metamitron, methabenzthiazuron, metobenzuron, metoxuron, monolinuron, neburon, siduron, tebuthiuron and thiadiazuron, phenyl carbamates such as desmedipham, karbutilat, phenmedipham, phenmedipham-ethyl, nitrile herbicides such as bromofenoxim, bromoxynil and its salts and esters, ioxynil and its salts and esters, uraciles such as bromacil, lenacil and terbacil, and bentazon and bentazon-sodium, pyridate, pyridafol, pentanochlor and propanil and inhibitors of the photosystem I such as diquat, diquat-dibromide, paraquat, paraquat-dichloride and paraquat-dimetilsulfate. Among these, a preferred embodiment of the invention relates to those compositions comprising at least one aryl urea herbicide. Among these, likewise a preferred embodiment of the invention relates to those compositions comprising at least one triazine herbicide. Among these, likewise a preferred embodiment of the invention relates to those compositions comprising at least one nitrile herbicide;

b4) from the group of the protoporphyrinogen-IX oxidase inhibitors:

acifluorfen, acifluorfen-sodium, azafenidin, bencarbazone, benzfendizone, bifenox, butafenacil, carfentrazone, carfentrazone-ethyl, chlomethoxyfen, chlorphthalim, cinidon-ethyl, fluazolate, flufenpyr, flufenpyr-ethyl, flumiclorac, flumiclorac-pentyl, flumioxazin, fluoroglycofen, fluoroglycofen-ethyl, fluthiacet, fluthiacet-methyl, fomesafen, halosafen, lactofen, oxadiargyl, oxadiazon, oxyfluorfen, pentoxazone, profluazol, pyraclonil, pyraflufen, pyraflufen-ethyl, saflufenacil, sulfentrazone, thidiazimin, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydroisoindole-1,3-dione (CAS 1300118-96-0), 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), methyl (E)-4-[2-chloro-5-[4-chloro-5-(difluoromethoxy)-1H-methyl-pyrazol-3-yl]-4-fluorophenoxy]-3-methoxy-but-2-enoate (CAS 948893-00-3), and 3-[7-chloro-5-fluoro-2-(trifluoromethyl)-1H-benzimidazol-4-yl]-1-methyl-6-(trifluoromethyl)-1H-pyrimidine-2,4-dione (CAS 212754-02-4);

b5) from the group of the bleacher herbicides:

PDS inhibitors: beflubutamid, diflufenican, fluridone, flurochloridone, flurtamone, norflurazon, picolinafen, and 4-(3-trifluoromethylphenoxy)-2-(4-trifluoromethylphenyl)pyrimidine (CAS 180608-33-7), HPPD inhibitors: benzobicyclon, benzofenap, bicyclopyrone, clomazone, fenquinotrione, isoxaflutole, mesotrione, oxotrione (CAS 1486617-21-3), pyrasulfotole, pyrazolynate, pyrazoxyfen, sulcotrione, tefuryltrione, tembotrione, tolpyralate, topramezone, bleacher, unknown target: aclonifen, amitrole flumeturon, 2-chloro-3-methylsulfanyl-N-(1-methyltetrazol-5-yl)-4-(trifluoromethyl)benzamide (CAS 1361139-71-0), 2-(2,4-dichlorophenyl)methyl-4,4-dimethyl-3-isoxazolidone (CAS 81777-95-9) and 2-(2,5-dichlorophenyl)methyl-4,4-dimethyl-3-isoxazolidinone (CAS 81778-66-7);

b6) from the group of the EPSP synthase inhibitors: glyphosate, glyphosate-isopropylammonium, glyposate-potassium and glyphosate-trimesium (sulfosate);

b7) from the group of the glutamine synthase inhibitors: bilanaphos (bialaphos), bilanaphossodium, glufosinate, glufosinate-P and glufosinate-ammonium;

b8) from the group of the DHP synthase inhibitors: asulam;

b9) from the group of the mitosis inhibitors:

compounds of group K1: dinitroanilines such as benfluralin, butralin, dinitramine, ethalfluralin, fluchloralin, oryzalin, pendimethalin, prodiamine and trifluralin, phosphoramidates such as amiprophos, amiprophos-methyl, and butamiphos, benzoic acid herbicides such as chlorthal, chlorthal-dimethyl, pyridines such as dithiopyr and thiazopyr, benzamides such as propyzamide and tebutam; compounds of group K2: carbetamide, chlorpropham, flamprop, flamprop-isopropyl, flamprop-methyl, flamprop-M-isopropyl, flamprop-M-methyl and propham; among these, compounds of group K1, in particular dinitroanilines are preferred;

b10) from the group of the VLCFA inhibitors:

chloroacetamides such as acetochlor, alachlor, amidochlor, butachlor, dimethachlor, dimethenamid, dimethenamid-P, metazachlor, metolachlor, metolachlor-S, pethoxamid, pretilachlor, propachlor, propisochlor and thenylchlor, oxyacetanilides such as flufenacet and mefenacet, acetanilides such as diphenamid, naproanilide, napropamide and napropamide-M, tetrazolinones such fentrazamide, and other herbicides such as anilofos, cafenstrole, fenoxasulfone, ipfencarbazone, piperophos, pyroxasulfone and isoxazoline compounds of the formulae II.1, II.2, II.3, II.4, II.5, II.6, II.7, II.8 and II.9

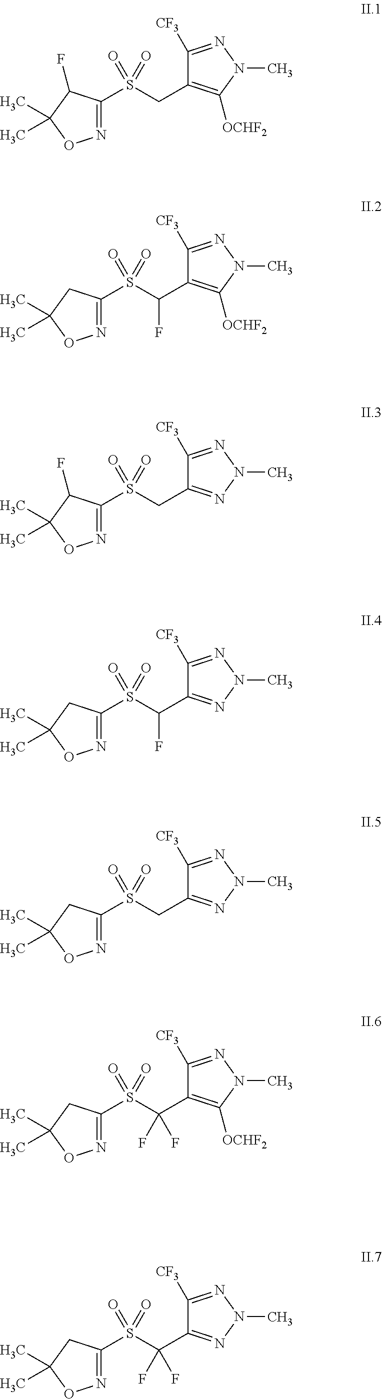

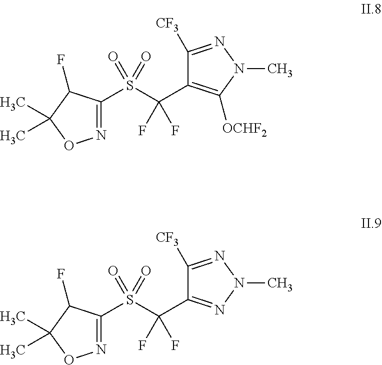

the isoxazoline compounds are known in the art, e.g. from WO 2006/024820, WO 2006/037945, WO 2007/071900 and WO 2007/096576;

among the VLCFA inhibitors, preference is given to chloroacetamides and oxyacetamides;

b11) from the group of the cellulose biosynthesis inhibitors: chlorthiamid, dichlobenil, flupoxam, indaziflam, isoxaben, triaziflam and 1-cyclohexyl-5-pentafluorphenyloxy-1⁴-[1,2,4,6]thiatriazin-3-ylamine (CAS 175899-01-1);

b12) from the group of the decoupler herbicides: dinoseb, dinoterb and DNOC and its salts;

b13) from the group of the auxinic herbicides:

2,4-D and its salts and esters such as clacyfos, 2,4-DB and its salts and esters, aminocyclopyrachlor and its salts and esters, aminopyralid and its salts such as aminopyralid-dimethylammonium, aminopyralid-tris(2-hydroxypropyl)ammonium and its esters, benazolin, benazolin-ethyl, chloramben and its salts and esters, clomeprop, clopyralid and its salts and esters, dicamba and its salts and esters, dichlorprop and its salts and esters, dichlorprop-P and its salts and esters, flopyrauxifen, fluroxypyr, fluroxypyr-butometyl, fluroxypyr-meptyl, halauxifen and its salts and esters (CAS 943832-60-8); MCPA and its salts and esters, MCPA-thioethyl, MCPB and its salts and esters, mecoprop and its salts and esters, mecoprop-P and its salts and esters, picloram and its salts and esters, quinclorac, quinmerac, TBA (2,3,6) and its salts and esters, triclopyr and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr, diflufenzopyr-sodium, naptalam and naptalam-sodium;

b15) from the group of the other herbicides: bromobutide, chlorflurenol, chlorflurenol-methyl, cinmethylin, cumyluron, cyclopyrimorate (CAS 499223-49-3 and its salts and esters, dalapon, dazomet, difenzoquat, difenzoquat-metilsulfate, dimethipin, DSMA, dymron, endothal and its salts, etobenzanid, flurenol, flurenol-butyl, flurprimidol, fosamine, fosamine-ammonium, indanofan, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine and tridiphane.

Preferred herbicides B that can be used in combination with the pyrimidine compounds of the formula (I) according to the present invention are:

b1) from the group of the lipid biosynthesis inhibitors:

clethodim, clodinafop-propargyl, cycloxydim, cyhalofop-butyl, diclofop-methyl, fenoxaprop-P-ethyl, fluazifop-P-butyl, haloxyfop-P-methyl, metamifop, pinoxaden, profoxydim, propaquizafop, quizalofop-P-ethyl, quizalofop-P-tefuryl, sethoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2″,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2″,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); benfuresate, dimepiperate, EPTC, esprocarb, ethofumesate, molinate, orbencarb, prosulfocarb, thiobencarb and triallate;

b2) from the group of the ALS inhibitors:

amidosulfuron, azimsulfuron, bensulfuron-methyl, bispyribac-sodium, chlorimuron-ethyl, chlorsulfuron, cloransulam-methyl, cyclosulfamuron, diclosulam, ethametsulfuron-methyl, ethoxysulfuron, flazasulfuron, florasulam, flucarbazone-sodium, flucetosulfuron, flumetsulam, flupyrsulfuron-methyl-sodium, foramsulfuron, halosulfuron-methyl, imazamethabenz-methyl, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, metosulam, metsulfuron-methyl, nicosulfuron, orthosulfamuron, oxasulfuron, penoxsulam, primisulfuron-methyl, propoxycarbazon-sodium, propyrisulfuron, prosulfuron, pyrazosulfuron-ethyl, pyribenzoxim, pyrimisulfan, pyriftalid, pyriminobac-methyl, pyrithiobac-sodium, pyroxsulam, rimsulfuron, sulfometuron-methyl, sulfosulfuron, thiencarbazone-methyl, thifensulfuron-methyl, triasulfuron, tribenuron-methyl, trifloxysulfuron, triflusulfuron-methyl, tritosulfuron and triafamone;

b3) from the group of the photosynthesis inhibitors:

ametryn, amicarbazone, atrazine, bentazone, bentazone-sodium, bromoxynil and its salts and esters, chloridazone, chlorotoluron, cyanazine, desmedipham, diquat-dibromide, diuron, fluometuron, hexazinone, ioxynil and its salts and esters, isoproturon, lenacil, linuron, metamitron, methabenzthiazuron, metribuzin, paraquat, paraquat-dichloride, phenmedipham, propanil, pyridate, simazine, terbutryn, terbuthylazine, thidiazuron, 1-(6-tert-butylpyrimidin-4-yl)-2-hydroxy-4-methoxy-3-methyl-2H-pyrrol-5-one (CAS 1654744-66-7), 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butyl-1-methyl-pyrazol-3-yl)-4-chloro-2-hydroxy-3-methyl-2H-pyrrol-5-one (CAS 1654057-29-0), 1-(5-tert-butyl-1-methylpyrazol-3-yl)-3-chloro-2-hydroxy-4-methyl-2H-pyrrol-5-one (CAS 1654747-80-4), 4-hydroxy-1-methoxy-5-methyl-3-[4-(trifluoromethyl)-2-pyridyl]imidazolidin-2-one; (CAS 2023785-78-4), 4-hydroxy-1,5-dimethyl-3-[4-(trifluoromethyl)-2-pyridyl]imidazolidin-2-one (CAS 2023785-79-5), 5-ethoxy-4-hydroxy-1-methyl-3-[4-(trifluoromethyl)-2-pyridyl]imidazolidin-2-one (CAS 1701416-69-4), 4-hydroxy-1-methyl-3-[4-(trifluoromethyl)-2-pyridyl]imidazolidin-2-one (CAS 1708087-22-2), 4-hydroxy-1,5-dimethyl-3-[1-methyl-5-(trifluoromethyl)pyrazol-3-yl]imidazolidin-2-one (CAS 2023785-80-8) and 1-(5-tert-butylisoxazol-3-yl)-4-ethoxy-5-hydroxy-3-methyl-imidazolidin-2-one (CAS 1844836-64-1);

b4) from the group of the protoporphyrinogen-IX oxidase inhibitors:

acifluorfen-sodium, bencarbazone, benzfendizone, butafenacil, carfentrazone-ethyl, cinidon-ethyl, flufenpyr-ethyl, flumiclorac-pentyl, flumioxazin, fluoroglycofen-ethyl, fomesafen, lactofen, oxadiargyl, oxadiazon, oxyfluorfen, pentoxazone, pyraflufen, pyraflufen-ethyl, saflufenacil, sulfentrazone, tiafenacil, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S3100), N-ethyl-3-(2,6-dichloro-4-trifluoromethylphenoxy)-5-methyl-1H-pyrazole-1-carboxamide (CAS 452098-92-9), N-tetrahydrofurfuryl-3-(2,6-dichloro-4-trifluoromethylphenoxy)-5-methyl-1H-pyrazole-1-carboxamide (CAS 915396-43-9), N-ethyl-3-(2-chloro-6-fluoro-4-trifluoromethylphenoxy)-5-methyl-1H-pyrazole-1-carboxamide (CAS 452099-05-7), N-tetrahydrofurfuryl-3-(2-chloro-6-fluoro-4-trifluoromethylphenoxy)-5-methyl-1H-pyrazole-1-carboxamide (CAS 452100-03-7), 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0); 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0), and 3-[7-chloro-5-fluoro-2-(trifluoromethyl)-1H-benzimidazol-4-yl]-1-methyl-6-(trifluoromethyl)-1H-pyrimidine-2,4-dione

(CAS 212754-02-4);

b5) from the group of the bleacher herbicides:

aclonifen, amitrole, beflubutamid, benzobicyclon, bicyclopyrone, clomazone, diflufenican, fenquinotrione, flumeturon, flurochloridone, flurtamone, isoxaflutole, mesotrione, oxotrione (CAS 1486617-21-3), norflurazon, picolinafen, pyrasulfotole, pyrazolynate, sulcotrione, tefuryltrione, tembotrione, tolpyralate, topramezone, 4-(3-trifluoromethylphenoxy)-2-(4-trifluoromethylphenyl)pyrimidine (CAS 180608-33-7), 2-chloro-3-methylsulfanyl-N-(1-methyltetrazol-5-yl)-4-(trifluoromethyl)benzamide (CAS 1361139-71-0, 2-(2,4-dichlorophenyl)methyl-4,4-dimethyl-3-isoxazolidone (CAS 81777-95-9) and 2-(2,5-dichlorophenyl)methyl-4,4-dimethyl-3-isoxazolidinone (CAS 81778-66-7);

b6) from the group of the EPSP synthase inhibitors:

glyphosate, glyphosate-isopropylammonium, glyphosate-potassium and glyphosate-trimesium (sulfosate);

b7) from the group of the glutamine synthase inhibitors:

glufosinate, glufosinate-P, glufosinate-ammonium;

b8) from the group of the DHP synthase inhibitors: asulam;

b9) from the group of the mitosis inhibitors:

benfluralin, dithiopyr, ethalfluralin, flamprop, flamprop-isopropyl, flamprop-methyl, flamprop-M-isopropyl, flamprop-M-methyl, oryzalin, pendimethalin, thiazopyr and trifluralin;

b10) from the group of the VLCFA inhibitors: acetochlor, alachlor, amidochlor, anilofos, butachlor, cafenstrole, dimethenamid, dimethenamid-P, fentrazamide, flufenacet, mefenacet, metazachlor, metolachlor, S-metolachlor, naproanilide, napropamide, napropamide-M, pretilachlor, fenoxasulfone, ipfencarbazone, pyroxasulfone thenylchlor and isoxazoline-compounds of the formulae II.1, II.2, II.3, II.4, II.5, II.6, II.7, II.8 and II.9 as mentioned above;

b11) from the group of the cellulose biosynthesis inhibitors: dichlobenil, flupoxam, indaziflam, isoxaben, triaziflam and 1-cyclohexyl-5-pentafluorphenyloxy-1⁴-[1,2,4,6]thiatriazin-3-ylamine (CAS 175899-01-1);

b13) from the group of the auxinic herbicides:

2,4-D and its salts and esters, aminocyclopyrachlor and its salts and esters, aminopyralid and its salts such as aminopyralid-dimethylammonium, aminopyralid-tris(2-hydroxypropyl)ammonium and its esters, clopyralid and its salts and esters, dicamba and its salts and esters, dichlorprop-P and its salts and esters, flopyrauxifen, fluroxypyr-meptyl, halauxifen and its salts and esters (CAS 943832-60-8), MCPA and its salts and esters, MCPB and its salts and esters, mecoprop-P and its salts and esters, picloram and its salts and esters, quinclorac, quinmerac, triclopyr and its salts and esters, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr and diflufenzopyr-sodium;

b15) from the group of the other herbicides: bromobutide, cinmethylin, cumyluron, cyclopyrimorate (CAS 499223-49-3) and its salts and esters, dalapon, difenzoquat, difenzoquat-metilsulfate, DSMA, dymron (=daimuron), indanofan, metam, methylbromide, MSMA, oxaziclomefone, pyributicarb and tridiphane.

Particularly preferred herbicides B that can be used in combination with the pyrimidine compounds of the formula (I) according to the present invention are:

b1) from the group of the lipid biosynthesis inhibitors: clodinafop-propargyl, cycloxydim, cyhalofop-butyl, fenoxaprop-P-ethyl, pinoxaden, profoxydim, tepraloxydim, tralkoxydim, 4-(4′-Chloro-4-cyclopropyl-2′-fluoro[1,1′-biphenyl]-3-yl)-5-hydroxy-2,2,6,6-tetramethyl-2H-pyran-3(6H)-one (CAS 1312337-72-6); 4-(2′,4′-Dichloro-4-cyclopropyl[1,1′-biphenyl]-3-yl)-5-hydroxy-2,2,6,6-tetramethyl-2H-pyran-3(6H)-one (CAS 1312337-45-3); 4-(4′-Chloro-4-ethyl-2′-fluoro[1,1′-biphenyl]-3-yl)-5-hydroxy-2,2,6,6-tetramethyl-2H-pyran-3(6H)-one (CAS 1033757-93-5); 4-(2′,4′-Dichloro-4-ethyl[1,1′-biphenyl]-3-yl)-2,2,6,6-tetramethyl-2H-pyran-3,5(4H,6H)-dione (CAS 1312340-84-3); 5-(Acetyloxy)-4-(4′-chloro-4-cyclopropyl-2′-fluoro[1,1′-biphenyl]-3-yl)-3,6-dihydro-2,2,6,6-tetramethyl-2H-pyran-3-one (CAS 1312337-48-6); 5-(Acetyloxy)-4-(2″,4′-dichloro-4-cyclopropyl-[1,1′-biphenyl]-3-yl)-3,6-dihydro-2,2,6,6-tetramethyl-2H-pyran-3-one; 5-(Acetyloxy)-4-(4′-chloro-4-ethyl-2′-fluoro[1,1′-biphenyl]-3-yl)-3,6-dihydro-2,2,6,6-tetramethyl-2H-pyran-3-one (CAS 1312340-82-1); 5-(Acetyloxy)-4-(2′,4′-dichloro-4-ethyl[1,1′-biphenyl]-3-yl)-3,6-dihydro-2,2,6,6-tetramethyl-2H-pyran-3-one (CAS 1033760-55-2); 4-(4′-Chloro-4-cyclopropyl-2′-fluoro[1,1′-biphenyl]-3-yl)-5,6-dihydro-2,2,6,6-tetramethyl-5-oxo-2H-pyran-3-yl carbonic acid methyl ester (CAS 1312337-51-1); 4-(2″,4′-Dichloro-4-cyclopropyl-[1,1′-biphenyl]-3-yl)-5,6-dihydro-2,2,6,6-tetramethyl-5-oxo-2H-pyran-3-yl carbonic acid methyl ester; 4-(4′-Chloro-4-ethyl-2′-fluoro[1,1′-biphenyl]-3-yl)-5,6-dihydro-2,2,6,6-tetramethyl-5-oxo-2H-pyran-3-yl carbonic acid methyl ester (CAS 1312340-83-2); 4-(2′,4′-Dichloro-4-ethyl[1,1′-biphenyl]-3-yl)-5,6-dihydro-2,2,6,6-tetramethyl-5-oxo-2H-pyran-3-yl carbonic acid methyl ester (CAS 1033760-58-5); esprocarb, prosulfocarb, thiobencarb and triallate;

b2) from the group of the ALS inhibitors: bensulfuron-methyl, bispyribac-sodium, cyclosulfamuron, diclosulam, flumetsulam, flupyrsulfuron-methyl-sodium, foramsulfuron, imazamox, imazapic, imazapyr, imazaquin, imazethapyr, imazosulfuron, iodosulfuron, iodosulfuron-methyl-sodium, iofensulfuron, iofensulfuron-sodium, mesosulfuron, metazosulfuron, nicosulfuron, penoxsulam, propoxycarbazon-sodium, propyrisulfuron, pyrazosulfuron-ethyl, pyroxsulam, rimsulfuron, sulfosulfuron, thiencarbazon-methyl, tritosulfuron and triafamone;

b3) from the group of the photosynthesis inhibitors: ametryn, atrazine, diuron, fluometuron, hexazinone, isoproturon, linuron, metribuzin, paraquat, paraquat-dichloride, propanil, terbutryn, terbuthylazine, 1-(5-tert-butylisoxazol-3-yl)-2-hydroxy-4-methoxy-3-methyl-2H-pyrrol-5-one (CAS 1637455-12-9), 1-(5-tert-butylisoxazol-3-yl)-4-chloro-2-hydroxy-3-methyl-2H-pyrrol-5-one (CAS 1637453-94-1), 1-(5-tert-butylisoxazol-3-yl)-4-ethoxy-5-hydroxy-3-methyl-imidazolidin-2-one (CAS 1844836-64-1);

b4) from the group of the protoporphyrinogen-IX oxidase inhibitors: flumioxazin, oxyfluorfen, pyraflufen, pyraflufen-ethyl, saflufenacil, sulfentrazone, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100, 3-[7-fluoro-3-oxo-4-(prop-2-ynyl)-3,4-dihydro-2H-benzo[1,4]oxazin-6-yl]-1,5-dimethyl-6-thioxo-[1,3,5]triazinan-2,4-dione (CAS 451484-50-7), 2-(2,2,7-trifluoro-3-oxo-4-prop-2-ynyl-3,4-dihydro-2H-benzo[1,4]oxazin-6-yl)-4,5,6,7-tetrahydro-isoindole-1,3-dione (CAS 1300118-96-0), and 1-methyl-6-trifluoromethyl-3-(2,2,7-trifluoro-3-oxo-4-prop-2-ynyl-3,4-dihydro-2H-benzo[1,4]oxazin-6-yl)-1H-pyrimidine-2,4-dione (CAS 1304113-05-0);

b5) from the group of the bleacher herbicides: amitrole, bicyclopyrone, clomazone, diflufenican, fenquinotrione, flumeturon, flurochloridone, isoxaflutole, mesotrione, oxotrione (CAS 1486617-21-3), picolinafen, sulcotrione, tefuryltrione, tembotrione, tolpyralate, topramezone, 2-chloro-3-methylsulfanyl-N-(1-methyltetrazol-5-yl)-4-(trifluoromethyl)benzamide (CAS 1361139-71-0), 2-(2,4-dichlorophenyl)methyl-4,4-dimethyl-3-isoxazolidone (CAS 81777-95-9); and 2-(2,5-dichlorophenyl)methyl-4,4-dimethyl-3-isoxazolidinone (CAS 81778-66-7);

b6) from the group of the EPSP synthase inhibitors: glyphosate, glyphosate-isopropylammonium and glyphosate-trimesium (sulfosate);

b7) from the group of the glutamine synthase inhibitors: glufosinate, glufosinate-P and glufosinate-ammonium;

b9) from the group of the mitosis inhibitors: pendimethalin and trifluralin;

b10) from the group of the VLCFA inhibitors: acetochlor, cafenstrole, dimethenamid-P, fentrazamide, flufenacet, mefenacet, metazachlor, metolachlor, S-metolachlor, fenoxasulfone, ipfencarbazone and pyroxasulfone; likewise, preference is given to isoxazoline compounds of the formulae II.1, II.2, II.3, II.4, II.5, II.6, II.7, II.8 and II.9 as mentioned above;

b11) from the group of the cellulose biosynthesis inhibitors: indaziflam, isoxaben and triaziflam;

b13) from the group of the auxinic herbicides: 2,4-D and its salts and esters such as clacyfos, and aminocyclopyrachlor and its salts and esters, aminopyralid and its salts and its esters, clopyralid and its salts and esters, dicamba and its salts and esters, flopyrauxifen, fluroxypyr-meptyl, halauxifen, halauxifen-methyl, quinclorac, quinmerac, florpyrauxifen, florpyrauxifen-benzyl (CAS 1390661-72-9) and 4-amino-3-chloro-5-fluoro-6-(7-fluoro-1H-indol-6-yl)picolinic acid (CAS 1629965-65-6);

b14) from the group of the auxin transport inhibitors: diflufenzopyr and diflufenzopyr-sodium,

b15) from the group of the other herbicides: cinmethylin, dymon (=daimuron), indanofan, oxaziclomefone.

Particularly preferred herbicides B are the herbicides B as defined above; in particular, the herbicides B.1-B.202 listed below in table B:

TABLE B

| B | Herbicide B |
|---|---|
| B.1 | clethodim |
| B.2 | clodinafop-propargyl |
| B.3 | cycloxydim |
| B.4 | cyhalofop-butyl |
| B.5 | fenoxaprop-ethyl |
| B.6 | fenoxaprop-P-ethyl |
| B.7 | metamifop |
| B.8 | pinoxaden |
| B.9 | profoxydim |
| B.10 | sethoxydim |
| B.11 | tepraloxydim |
| B.12 | tralkoxydim |
| B.13 | esprocarb |
| B.14 | ethofumesate |
| B.15 | molinate |
| B.16 | prosulfocarb |
| B.17 | thiobencarb |
| B.18 | triallate |
| B.19 | bensulfuron-methyl |
| B.20 | bispyribac-sodium |
| B.21 | cloransulam-methyl |
| B.22 | chlorsulfuron |
| B.23 | clorimuron |
| B.24 | cyclosulfamuron |
| B.25 | diclosulam |
| B.26 | florasulam |
| B.27 | flumetsulam |
| B.28 | flupyrsulfuron-methyl-sodium |
| B.29 | foramsulfuron |
| B.30 | imazamox |
| B.31 | imazamox-ammonium |
| B.32 | imazapic |
| B.33 | imazapic-ammonium |
| B.34 | imazapic-isopropylammonium |
| B.35 | imazapyr |
| B.36 | imazapyr-ammonium |
| B.37 | imazapyr-isopropylammonium |
| B.38 | imazaguin |
| B.39 | imazaguin-ammonium |
| B.40 | imazethapyr |
| B.41 | imazethapyr-ammonium |
| B.42 | imazethapyr-isopropylammonium |
| B.43 | imazosulfuron |
| B.44 | iodosulfuron-methyl-sodium |
| B.45 | iofensulfuron |
| B.46 | iofensulfuron-sodium |
| B.47 | mesosulfuron-methyl |
| B.48 | metazosulfuron |
| B.49 | metsulfuron-methyl |
| B.50 | metosulam |
| B.51 | nicosulfuron |
| B.52 | penoxsulam |
| B.53 | propoxycarbazon-sodium |
| B.54 | pyrazosulfuron-ethyl |
| B.55 | pyribenzoxim |
| B.56 | pyriftalid |
| B.57 | pyroxsulam |
| B.58 | propyrisulfuron |
| B.59 | rimsulfuron |
| B.60 | sulfosulfuron |
| B.61 | thiencarbazone-methyl |
| B.62 | thifensulfuron-methyl |
| B.63 | tribenuron-methyl |
| B.64 | tritosulfuron |
| B.65 | triafamone |
| B.66 | ametryne |
| B.67 | atrazine |
| B.68 | bentazon |
| B.69 | bromoxynil |
| B.70 | bromoxynil-octanoate |
| B.71 | bromoxynil-heptanoate |
| B.72 | bromoxynil-potassium |
| B.73 | diuron |
| B.74 | fluometuron |
| B.75 | hexazinone |
| B.76 | isoproturon |
| B.77 | linuron |
| B.78 | metamitron |
| B.79 | metribuzin |
| B.80 | propanil |
| B.81 | simazin |
| B.82 | terbuthylazine |
| B.83 | terbutryn |
| B.84 | paraquat-dichloride |
| B.85 | acifluorfen |
| B.86 | butafenacil |
| B.87 | carfentrazone-ethyl |
| B.88 | flumioxazin |
| B.89 | fomesafen |
| B.90 | oxadiargyl |
| B.91 | oxyfluorfen |
| B.92 | pyraflufen |
| B.93 | pyraflufen-ethyl |
| B.94 | saflufenacil |
| B.95 | sulfentrazone |
| B.96 | trifludimoxazin |
| B.97 | ethyl [3-[2-chloro-4-fluoro-5-(1- |
| | methyl-6-trifluoromethyl-2,4-di- |
| | oxo-1,2,3,4-tetrahydropyrimidin- |
| | 3-yl)phenoxy]-2-pyridyl- |
| | oxy]acetate (CAS 353292-31-6) |
| B.98 | benzobicyclon |
| B.99 | bicyclopyrone |
| B.100 | clomazone |
| B.101 | diflufenican |
| B.102 | flurochloridone |
| B.103 | isoxaflutole |
| B.104 | mesotrione |
| B.105 | norflurazone |
| B.106 | picolinafen |
| B.107 | sulcotrione |
| B.108 | tefuryltrione |
| B.109 | tembotrione |
| B.110 | tolpyralate |
| B.111 | topramezone |
| B.112 | topramezone-sodium |
| B.113 | amitrole |
| B.114 | fluometuron |
| B.115 | fenquinotrione |
| B.116 | glyphosate |
| B.117 | glyphosate-ammonium |
| B.118 | glyphosate-dimethylammonium |
| B.119 | glyphosate-isopropylammonium |
| B.120 | glyphosate-trimesium (sulfosate) |
| B.121 | glyphosate-potassium |
| B.122 | glufosinate |
| B.123 | glufosinate-ammonium |
| B.124 | glufosinate-P |
| B.125 | glufosinate-P-ammonium |
| B.126 | pendimethalin |
| B.127 | trifluralin |
| B.128 | acetochlor |
| B.129 | butachlor |
| B.130 | cafenstrole |
| B.131 | dimethenamid-P |
| B.132 | fentrazamide |
| B.133 | flufenacet |
| B.134 | mefenacet |
| B.135 | metazachlor |
| B.136 | metolachlor |
| B.137 | S-metolachlor |
| B.138 | pretilachlor |
| B.139 | fenoxasulfone |
| B.140 | indaziflam |
| B.141 | isoxaben |
| B.142 | triaziflam |
| B.143 | ipfencarbazone |
| B.144 | pyroxasulfone |
| B.145 | 2,4-D |
| B.146 | 2,4-D-isobutyl |
| B.147 | 2,4-D-dimethylammonium |
| B.148 | 2,4-D-N,N,N- |
| | trimethylethanolammonium |
| B.149 | aminopyralid |
| B.150 | aminopyralid-methyl |
| B.151 | aminopyralid-dimethylammonium |
| B.152 | aminopyralid-tris(2- |
| | hydroxypropyl)ammonium |
| B.153 | clopyralid |
| B.154 | clopyralid-methyl |
| B.155 | clopyralid-olamine |
| B.156 | dicamba |
| B.157 | dicamba-butotyl |
| B.158 | dicamba-diglycolamine |
| B.159 | dicamba-dimethylammonium |
| B.160 | dicamba-diolamine |
| B.161 | dicamba-isopropylammonium |
| B.162 | dicamba-potassium |
| B.163 | dicamba-sodium |
| B.164 | dicamba-trolamine |
| B.165 | dicamba-N,N-bis-(3- |
| | aminopropyl)methylamine |
| B.166 | dicamba-diethylenetriamine |
| B.167 | fluroxypyr |
| B.168 | fluroxypyr-meptyl |
| B.169 | halauxifen |
| B.170 | halauxifen-methyl |
| B.171 | MCPA |
| B.172 | MCPA-2-ethylhexyl |
| B.173 | MCPA-dimethylammonium |
| B.174 | quinclorac |
| B.175 | quinclorac-dimethylammonium |
| B.176 | quinmerac |
| B.177 | quinmerac-dimethylammonium |
| B.178 | florpyrauxifen |
| B.179 | florpyrauxifen-benzyl (CAS |
| | 1390661-72-9) |
| B.180 | aminocyclopyrachlor |
| B.181 | aminocyclopyrachlor-potassium |
| B.182 | aminocyclopyrachlor-methyl |
| B.183 | diflufenzopyr |
| B.184 | diflufenzopyr-sodium |
| B.185 | dymron |
| B.186 | indanofan |
| B.187 | oxaziclomefone |
| B.188 | II.1 |
| B.189 | II.2 |
| B.190 | II.3 |
| B.191 | II.4 |
| B.192 | II.5 |
| B.193 | II.6 |
| B.194 | II.7 |
| B.195 | II.8 |
| B.196 | II.9 |
| B.197 | 4-amino-3-chloro-5-fluoro-6-(7- |
| | fluoro-1H-indol-6-yl)picolinic acid |
| | (CAS 1629965-65-6) |
| B.198 | flopyrauxifen |
| B.199 | oxotrione (CAS 1486617-21-3) |
| B.200 | cinmethylin |
| B.201 | 2-chloro-3-methylsulfanyl-N-(1- |
| | methyltetrazol-5-yl)-4- |
| | (trifluoromethyl)benzamide (CAS |
| | 1361139-71-0) |
| B.202 | 2-(2,4-dichlorophenyl)methyl-4,4- |
| | dimethyl-3-isoxazolidone (CAS |
| | 81777-95-9) |

In another embodiment of the present invention the compositions according to the present invention comprise at least one pyrimidine compound of formula (I) and at least one safener C.

Safeners are chemical compounds which prevent or reduce damage on useful plants without having a major impact on the herbicidal action of the herbicidal active components of the present compositions towards unwanted plants. They can be applied either before sowings (e.g. on seed treatments, shoots or seedlings) or in the pre-emergence application or post-emergence application of the useful plant. The safeners and the pyrimidine compounds of formula (I) and/or the herbicides B can be applied simultaneously or in succession.

Suitable safeners are e.g. (quinolin-8-oxy)acetic acids, 1-phenyl-5-haloalkyl-1H-1,2,4-triazol-3-carboxylic acids, 1-phenyl-4,5-dihydro-5-alkyl-1H-pyrazol-3,5-dicarboxylic acids, 4,5-dihydro-5,5-diaryl-3-isoxazol carboxylic acids, dichloroacetamides, alpha-oximinophenylacetonitriles, acetophenonoximes, 4,6-dihalo-2-phenylpyrimidines, N-[[4-(aminocarbonyl)phenyl]sulfonyl]-2-benzoic amides, 1,8-naphthalic anhydride, 2-halo-4-(haloalkyl)-5-thiazol carboxylic acids, phosphorthiolates and N-alkyl-O-phenylcarbamates and their agriculturally acceptable salts and their agriculturally acceptable derivatives such amides, esters, and thioesters, provided they have an acid group.

Examples of preferred safeners C are benoxacor, cloquintocet, cyometrinil, cyprosulfamide, dichlormid, dicyclonon, dietholate, fenchlorazole, fenclorim, flurazole, fluxofenim, furilazole, isoxadifen, mefenpyr, mephenate, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4), metcamifen and BPCMS (CAS 54091-06-4). Especially preferred safeners C are benoxacor, cloquintocet, cyprosulfamide, dichlormid, fenchlorazole, fenclorim, flurazole, fluxofenim, furilazole, isoxadifen, mefenpyr, naphthalic anhydride, oxabetrinil, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and metcamifen.

Particularly preferred safeners C are benoxacor, cloquintocet, cyprosulfamide, dichlormid, fenchlorazole, fenclorim, furilazole, isoxadifen, mefenpyr, naphtalic anhydride, 4-(dichloroacetyl)-1-oxa-4-azaspiro[4.5]decane (MON4660, CAS 71526-07-3), 2,2,5-trimethyl-3-(dichloroacetyl)-1,3-oxazolidine (R-29148, CAS 52836-31-4) and metcamifen.

Particularly preferred safeners C, which, as component C, are constituent of the composition according to the invention are the safeners C as defined above; in particular the safeners C.1-C.17 listed below in table C:

TABLE C

| C | Safener C |
|---|---|
| C.1 | benoxacor |
| C.2 | cloquintocet |
| C.3 | cloquintocet-mexyl |
| C.4 | cyprosulfamide |
| C.5 | dichlormid |
| C.6 | fenchlorazole |
| C.7 | fenchlorazole-ethyl |
| C.8 | fenclorim |
| C.9 | furilazole |
| C.10 | isoxadifen |
| C.11 | isoxadifen-ethyl |
| C.12 | mefenpyr |
| C.13 | mefenpyr-diethyl |
| C.14 | naphtalic acid anhydride |
| C.15 | 4-(dichloroacetyl)-1-oxa-4- |
| | azaspiro[4.5]decane |
| | (MON4660, CAS 71526-07-3) |
| C.16 | 2,2,5-trimethyl-3-(dichloro- |
| | acetyl)-1,3-oxazolidine (R- |
| | 29148, CAS 52836-31-4) |
| C.17 | metcamifen |

The active compounds B of groups b1) to b15) and the active compounds C are known herbicides and safeners, see, e.g., The Compendium of Pesticide Common Names (http://www.alanwood.net/pesticides/); Farm Chemicals Handbook 2000 volume 86, Meister Publishing Company, 2000; B. Hock, C. Fedtke, R. R. Schmidt, Herbizide [Herbicides], Georg Thieme Verlag, Stuttgart 1995; W. H. Ahrens, Herbicide Handbook, 7th edition, Weed Science Society of America, 1994; and K. K. Hatzios, Herbicide Handbook, Supplement for the 7th edition, Weed Science Society of America, 1998. 2,2,5-Trimethyl-3-(dichloroacetyl)-1,3-oxazolidine [CAS No. 52836-31-4] is also referred to as R-29148. 4-(Dichloroacetyl)-1-oxa-4-azaspiro[4.5]decane [CAS No. 71526-07-3] is also referred to as AD-67 and MON 4660.

The assignment of the active compounds to the respective mechanisms of action is based on current knowledge. If several mechanisms of action apply to one active compound, this substance was only assigned to one mechanism of action.

Active compounds B and C having a carboxyl group can be employed in the form of the acid, in the form of an agriculturally suitable salt as mentioned above or else in the form of an agriculturally acceptable derivative in the compositions according to the invention.

In the case of dicamba, suitable salts include those, where the counterion is an agriculturally acceptable cation. E.g., suitable salts of dicamba are dicamba-sodium, dicamba-potassium, dicamba-methylammonium, dicamba-dimethylammonium, dicamba-isopropylammonium, dicamba-diglycolamine, dicamba-olamine, dicamba-diolamine, dicamba-trolamine, dicamba-N,N-bis-(3-aminopropyl)methylamine and dicamba-diethylenetriamine. Examples of a suitable ester are dicamba-methyl and dicamba-butotyl.

Suitable salts of 2,4-D are 2,4-D-ammonium, 2,4-D-dimethylammonium, 2,4-D-diethylammonium, 2,4-D-diethanolammonium (2,4-D-diolamine), 2,4-D-triethanolammonium, 2,4-D-isopropylammonium, 2,4-D-triisopropanolammonium, 2,4-D-heptylammonium, 2,4-D-dodecylammonium, 2,4-D-tetradecylammonium, 2,4-D-triethylammonium, 2,4-D-tris(2-hydroxypropyl)ammonium, 2,4-D-tris(isopropyl)ammonium, 2,4-D-trolamine, 2,4-D-lithium, 2,4-D-sodium.

Examples of suitable esters of 2,4-D are 2,4-D-butotyl, 2,4-D-2-butoxypropyl, 2,4-D-3-butoxypropyl, 2,4-D-butyl, 2,4-D-ethyl, 2,4-D-ethylhexyl, 2,4-D-isobutyl, 2,4-D-isooctyl, 2,4-Disopropyl, 2,4-D-meptyl, 2,4-D-methyl, 2,4-D-octyl, 2,4-D-pentyl, 2,4-D-propyl, 2,4-D-tefuryl and clacyfos.

Suitable salts of 2,4-DB are e.g. 2,4-DB-sodium, 2,4-DB-potassium and 2,4-DB-dimethylammonium.

Suitable esters of 2,4-DB are e.g. 2,4-DB-butyl and 2,4-DB-isoctyl.

Suitable salts of dichlorprop are e.g. dichlorprop-sodium, dichlorprop-potassium and dichlorprop-dimethylammonium. Examples of suitable esters of dichlorprop are dichlorprop-butotyl and dichlorprop-isoctyl.

Suitable salts and esters of MCPA include MCPA-butotyl, MCPA-butyl, MCPA-dimethylammonium, MCPA-diolamine, MCPA-ethyl, MCPA-thioethyl, MCPA-2-ethylhexyl, MCPA-isobutyl, MCPA-isoctyl, MCPA-isopropyl, MCPA-isopropylammonium, MCPA-methyl, MCPA-olamine, MCPA-potassium, MCPA-sodium and MCPA-trolamine.

A suitable salt of MCPB is MCPB sodium. A suitable ester of MCPB is MCPB-ethyl.

Suitable salts of clopyralid are clopyralid-potassium, clopyralid-olamine and clopyralid-tris-(2-hydroxypropyl)ammonium. Example of suitable esters of clopyralid is clopyralid-methyl.

Examples of a suitable ester of fluroxypyr are fluroxypyr-meptyl and fluroxypyr-2-butoxy-1-methylethyl, wherein fluroxypyr-meptyl is preferred.

Suitable salts of picloram are picloram-dimethylammonium, picloram-potassium, picloram-triisopropanolammonium, picloram-triisopropylammonium and picloram-trolamine. A suitable ester of picloram is picloram-isoctyl.

A suitable salt of triclopyr is triclopyr-triethylammonium. Suitable esters of triclopyr are e.g. triclopyr-ethyl and triclopyr-butotyl.

Suitable salts and esters of chloramben include chloramben-ammonium, chloramben-diolamine, chloramben-methyl, chloramben-methylammonium and chloramben-sodium. Suitable salts and esters of 2,3,6-TBA include 2,3,6-TBA-dimethylammonium, 2,3,6-TBA-lithium, 2,3,6-TBA-potassium and 2,3,6-TBA-sodium.

Suitable salts and esters of aminopyralid include aminopyralid-potassium, aminopyralid-dimethylammonium, and aminopyralid-tris(2-hydroxypropyl)ammonium.

Suitable salts of glyphosate are e.g. glyphosate-ammonium, glyphosate-diammonium, glyphoste-dimethylammonium, glyphosate-isopropylammonium, glyphosate-potassium, glyphosate-sodium, glyphosate-trimesium as well as the ethanolamine and diethanolamine salts, preferably glyphosate-diammonium, glyphosate-isopropylammonium and glyphosate-trimesium (sulfosate).

A suitable salt of glufosinate is e.g. glufosinate-ammonium.

A suitable salt of glufosinate-P is e.g. glufosinate-P-ammonium.

Suitable salts and esters of bromoxynil are e.g. bromoxynil-butyrate, bromoxynil-heptanoate, bromoxynil-octanoate, bromoxynil-potassium and bromoxynil-sodium.

Suitable salts and esters of ioxonil are e.g. ioxonil-octanoate, ioxonil-potassium and ioxonil-sodium.

Suitable salts and esters of mecoprop include mecoprop-butotyl, mecoprop-dimethylammonium, mecoprop-diolamine, mecoprop-ethadyl, mecoprop-2-ethylhexyl, mecoprop-isoctyl, mecoprop-methyl, mecoprop-potassium, mecoprop-sodium and mecoprop-trolamine.

Suitable salts of mecoprop-P are e.g. mecoprop-P-butotyl, mecoprop-P-dimethylammonium, mecoprop-P-2-ethylhexyl, mecoprop-P-isobutyl, mecoprop-P-potassium and mecoprop-P-sodium.

A suitable salt of diflufenzopyr is e.g. diflufenzopyr-sodium.

A suitable salt of naptalam is e.g. naptalam-sodium.

Suitable salts and esters of aminocyclopyrachlor are e.g. aminocyclopyrachlor-dimethylammonium, aminocyclopyrachlor-methyl, aminocyclopyrachlor-triisopropanolammonium, aminocyclopyrachlor-sodium and aminocyclopyrachlor-potassium.

A suitable salt of quinclorac is e.g. quinclorac-dimethylammonium.

A suitable salt of quinmerac is e.g. quinmerac-dimethylammonium.

A suitable salt of imazamox is e.g. imazamox-ammonium.

Suitable salts of imazapic are e.g. imazapic-ammonium and imazapic-isopropylammonium.

Suitable salts of imazapyr are e.g. imazapyr-ammonium and imazapyr-isopropylammonium.

A suitable salt of imazaquin is e.g. imazaquin-ammonium.

Suitable salts of imazethapyr are e.g. imazethapyr-ammonium and imazethapyr-isopropylammonium.

A suitable salt of topramezone is e.g. topramezone-sodium.

According to a preferred embodiment of the invention, the composition comprises as herbicidal active compound B or component B at least one, preferably exactly one herbicide B.

According to another preferred embodiment of the invention, the composition comprises as herbicidal active compounds B or component B at least two, preferably exactly two herbicides B different from each other.

According to another preferred embodiment of the invention, the composition comprises as herbicidal active compounds B or component B at least three, preferably exactly three herbicides B different from each other.

According to another preferred embodiment of the invention, the composition comprises as safening component C or component C at least one, preferably exactly one safener C.

According to another preferred embodiment of the invention, the composition comprises as component B at least one, preferably exactly one herbicide B, and as component C at least one, preferably exactly one, safener C.

According to another preferred embodiment of the invention, the composition comprises at least two, preferably exactly two, herbicides B different from each other, and as component C at least one, preferably exactly one, safener C.

According to another preferred embodiment of the invention, the composition comprises at least three, preferably exactly three, herbicides B different from each other, and as component C at least one, preferably exactly one, safener C.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), and as component B at least one, preferably exactly one, herbicide B.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), and at least two, preferably exactly two, herbicides B different from each other.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.1.I-45), (1.3.I-46), (1.3.I-52), (1.3.I-53), (1.3.I-59), (1.3.I-60), (1.3.I-66), or (1.3.I-67), and at least three, preferably exactly three, herbicides B different from each other.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), and as component C at least one, preferably exactly one, safener C.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (1.3), especially preferred the compound (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), or (1.3.I-67), at least three, preferably exactly three herbicides B different from each other, and as component C at least one, preferably exactly one, safener C.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), and as component B at least one, preferably exactly one, herbicide B.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), and at least two, preferably exactly two, herbicides B different from each other.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), and at least three, preferably exactly three, herbicides B different from each other.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), and as component C at least one, preferably exactly one, safener C.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (1.15), especially preferred the compound (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), or (1.15.I-67), at least three, preferably exactly three herbicides B different from each other, and as component C at least one, preferably exactly one, safener C.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), and as component B at least one, preferably exactly one, herbicide B.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), and at least two, preferably exactly two, herbicides B different from each other.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), and at least three, preferably exactly three, herbicides B different from each other.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), and as component C at least one, preferably exactly one, safener C.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), as component B at least one, preferably exactly one, herbicide B, and as component C at least one, preferably exactly one safener C.

According to another preferred embodiment of the invention, the composition comprises as component A at least one, preferably exactly pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), at least two, preferably exactly two herbicides B different from each other, and as component C at least one, preferably exactly one, safener C.

According to another preferred embodiment of the invention, the composition comprises as component A at least one, preferably exactly one pyrimidine compound of formula (I), preferably of formula (1.16), especially preferred the compound (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), or (1.16.I-67), at least three, preferably exactly three herbicides B different from each other, and as component C at least one, preferably exactly one, safener C.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b1), in particular selected from the group consisting of clethodim, clodinafop-propargyl, cycloxydim, cyhalofop-butyl, fenoxaprop-ethyl, fenoxaprop-P-ethyl, metamifop, pinoxaden, profoxydim, sethoxydim, tepraloxydim, tralkoxydim, esprocarb, ethofumesate, molinate, prosulfocarb, thiobencarb and triallate.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b2), in particular selected from the group consisting of bensulfuron-methyl, bispyribac-sodium, cloransulam-methyl, chlorsulfuron, clorimuron, cyclosulfamuron, diclosulam, florasulam, flumetsulam, flupyrsulfuron-methyl-sodium, foramsulfuron, imazamox, imazamox-ammonium, imazapic, imazapic-ammonium, imazapic-isopropylammonium, imazapyr, imazapyr-ammonium, imazethapyr-isopropylammonium, imazaquin, imazaquin-ammonium, imazethapyr, imazethapyr-ammonium, imazethapyr-isopropylammonium, imazosulfuron, iodosulfuron-methyl-sodium, iofensulfuron, iofensulfuron-sodium, mesosulfuron-methyl, metazosulfuron, metsulfuron-methyl, metosulam, nicosulfuron, penoxsulam, propoxycarbazon-sodium, pyrazosulfuron-ethyl, pyribenzoxim, pyriftalid, pyroxsulam, propyrisulfuron, rimsulfuron, sulfosulfuron, thiencarbazon-methyl, thifensulfuron-methyl, tribenuron-methyl, tritosulfuron and triafamone.

According to another preferred embodiment of the invention, the composition comprises, in addition to a pyrimidine compounds of formula (I), especially an active compound from the group consisting (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b3), in particular selected from the group consisting of ametryn, atrazine, bentazon, bromoxynil, bromoxynil-octanoate, bromoxynil-heptanoate, bromoxynil-potassium, diuron, fluometuron, hexazinone, isoproturon, linuron, metamitron, metribuzin, paraquat-dichloride, propanil, simazin, terbutryn and terbuthylazine.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b4), in particular selected from the group consisting of acifluorfen, butafencil, carfenetrazone-ethyl, flumioxazin, fomesafen, oxadiargyl, oxyfluorfen, pyraflufen, pyraflufen-ethyl, saflufenacil, sulfentrazone, trifludimoxazin, ethyl [3-[2-chloro-4-fluoro-5-(1-methyl-6-trifluoromethyl-2,4-dioxo-1,2,3,4-tetrahydropyrimidin-3-yl)phenoxy]-2-pyridyloxy]acetate (CAS 353292-31-6; S-3100).

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b5), in particular selected from the group consisting of amitrole, benzobicyclon, bicyclopyrone, clomazone, diflufenican, fenquintrone, fluometuron, flurochloridone, isoxaflutole, mesotrione, norflurazone, oxotrione (CAS 1486617-21-3), picolinafen, sulcotrione, tefuryltrione, tembotrione, tolpyralate, topramezone, topramezone-sodium and 2-chloro-3-methylsulfanyl-N-(1-methyltetrazol-5-yl)-4-(trifluoromethyl)benzamide (CAS 1361139-71-0).

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b6), in particular selected from the group consisting of glyphosate, glyphosate-ammonium, glyphosate-dimethylammonium, glyphosate-isopropylammonium and glyphosate-trimesium (sulfosate) and glyphosate-potassium.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b7), in particular selected from the group consisting of glufosinate, glufosinate-ammonium, glufosinate-P and glufosinate-P-ammonium.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b9), in particular selected from the group consisting of pendimethalin and trifluralin.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.1-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b10), in particular selected from the group consisting of acetochlor, butachlor, cafenstrole, dimethenamid-P, fentrazamide, flufenacet, mefenacet, metazachlor, metolachlor, S-metolachlor, fenoxasulfone, ipfencarbazone and pyroxasulfone. Likewise, preference is given to compositions comprising in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b10), in particular selected from the group consisting of isoxazoline compounds of the formulae II.1, II.2, II.3, II.4, II.5, II.6, II.7, II.8 and II.9, as defined above.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b11), in particular indaziflam, isoxaben and triaziflam.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b13), in particular selected from the group consisting of 2,4-D, 2,4-D-isobutyl, 2,4-D-dimethylammonium, 2,4-D-N,N,N-trimethylethanolammonium, aminocyclopyrachlor, aminocyclopyrachlor-potassium, aminocyclopyrachlor-methyl, aminopyralid, aminopyralid-methyl, aminopyralid-dimethylammonium, aminopyralid-tris(2-hydroxypropyl)ammonium, clopyralid, clopyralid-methyl, clopyralid-olamine, dicamba, dicamba-butotyl, dicamba-diglycolamine, dicamba-dimethylammonium, dicamba-diolamine, dicamba-isopropylammonium, dicamba-potassium, dicamba-sodium, dicamba-trolamine, dicamba-N,N-bis-(3-aminopropyl)methylamine, dicamba-diethylenetriamine, flopyrauxifen, fluroxypyr, fluroxypyr-meptyl, halauxifen, halauxifen-methyl, MCPA, MCPA-2-ethylhexyl, MCPA-dimethylammonium, quinclorac, quinclorac-dimethylammonium, quinmerac, quinmerac-dimethylammonium, florpyrauxifen, florpyrauxifen-benzyl (CAS 1390661-72-9), and 4-amino-3-chloro-5-fluoro-6-(7-fluoro-1H-indol-6-yl)picolinic acid (CAS 1629965-65-6).

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b14), in particular selected from the group consisting of diflufenzopyr, diflufenzopyr-sodium, dymron, indanofan and diflufenzopyr-sodium.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one herbicidally active compound from group b15), in particular selected from the group consisting of cinmethylin, dymron (=daimuron), indanofan and oxaziclomefone.

According to another preferred embodiment of the invention, the composition comprises, in addition to a pyrimidine compounds of formula (I), especially an active compound from the group consisting of (1.3.I-3), (1.3.I-4), (1.3.I-10), (1.3.I-11), (1.3.I-17), (1.3.I-18), (1.3.I-24), (1.3.I-25), (1.3.I-31), (1.3.I-32), (1.3.I-38), (1.3.I-39), (1.3.I-45), (1.3.I-46), (1.3.I-52), (1.3.I-53), (1.3.I-59), (1.3.I-60), (1.3.I-66), (1.3.I-67), (1.15.I-3), (1.15.I-4), (1.15.I-10), (1.15.I-11), (1.15.I-17), (1.15.I-18), (1.15.I-24), (1.15.I-25), (1.15.I-31), (1.15.I-32), (1.15.I-38), (1.15.I-39), (1.15.I-45), (1.15.I-46), (1.15.I-52), (1.15.I-53), (1.15.I-59), (1.15.I-60), (1.15.I-66), (1.15.I-67), (1.16.I-3), (1.16.I-4), (1.16.I-10), (1.16.I-11), (1.16.I-17), (1.16.I-18), (1.16.I-24), (1.16.I-25), (1.16.I-31), (1.16.I-32), (1.16.I-38), (1.16.I-39), (1.16.I-45), (1.16.I-46), (1.16.I-52), (1.16.I-53), (1.16.I-59), (1.16.I-60), (1.16.I-66), and (1.16.I-67), at least one and especially exactly one safener C, in particular selected from the group consisting of benoxacor, cloquintocet, cyprosulfamide, dichlormid, fenchlorazole, fenclorim, furilazole, isoxadifen, mefenpyr, 4-(dichloroacetyl)-1-oxa-4-azaspiro[4.5]decane (MON4660, CAS 71526-07-3) and 2,2,5-trimethyl-3-(dichloroacetyl)-1,3-oxazolidine (R-29148, CAS 52836-31-4).

Here and below, the term “binary compositions” includes compositions comprising one or more, e.g. 1, 2 or 3, active compounds of the formula (I) and either one or more, e.g. 1, 2 or 3, herbicides B or one or more safeners C.

Correspondingly, the term “ternary compositions” includes compositions comprising one or more, e.g. 1, 2 or 3, active compounds of the formula (I), one or more, e.g. 1, 2 or 3, herbicides B and one or more, e.g. 1, 2 or 3, safeners C.

In binary compositions comprising at least one pyrimidine compound of formula (I) as component A and at least one herbicide B, the weight ratio of the active compounds A:B is generally in the range of from 1:1000 to 1000:1, preferably in the range of from 1:500 to 500:1, in particular in the range of from 1:250 to 250:1 and particularly preferably in the range of from 1:75 to 75:1.

In binary compositions comprising at least one pyrimidine compound of formula (I) as component A and at least one safener C, the weight ratio of the active compounds A:C is generally in the range of from 1:1000 to 1000:1, preferably in the range of from 1:500 to 500:1, in particular in the range of from 1:250 to 250:1 and particularly preferably in the range of from 1:75 to 75:1.

In ternary compositions comprising at least one pyrimidine compound of formula (I) as component A, at least one herbicide B and at least one safener C, the relative proportions by weight of the components A:B are generally in the range of from 1:1000 to 1000:1, preferably in the range of from 1:500 to 500:1, in particular in the range of from 1:250 to 250:1 and particularly preferably in the range of from 1:75 to 75:1, the weight ratio of the components A:C is generally in the range of from 1:1000 to 1000:1, preferably in the range of from 1:500 to 500:1, in particular in the range of from 1:250 to 250:1 and particularly preferably in the range of from 1:75 to 75:1, and the weight ratio of the components B:C is generally in the range of from 1:1000 to 1000:1, preferably in the range of from 1:500 to 500:1, in particular in the range of from 1:250 to 250:1 and particularly preferably in the range of from 1:75 to 75:1. The weight ratio of components A+B to component C is preferably in the range of from 1:500 to 500:1, in particular in the range of from 1:250 to 250:1 and particularly preferably in the range of from 1:75 to 75:1.

The weight ratios of the individual components in the preferred mixtures mentioned below are within the limits given above, in particular within the preferred limits.

Particularly preferred are the compositions mentioned below comprising the pyrimidine compounds of formula I as defined and the substance(s) as defined in the respective row of table T;

especially preferred comprising as only herbicidal active compounds the pyrimidine compounds of formula I as defined and the substance(s) as defined in the respective row of table T;

most preferably comprising as only active compounds the pyrimidine compounds of formula I as defined and the substance(s) as defined in the respective row of table T.

Particularly preferred are compositions 1.1 to 1.3653, comprising the compound (1.3.I-3) and the substance(s) as defined in the respective row of table T:

TABLE T
(compositions 1.1 to 1.3653):

| comp. | | |
|---|---|---|
| no. | herbicide B | safener C |
| 1.1 | B.1 | — |
| 1.2 | B.2 | — |
| 1.3 | B.3 | — |
| 1.4 | B.4 | — |
| 1.5 | B.5 | — |
| 1.6 | B.6 | — |
| 1.7 | B.7 | — |
| 1.8 | B.8 | — |
| 1.9 | B.9 | — |
| 1.10 | B.10 | — |
| 1.11 | B.11 | — |
| 1.12 | B.12 | — |
| 1.13 | B.13 | — |
| 1.14 | B.14 | — |
| 1.15 | B.15 | — |
| 1.16 | B.16 | — |
| 1.17 | B.17 | — |
| 1.18 | B.18 | — |
| 1.19 | B.19 | — |
| 1.20 | B.20 | — |
| 1.21 | B.21 | — |
| 1.22 | B.22 | — |
| 1.23 | B.23 | — |
| 1.24 | B.24 | — |
| 1.25 | B.25 | — |
| 1.26 | B.26 | — |
| 1.27 | B.27 | — |
| 1.28 | B.28 | — |
| 1.29 | B.29 | — |
| 1.30 | B.30 | — |
| 1.31 | B.31 | — |
| 1.32 | B.32 | — |
| 1.33 | B.33 | — |
| 1.34 | B.34 | — |
| 1.35 | B.35 | — |
| 1.36 | B.36 | — |
| 1.37 | B.37 | — |
| 1.38 | B.38 | — |
| 1.39 | B.39 | — |
| 1.40 | B.40 | — |
| 1.41 | B.41 | — |
| 1.42 | B.42 | — |
| 1.43 | B.43 | — |
| 1.44 | B.44 | — |
| 1.45 | B.45 | — |
| 1.46 | B.46 | — |
| 1.47 | B.47 | — |
| 1.48 | B.48 | — |
| 1.49 | B.49 | — |
| 1.50 | B.50 | — |
| 1.51 | B.51 | — |
| 1.52 | B.52 | — |
| 1.53 | B.53 | — |
| 1.54 | B.54 | — |
| 1.55 | B.55 | — |
| 1.56 | B.56 | — |
| 1.57 | B.57 | — |
| 1.58 | B.58 | — |
| 1.59 | B.59 | — |
| 1.60 | B.60 | — |
| 1.61 | B.61 | — |
| 1.62 | B.62 | — |
| 1.63 | B.63 | — |
| 1.64 | B.64 | — |
| 1.65 | B.65 | — |
| 1.66 | B.66 | — |
| 1.67 | B.67 | — |
| 1.68 | B.68 | — |
| 1.69 | B.69 | — |
| 1.70 | B.70 | — |
| 1.71 | B.71 | — |
| 1.72 | B.72 | — |
| 1.73 | B.73 | — |
| 1.74 | B.74 | — |
| 1.75 | B.75 | — |
| 1.76 | B.76 | — |
| 1.77 | B.77 | — |
| 1.78 | B.78 | — |
| 1.79 | B.79 | — |
| 1.80 | B.80 | — |
| 1.81 | B.81 | — |
| 1.82 | B.82 | — |
| 1.83 | B.83 | — |
| 1.84 | B.84 | — |
| 1.85 | B.85 | — |
| 1.86 | B.86 | — |
| 1.87 | B.87 | — |
| 1.88 | B.88 | — |
| 1.89 | B.89 | — |
| 1.90 | B.90 | — |
| 1.91 | B.91 | — |
| 1.92 | B.92 | — |
| 1.93 | B.93 | — |
| 1.94 | B.94 | — |
| 1.95 | B.95 | — |
| 1.96 | B.96 | — |
| 1.97 | B.97 | — |
| 1.98 | B.98 | — |
| 1.99 | B.99 | — |
| 1.100 | B.100 | — |
| 1.101 | B.101 | — |
| 1.102 | B.102 | — |
| 1.103 | B.103 | — |
| 1.104 | B.104 | — |
| 1.105 | B.105 | — |
| 1.106 | B.106 | — |
| 1.107 | B.107 | — |
| 1.108 | B.108 | — |
| 1.109 | B.109 | — |
| 1.110 | B.110 | — |
| 1.111 | B.111 | — |
| 1.112 | B.112 | — |
| 1.113 | B.113 | — |
| 1.114 | B.114 | — |
| 1.115 | B.115 | — |
| 1.116 | B.116 | — |
| 1.117 | B.117 | — |
| 1.118 | B.118 | — |
| 1.119 | B.119 | — |
| 1.120 | B.120 | — |
| 1.121 | B.121 | — |
| 1.122 | B.122 | — |
| 1.123 | B.123 | — |
| 1.124 | B.124 | — |
| 1.125 | B.125 | — |
| 1.126 | B.126 | — |
| 1.127 | B.127 | — |
| 1.128 | B.128 | — |
| 1.129 | B.129 | — |
| 1.130 | B.130 | — |
| 1.131 | B.131 | — |
| 1.132 | B.132 | — |
| 1.133 | B.133 | — |
| 1.134 | B.134 | — |
| 1.135 | B.135 | — |
| 1.136 | B.136 | — |
| 1.137 | B.137 | — |
| 1.138 | B.138 | — |
| 1.139 | B.139 | — |
| 1.140 | B.140 | — |
| 1.141 | B.141 | — |
| 1.142 | B.142 | — |
| 1.143 | B.143 | — |
| 1.144 | B.144 | — |
| 1.145 | B.145 | — |
| 1.146 | B.146 | — |
| 1.147 | B.147 | — |
| 1.148 | B.148 | — |
| 1.149 | B.149 | — |
| 1.150 | B.150 | — |
| 1.151 | B.151 | — |
| 1.152 | B.152 | — |
| 1.153 | B.153 | — |
| 1.154 | B.154 | — |
| 1.155 | B.155 | — |
| 1.156 | B.156 | — |
| 1.157 | B.157 | — |
| 1.158 | B.158 | — |
| 1.159 | B.159 | — |
| 1.160 | B.160 | — |
| 1.161 | B.161 | — |
| 1.162 | B.162 | — |
| 1.163 | B.163 | — |
| 1.164 | B.164 | — |
| 1.165 | B.165 | — |
| 1.166 | B.166 | — |
| 1.167 | B.167 | — |
| 1.168 | B.168 | — |
| 1.169 | B.169 | — |
| 1.170 | B.170 | — |
| 1.171 | B.171 | — |
| 1.172 | B.172 | — |
| 1.173 | B.173 | — |
| 1.174 | B.174 | — |
| 1.175 | B.175 | — |
| 1.176 | B.176 | — |
| 1.177 | B.177 | — |
| 1.178 | B.178 | — |
| 1.179 | B.179 | — |
| 1.180 | B.180 | — |
| 1.181 | B.181 | — |
| 1.182 | B.182 | — |
| 1.183 | B.183 | — |
| 1.184 | B.184 | — |
| 1.185 | B.185 | — |
| 1.186 | B.186 | — |
| 1.187 | B.187 | — |
| 1.188 | B.188 | — |
| 1.189 | B.189 | — |
| 1.190 | B.190 | — |
| 1.191 | B.191 | — |
| 1.192 | B.192 | — |
| 1.193 | B.193 | — |
| 1.194 | B.194 | — |
| 1.195 | B.195 | — |
| 1.196 | B.196 | — |
| 1.197 | B.197 | — |
| 1.198 | B.198 | — |
| 1.199 | B.199 | — |
| 1.200 | B.200 | — |
| 1.201 | B.201 | — |
| 1.202 | B.202 | — |
| 1.203 | B.1 | C.1 |
| 1.204 | B.2 | C.1 |
| 1.205 | B.3 | C.1 |
| 1.206 | B.4 | C.1 |
| 1.207 | B.5 | C.1 |
| 1.208 | B.6 | C.1 |
| 1.209 | B.7 | C.1 |
| 1.210 | B.8 | C.1 |
| 1.211 | B.9 | C.1 |
| 1.212 | B.10 | C.1 |
| 1.213 | B.11 | C.1 |
| 1.214 | B.12 | C.1 |
| 1.215 | B.13 | C.1 |
| 1.216 | B.14 | C.1 |
| 1.217 | B.15 | C.1 |
| 1.218 | B.16 | C.1 |
| 1.219 | B.17 | C.1 |
| 1.220 | B.18 | C.1 |
| 1.221 | B.19 | C.1 |
| 1.222 | B.20 | C.1 |
| 1.223 | B.21 | C.1 |
| 1.224 | B.22 | C.1 |
| 1.225 | B.23 | C.1 |
| 1.226 | B.24 | C.1 |
| 1.227 | B.25 | C.1 |
| 1.228 | B.26 | C.1 |
| 1.229 | B.27 | C.1 |
| 1.230 | B.28 | C.1 |
| 1.231 | B.29 | C.1 |
| 1.232 | B.30 | C.1 |
| 1.233 | B.31 | C.1 |
| 1.234 | B.32 | C.1 |
| 1.235 | B.33 | C.1 |
| 1.236 | B.34 | C.1 |
| 1.237 | B.35 | C.1 |
| 1.238 | B.36 | C.1 |
| 1.239 | B.37 | C.1 |
| 1.240 | B.38 | C.1 |
| 1.241 | B.39 | C.1 |
| 1.242 | B.40 | C.1 |
| 1.243 | B.41 | C.1 |
| 1.244 | B.42 | C.1 |
| 1.245 | B.43 | C.1 |
| 1.246 | B.44 | C.1 |
| 1.247 | B.45 | C.1 |
| 1.248 | B.46 | C.1 |
| 1.249 | B.47 | C.1 |
| 1.250 | B.48 | C.1 |
| 1.251 | B.49 | C.1 |
| 1.252 | B.50 | C.1 |
| 1.253 | B.51 | C.1 |
| 1.254 | B.52 | C.1 |
| 1.255 | B.53 | C.1 |
| 1.256 | B.54 | C.1 |
| 1.257 | B.55 | C.1 |
| 1.258 | B.56 | C.1 |
| 1.259 | B.57 | C.1 |
| 1.260 | B.58 | C.1 |
| 1.261 | B.59 | C.1 |
| 1.262 | B.60 | C.1 |
| 1.263 | B.61 | C.1 |
| 1.264 | B.62 | C.1 |
| 1.265 | B.63 | C.1 |
| 1.266 | B.64 | C.1 |
| 1.267 | B.65 | C.1 |
| 1.268 | B.66 | C.1 |
| 1.269 | B.67 | C.1 |
| 1.270 | B.68 | C.1 |
| 1.271 | B.69 | C.1 |
| 1.272 | B.70 | C.1 |
| 1.273 | B.71 | C.1 |
| 1.274 | B.72 | C.1 |
| 1.275 | B.73 | C.1 |
| 1.276 | B.74 | C.1 |
| 1.277 | B.75 | C.1 |
| 1.278 | B.76 | C.1 |
| 1.279 | B.77 | C.1 |
| 1.280 | B.78 | C.1 |
| 1.281 | B.79 | C.1 |
| 1.282 | B.80 | C.1 |
| 1.283 | B.81 | C.1 |
| 1.284 | B.82 | C.1 |
| 1.285 | B.83 | C.1 |
| 1.286 | B.84 | C.1 |
| 1.287 | B.85 | C.1 |
| 1.288 | B.86 | C.1 |
| 1.289 | B.87 | C.1 |
| 1.290 | B.88 | C.1 |
| 1.291 | B.89 | C.1 |
| 1.292 | B.90 | C.1 |
| 1.293 | B.91 | C.1 |
| 1.294 | B.92 | C.1 |
| 1.295 | B.93 | C.1 |
| 1.296 | B.94 | C.1 |
| 1.297 | B.95 | C.1 |
| 1.298 | B.96 | C.1 |
| 1.299 | B.97 | C.1 |
| 1.300 | B.98 | C.1 |
| 1.301 | B.99 | C.1 |
| 1.302 | B.100 | C.1 |
| 1.303 | B.101 | C.1 |
| 1.304 | B.102 | C.1 |
| 1.305 | B.103 | C.1 |
| 1.306 | B.104 | C.1 |
| 1.307 | B.105 | C.1 |
| 1.308 | B.106 | C.1 |
| 1.309 | B.107 | C.1 |
| 1.310 | B.108 | C.1 |
| 1.311 | B.109 | C.1 |
| 1.312 | B.110 | C.1 |
| 1.313 | B.111 | C.1 |
| 1.314 | B.112 | C.1 |
| 1.315 | B.113 | C.1 |
| 1.316 | B.114 | C.1 |
| 1.317 | B.115 | C.1 |
| 1.318 | B.116 | C.1 |
| 1.319 | B.117 | C.1 |
| 1.320 | B.118 | C.1 |
| 1.321 | B.119 | C.1 |
| 1.322 | B.120 | C.1 |
| 1.323 | B.121 | C.1 |
| 1.324 | B.122 | C.1 |
| 1.325 | B.123 | C.1 |
| 1.326 | B.124 | C.1 |
| 1.327 | B.125 | C.1 |
| 1.328 | B.126 | C.1 |
| 1.329 | B.127 | C.1 |
| 1.330 | B.128 | C.1 |
| 1.331 | B.129 | C.1 |
| 1.332 | B.130 | C.1 |
| 1.333 | B.131 | C.1 |
| 1.334 | B.132 | C.1 |
| 1.335 | B.133 | C.1 |
| 1.336 | B.134 | C.1 |
| 1.337 | B.135 | C.1 |
| 1.338 | B.136 | C.1 |
| 1.339 | B.137 | C.1 |
| 1.340 | B.138 | C.1 |
| 1.341 | B.139 | C.1 |
| 1.342 | B.140 | C.1 |
| 1.343 | B.141 | C.1 |
| 1.344 | B.142 | C.1 |
| 1.345 | B.143 | C.1 |
| 1.346 | B.144 | C.1 |
| 1.347 | B.145 | C.1 |
| 1.348 | B.146 | C.1 |
| 1.349 | B.147 | C.1 |
| 1.350 | B.148 | C.1 |
| 1.351 | B.149 | C.1 |
| 1.352 | B.150 | C.1 |
| 1.353 | B.151 | C.1 |
| 1.354 | B.152 | C.1 |
| 1.355 | B.153 | C.1 |
| 1.356 | B.154 | C.1 |
| 1.357 | B.155 | C.1 |
| 1.358 | B.156 | C.1 |
| 1.359 | B.157 | C.1 |
| 1.360 | B.158 | C.1 |
| 1.361 | B.159 | C.1 |
| 1.362 | B.160 | C.1 |
| 1.363 | B.161 | C.1 |
| 1.364 | B.162 | C.1 |
| 1.365 | B.163 | C.1 |
| 1.366 | B.164 | C.1 |
| 1.367 | B.165 | C.1 |
| 1.368 | B.166 | C.1 |
| 1.369 | B.167 | C.1 |
| 1.370 | B.168 | C.1 |
| 1.371 | B.169 | C.1 |
| 1.372 | B.170 | C.1 |
| 1.373 | B.171 | C.1 |
| 1.374 | B.172 | C.1 |
| 1.375 | B.173 | C.1 |
| 1.376 | B.174 | C.1 |
| 1.377 | B.175 | C.1 |
| 1.378 | B.176 | C.1 |
| 1.379 | B.177 | C.1 |
| 1.380 | B.178 | C.1 |
| 1.381 | B.179 | C.1 |
| 1.382 | B.180 | C.1 |
| 1.383 | B.181 | C.1 |
| 1.384 | B.182 | C.1 |
| 1.385 | B.183 | C.1 |
| 1.386 | B.184 | C.1 |
| 1.387 | B.185 | C.1 |
| 1.388 | B.186 | C.1 |
| 1.389 | B.187 | C.1 |
| 1.390 | B.188 | C.1 |
| 1.391 | B.189 | C.1 |
| 1.392 | B.190 | C.1 |
| 1.393 | B.191 | C.1 |
| 1.394 | B.192 | C.1 |
| 1.395 | B.193 | C.1 |
| 1.396 | B.194 | C.1 |
| 1.397 | B.195 | C.1 |
| 1.398 | B.196 | C.1 |
| 1.399 | B.197 | C.1 |
| 1.400 | B.198 | C.1 |
| 1.401 | B.199 | C.1 |
| 1.402 | B.200 | C.1 |
| 1.403 | B.201 | C.1 |
| 1.404 | B.202 | C.1 |
| 1.405 | B.1 | C.2 |
| 1.406 | B.2 | C.2 |
| 1.407 | B.3 | C.2 |
| 1.408 | B.4 | C.2 |
| 1.409 | B.5 | C.2 |
| 1.410 | B.6 | C.2 |
| 1.411 | B.7 | C.2 |
| 1.412 | B.8 | C.2 |
| 1.413 | B.9 | C.2 |
| 1.414 | B.10 | C.2 |
| 1.415 | B.11 | C.2 |
| 1.416 | B.12 | C.2 |
| 1.417 | B.13 | C.2 |
| 1.418 | B.14 | C.2 |
| 1.419 | B.15 | C.2 |
| 1.420 | B.16 | C.2 |
| 1.421 | B.17 | C.2 |
| 1.422 | B.18 | C.2 |
| 1.423 | B.19 | C.2 |
| 1.424 | B.20 | C.2 |
| 1.425 | B.21 | C.2 |
| 1.426 | B.22 | C.2 |
| 1.427 | B.23 | C.2 |
| 1.428 | B.24 | C.2 |
| 1.429 | B.25 | C.2 |
| 1.430 | B.26 | C.2 |
| 1.431 | B.27 | C.2 |
| 1.432 | B.28 | C.2 |
| 1.433 | B.29 | C.2 |
| 1.434 | B.30 | C.2 |
| 1.435 | B.31 | C.2 |
| 1.436 | B.32 | C.2 |
| 1.437 | B.33 | C.2 |
| 1.438 | B.34 | C.2 |
| 1.439 | B.35 | C.2 |
| 1.440 | B.36 | C.2 |
| 1.441 | B.37 | C.2 |
| 1.442 | B.38 | C.2 |
| 1.443 | B.39 | C.2 |
| 1.444 | B.40 | C.2 |
| 1.445 | B.41 | C.2 |
| 1.446 | B.42 | C.2 |
| 1.447 | B.43 | C.2 |
| 1.448 | B.44 | C.2 |
| 1.449 | B.45 | C.2 |
| 1.450 | B.46 | C.2 |
| 1.451 | B.47 | C.2 |
| 1.452 | B.48 | C.2 |
| 1.453 | B.49 | C.2 |
| 1.454 | B.50 | C.2 |
| 1.455 | B.51 | C.2 |
| 1.456 | B.52 | C.2 |
| 1.457 | B.53 | C.2 |
| 1.458 | B.54 | C.2 |
| 1.459 | B.55 | C.2 |
| 1.460 | B.56 | C.2 |
| 1.461 | B.57 | C.2 |
| 1.462 | B.58 | C.2 |
| 1.463 | B.59 | C.2 |
| 1.464 | B.60 | C.2 |
| 1.465 | B.61 | C.2 |
| 1.466 | B.62 | C.2 |
| 1.467 | B.63 | C.2 |
| 1.468 | B.64 | C.2 |
| 1.469 | B.65 | C.2 |
| 1.470 | B.66 | C.2 |
| 1.471 | B.67 | C.2 |
| 1.472 | B.68 | C.2 |
| 1.473 | B.69 | C.2 |
| 1.474 | B.70 | C.2 |
| 1.475 | B.71 | C.2 |
| 1.476 | B.72 | C.2 |
| 1.477 | B.73 | C.2 |
| 1.478 | B.74 | C.2 |
| 1.479 | B.75 | C.2 |
| 1.480 | B.76 | C.2 |
| 1.481 | B.77 | C.2 |
| 1.482 | B.78 | C.2 |
| 1.483 | B.79 | C.2 |
| 1.484 | B.80 | C.2 |
| 1.485 | B.81 | C.2 |
| 1.486 | B.82 | C.2 |
| 1.487 | B.83 | C.2 |
| 1.488 | B.84 | C.2 |
| 1.489 | B.85 | C.2 |
| 1.490 | B.86 | C.2 |
| 1.491 | B.87 | C.2 |
| 1.492 | B.88 | C.2 |
| 1.493 | B.89 | C.2 |
| 1.494 | B.90 | C.2 |
| 1.495 | B.91 | C.2 |
| 1.496 | B.92 | C.2 |
| 1.497 | B.93 | C.2 |
| 1.498 | B.94 | C.2 |
| 1.499 | B.95 | C.2 |
| 1.500 | B.96 | C.2 |
| 1.501 | B.97 | C.2 |
| 1.502 | B.98 | C.2 |
| 1.503 | B.99 | C.2 |
| 1.504 | B.100 | C.2 |
| 1.505 | B.101 | C.2 |
| 1.506 | B.102 | C.2 |
| 1.507 | B.103 | C.2 |
| 1.508 | B.104 | C.2 |
| 1.509 | B.105 | C.2 |
| 1.510 | B.106 | C.2 |
| 1.511 | B.107 | C.2 |
| 1.512 | B.108 | C.2 |
| 1.513 | B.109 | C.2 |
| 1.514 | B.110 | C.2 |
| 1.515 | B.111 | C.2 |
| 1.516 | B.112 | C.2 |
| 1.517 | B.113 | C.2 |
| 1.518 | B.114 | C.2 |
| 1.519 | B.115 | C.2 |
| 1.520 | B.116 | C.2 |
| 1.521 | B.117 | C.2 |
| 1.522 | B.118 | C.2 |
| 1.523 | B.119 | C.2 |
| 1.524 | B.120 | C.2 |
| 1.525 | B.121 | C.2 |
| 1.526 | B.122 | C.2 |
| 1.527 | B.123 | C.2 |
| 1.528 | B.124 | C.2 |
| 1.529 | B.125 | C.2 |
| 1.530 | B.126 | C.2 |
| 1.531 | B.127 | C.2 |
| 1.532 | B.128 | C.2 |
| 1.533 | B.129 | C.2 |
| 1.534 | B.130 | C.2 |
| 1.535 | B.131 | C.2 |
| 1.536 | B.132 | C.2 |
| 1.537 | B.133 | C.2 |
| 1.538 | B.134 | C.2 |
| 1.539 | B.135 | C.2 |
| 1.540 | B.136 | C.2 |
| 1.541 | B.137 | C.2 |
| 1.542 | B.138 | C.2 |
| 1.543 | B.139 | C.2 |
| 1.544 | B.140 | C.2 |
| 1.545 | B.141 | C.2 |
| 1.546 | B.142 | C.2 |
| 1.547 | B.143 | C.2 |
| 1.548 | B.144 | C.2 |
| 1.549 | B.145 | C.2 |
| 1.550 | B.146 | C.2 |
| 1.551 | B.147 | C.2 |
| 1.552 | B.148 | C.2 |
| 1.553 | B.149 | C.2 |
| 1.554 | B.150 | C.2 |
| 1.555 | B.151 | C.2 |
| 1.556 | B.152 | C.2 |
| 1.557 | B.153 | C.2 |
| 1.558 | B.154 | C.2 |
| 1.559 | B.155 | C.2 |
| 1.560 | B.156 | C.2 |
| 1.561 | B.157 | C.2 |
| 1.562 | B.158 | C.2 |
| 1.563 | B.159 | C.2 |
| 1.564 | B.160 | C.2 |
| 1.565 | B.161 | C.2 |
| 1.566 | B.162 | C.2 |
| 1.567 | B.163 | C.2 |
| 1.568 | B.164 | C.2 |
| 1.569 | B.165 | C.2 |
| 1.570 | B.166 | C.2 |
| 1.571 | B.167 | C.2 |
| 1.572 | B.168 | C.2 |
| 1.573 | B.169 | C.2 |
| 1.574 | B.170 | C.2 |
| 1.575 | B.171 | C.2 |
| 1.576 | B.172 | C.2 |
| 1.577 | B.173 | C.2 |
| 1.578 | B.174 | C.2 |
| 1.579 | B.175 | C.2 |
| 1.580 | B.176 | C.2 |
| 1.581 | B.177 | C.2 |
| 1.582 | B.178 | C.2 |
| 1.583 | B.179 | C.2 |
| 1.584 | B.180 | C.2 |
| 1.585 | B.181 | C.2 |
| 1.586 | B.182 | C.2 |
| 1.587 | B.183 | C.2 |
| 1.588 | B.184 | C.2 |
| 1.589 | B.185 | C.2 |
| 1.590 | B.186 | C.2 |
| 1.591 | B.187 | C.2 |
| 1.592 | B.188 | C.2 |
| 1.593 | B.189 | C.2 |
| 1.594 | B.190 | C.2 |
| 1.595 | B.191 | C.2 |
| 1.596 | B.192 | C.2 |
| 1.597 | B.193 | C.2 |
| 1.598 | B.194 | C.2 |
| 1.599 | B.195 | C.2 |
| 1.600 | B.196 | C.2 |
| 1.601 | B.197 | C.2 |
| 1.602 | B.198 | C.2 |
| 1.603 | B.199 | C.2 |
| 1.604 | B.200 | C.2 |
| 1.605 | B.201 | C.2 |
| 1.606 | B.202 | C.2 |
| 1.607 | B.1 | C.3 |
| 1.608 | B.2 | C.3 |
| 1.609 | B.3 | C.3 |
| 1.610 | B.4 | C.3 |
| 1.611 | B.5 | C.3 |
| 1.612 | B.6 | C.3 |
| 1.613 | B.7 | C.3 |
| 1.614 | B.8 | C.3 |
| 1.615 | B.9 | C.3 |
| 1.616 | B.10 | C.3 |
| 1.617 | B.11 | C.3 |
| 1.618 | B.12 | C.3 |
| 1.619 | B.13 | C.3 |
| 1.620 | B.14 | C.3 |
| 1.621 | B.15 | C.3 |
| 1.622 | B.16 | C.3 |
| 1.623 | B.17 | C.3 |
| 1.624 | B.18 | C.3 |
| 1.625 | B.19 | C.3 |
| 1.626 | B.20 | C.3 |
| 1.627 | B.21 | C.3 |
| 1.628 | B.22 | C.3 |
| 1.629 | B.23 | C.3 |
| 1.630 | B.24 | C.3 |
| 1.631 | B.25 | C.3 |
| 1.632 | B.26 | C.3 |
| 1.633 | B.27 | C.3 |
| 1.634 | B.28 | C.3 |
| 1.635 | B.29 | C.3 |
| 1.636 | B.30 | C.3 |
| 1.637 | B.31 | C.3 |
| 1.638 | B.32 | C.3 |
| 1.639 | B.33 | C.3 |
| 1.640 | B.34 | C.3 |
| 1.641 | B.35 | C.3 |
| 1.642 | B.36 | C.3 |
| 1.643 | B.37 | C.3 |
| 1.644 | B.38 | C.3 |
| 1.645 | B.39 | C.3 |
| 1.646 | B.40 | C.3 |
| 1.647 | B.41 | C.3 |
| 1.648 | B.42 | C.3 |
| 1.649 | B.43 | C.3 |
| 1.650 | B.44 | C.3 |
| 1.651 | B.45 | C.3 |
| 1.652 | B.46 | C.3 |
| 1.653 | B.47 | C.3 |
| 1.654 | B.48 | C.3 |
| 1.655 | B.49 | C.3 |
| 1.656 | B.50 | C.3 |
| 1.657 | B.51 | C.3 |
| 1.658 | B.52 | C.3 |
| 1.659 | B.53 | C.3 |
| 1.660 | B.54 | C.3 |
| 1.661 | B.55 | C.3 |
| 1.662 | B.56 | C.3 |
| 1.663 | B.57 | C.3 |
| 1.664 | B.58 | C.3 |
| 1.665 | B.59 | C.3 |
| 1.666 | B.60 | C.3 |
| 1.667 | B.61 | C.3 |
| 1.668 | B.62 | C.3 |
| 1.669 | B.63 | C.3 |
| 1.670 | B.64 | C.3 |
| 1.671 | B.65 | C.3 |
| 1.672 | B.66 | C.3 |
| 1.673 | B.67 | C.3 |
| 1.674 | B.68 | C.3 |
| 1.675 | B.69 | C.3 |
| 1.676 | B.70 | C.3 |
| 1.677 | B.71 | C.3 |
| 1.678 | B.72 | C.3 |
| 1.679 | B.73 | C.3 |
| 1.680 | B.74 | C.3 |
| 1.681 | B.75 | C.3 |
| 1.682 | B.76 | C.3 |
| 1.683 | B.77 | C.3 |
| 1.684 | B.78 | C.3 |
| 1.685 | B.79 | C.3 |
| 1.686 | B.80 | C.3 |
| 1.687 | B.81 | C.3 |
| 1.688 | B.82 | C.3 |
| 1.689 | B.83 | C.3 |
| 1.690 | B.84 | C.3 |
| 1.691 | B.85 | C.3 |
| 1.692 | B.86 | C.3 |
| 1.693 | B.87 | C.3 |
| 1.694 | B.88 | C.3 |
| 1.695 | B.89 | C.3 |
| 1.696 | B.90 | C.3 |
| 1.697 | B.91 | C.3 |
| 1.698 | B.92 | C.3 |
| 1.699 | B.93 | C.3 |
| 1.700 | B.94 | C.3 |
| 1.701 | B.95 | C.3 |
| 1.702 | B.96 | C.3 |
| 1.703 | B.97 | C.3 |
| 1.704 | B.98 | C.3 |
| 1.705 | B.99 | C.3 |
| 1.706 | B.100 | C.3 |
| 1.707 | B.101 | C.3 |
| 1.708 | B.102 | C.3 |
| 1.709 | B.103 | C.3 |
| 1.710 | B.104 | C.3 |
| 1.711 | B.105 | C.3 |
| 1.712 | B.106 | C.3 |
| 1.713 | B.107 | C.3 |
| 1.714 | B.108 | C.3 |
| 1.715 | B.109 | C.3 |
| 1.716 | B.110 | C.3 |
| 1.717 | B.111 | C.3 |
| 1.718 | B.112 | C.3 |
| 1.719 | B.113 | C.3 |
| 1.720 | B.114 | C.3 |
| 1.721 | B.115 | C.3 |
| 1.722 | B.116 | C.3 |
| 1.723 | B.117 | C.3 |
| 1.724 | B.118 | C.3 |
| 1.725 | B.119 | C.3 |
| 1.726 | B.120 | C.3 |
| 1.727 | B.121 | C.3 |
| 1.728 | B.122 | C.3 |
| 1.729 | B.123 | C.3 |
| 1.730 | B.124 | C.3 |
| 1.731 | B.125 | C.3 |
| 1.732 | B.126 | C.3 |
| 1.733 | B.127 | C.3 |
| 1.734 | B.128 | C.3 |
| 1.735 | B.129 | C.3 |
| 1.736 | B.130 | C.3 |
| 1.737 | B.131 | C.3 |
| 1.738 | B.132 | C.3 |
| 1.739 | B.133 | C.3 |
| 1.740 | B.134 | C.3 |
| 1.741 | B.135 | C.3 |
| 1.742 | B.136 | C.3 |
| 1.743 | B.137 | C.3 |
| 1.744 | B.138 | C.3 |
| 1.745 | B.139 | C.3 |
| 1.746 | B.140 | C.3 |
| 1.747 | B.141 | C.3 |
| 1.748 | B.142 | C.3 |
| 1.749 | B.143 | C.3 |
| 1.750 | B.144 | C.3 |
| 1.751 | B.145 | C.3 |
| 1.752 | B.146 | C.3 |
| 1.753 | B.147 | C.3 |
| 1.754 | B.148 | C.3 |
| 1.755 | B.149 | C.3 |
| 1.756 | B.150 | C.3 |
| 1.757 | B.151 | C.3 |
| 1.758 | B.152 | C.3 |
| 1.759 | B.153 | C.3 |
| 1.760 | B.154 | C.3 |
| 1.761 | B.155 | C.3 |
| 1.762 | B.156 | C.3 |
| 1.763 | B.157 | C.3 |
| 1.764 | B.158 | C.3 |
| 1.765 | B.159 | C.3 |
| 1.766 | B.160 | C.3 |
| 1.767 | B.161 | C.3 |
| 1.768 | B.162 | C.3 |
| 1.769 | B.163 | C.3 |
| 1.770 | B.164 | C.3 |
| 1.771 | B.165 | C.3 |
| 1.772 | B.166 | C.3 |
| 1.773 | B.167 | C.3 |
| 1.774 | B.168 | C.3 |
| 1.775 | B.169 | C.3 |
| 1.776 | B.170 | C.3 |
| 1.777 | B.171 | C.3 |
| 1.778 | B.172 | C.3 |
| 1.779 | B.173 | C.3 |
| 1.780 | B.174 | C.3 |
| 1.781 | B.175 | C.3 |
| 1.782 | B.176 | C.3 |
| 1.783 | B.177 | C.3 |
| 1.784 | B.178 | C.3 |
| 1.785 | B.179 | C.3 |
| 1.786 | B.180 | C.3 |
| 1.787 | B.181 | C.3 |
| 1.788 | B.182 | C.3 |
| 1.789 | B.183 | C.3 |
| 1.790 | B.184 | C.3 |
| 1.791 | B.185 | C.3 |
| 1.792 | B.186 | C.3 |
| 1.793 | B.187 | C.3 |
| 1.794 | B.188 | C.3 |
| 1.795 | B.189 | C.3 |
| 1.796 | B.190 | C.3 |
| 1.797 | B.191 | C.3 |
| 1.798 | B.192 | C.3 |
| 1.799 | B.193 | C.3 |
| 1.800 | B.194 | C.3 |
| 1.801 | B.195 | C.3 |
| 1.802 | B.196 | C.3 |
| 1.803 | B.197 | C.3 |
| 1.804 | B.198 | C.3 |
| 1.805 | B.199 | C.3 |
| 1.806 | B.200 | C.3 |
| 1.807 | B.201 | C.3 |
| 1.808 | B.202 | C.3 |
| 1.809 | B.1 | C.4 |
| 1.810 | B.2 | C.4 |
| 1.811 | B.3 | C.4 |
| 1.812 | B.4 | C.4 |
| 1.813 | B.5 | C.4 |
| 1.814 | B.6 | C.4 |
| 1.815 | B.7 | C.4 |
| 1.816 | B.8 | C.4 |
| 1.817 | B.9 | C.4 |
| 1.818 | B.10 | C.4 |
| 1.819 | B.11 | C.4 |
| 1.820 | B.12 | C.4 |
| 1.821 | B.13 | C.4 |
| 1.822 | B.14 | C.4 |
| 1.823 | B.15 | C.4 |
| 1.824 | B.16 | C.4 |
| 1.825 | B.17 | C.4 |
| 1.826 | B.18 | C.4 |
| 1.827 | B.19 | C.4 |
| 1.828 | B.20 | C.4 |
| 1.829 | B.21 | C.4 |
| 1.830 | B.22 | C.4 |
| 1.831 | B.23 | C.4 |
| 1.832 | B.24 | C.4 |
| 1.833 | B.25 | C.4 |
| 1.834 | B.26 | C.4 |
| 1.835 | B.27 | C.4 |
| 1.836 | B.28 | C.4 |
| 1.837 | B.29 | C.4 |
| 1.838 | B.30 | C.4 |
| 1.839 | B.31 | C.4 |
| 1.840 | B.32 | C.4 |
| 1.841 | B.33 | C.4 |
| 1.842 | B.34 | C.4 |
| 1.843 | B.35 | C.4 |
| 1.844 | B.36 | C.4 |
| 1.845 | B.37 | C.4 |
| 1.846 | B.38 | C.4 |
| 1.847 | B.39 | C.4 |
| 1.848 | B.40 | C.4 |
| 1.849 | B.41 | C.4 |
| 1.850 | B.42 | C.4 |
| 1.851 | B.43 | C.4 |
| 1.852 | B.44 | C.4 |
| 1.853 | B.45 | C.4 |
| 1.854 | B.46 | C.4 |
| 1.855 | B.47 | C.4 |
| 1.856 | B.48 | C.4 |
| 1.857 | B.49 | C.4 |
| 1.858 | B.50 | C.4 |
| 1.859 | B.51 | C.4 |
| 1.860 | B.52 | C.4 |
| 1.861 | B.53 | C.4 |
| 1.862 | B.54 | C.4 |
| 1.863 | B.55 | C.4 |
| 1.864 | B.56 | C.4 |
| 1.865 | B.57 | C.4 |
| 1.866 | B.58 | C.4 |
| 1.867 | B.59 | C.4 |
| 1.868 | B.60 | C.4 |
| 1.869 | B.61 | C.4 |
| 1.870 | B.62 | C.4 |
| 1.871 | B.63 | C.4 |
| 1.872 | B.64 | C.4 |
| 1.873 | B.65 | C.4 |
| 1.874 | B.66 | C.4 |
| 1.875 | B.67 | C.4 |
| 1.876 | B.68 | C.4 |
| 1.877 | B.69 | C.4 |
| 1.878 | B.70 | C.4 |
| 1.879 | B.71 | C.4 |
| 1.880 | B.72 | C.4 |
| 1.881 | B.73 | C.4 |
| 1.882 | B.74 | C.4 |
| 1.883 | B.75 | C.4 |
| 1.884 | B.76 | C.4 |
| 1.885 | B.77 | C.4 |
| 1.886 | B.78 | C.4 |
| 1.887 | B.79 | C.4 |
| 1.888 | B.80 | C.4 |
| 1.889 | B.81 | C.4 |
| 1.890 | B.82 | C.4 |
| 1.891 | B.83 | C.4 |
| 1.892 | B.84 | C.4 |
| 1.893 | B.85 | C.4 |
| 1.894 | B.86 | C.4 |
| 1.895 | B.87 | C.4 |
| 1.896 | B.88 | C.4 |
| 1.897 | B.89 | C.4 |
| 1.898 | B.90 | C.4 |
| 1.899 | B.91 | C.4 |
| 1.900 | B.92 | C.4 |
| 1.901 | B.93 | C.4 |
| 1.902 | B.94 | C.4 |
| 1.903 | B.95 | C.4 |
| 1.904 | B.96 | C.4 |
| 1.905 | B.97 | C.4 |
| 1.906 | B.98 | C.4 |
| 1.907 | B.99 | C.4 |
| 1.908 | B.100 | C.4 |
| 1.909 | B.101 | C.4 |
| 1.910 | B.102 | C.4 |
| 1.911 | B.103 | C.4 |
| 1.912 | B.104 | C.4 |
| 1.913 | B.105 | C.4 |
| 1.914 | B.106 | C.4 |
| 1.915 | B.107 | C.4 |
| 1.916 | B.108 | C.4 |
| 1.917 | B.109 | C.4 |
| 1.918 | B.110 | C.4 |
| 1.919 | B.111 | C.4 |
| 1.920 | B.112 | C.4 |
| 1.921 | B.113 | C.4 |
| 1.922 | B.114 | C.4 |
| 1.923 | B.115 | C.4 |
| 1.924 | B.116 | C.4 |
| 1.925 | B.117 | C.4 |
| 1.926 | B.118 | C.4 |
| 1.927 | B.119 | C.4 |
| 1.928 | B.120 | C.4 |
| 1.929 | B.121 | C.4 |
| 1.930 | B.122 | C.4 |
| 1.931 | B.123 | C.4 |
| 1.932 | B.124 | C.4 |
| 1.933 | B.125 | C.4 |
| 1.934 | B.126 | C.4 |
| 1.935 | B.127 | C.4 |
| 1.936 | B.128 | C.4 |
| 1.937 | B.129 | C.4 |
| 1.938 | B.130 | C.4 |
| 1.939 | B.131 | C.4 |
| 1.940 | B.132 | C.4 |
| 1.941 | B.133 | C.4 |
| 1.942 | B.134 | C.4 |
| 1.943 | B.135 | C.4 |
| 1.944 | B.136 | C.4 |
| 1.945 | B.137 | C.4 |
| 1.946 | B.138 | C.4 |
| 1.947 | B.139 | C.4 |
| 1.948 | B.140 | C.4 |
| 1.949 | B.141 | C.4 |
| 1.950 | B.142 | C.4 |
| 1.951 | B.143 | C.4 |
| 1.952 | B.144 | C.4 |
| 1.953 | B.145 | C.4 |
| 1.954 | B.146 | C.4 |
| 1.955 | B.147 | C.4 |
| 1.956 | B.148 | C.4 |
| 1.957 | B.149 | C.4 |
| 1.958 | B.150 | C.4 |
| 1.959 | B.151 | C.4 |
| 1.960 | B.152 | C.4 |
| 1.961 | B.153 | C.4 |
| 1.962 | B.154 | C.4 |
| 1.963 | B.155 | C.4 |
| 1.964 | B.156 | C.4 |
| 1.965 | B.157 | C.4 |
| 1.966 | B.158 | C.4 |
| 1.967 | B.159 | C.4 |
| 1.968 | B.160 | C.4 |
| 1.969 | B.161 | C.4 |
| 1.970 | B.162 | C.4 |
| 1.971 | B.163 | C.4 |
| 1.972 | B.164 | C.4 |
| 1.973 | B.165 | C.4 |
| 1.974 | B.166 | C.4 |
| 1.975 | B.167 | C.4 |
| 1.976 | B.168 | C.4 |
| 1.977 | B.169 | C.4 |
| 1.978 | B.170 | C.4 |
| 1.979 | B.171 | C.4 |
| 1.980 | B.172 | C.4 |
| 1.981 | B.173 | C.4 |
| 1.982 | B.174 | C.4 |
| 1.983 | B.175 | C.4 |
| 1.984 | B.176 | C.4 |
| 1.985 | B.177 | C.4 |
| 1.986 | B.178 | C.4 |
| 1.987 | B.179 | C.4 |
| 1.988 | B.180 | C.4 |
| 1.989 | B.181 | C.4 |
| 1.990 | B.182 | C.4 |
| 1.991 | B.183 | C.4 |
| 1.992 | B.184 | C.4 |
| 1.993 | B.185 | C.4 |
| 1.994 | B.186 | C.4 |
| 1.995 | B.187 | C.4 |
| 1.996 | B.188 | C.4 |
| 1.997 | B.189 | C.4 |
| 1.998 | B.190 | C.4 |
| 1.999 | B.191 | C.4 |
| 1.1000 | B.192 | C.4 |
| 1.1001 | B.193 | C.4 |
| 1.1002 | B.194 | C.4 |
| 1.1003 | B.195 | C.4 |
| 1.1004 | B.196 | C.4 |
| 1.1005 | B.197 | C.4 |
| 1.1006 | B.198 | C.4 |
| 1.1007 | B.199 | C.4 |
| 1.1008 | B.200 | C.4 |
| 1.1009 | B.201 | C.4 |
| 1.1010 | B.202 | C.4 |
| 1.1011 | B.1 | C.5 |
| 1.1012 | B.2 | C.5 |
| 1.1013 | B.3 | C.5 |
| 1.1014 | B.4 | C.5 |
| 1.1015 | B.5 | C.5 |
| 1.1016 | B.6 | C.5 |
| 1.1017 | B.7 | C.5 |
| 1.1018 | B.8 | C.5 |
| 1.1019 | B.9 | C.5 |
| 1.1020 | B.10 | C.5 |
| 1.1021 | B.11 | C.5 |
| 1.1022 | B.12 | C.5 |
| 1.1023 | B.13 | C.5 |
| 1.1024 | B.14 | C.5 |
| 1.1025 | B.15 | C.5 |
| 1.1026 | B.16 | C.5 |
| 1.1027 | B.17 | C.5 |
| 1.1028 | B.18 | C.5 |
| 1.1029 | B.19 | C.5 |
| 1.1030 | B.20 | C.5 |
| 1.1031 | B.21 | C.5 |
| 1.1032 | B.22 | C.5 |
| 1.1033 | B.23 | C.5 |
| 1.1034 | B.24 | C.5 |
| 1.1035 | B.25 | C.5 |
| 1.1036 | B.26 | C.5 |
| 1.1037 | B.27 | C.5 |
| 1.1038 | B.28 | C.5 |
| 1.1039 | B.29 | C.5 |
| 1.1040 | B.30 | C.5 |
| 1.1041 | B.31 | C.5 |
| 1.1042 | B.32 | C.5 |
| 1.1043 | B.33 | C.5 |
| 1.1044 | B.34 | C.5 |
| 1.1045 | B.35 | C.5 |
| 1.1046 | B.36 | C.5 |
| 1.1047 | B.37 | C.5 |
| 1.1048 | B.38 | C.5 |
| 1.1049 | B.39 | C.5 |
| 1.1050 | B.40 | C.5 |
| 1.1051 | B.41 | C.5 |
| 1.1052 | B.42 | C.5 |
| 1.1053 | B.43 | C.5 |
| 1.1054 | B.44 | C.5 |
| 1.1055 | B.45 | C.5 |
| 1.1056 | B.46 | C.5 |
| 1.1057 | B.47 | C.5 |
| 1.1058 | B.48 | C.5 |
| 1.1059 | B.49 | C.5 |
| 1.1060 | B.50 | C.5 |
| 1.1061 | B.51 | C.5 |
| 1.1062 | B.52 | C.5 |
| 1.1063 | B.53 | C.5 |
| 1.1064 | B.54 | C.5 |
| 1.1065 | B.55 | C.5 |
| 1.1066 | B.56 | C.5 |
| 1.1067 | B.57 | C.5 |
| 1.1068 | B.58 | C.5 |
| 1.1069 | B.59 | C.5 |
| 1.1070 | B.60 | C.5 |
| 1.1071 | B.61 | C.5 |
| 1.1072 | B.62 | C.5 |
| 1.1073 | B.63 | C.5 |
| 1.1074 | B.64 | C.5 |
| 1.1075 | B.65 | C.5 |
| 1.1076 | B.66 | C.5 |
| 1.1077 | B.67 | C.5 |
| 1.1078 | B.68 | C.5 |
| 1.1079 | B.69 | C.5 |
| 1.1080 | B.70 | C.5 |
| 1.1081 | B.71 | C.5 |
| 1.1082 | B.72 | C.5 |
| 1.1083 | B.73 | C.5 |
| 1.1084 | B.74 | C.5 |
| 1.1085 | B.75 | C.5 |
| 1.1086 | B.76 | C.5 |
| 1.1087 | B.77 | C.5 |
| 1.1088 | B.78 | C.5 |
| 1.1089 | B.79 | C.5 |
| 1.1090 | B.80 | C.5 |
| 1.1091 | B.81 | C.5 |
| 1.1092 | B.82 | C.5 |
| 1.1093 | B.83 | C.5 |
| 1.1094 | B.84 | C.5 |
| 1.1095 | B.85 | C.5 |
| 1.1096 | B.86 | C.5 |
| 1.1097 | B.87 | C.5 |
| 1.1098 | B.88 | C.5 |
| 1.1099 | B.89 | C.5 |
| 1.1100 | B.90 | C.5 |
| 1.1101 | B.91 | C.5 |
| 1.1102 | B.92 | C.5 |
| 1.1103 | B.93 | C.5 |
| 1.1104 | B.94 | C.5 |
| 1.1105 | B.95 | C.5 |
| 1.1106 | B.96 | C.5 |
| 1.1107 | B.97 | C.5 |
| 1.1108 | B.98 | C.5 |
| 1.1109 | B.99 | C.5 |
| 1.1110 | B.100 | C.5 |
| 1.1111 | B.101 | C.5 |
| 1.1112 | B.102 | C.5 |
| 1.1113 | B.103 | C.5 |
| 1.1114 | B.104 | C.5 |
| 1.1115 | B.105 | C.5 |
| 1.1116 | B.106 | C.5 |
| 1.1117 | B.107 | C.5 |
| 1.1118 | B.108 | C.5 |
| 1.1119 | B.109 | C.5 |
| 1.1120 | B.110 | C.5 |
| 1.1121 | B.111 | C.5 |
| 1.1122 | B.112 | C.5 |
| 1.1123 | B.113 | C.5 |
| 1.1124 | B.114 | C.5 |
| 1.1125 | B.115 | C.5 |
| 1.1126 | B.116 | C.5 |
| 1.1127 | B.117 | C.5 |
| 1.1128 | B.118 | C.5 |
| 1.1129 | B.119 | C.5 |
| 1.1130 | B.120 | C.5 |
| 1.1131 | B.121 | C.5 |
| 1.1132 | B.122 | C.5 |
| 1.1133 | B.123 | C.5 |
| 1.1134 | B.124 | C.5 |
| 1.1135 | B.125 | C.5 |
| 1.1136 | B.126 | C.5 |
| 1.1137 | B.127 | C.5 |
| 1.1138 | B.128 | C.5 |
| 1.1139 | B.129 | C.5 |
| 1.1140 | B.130 | C.5 |
| 1.1141 | B.131 | C.5 |
| 1.1142 | B.132 | C.5 |
| 1.1143 | B.133 | C.5 |
| 1.1144 | B.134 | C.5 |
| 1.1145 | B.135 | C.5 |
| 1.1146 | B.136 | C.5 |
| 1.1147 | B.137 | C.5 |
| 1.1148 | B.138 | C.5 |
| 1.1149 | B.139 | C.5 |
| 1.1150 | B.140 | C.5 |
| 1.1151 | B.141 | C.5 |
| 1.1152 | B.142 | C.5 |
| 1.1153 | B.143 | C.5 |
| 1.1154 | B.144 | C.5 |
| 1.1155 | B.145 | C.5 |
| 1.1156 | B.146 | C.5 |
| 1.1157 | B.147 | C.5 |
| 1.1158 | B.148 | C.5 |
| 1.1159 | B.149 | C.5 |
| 1.1160 | B.150 | C.5 |
| 1.1161 | B.151 | C.5 |
| 1.1162 | B.152 | C.5 |
| 1.1163 | B.153 | C.5 |
| 1.1164 | B.154 | C.5 |
| 1.1165 | B.155 | C.5 |
| 1.1166 | B.156 | C.5 |
| 1.1167 | B.157 | C.5 |
| 1.1168 | B.158 | C.5 |
| 1.1169 | B.159 | C.5 |
| 1.1170 | B.160 | C.5 |
| 1.1171 | B.161 | C.5 |
| 1.1172 | B.162 | C.5 |
| 1.1173 | B.163 | C.5 |
| 1.1174 | B.164 | C.5 |
| 1.1175 | B.165 | C.5 |
| 1.1176 | B.166 | C.5 |
| 1.1177 | B.167 | C.5 |
| 1.1178 | B.168 | C.5 |
| 1.1179 | B.169 | C.5 |
| 1.1180 | B.170 | C.5 |
| 1.1181 | B.171 | C.5 |
| 1.1182 | B.172 | C.5 |
| 1.1183 | B.173 | C.5 |
| 1.1184 | B.174 | C.5 |
| 1.1185 | B.175 | C.5 |
| 1.1186 | B.176 | C.5 |
| 1.1187 | B.177 | C.5 |
| 1.1188 | B.178 | C.5 |
| 1.1189 | B.179 | C.5 |
| 1.1190 | B.180 | C.5 |
| 1.1191 | B.181 | C.5 |
| 1.1192 | B.182 | C.5 |
| 1.1193 | B.183 | C.5 |
| 1.1194 | B.184 | C.5 |
| 1.1195 | B.185 | C.5 |
| 1.1196 | B.186 | C.5 |
| 1.1197 | B.187 | C.5 |
| 1.1198 | B.188 | C.5 |
| 1.1199 | B.189 | C.5 |
| 1.1200 | B.190 | C.5 |
| 1.1201 | B.191 | C.5 |
| 1.1202 | B.192 | C.5 |
| 1.1203 | B.193 | C.5 |
| 1.1204 | B.194 | C.5 |
| 1.1205 | B.195 | C.5 |
| 1.1206 | B.196 | C.5 |
| 1.1207 | B.197 | C.5 |
| 1.1208 | B.198 | C.5 |
| 1.1209 | B.199 | C.5 |
| 1.1210 | B.200 | C.5 |
| 1.1211 | B.201 | C.5 |
| 1.1212 | B.202 | C.5 |
| 1.1213 | B.1 | C.6 |
| 1.1214 | B.2 | C.6 |
| 1.1215 | B.3 | C.6 |
| 1.1216 | B.4 | C.6 |
| 1.1217 | B.5 | C.6 |
| 1.1218 | B.6 | C.6 |
| 1.1219 | B.7 | C.6 |
| 1.1220 | B.8 | C.6 |
| 1.1221 | B.9 | C.6 |
| 1.1222 | B.10 | C.6 |
| 1.1223 | B.11 | C.6 |
| 1.1224 | B.12 | C.6 |
| 1.1225 | B.13 | C.6 |
| 1.1226 | B.14 | C.6 |
| 1.1227 | B.15 | C.6 |
| 1.1228 | B.16 | C.6 |
| 1.1229 | B.17 | C.6 |
| 1.1230 | B.18 | C.6 |
| 1.1231 | B.19 | C.6 |
| 1.1232 | B.20 | C.6 |
| 1.1233 | B.21 | C.6 |
| 1.1234 | B.22 | C.6 |
| 1.1235 | B.23 | C.6 |
| 1.1236 | B.24 | C.6 |
| 1.1237 | B.25 | C.6 |
| 1.1238 | B.26 | C.6 |
| 1.1239 | B.27 | C.6 |
| 1.1240 | B.28 | C.6 |
| 1.1241 | B.29 | C.6 |
| 1.1242 | B.30 | C.6 |
| 1.1243 | B.31 | C.6 |
| 1.1244 | B.32 | C.6 |
| 1.1245 | B.33 | C.6 |
| 1.1246 | B.34 | C.6 |
| 1.1247 | B.35 | C.6 |
| 1.1248 | B.36 | C.6 |
| 1.1249 | B.37 | C.6 |
| 1.1250 | B.38 | C.6 |
| 1.1251 | B.39 | C.6 |
| 1.1252 | B.40 | C.6 |
| 1.1253 | B.41 | C.6 |
| 1.1254 | B.42 | C.6 |
| 1.1255 | B.43 | C.6 |
| 1.1256 | B.44 | C.6 |
| 1.1257 | B.45 | C.6 |
| 1.1258 | B.46 | C.6 |
| 1.1259 | B.47 | C.6 |
| 1.1260 | B.48 | C.6 |
| 1.1261 | B.49 | C.6 |
| 1.1262 | B.50 | C.6 |
| 1.1263 | B.51 | C.6 |
| 1.1264 | B.52 | C.6 |
| 1.1265 | B.53 | C.6 |
| 1.1266 | B.54 | C.6 |
| 1.1267 | B.55 | C.6 |
| 1.1268 | B.56 | C.6 |
| 1.1269 | B.57 | C.6 |
| 1.1270 | B.58 | C.6 |
| 1.1271 | B.59 | C.6 |
| 1.1272 | B.60 | C.6 |
| 1.1273 | B.61 | C.6 |
| 1.1274 | B.62 | C.6 |
| 1.1275 | B.63 | C.6 |
| 1.1276 | B.64 | C.6 |
| 1.1277 | B.65 | C.6 |
| 1.1278 | B.66 | C.6 |
| 1.1279 | B.67 | C.6 |
| 1.1280 | B.68 | C.6 |
| 1.1281 | B.69 | C.6 |
| 1.1282 | B.70 | C.6 |
| 1.1283 | B.71 | C.6 |
| 1.1284 | B.72 | C.6 |
| 1.1285 | B.73 | C.6 |
| 1.1286 | B.74 | C.6 |
| 1.1287 | B.75 | C.6 |
| 1.1288 | B.76 | C.6 |
| 1.1289 | B.77 | C.6 |
| 1.1290 | B.78 | C.6 |
| 1.1291 | B.79 | C.6 |
| 1.1292 | B.80 | C.6 |
| 1.1293 | B.81 | C.6 |
| 1.1294 | B.82 | C.6 |
| 1.1295 | B.83 | C.6 |
| 1.1296 | B.84 | C.6 |
| 1.1297 | B.85 | C.6 |
| 1.1298 | B.86 | C.6 |
| 1.1299 | B.87 | C.6 |
| 1.1300 | B.88 | C.6 |
| 1.1301 | B.89 | C.6 |
| 1.1302 | B.90 | C.6 |
| 1.1303 | B.91 | C.6 |
| 1.1304 | B.92 | C.6 |
| 1.1305 | B.93 | C.6 |
| 1.1306 | B.94 | C.6 |
| 1.1307 | B.95 | C.6 |
| 1.1308 | B.96 | C.6 |
| 1.1309 | B.97 | C.6 |
| 1.1310 | B.98 | C.6 |
| 1.1311 | B.99 | C.6 |
| 1.1312 | B.100 | C.6 |
| 1.1313 | B.101 | C.6 |
| 1.1314 | B.102 | C.6 |
| 1.1315 | B.103 | C.6 |
| 1.1316 | B.104 | C.6 |
| 1.1317 | B.105 | C.6 |
| 1.1318 | B.106 | C.6 |
| 1.1319 | B.107 | C.6 |
| 1.1320 | B.108 | C.6 |
| 1.1321 | B.109 | C.6 |
| 1.1322 | B.110 | C.6 |
| 1.1323 | B.111 | C.6 |
| 1.1324 | B.112 | C.6 |
| 1.1325 | B.113 | C.6 |
| 1.1326 | B.114 | C.6 |
| 1.1327 | B.115 | C.6 |
| 1.1328 | B.116 | C.6 |
| 1.1329 | B.117 | C.6 |
| 1.1330 | B.118 | C.6 |
| 1.1331 | B.119 | C.6 |
| 1.1332 | B.120 | C.6 |
| 1.1333 | B.121 | C.6 |
| 1.1334 | B.122 | C.6 |
| 1.1335 | B.123 | C.6 |
| 1.1336 | B.124 | C.6 |
| 1.1337 | B.125 | C.6 |
| 1.1338 | B.126 | C.6 |
| 1.1339 | B.127 | C.6 |
| 1.1340 | B.128 | C.6 |
| 1.1341 | B.129 | C.6 |
| 1.1342 | B.130 | C.6 |
| 1.1343 | B.131 | C.6 |
| 1.1344 | B.132 | C.6 |
| 1.1345 | B.133 | C.6 |
| 1.1346 | B.134 | C.6 |
| 1.1347 | B.135 | C.6 |
| 1.1348 | B.136 | C.6 |
| 1.1349 | B.137 | C.6 |
| 1.1350 | B.138 | C.6 |
| 1.1351 | B.139 | C.6 |
| 1.1352 | B.140 | C.6 |
| 1.1353 | B.141 | C.6 |
| 1.1354 | B.142 | C.6 |
| 1.1355 | B.143 | C.6 |
| 1.1356 | B.144 | C.6 |
| 1.1357 | B.145 | C.6 |
| 1.1358 | B.146 | C.6 |
| 1.1359 | B.147 | C.6 |
| 1.1360 | B.148 | C.6 |
| 1.1361 | B.149 | C.6 |
| 1.1362 | B.150 | C.6 |
| 1.1363 | B.151 | C.6 |
| 1.1364 | B.152 | C.6 |
| 1.1365 | B.153 | C.6 |
| 1.1366 | B.154 | C.6 |
| 1.1367 | B.155 | C.6 |
| 1.1368 | B.156 | C.6 |
| 1.1369 | B.157 | C.6 |
| 1.1370 | B.158 | C.6 |
| 1.1371 | B.159 | C.6 |
| 1.1372 | B.160 | C.6 |
| 1.1373 | B.161 | C.6 |
| 1.1374 | B.162 | C.6 |
| 1.1375 | B.163 | C.6 |
| 1.1376 | B.164 | C.6 |
| 1.1377 | B.165 | C.6 |
| 1.1378 | B.166 | C.6 |
| 1.1379 | B.167 | C.6 |
| 1.1380 | B.168 | C.6 |
| 1.1381 | B.169 | C.6 |
| 1.1382 | B.170 | C.6 |
| 1.1383 | B.171 | C.6 |
| 1.1384 | B.172 | C.6 |
| 1.1385 | B.173 | C.6 |
| 1.1386 | B.174 | C.6 |
| 1.1387 | B.175 | C.6 |
| 1.1388 | B.176 | C.6 |
| 1.1389 | B.177 | C.6 |
| 1.1390 | B.178 | C.6 |
| 1.1391 | B.179 | C.6 |
| 1.1392 | B.180 | C.6 |
| 1.1393 | B.181 | C.6 |
| 1.1394 | B.182 | C.6 |
| 1.1395 | B.183 | C.6 |
| 1.1396 | B.184 | C.6 |
| 1.1397 | B.185 | C.6 |
| 1.1398 | B.186 | C.6 |
| 1.1399 | B.187 | C.6 |
| 1.1400 | B.188 | C.6 |
| 1.1401 | B.189 | C.6 |
| 1.1402 | B.190 | C.6 |
| 1.1403 | B.191 | C.6 |
| 1.1404 | B.192 | C.6 |
| 1.1405 | B.193 | C.6 |
| 1.1406 | B.194 | C.6 |
| 1.1407 | B.195 | C.6 |
| 1.1408 | B.196 | C.6 |
| 1.1409 | B.197 | C.6 |
| 1.1410 | B.198 | C.6 |
| 1.1411 | B.199 | C.6 |
| 1.1412 | B.200 | C.6 |
| 1.1413 | B.201 | C.6 |
| 1.1414 | B.202 | C.6 |
| 1.1415 | B.1 | C.7 |
| 1.1416 | B.2 | C.7 |
| 1.1417 | B.3 | C.7 |
| 1.1418 | B.4 | C.7 |
| 1.1419 | B.5 | C.7 |
| 1.1420 | B.6 | C.7 |
| 1.1421 | B.7 | C.7 |
| 1.1422 | B.8 | C.7 |
| 1.1423 | B.9 | C.7 |
| 1.1424 | B.10 | C.7 |
| 1.1425 | B.11 | C.7 |
| 1.1426 | B.12 | C.7 |
| 1.1427 | B.13 | C.7 |
| 1.1428 | B.14 | C.7 |
| 1.1429 | B.15 | C.7 |
| 1.1430 | B.16 | C.7 |
| 1.1431 | B.17 | C.7 |
| 1.1432 | B.18 | C.7 |
| 1.1433 | B.19 | C.7 |
| 1.1434 | B.20 | C.7 |
| 1.1435 | B.21 | C.7 |
| 1.1436 | B.22 | C.7 |
| 1.1437 | B.23 | C.7 |
| 1.1438 | B.24 | C.7 |
| 1.1439 | B.25 | C.7 |
| 1.1440 | B.26 | C.7 |
| 1.1441 | B.27 | C.7 |
| 1.1442 | B.28 | C.7 |
| 1.1443 | B.29 | C.7 |
| 1.1444 | B.30 | C.7 |
| 1.1445 | B.31 | C.7 |
| 1.1446 | B.32 | C.7 |
| 1.1447 | B.33 | C.7 |
| 1.1448 | B.34 | C.7 |
| 1.1449 | B.35 | C.7 |
| 1.1450 | B.36 | C.7 |
| 1.1451 | B.37 | C.7 |
| 1.1452 | B.38 | C.7 |
| 1.1453 | B.39 | C.7 |
| 1.1454 | B.40 | C.7 |
| 1.1455 | B.41 | C.7 |
| 1.1456 | B.42 | C.7 |
| 1.1457 | B.43 | C.7 |
| 1.1458 | B.44 | C.7 |
| 1.1459 | B.45 | C.7 |
| 1.1460 | B.46 | C.7 |
| 1.1461 | B.47 | C.7 |
| 1.1462 | B.48 | C.7 |
| 1.1463 | B.49 | C.7 |
| 1.1464 | B.50 | C.7 |
| 1.1465 | B.51 | C.7 |
| 1.1466 | B.52 | C.7 |
| 1.1467 | B.53 | C.7 |
| 1.1468 | B.54 | C.7 |
| 1.1469 | B.55 | C.7 |
| 1.1470 | B.56 | C.7 |
| 1.1471 | B.57 | C.7 |
| 1.1472 | B.58 | C.7 |
| 1.1473 | B.59 | C.7 |
| 1.1474 | B.60 | C.7 |
| 1.1475 | B.61 | C.7 |
| 1.1476 | B.62 | C.7 |
| 1.1477 | B.63 | C.7 |
| 1.1478 | B.64 | C.7 |
| 1.1479 | B.65 | C.7 |
| 1.1480 | B.66 | C.7 |
| 1.1481 | B.67 | C.7 |
| 1.1482 | B.68 | C.7 |
| 1.1483 | B.69 | C.7 |
| 1.1484 | B.70 | C.7 |
| 1.1485 | B.71 | C.7 |
| 1.1486 | B.72 | C.7 |
| 1.1487 | B.73 | C.7 |
| 1.1488 | B.74 | C.7 |
| 1.1489 | B.75 | C.7 |
| 1.1490 | B.76 | C.7 |
| 1.1491 | B.77 | C.7 |
| 1.1492 | B.78 | C.7 |
| 1.1493 | B.79 | C.7 |
| 1.1494 | B.80 | C.7 |
| 1.1495 | B.81 | C.7 |
| 1.1496 | B.82 | C.7 |
| 1.1497 | B.83 | C.7 |
| 1.1498 | B.84 | C.7 |
| 1.1499 | B.85 | C.7 |
| 1.1500 | B.86 | C.7 |
| 1.1501 | B.87 | C.7 |
| 1.1502 | B.88 | C.7 |
| 1.1503 | B.89 | C.7 |
| 1.1504 | B.90 | C.7 |
| 1.1505 | B.91 | C.7 |
| 1.1506 | B.92 | C.7 |
| 1.1507 | B.93 | C.7 |
| 1.1508 | B.94 | C.7 |
| 1.1509 | B.95 | C.7 |
| 1.1510 | B.96 | C.7 |
| 1.1511 | B.97 | C.7 |
| 1.1512 | B.98 | C.7 |
| 1.1513 | B.99 | C.7 |
| 1.1514 | B.100 | C.7 |
| 1.1515 | B.101 | C.7 |
| 1.1516 | B.102 | C.7 |
| 1.1517 | B.103 | C.7 |
| 1.1518 | B.104 | C.7 |
| 1.1519 | B.105 | C.7 |
| 1.1520 | B.106 | C.7 |
| 1.1521 | B.107 | C.7 |
| 1.1522 | B.108 | C.7 |
| 1.1523 | B.109 | C.7 |
| 1.1524 | B.110 | C.7 |
| 1.1525 | B.111 | C.7 |
| 1.1526 | B.112 | C.7 |
| 1.1527 | B.113 | C.7 |
| 1.1528 | B.114 | C.7 |
| 1.1529 | B.115 | C.7 |
| 1.1530 | B.116 | C.7 |
| 1.1531 | B.117 | C.7 |
| 1.1532 | B.118 | C.7 |
| 1.1533 | B.119 | C.7 |
| 1.1534 | B.120 | C.7 |
| 1.1535 | B.121 | C.7 |
| 1.1536 | B.122 | C.7 |
| 1.1537 | B.123 | C.7 |
| 1.1538 | B.124 | C.7 |
| 1.1539 | B.125 | C.7 |
| 1.1540 | B.126 | C.7 |
| 1.1541 | B.127 | C.7 |
| 1.1542 | B.128 | C.7 |
| 1.1543 | B.129 | C.7 |
| 1.1544 | B.130 | C.7 |
| 1.1545 | B.131 | C.7 |
| 1.1546 | B.132 | C.7 |
| 1.1547 | B.133 | C.7 |
| 1.1548 | B.134 | C.7 |
| 1.1549 | B.135 | C.7 |
| 1.1550 | B.136 | C.7 |
| 1.1551 | B.137 | C.7 |
| 1.1552 | B.138 | C.7 |
| 1.1553 | B.139 | C.7 |
| 1.1554 | B.140 | C.7 |
| 1.1555 | B.141 | C.7 |
| 1.1556 | B.142 | C.7 |
| 1.1557 | B.143 | C.7 |
| 1.1558 | B.144 | C.7 |
| 1.1559 | B.145 | C.7 |
| 1.1560 | B.146 | C.7 |
| 1.1561 | B.147 | C.7 |
| 1.1562 | B.148 | C.7 |
| 1.1563 | B.149 | C.7 |
| 1.1564 | B.150 | C.7 |
| 1.1565 | B.151 | C.7 |
| 1.1566 | B.152 | C.7 |
| 1.1567 | B.153 | C.7 |
| 1.1568 | B.154 | C.7 |
| 1.1569 | B.155 | C.7 |
| 1.1570 | B.156 | C.7 |
| 1.1571 | B.157 | C.7 |
| 1.1572 | B.158 | C.7 |
| 1.1573 | B.159 | C.7 |
| 1.1574 | B.160 | C.7 |
| 1.1575 | B.161 | C.7 |
| 1.1576 | B.162 | C.7 |
| 1.1577 | B.163 | C.7 |
| 1.1578 | B.164 | C.7 |
| 1.1579 | B.165 | C.7 |
| 1.1580 | B.166 | C.7 |
| 1.1581 | B.167 | C.7 |
| 1.1582 | B.168 | C.7 |
| 1.1583 | B.169 | C.7 |
| 1.1584 | B.170 | C.7 |
| 1.1585 | B.171 | C.7 |
| 1.1586 | B.172 | C.7 |
| 1.1587 | B.173 | C.7 |
| 1.1588 | B.174 | C.7 |
| 1.1589 | B.175 | C.7 |
| 1.1590 | B.176 | C.7 |
| 1.1591 | B.177 | C.7 |
| 1.1592 | B.178 | C.7 |
| 1.1593 | B.179 | C.7 |
| 1.1594 | B.180 | C.7 |
| 1.1595 | B.181 | C.7 |
| 1.1596 | B.182 | C.7 |
| 1.1597 | B.183 | C.7 |
| 1.1598 | B.184 | C.7 |
| 1.1599 | B.185 | C.7 |
| 1.1600 | B.186 | C.7 |
| 1.1601 | B.187 | C.7 |
| 1.1602 | B.188 | C.7 |
| 1.1603 | B.189 | C.7 |
| 1.1604 | B.190 | C.7 |
| 1.1605 | B.191 | C.7 |
| 1.1606 | B.192 | C.7 |
| 1.1607 | B.193 | C.7 |
| 1.1608 | B.194 | C.7 |
| 1.1609 | B.195 | C.7 |
| 1.1610 | B.196 | C.7 |
| 1.1611 | B.197 | C.7 |
| 1.1612 | B.198 | C.7 |
| 1.1613 | B.199 | C.7 |
| 1.1614 | B.200 | C.7 |
| 1.1615 | B.201 | C.7 |
| 1.1616 | B.202 | C.7 |
| 1.1617 | B.1 | C.8 |
| 1.1618 | B.2 | C.8 |
| 1.1619 | B.3 | C.8 |
| 1.1620 | B.4 | C.8 |
| 1.1621 | B.5 | C.8 |
| 1.1622 | B.6 | C.8 |
| 1.1623 | B.7 | C.8 |
| 1.1624 | B.8 | C.8 |
| 1.1625 | B.9 | C.8 |
| 1.1626 | B.10 | C.8 |
| 1.1627 | B.11 | C.8 |
| 1.1628 | B.12 | C.8 |
| 1.1629 | B.13 | C.8 |
| 1.1630 | B.14 | C.8 |
| 1.1631 | B.15 | C.8 |
| 1.1632 | B.16 | C.8 |
| 1.1633 | B.17 | C.8 |
| 1.1634 | B.18 | C.8 |
| 1.1635 | B.19 | C.8 |
| 1.1636 | B.20 | C.8 |
| 1.1637 | B.21 | C.8 |
| 1.1638 | B.22 | C.8 |
| 1.1639 | B.23 | C.8 |
| 1.1640 | B.24 | C.8 |
| 1.1641 | B.25 | C.8 |
| 1.1642 | B.26 | C.8 |
| 1.1643 | B.27 | C.8 |
| 1.1644 | B.28 | C.8 |
| 1.1645 | B.29 | C.8 |
| 1.1646 | B.30 | C.8 |
| 1.1647 | B.31 | C.8 |
| 1.1648 | B.32 | C.8 |
| 1.1649 | B.33 | C.8 |
| 1.1650 | B.34 | C.8 |
| 1.1651 | B.35 | C.8 |
| 1.1652 | B.36 | C.8 |
| 1.1653 | B.37 | C.8 |
| 1.1654 | B.38 | C.8 |
| 1.1655 | B.39 | C.8 |
| 1.1656 | B.40 | C.8 |
| 1.1657 | B.41 | C.8 |
| 1.1658 | B.42 | C.8 |
| 1.1659 | B.43 | C.8 |
| 1.1660 | B.44 | C.8 |
| 1.1661 | B.45 | C.8 |
| 1.1662 | B.46 | C.8 |
| 1.1663 | B.47 | C.8 |
| 1.1664 | B.48 | C.8 |
| 1.1665 | B.49 | C.8 |
| 1.1666 | B.50 | C.8 |
| 1.1667 | B.51 | C.8 |
| 1.1668 | B.52 | C.8 |
| 1.1669 | B.53 | C.8 |
| 1.1670 | B.54 | C.8 |
| 1.1671 | B.55 | C.8 |
| 1.1672 | B.56 | C.8 |
| 1.1673 | B.57 | C.8 |
| 1.1674 | B.58 | C.8 |
| 1.1675 | B.59 | C.8 |
| 1.1676 | B.60 | C.8 |
| 1.1677 | B.61 | C.8 |
| 1.1678 | B.62 | C.8 |
| 1.1679 | B.63 | C.8 |
| 1.1680 | B.64 | C.8 |
| 1.1681 | B.65 | C.8 |
| 1.1682 | B.66 | C.8 |
| 1.1683 | B.67 | C.8 |
| 1.1684 | B.68 | C.8 |
| 1.1685 | B.69 | C.8 |
| 1.1686 | B.70 | C.8 |
| 1.1687 | B.71 | C.8 |
| 1.1688 | B.72 | C.8 |
| 1.1689 | B.73 | C.8 |
| 1.1690 | B.74 | C.8 |
| 1.1691 | B.75 | C.8 |
| 1.1692 | B.76 | C.8 |
| 1.1693 | B.77 | C.8 |
| 1.1694 | B.78 | C.8 |
| 1.1695 | B.79 | C.8 |
| 1.1696 | B.80 | C.8 |
| 1.1697 | B.81 | C.8 |
| 1.1698 | B.82 | C.8 |
| 1.1699 | B.83 | C.8 |
| 1.1700 | B.84 | C.8 |
| 1.1701 | B.85 | C.8 |
| 1.1702 | B.86 | C.8 |
| 1.1703 | B.87 | C.8 |
| 1.1704 | B.88 | C.8 |
| 1.1705 | B.89 | C.8 |
| 1.1706 | B.90 | C.8 |
| 1.1707 | B.91 | C.8 |
| 1.1708 | B.92 | C.8 |
| 1.1709 | B.93 | C.8 |
| 1.1710 | B.94 | C.8 |
| 1.1711 | B.95 | C.8 |
| 1.1712 | B.96 | C.8 |
| 1.1713 | B.97 | C.8 |
| 1.1714 | B.98 | C.8 |
| 1.1715 | B.99 | C.8 |
| 1.1716 | B.100 | C.8 |
| 1.1717 | B.101 | C.8 |
| 1.1718 | B.102 | C.8 |
| 1.1719 | B.103 | C.8 |
| 1.1720 | B.104 | C.8 |
| 1.1721 | B.105 | C.8 |
| 1.1722 | B.106 | C.8 |
| 1.1723 | B.107 | C.8 |
| 1.1724 | B.108 | C.8 |
| 1.1725 | B.109 | C.8 |
| 1.1726 | B.110 | C.8 |
| 1.1727 | B.111 | C.8 |
| 1.1728 | B.112 | C.8 |
| 1.1729 | B.113 | C.8 |
| 1.1730 | B.114 | C.8 |
| 1.1731 | B.115 | C.8 |
| 1.1732 | B.116 | C.8 |
| 1.1733 | B.117 | C.8 |
| 1.1734 | B.118 | C.8 |
| 1.1735 | B.119 | C.8 |
| 1.1736 | B.120 | C.8 |
| 1.1737 | B.121 | C.8 |
| 1.1738 | B.122 | C.8 |
| 1.1739 | B.123 | C.8 |
| 1.1740 | B.124 | C.8 |
| 1.1741 | B.125 | C.8 |
| 1.1742 | B.126 | C.8 |
| 1.1743 | B.127 | C.8 |
| 1.1744 | B.128 | C.8 |
| 1.1745 | B.129 | C.8 |
| 1.1746 | B.130 | C.8 |
| 1.1747 | B.131 | C.8 |
| 1.1748 | B.132 | C.8 |
| 1.1749 | B.133 | C.8 |
| 1.1750 | B.134 | C.8 |
| 1.1751 | B.135 | C.8 |
| 1.1752 | B.136 | C.8 |
| 1.1753 | B.137 | C.8 |
| 1.1754 | B.138 | C.8 |
| 1.1755 | B.139 | C.8 |
| 1.1756 | B.140 | C.8 |
| 1.1757 | B.141 | C.8 |
| 1.1758 | B.142 | C.8 |
| 1.1759 | B.143 | C.8 |
| 1.1760 | B.144 | C.8 |
| 1.1761 | B.145 | C.8 |
| 1.1762 | B.146 | C.8 |
| 1.1763 | B.147 | C.8 |
| 1.1764 | B.148 | C.8 |
| 1.1765 | B.149 | C.8 |
| 1.1766 | B.150 | C.8 |
| 1.1767 | B.151 | C.8 |
| 1.1768 | B.152 | C.8 |
| 1.1769 | B.153 | C.8 |
| 1.1770 | B.154 | C.8 |
| 1.1771 | B.155 | C.8 |
| 1.1772 | B.156 | C.8 |
| 1.1773 | B.157 | C.8 |
| 1.1774 | B.158 | C.8 |
| 1.1775 | B.159 | C.8 |
| 1.1776 | B.160 | C.8 |
| 1.1777 | B.161 | C.8 |
| 1.1778 | B.162 | C.8 |
| 1.1779 | B.163 | C.8 |
| 1.1780 | B.164 | C.8 |
| 1.1781 | B.165 | C.8 |
| 1.1782 | B.166 | C.8 |
| 1.1783 | B.167 | C.8 |
| 1.1784 | B.168 | C.8 |
| 1.1785 | B.169 | C.8 |
| 1.1786 | B.170 | C.8 |
| 1.1787 | B.171 | C.8 |
| 1.1788 | B.172 | C.8 |
| 1.1789 | B.173 | C.8 |
| 1.1790 | B.174 | C.8 |
| 1.1791 | B.175 | C.8 |
| 1.1792 | B.176 | C.8 |
| 1.1793 | B.177 | C.8 |
| 1.1794 | B.178 | C.8 |
| 1.1795 | B.179 | C.8 |
| 1.1796 | B.180 | C.8 |
| 1.1797 | B.181 | C.8 |
| 1.1798 | B.182 | C.8 |
| 1.1799 | B.183 | C.8 |
| 1.1800 | B.184 | C.8 |
| 1.1801 | B.185 | C.8 |
| 1.1802 | B.186 | C.8 |
| 1.1803 | B.187 | C.8 |
| 1.1804 | B.188 | C.8 |
| 1.1805 | B.189 | C.8 |
| 1.1806 | B.190 | C.8 |
| 1.1807 | B.191 | C.8 |
| 1.1808 | B.192 | C.8 |
| 1.1809 | B.193 | C.8 |
| 1.1810 | B.194 | C.8 |
| 1.1811 | B.195 | C.8 |
| 1.1812 | B.196 | C.8 |
| 1.1813 | B.197 | C.8 |
| 1.1814 | B.198 | C.8 |
| 1.1815 | B.199 | C.8 |
| 1.1816 | B.200 | C.8 |
| 1.1817 | B.201 | C.8 |
| 1.1818 | B.202 | C.8 |
| 1.1819 | B.1 | C.9 |
| 1.1820 | B.2 | C.9 |
| 1.1821 | B.3 | C.9 |
| 1.1822 | B.4 | C.9 |
| 1.1823 | B.5 | C.9 |
| 1.1824 | B.6 | C.9 |
| 1.1825 | B.7 | C.9 |
| 1.1826 | B.8 | C.9 |
| 1.1827 | B.9 | C.9 |
| 1.1828 | B.10 | C.9 |
| 1.1829 | B.11 | C.9 |
| 1.1830 | B.12 | C.9 |
| 1.1831 | B.13 | C.9 |
| 1.1832 | B.14 | C.9 |
| 1.1833 | B.15 | C.9 |
| 1.1834 | B.16 | C.9 |
| 1.1835 | B.17 | C.9 |
| 1.1836 | B.18 | C.9 |
| 1.1837 | B.19 | C.9 |
| 1.1838 | B.20 | C.9 |
| 1.1839 | B.21 | C.9 |
| 1.1840 | B.22 | C.9 |
| 1.1841 | B.23 | C.9 |
| 1.1842 | B.24 | C.9 |
| 1.1843 | B.25 | C.9 |
| 1.1844 | B.26 | C.9 |
| 1.1845 | B.27 | C.9 |
| 1.1846 | B.28 | C.9 |
| 1.1847 | B.29 | C.9 |
| 1.1848 | B.30 | C.9 |
| 1.1849 | B.31 | C.9 |
| 1.1850 | B.32 | C.9 |
| 1.1851 | B.33 | C.9 |
| 1.1852 | B.34 | C.9 |
| 1.1853 | B.35 | C.9 |
| 1.1854 | B.36 | C.9 |
| 1.1855 | B.37 | C.9 |
| 1.1856 | B.38 | C.9 |
| 1.1857 | B.39 | C.9 |
| 1.1858 | B.40 | C.9 |
| 1.1859 | B.41 | C.9 |
| 1.1860 | B.42 | C.9 |
| 1.1861 | B.43 | C.9 |
| 1.1862 | B.44 | C.9 |
| 1.1863 | B.45 | C.9 |
| 1.1864 | B.46 | C.9 |
| 1.1865 | B.47 | C.9 |
| 1.1866 | B.48 | C.9 |
| 1.1867 | B.49 | C.9 |
| 1.1868 | B.50 | C.9 |
| 1.1869 | B.51 | C.9 |
| 1.1870 | B.52 | C.9 |
| 1.1871 | B.53 | C.9 |
| 1.1872 | B.54 | C.9 |
| 1.1873 | B.55 | C.9 |
| 1.1874 | B.56 | C.9 |
| 1.1875 | B.57 | C.9 |
| 1.1876 | B.58 | C.9 |
| 1.1877 | B.59 | C.9 |
| 1.1878 | B.60 | C.9 |
| 1.1879 | B.61 | C.9 |
| 1.1880 | B.62 | C.9 |
| 1.1881 | B.63 | C.9 |
| 1.1882 | B.64 | C.9 |
| 1.1883 | B.65 | C.9 |
| 1.1884 | B.66 | C.9 |
| 1.1885 | B.67 | C.9 |
| 1.1886 | B.68 | C.9 |
| 1.1887 | B.69 | C.9 |
| 1.1888 | B.70 | C.9 |
| 1.1889 | B.71 | C.9 |
| 1.1890 | B.72 | C.9 |
| 1.1891 | B.73 | C.9 |
| 1.1892 | B.74 | C.9 |
| 1.1893 | B.75 | C.9 |
| 1.1894 | B.76 | C.9 |
| 1.1895 | B.77 | C.9 |
| 1.1896 | B.78 | C.9 |
| 1.1897 | B.79 | C.9 |
| 1.1898 | B.80 | C.9 |
| 1.1899 | B.81 | C.9 |
| 1.1900 | B.82 | C.9 |
| 1.1901 | B.83 | C.9 |
| 1.1902 | B.84 | C.9 |
| 1.1903 | B.85 | C.9 |
| 1.1904 | B.86 | C.9 |
| 1.1905 | B.87 | C.9 |
| 1.1906 | B.88 | C.9 |
| 1.1907 | B.89 | C.9 |
| 1.1908 | B.90 | C.9 |
| 1.1909 | B.91 | C.9 |
| 1.1910 | B.92 | C.9 |
| 1.1911 | B.93 | C.9 |
| 1.1912 | B.94 | C.9 |
| 1.1913 | B.95 | C.9 |
| 1.1914 | B.96 | C.9 |
| 1.1915 | B.97 | C.9 |
| 1.1916 | B.98 | C.9 |
| 1.1917 | B.99 | C.9 |
| 1.1918 | B.100 | C.9 |
| 1.1919 | B.101 | C.9 |
| 1.1920 | B.102 | C.9 |
| 1.1921 | B.103 | C.9 |
| 1.1922 | B.104 | C.9 |
| 1.1923 | B.105 | C.9 |
| 1.1924 | B.106 | C.9 |
| 1.1925 | B.107 | C.9 |
| 1.1926 | B.108 | C.9 |
| 1.1927 | B.109 | C.9 |
| 1.1928 | B.110 | C.9 |
| 1.1929 | B.111 | C.9 |
| 1.1930 | B.112 | C.9 |
| 1.1931 | B.113 | C.9 |
| 1.1932 | B.114 | C.9 |
| 1.1933 | B.115 | C.9 |
| 1.1934 | B.116 | C.9 |
| 1.1935 | B.117 | C.9 |
| 1.1936 | B.118 | C.9 |
| 1.1937 | B.119 | C.9 |
| 1.1938 | B.120 | C.9 |
| 1.1939 | B.121 | C.9 |
| 1.1940 | B.122 | C.9 |
| 1.1941 | B.123 | C.9 |
| 1.1942 | B.124 | C.9 |
| 1.1943 | B.125 | C.9 |
| 1.1944 | B.126 | C.9 |
| 1.1945 | B.127 | C.9 |
| 1.1946 | B.128 | C.9 |
| 1.1947 | B.129 | C.9 |
| 1.1948 | B.130 | C.9 |
| 1.1949 | B.131 | C.9 |
| 1.1950 | B.132 | C.9 |
| 1.1951 | B.133 | C.9 |
| 1.1952 | B.134 | C.9 |
| 1.1953 | B.135 | C.9 |
| 1.1954 | B.136 | C.9 |
| 1.1955 | B.137 | C.9 |
| 1.1956 | B.138 | C.9 |
| 1.1957 | B.139 | C.9 |
| 1.1958 | B.140 | C.9 |
| 1.1959 | B.141 | C.9 |
| 1.1960 | B.142 | C.9 |
| 1.1961 | B.143 | C.9 |
| 1.1962 | B.144 | C.9 |
| 1.1963 | B.145 | C.9 |
| 1.1964 | B.146 | C.9 |
| 1.1965 | B.147 | C.9 |
| 1.1966 | B.148 | C.9 |
| 1.1967 | B.149 | C.9 |
| 1.1968 | B.150 | C.9 |
| 1.1969 | B.151 | C.9 |
| 1.1970 | B.152 | C.9 |
| 1.1971 | B.153 | C.9 |
| 1.1972 | B.154 | C.9 |
| 1.1973 | B.155 | C.9 |
| 1.1974 | B.156 | C.9 |
| 1.1975 | B.157 | C.9 |
| 1.1976 | B.158 | C.9 |
| 1.1977 | B.159 | C.9 |
| 1.1978 | B.160 | C.9 |
| 1.1979 | B.161 | C.9 |
| 1.1980 | B.162 | C.9 |
| 1.1981 | B.163 | C.9 |
| 1.1982 | B.164 | C.9 |
| 1.1983 | B.165 | C.9 |
| 1.1984 | B.166 | C.9 |
| 1.1985 | B.167 | C.9 |
| 1.1986 | B.168 | C.9 |
| 1.1987 | B.169 | C.9 |
| 1.1988 | B.170 | C.9 |
| 1.1989 | B.171 | C.9 |
| 1.1990 | B.172 | C.9 |
| 1.1991 | B.173 | C.9 |
| 1.1992 | B.174 | C.9 |
| 1.1993 | B.175 | C.9 |
| 1.1994 | B.176 | C.9 |
| 1.1995 | B.177 | C.9 |
| 1.1996 | B.178 | C.9 |
| 1.1997 | B.179 | C.9 |
| 1.1998 | B.180 | C.9 |
| 1.1999 | B.181 | C.9 |
| 1.2000 | B.182 | C.9 |
| 1.2001 | B.183 | C.9 |
| 1.2002 | B.184 | C.9 |
| 1.2003 | B.185 | C.9 |
| 1.2004 | B.186 | C.9 |
| 1.2005 | B.187 | C.9 |
| 1.2006 | B.188 | C.9 |
| 1.2007 | B.189 | C.9 |
| 1.2008 | B.190 | C.9 |
| 1.2009 | B.191 | C.9 |
| 1.2010 | B.192 | C.9 |
| 1.2011 | B.193 | C.9 |
| 1.2012 | B.194 | C.9 |
| 1.2013 | B.195 | C.9 |
| 1.2014 | B.196 | C.9 |
| 1.2015 | B.197 | C.9 |
| 1.2016 | B.198 | C.9 |
| 1.2017 | B.199 | C.9 |
| 1.2018 | B.200 | C.9 |
| 1.2019 | B.201 | C.9 |
| 1.2020 | B.202 | C.9 |
| 1.2021 | B.1 | C.10 |
| 1.2022 | B.2 | C.10 |
| 1.2023 | B.3 | C.10 |
| 1.2024 | B.4 | C.10 |
| 1.2025 | B.5 | C.10 |
| 1.2026 | B.6 | C.10 |
| 1.2027 | B.7 | C.10 |
| 1.2028 | B.8 | C.10 |
| 1.2029 | B.9 | C.10 |
| 1.2030 | B.10 | C.10 |
| 1.2031 | B.11 | C.10 |
| 1.2032 | B.12 | C.10 |
| 1.2033 | B.13 | C.10 |
| 1.2034 | B.14 | C.10 |
| 1.2035 | B.15 | C.10 |
| 1.2036 | B.16 | C.10 |
| 1.2037 | B.17 | C.10 |
| 1.2038 | B.18 | C.10 |
| 1.2039 | B.19 | C.10 |
| 1.2040 | B.20 | C.10 |
| 1.2041 | B.21 | C.10 |
| 1.2042 | B.22 | C.10 |
| 1.2043 | B.23 | C.10 |
| 1.2044 | B.24 | C.10 |
| 1.2045 | B.25 | C.10 |
| 1.2046 | B.26 | C.10 |
| 1.2047 | B.27 | C.10 |
| 1.2048 | B.28 | C.10 |
| 1.2049 | B.29 | C.10 |
| 1.2050 | B.30 | C.10 |
| 1.2051 | B.31 | C.10 |
| 1.2052 | B.32 | C.10 |
| 1.2053 | B.33 | C.10 |
| 1.2054 | B.34 | C.10 |
| 1.2055 | B.35 | C.10 |
| 1.2056 | B.36 | C.10 |
| 1.2057 | B.37 | C.10 |
| 1.2058 | B.38 | C.10 |
| 1.2059 | B.39 | C.10 |
| 1.2060 | B.40 | C.10 |
| 1.2061 | B.41 | C.10 |
| 1.2062 | B.42 | C.10 |
| 1.2063 | B.43 | C.10 |
| 1.2064 | B.44 | C.10 |
| 1.2065 | B.45 | C.10 |
| 1.2066 | B.46 | C.10 |
| 1.2067 | B.47 | C.10 |
| 1.2068 | B.48 | C.10 |
| 1.2069 | B.49 | C.10 |
| 1.2070 | B.50 | C.10 |
| 1.2071 | B.51 | C.10 |
| 1.2072 | B.52 | C.10 |
| 1.2073 | B.53 | C.10 |
| 1.2074 | B.54 | C.10 |
| 1.2075 | B.55 | C.10 |
| 1.2076 | B.56 | C.10 |
| 1.2077 | B.57 | C.10 |
| 1.2078 | B.58 | C.10 |
| 1.2079 | B.59 | C.10 |
| 1.2080 | B.60 | C.10 |
| 1.2081 | B.61 | C.10 |
| 1.2082 | B.62 | C.10 |
| 1.2083 | B.63 | C.10 |
| 1.2084 | B.64 | C.10 |
| 1.2085 | B.65 | C.10 |
| 1.2086 | B.66 | C.10 |
| 1.2087 | B.67 | C.10 |
| 1.2088 | B.68 | C.10 |
| 1.2089 | B.69 | C.10 |
| 1.2090 | B.70 | C.10 |
| 1.2091 | B.71 | C.10 |
| 1.2092 | B.72 | C.10 |
| 1.2093 | B.73 | C.10 |
| 1.2094 | B.74 | C.10 |
| 1.2095 | B.75 | C.10 |
| 1.2096 | B.76 | C.10 |
| 1.2097 | B.77 | C.10 |
| 1.2098 | B.78 | C.10 |
| 1.2099 | B.79 | C.10 |
| 1.2100 | B.80 | C.10 |
| 1.2101 | B.81 | C.10 |
| 1.2102 | B.82 | C.10 |
| 1.2103 | B.83 | C.10 |
| 1.2104 | B.84 | C.10 |
| 1.2105 | B.85 | C.10 |
| 1.2106 | B.86 | C.10 |
| 1.2107 | B.87 | C.10 |
| 1.2108 | B.88 | C.10 |
| 1.2109 | B.89 | C.10 |
| 1.2110 | B.90 | C.10 |
| 1.2111 | B.91 | C.10 |
| 1.2112 | B.92 | C.10 |
| 1.2113 | B.93 | C.10 |
| 1.2114 | B.94 | C.10 |
| 1.2115 | B.95 | C.10 |
| 1.2116 | B.96 | C.10 |
| 1.2117 | B.97 | C.10 |
| 1.2118 | B.98 | C.10 |
| 1.2119 | B.99 | C.10 |
| 1.2120 | B.100 | C.10 |
| 1.2121 | B.101 | C.10 |
| 1.2122 | B.102 | C.10 |
| 1.2123 | B.103 | C.10 |
| 1.2124 | B.104 | C.10 |
| 1.2125 | B.105 | C.10 |
| 1.2126 | B.106 | C.10 |
| 1.2127 | B.107 | C.10 |
| 1.2128 | B.108 | C.10 |
| 1.2129 | B.109 | C.10 |
| 1.2130 | B.110 | C.10 |
| 1.2131 | B.111 | C.10 |
| 1.2132 | B.112 | C.10 |
| 1.2133 | B.113 | C.10 |
| 1.2134 | B.114 | C.10 |
| 1.2135 | B.115 | C.10 |
| 1.2136 | B.116 | C.10 |
| 1.2137 | B.117 | C.10 |
| 1.2138 | B.118 | C.10 |
| 1.2139 | B.119 | C.10 |
| 1.2140 | B.120 | C.10 |
| 1.2141 | B.121 | C.10 |
| 1.2142 | B.122 | C.10 |
| 1.2143 | B.123 | C.10 |
| 1.2144 | B.124 | C.10 |
| 1.2145 | B.125 | C.10 |
| 1.2146 | B.126 | C.10 |
| 1.2147 | B.127 | C.10 |
| 1.2148 | B.128 | C.10 |
| 1.2149 | B.129 | C.10 |
| 1.2150 | B.130 | C.10 |
| 1.2151 | B.131 | C.10 |
| 1.2152 | B.132 | C.10 |
| 1.2153 | B.133 | C.10 |
| 1.2154 | B.134 | C.10 |
| 1.2155 | B.135 | C.10 |
| 1.2156 | B.136 | C.10 |
| 1.2157 | B.137 | C.10 |
| 1.2158 | B.138 | C.10 |
| 1.2159 | B.139 | C.10 |
| 1.2160 | B.140 | C.10 |
| 1.2161 | B.141 | C.10 |
| 1.2162 | B.142 | C.10 |
| 1.2163 | B.143 | C.10 |
| 1.2164 | B.144 | C.10 |
| 1.2165 | B.145 | C.10 |
| 1.2166 | B.146 | C.10 |
| 1.2167 | B.147 | C.10 |
| 1.2168 | B.148 | C.10 |
| 1.2169 | B.149 | C.10 |
| 1.2170 | B.150 | C.10 |
| 1.2171 | B.151 | C.10 |
| 1.2172 | B.152 | C.10 |
| 1.2173 | B.153 | C.10 |
| 1.2174 | B.154 | C.10 |
| 1.2175 | B.155 | C.10 |
| 1.2176 | B.156 | C.10 |
| 1.2177 | B.157 | C.10 |
| 1.2178 | B.158 | C.10 |
| 1.2179 | B.159 | C.10 |
| 1.2180 | B.160 | C.10 |
| 1.2181 | B.161 | C.10 |
| 1.2182 | B.162 | C.10 |
| 1.2183 | B.163 | C.10 |
| 1.2184 | B.164 | C.10 |
| 1.2185 | B.165 | C.10 |
| 1.2186 | B.166 | C.10 |
| 1.2187 | B.167 | C.10 |
| 1.2188 | B.168 | C.10 |
| 1.2189 | B.169 | C.10 |
| 1.2190 | B.170 | C.10 |
| 1.2191 | B.171 | C.10 |
| 1.2192 | B.172 | C.10 |
| 1.2193 | B.173 | C.10 |
| 1.2194 | B.174 | C.10 |
| 1.2195 | B.175 | C.10 |
| 1.2196 | B.176 | C.10 |
| 1.2197 | B.177 | C.10 |
| 1.2198 | B.178 | C.10 |
| 1.2199 | B.179 | C.10 |
| 1.2200 | B.180 | C.10 |
| 1.2201 | B.181 | C.10 |
| 1.2202 | B.182 | C.10 |
| 1.2203 | B.183 | C.10 |
| 1.2204 | B.184 | C.10 |
| 1.2205 | B.185 | C.10 |
| 1.2206 | B.186 | C.10 |
| 1.2207 | B.187 | C.10 |
| 1.2208 | B.188 | C.10 |
| 1.2209 | B.189 | C.10 |
| 1.2210 | B.190 | C.10 |
| 1.2211 | B.191 | C.10 |
| 1.2212 | B.192 | C.10 |
| 1.2213 | B.193 | C.10 |
| 1.2214 | B.194 | C.10 |
| 1.2215 | B.195 | C.10 |
| 1.2216 | B.196 | C.10 |
| 1.2217 | B.197 | C.10 |
| 1.2218 | B.198 | C.10 |
| 1.2219 | B.199 | C.10 |
| 1.2220 | B.200 | C.10 |
| 1.2221 | B.201 | C.10 |
| 1.2222 | B.202 | C.10 |
| 1.2223 | B.1 | C.11 |
| 1.2224 | B.2 | C.11 |
| 1.2225 | B.3 | C.11 |
| 1.2226 | B.4 | C.11 |
| 1.2227 | B.5 | C.11 |
| 1.2228 | B.6 | C.11 |
| 1.2229 | B.7 | C.11 |
| 1.2230 | B.8 | C.11 |
| 1.2231 | B.9 | C.11 |
| 1.2232 | B.10 | C.11 |
| 1.2233 | B.11 | C.11 |
| 1.2234 | B.12 | C.11 |
| 1.2235 | B.13 | C.11 |
| 1.2236 | B.14 | C.11 |
| 1.2237 | B.15 | C.11 |
| 1.2238 | B.16 | C.11 |
| 1.2239 | B.17 | C.11 |
| 1.2240 | B.18 | C.11 |
| 1.2241 | B.19 | C.11 |
| 1.2242 | B.20 | C.11 |
| 1.2243 | B.21 | C.11 |
| 1.2244 | B.22 | C.11 |
| 1.2245 | B.23 | C.11 |
| 1.2246 | B.24 | C.11 |
| 1.2247 | B.25 | C.11 |
| 1.2248 | B.26 | C.11 |
| 1.2249 | B.27 | C.11 |
| 1.2250 | B.28 | C.11 |
| 1.2251 | B.29 | C.11 |
| 1.2252 | B.30 | C.11 |
| 1.2253 | B.31 | C.11 |
| 1.2254 | B.32 | C.11 |
| 1.2255 | B.33 | C.11 |
| 1.2256 | B.34 | C.11 |
| 1.2257 | B.35 | C.11 |
| 1.2258 | B.36 | C.11 |
| 1.2259 | B.37 | C.11 |
| 1.2260 | B.38 | C.11 |
| 1.2261 | B.39 | C.11 |
| 1.2262 | B.40 | C.11 |
| 1.2263 | B.41 | C.11 |
| 1.2264 | B.42 | C.11 |
| 1.2265 | B.43 | C.11 |
| 1.2266 | B.44 | C.11 |
| 1.2267 | B.45 | C.11 |
| 1.2268 | B.46 | C.11 |
| 1.2269 | B.47 | C.11 |
| 1.2270 | B.48 | C.11 |
| 1.2271 | B.49 | C.11 |
| 1.2272 | B.50 | C.11 |
| 1.2273 | B.51 | C.11 |
| 1.2274 | B.52 | C.11 |
| 1.2275 | B.53 | C.11 |
| 1.2276 | B.54 | C.11 |
| 1.2277 | B.55 | C.11 |
| 1.2278 | B.56 | C.11 |
| 1.2279 | B.57 | C.11 |
| 1.2280 | B.58 | C.11 |
| 1.2281 | B.59 | C.11 |
| 1.2282 | B.60 | C.11 |
| 1.2283 | B.61 | C.11 |
| 1.2284 | B.62 | C.11 |
| 1.2285 | B.63 | C.11 |
| 1.2286 | B.64 | C.11 |
| 1.2287 | B.65 | C.11 |
| 1.2288 | B.66 | C.11 |
| 1.2289 | B.67 | C.11 |
| 1.2290 | B.68 | C.11 |
| 1.2291 | B.69 | C.11 |
| 1.2292 | B.70 | C.11 |
| 1.2293 | B.71 | C.11 |
| 1.2294 | B.72 | C.11 |
| 1.2295 | B.73 | C.11 |
| 1.2296 | B.74 | C.11 |
| 1.2297 | B.75 | C.11 |
| 1.2298 | B.76 | C.11 |
| 1.2299 | B.77 | C.11 |
| 1.2300 | B.78 | C.11 |
| 1.2301 | B.79 | C.11 |
| 1.2302 | B.80 | C.11 |
| 1.2303 | B.81 | C.11 |
| 1.2304 | B.82 | C.11 |
| 1.2305 | B.83 | C.11 |
| 1.2306 | B.84 | C.11 |
| 1.2307 | B.85 | C.11 |
| 1.2308 | B.86 | C.11 |
| 1.2309 | B.87 | C.11 |
| 1.2310 | B.88 | C.11 |
| 1.2311 | B.89 | C.11 |
| 1.2312 | B.90 | C.11 |
| 1.2313 | B.91 | C.11 |
| 1.2314 | B.92 | C.11 |
| 1.2315 | B.93 | C.11 |
| 1.2316 | B.94 | C.11 |
| 1.2317 | B.95 | C.11 |
| 1.2318 | B.96 | C.11 |
| 1.2319 | B.97 | C.11 |
| 1.2320 | B.98 | C.11 |
| 1.2321 | B.99 | C.11 |
| 1.2322 | B.100 | C.11 |
| 1.2323 | B.101 | C.11 |
| 1.2324 | B.102 | C.11 |
| 1.2325 | B.103 | C.11 |
| 1.2326 | B.104 | C.11 |
| 1.2327 | B.105 | C.11 |
| 1.2328 | B.106 | C.11 |
| 1.2329 | B.107 | C.11 |
| 1.2330 | B.108 | C.11 |
| 1.2331 | B.109 | C.11 |
| 1.2332 | B.110 | C.11 |
| 1.2333 | B.111 | C.11 |
| 1.2334 | B.112 | C.11 |
| 1.2335 | B.113 | C.11 |
| 1.2336 | B.114 | C.11 |
| 1.2337 | B.115 | C.11 |
| 1.2338 | B.116 | C.11 |
| 1.2339 | B.117 | C.11 |
| 1.2340 | B.118 | C.11 |
| 1.2341 | B.119 | C.11 |
| 1.2342 | B.120 | C.11 |
| 1.2343 | B.121 | C.11 |
| 1.2344 | B.122 | C.11 |
| 1.2345 | B.123 | C.11 |
| 1.2346 | B.124 | C.11 |
| 1.2347 | B.125 | C.11 |
| 1.2348 | B.126 | C.11 |
| 1.2349 | B.127 | C.11 |
| 1.2350 | B.128 | C.11 |
| 1.2351 | B.129 | C.11 |
| 1.2352 | B.130 | C.11 |
| 1.2353 | B.131 | C.11 |
| 1.2354 | B.132 | C.11 |
| 1.2355 | B.133 | C.11 |
| 1.2356 | B.134 | C.11 |
| 1.2357 | B.135 | C.11 |
| 1.2358 | B.136 | C.11 |
| 1.2359 | B.137 | C.11 |
| 1.2360 | B.138 | C.11 |
| 1.2361 | B.139 | C.11 |
| 1.2362 | B.140 | C.11 |
| 1.2363 | B.141 | C.11 |
| 1.2364 | B.142 | C.11 |
| 1.2365 | B.143 | C.11 |
| 1.2366 | B.144 | C.11 |
| 1.2367 | B.145 | C.11 |
| 1.2368 | B.146 | C.11 |
| 1.2369 | B.147 | C.11 |
| 1.2370 | B.148 | C.11 |
| 1.2371 | B.149 | C.11 |
| 1.2372 | B.150 | C.11 |
| 1.2373 | B.151 | C.11 |
| 1.2374 | B.152 | C.11 |
| 1.2375 | B.153 | C.11 |
| 1.2376 | B.154 | C.11 |
| 1.2377 | B.155 | C.11 |
| 1.2378 | B.156 | C.11 |
| 1.2379 | B.157 | C.11 |
| 1.2380 | B.158 | C.11 |
| 1.2381 | B.159 | C.11 |
| 1.2382 | B.160 | C.11 |
| 1.2383 | B.161 | C.11 |
| 1.2384 | B.162 | C.11 |
| 1.2385 | B.163 | C.11 |
| 1.2386 | B.164 | C.11 |
| 1.2387 | B.165 | C.11 |
| 1.2388 | B.166 | C.11 |
| 1.2389 | B.167 | C.11 |
| 1.2390 | B.168 | C.11 |
| 1.2391 | B.169 | C.11 |
| 1.2392 | B.170 | C.11 |
| 1.2393 | B.171 | C.11 |
| 1.2394 | B.172 | C.11 |
| 1.2395 | B.173 | C.11 |
| 1.2396 | B.174 | C.11 |
| 1.2397 | B.175 | C.11 |
| 1.2398 | B.176 | C.11 |
| 1.2399 | B.177 | C.11 |
| 1.2400 | B.178 | C.11 |
| 1.2401 | B.179 | C.11 |
| 1.2402 | B.180 | C.11 |
| 1.2403 | B.181 | C.11 |
| 1.2404 | B.182 | C.11 |
| 1.2405 | B.183 | C.11 |
| 1.2406 | B.184 | C.11 |
| 1.2407 | B.185 | C.11 |
| 1.2408 | B.186 | C.11 |
| 1.2409 | B.187 | C.11 |
| 1.2410 | B.188 | C.11 |
| 1.2411 | B.189 | C.11 |
| 1.2412 | B.190 | C.11 |
| 1.2413 | B.191 | C.11 |
| 1.2414 | B.192 | C.11 |
| 1.2415 | B.193 | C.11 |
| 1.2416 | B.194 | C.11 |
| 1.2417 | B.195 | C.11 |
| 1.2418 | B.196 | C.11 |
| 1.2419 | B.197 | C.11 |
| 1.2420 | B.198 | C.11 |
| 1.2421 | B.199 | C.11 |
| 1.2422 | B.200 | C.11 |
| 1.2423 | B.201 | C.11 |
| 1.2424 | B.202 | C.11 |
| 1.2425 | B.1 | C.12 |
| 1.2426 | B.2 | C.12 |
| 1.2427 | B.3 | C.12 |
| 1.2428 | B.4 | C.12 |
| 1.2429 | B.5 | C.12 |
| 1.2430 | B.6 | C.12 |
| 1.2431 | B.7 | C.12 |
| 1.2432 | B.8 | C.12 |
| 1.2433 | B.9 | C.12 |
| 1.2434 | B.10 | C.12 |
| 1.2435 | B.11 | C.12 |
| 1.2436 | B.12 | C.12 |
| 1.2437 | B.13 | C.12 |
| 1.2438 | B.14 | C.12 |
| 1.2439 | B.15 | C.12 |
| 1.2440 | B.16 | C.12 |
| 1.2441 | B.17 | C.12 |
| 1.2442 | B.18 | C.12 |
| 1.2443 | B.19 | C.12 |
| 1.2444 | B.20 | C.12 |
| 1.2445 | B.21 | C.12 |
| 1.2446 | B.22 | C.12 |
| 1.2447 | B.23 | C.12 |
| 1.2448 | B.24 | C.12 |
| 1.2449 | B.25 | C.12 |
| 1.2450 | B.26 | C.12 |
| 1.2451 | B.27 | C.12 |
| 1.2452 | B.28 | C.12 |
| 1.2453 | B.29 | C.12 |
| 1.2454 | B.30 | C.12 |
| 1.2455 | B.31 | C.12 |
| 1.2456 | B.32 | C.12 |
| 1.2457 | B.33 | C.12 |
| 1.2458 | B.34 | C.12 |
| 1.2459 | B.35 | C.12 |
| 1.2460 | B.36 | C.12 |
| 1.2461 | B.37 | C.12 |
| 1.2462 | B.38 | C.12 |
| 1.2463 | B.39 | C.12 |
| 1.2464 | B.40 | C.12 |
| 1.2465 | B.41 | C.12 |
| 1.2466 | B.42 | C.12 |
| 1.2467 | B.43 | C.12 |
| 1.2468 | B.44 | C.12 |
| 1.2469 | B.45 | C.12 |
| 1.2470 | B.46 | C.12 |
| 1.2471 | B.47 | C.12 |
| 1.2472 | B.48 | C.12 |
| 1.2473 | B.49 | C.12 |
| 1.2474 | B.50 | C.12 |
| 1.2475 | B.51 | C.12 |
| 1.2476 | B.52 | C.12 |
| 1.2477 | B.53 | C.12 |
| 1.2478 | B.54 | C.12 |
| 1.2479 | B.55 | C.12 |
| 1.2480 | B.56 | C.12 |
| 1.2481 | B.57 | C.12 |
| 1.2482 | B.58 | C.12 |
| 1.2483 | B.59 | C.12 |
| 1.2484 | B.60 | C.12 |
| 1.2485 | B.61 | C.12 |
| 1.2486 | B.62 | C.12 |
| 1.2487 | B.63 | C.12 |
| 1.2488 | B.64 | C.12 |
| 1.2489 | B.65 | C.12 |
| 1.2490 | B.66 | C.12 |
| 1.2491 | B.67 | C.12 |
| 1.2492 | B.68 | C.12 |
| 1.2493 | B.69 | C.12 |
| 1.2494 | B.70 | C.12 |
| 1.2495 | B.71 | C.12 |
| 1.2496 | B.72 | C.12 |
| 1.2497 | B.73 | C.12 |
| 1.2498 | B.74 | C.12 |
| 1.2499 | B.75 | C.12 |
| 1.2500 | B.76 | C.12 |
| 1.2501 | B.77 | C.12 |
| 1.2502 | B.78 | C.12 |
| 1.2503 | B.79 | C.12 |
| 1.2504 | B.80 | C.12 |
| 1.2505 | B.81 | C.12 |
| 1.2506 | B.82 | C.12 |
| 1.2507 | B.83 | C.12 |
| 1.2508 | B.84 | C.12 |
| 1.2509 | B.85 | C.12 |
| 1.2510 | B.86 | C.12 |
| 1.2511 | B.87 | C.12 |
| 1.2512 | B.88 | C.12 |
| 1.2513 | B.89 | C.12 |
| 1.2514 | B.90 | C.12 |
| 1.2515 | B.91 | C.12 |
| 1.2516 | B.92 | C.12 |
| 1.2517 | B.93 | C.12 |
| 1.2518 | B.94 | C.12 |
| 1.2519 | B.95 | C.12 |
| 1.2520 | B.96 | C.12 |
| 1.2521 | B.97 | C.12 |
| 1.2522 | B.98 | C.12 |
| 1.2523 | B.99 | C.12 |
| 1.2524 | B.100 | C.12 |
| 1.2525 | B.101 | C.12 |
| 1.2526 | B.102 | C.12 |
| 1.2527 | B.103 | C.12 |
| 1.2528 | B.104 | C.12 |
| 1.2529 | B.105 | C.12 |
| 1.2530 | B.106 | C.12 |
| 1.2531 | B.107 | C.12 |
| 1.2532 | B.108 | C.12 |
| 1.2533 | B.109 | C.12 |
| 1.2534 | B.110 | C.12 |
| 1.2535 | B.111 | C.12 |
| 1.2536 | B.112 | C.12 |
| 1.2537 | B.113 | C.12 |
| 1.2538 | B.114 | C.12 |
| 1.2539 | B.115 | C.12 |
| 1.2540 | B.116 | C.12 |
| 1.2541 | B.117 | C.12 |
| 1.2542 | B.118 | C.12 |
| 1.2543 | B.119 | C.12 |
| 1.2544 | B.120 | C.12 |
| 1.2545 | B.121 | C.12 |
| 1.2546 | B.122 | C.12 |
| 1.2547 | B.123 | C.12 |
| 1.2548 | B.124 | C.12 |
| 1.2549 | B.125 | C.12 |
| 1.2550 | B.126 | C.12 |
| 1.2551 | B.127 | C.12 |
| 1.2552 | B.128 | C.12 |
| 1.2553 | B.129 | C.12 |
| 1.2554 | B.130 | C.12 |
| 1.2555 | B.131 | C.12 |
| 1.2556 | B.132 | C.12 |
| 1.2557 | B.133 | C.12 |
| 1.2558 | B.134 | C.12 |
| 1.2559 | B.135 | C.12 |
| 1.2560 | B.136 | C.12 |
| 1.2561 | B.137 | C.12 |
| 1.2562 | B.138 | C.12 |
| 1.2563 | B.139 | C.12 |
| 1.2564 | B.140 | C.12 |
| 1.2565 | B.141 | C.12 |
| 1.2566 | B.142 | C.12 |
| 1.2567 | B.143 | C.12 |
| 1.2568 | B.144 | C.12 |
| 1.2569 | B.145 | C.12 |
| 1.2570 | B.146 | C.12 |
| 1.2571 | B.147 | C.12 |
| 1.2572 | B.148 | C.12 |
| 1.2573 | B.149 | C.12 |
| 1.2574 | B.150 | C.12 |
| 1.2575 | B.151 | C.12 |
| 1.2576 | B.152 | C.12 |
| 1.2577 | B.153 | C.12 |
| 1.2578 | B.154 | C.12 |
| 1.2579 | B.155 | C.12 |
| 1.2580 | B.156 | C.12 |
| 1.2581 | B.157 | C.12 |
| 1.2582 | B.158 | C.12 |
| 1.2583 | B.159 | C.12 |
| 1.2584 | B.160 | C.12 |
| 1.2585 | B.161 | C.12 |
| 1.2586 | B.162 | C.12 |
| 1.2587 | B.163 | C.12 |
| 1.2588 | B.164 | C.12 |
| 1.2589 | B.165 | C.12 |
| 1.2590 | B.166 | C.12 |
| 1.2591 | B.167 | C.12 |
| 1.2592 | B.168 | C.12 |
| 1.2593 | B.169 | C.12 |
| 1.2594 | B.170 | C.12 |
| 1.2595 | B.171 | C.12 |
| 1.2596 | B.172 | C.12 |
| 1.2597 | B.173 | C.12 |
| 1.2598 | B.174 | C.12 |
| 1.2599 | B.175 | C.12 |
| 1.2600 | B.176 | C.12 |
| 1.2601 | B.177 | C.12 |
| 1.2602 | B.178 | C.12 |
| 1.2603 | B.179 | C.12 |
| 1.2604 | B.180 | C.12 |
| 1.2605 | B.181 | C.12 |
| 1.2606 | B.182 | C.12 |
| 1.2607 | B.183 | C.12 |
| 1.2608 | B.184 | C.12 |
| 1.2609 | B.185 | C.12 |
| 1.2610 | B.186 | C.12 |
| 1.2611 | B.187 | C.12 |
| 1.2612 | B.188 | C.12 |
| 1.2613 | B.189 | C.12 |
| 1.2614 | B.190 | C.12 |
| 1.2615 | B.191 | C.12 |
| 1.2616 | B.192 | C.12 |
| 1.2617 | B.193 | C.12 |
| 1.2618 | B.194 | C.12 |
| 1.2619 | B.195 | C.12 |
| 1.2620 | B.196 | C.12 |
| 1.2621 | B.197 | C.12 |
| 1.2622 | B.198 | C.12 |
| 1.2623 | B.199 | C.12 |
| 1.2624 | B.200 | C.12 |
| 1.2625 | B.201 | C.12 |
| 1.2626 | B.202 | C.12 |
| 1.2627 | B.1 | C.13 |
| 1.2628 | B.2 | C.13 |
| 1.2629 | B.3 | C.13 |
| 1.2630 | B.4 | C.13 |
| 1.2631 | B.5 | C.13 |
| 1.2632 | B.6 | C.13 |
| 1.2633 | B.7 | C.13 |
| 1.2634 | B.8 | C.13 |
| 1.2635 | B.9 | C.13 |
| 1.2636 | B.10 | C.13 |
| 1.2637 | B.11 | C.13 |
| 1.2638 | B.12 | C.13 |
| 1.2639 | B.13 | C.13 |
| 1.2640 | B.14 | C.13 |
| 1.2641 | B.15 | C.13 |
| 1.2642 | B.16 | C.13 |
| 1.2643 | B.17 | C.13 |
| 1.2644 | B.18 | C.13 |
| 1.2645 | B.19 | C.13 |
| 1.2646 | B.20 | C.13 |
| 1.2647 | B.21 | C.13 |
| 1.2648 | B.22 | C.13 |
| 1.2649 | B.23 | C.13 |
| 1.2650 | B.24 | C.13 |
| 1.2651 | B.25 | C.13 |
| 1.2652 | B.26 | C.13 |
| 1.2653 | B.27 | C.13 |
| 1.2654 | B.28 | C.13 |
| 1.2655 | B.29 | C.13 |
| 1.2656 | B.30 | C.13 |
| 1.2657 | B.31 | C.13 |
| 1.2658 | B.32 | C.13 |
| 1.2659 | B.33 | C.13 |
| 1.2660 | B.34 | C.13 |
| 1.2661 | B.35 | C.13 |
| 1.2662 | B.36 | C.13 |
| 1.2663 | B.37 | C.13 |
| 1.2664 | B.38 | C.13 |
| 1.2665 | B.39 | C.13 |
| 1.2666 | B.40 | C.13 |
| 1.2667 | B.41 | C.13 |
| 1.2668 | B.42 | C.13 |
| 1.2669 | B.43 | C.13 |
| 1.2670 | B.44 | C.13 |
| 1.2671 | B.45 | C.13 |
| 1.2672 | B.46 | C.13 |
| 1.2673 | B.47 | C.13 |
| 1.2674 | B.48 | C.13 |
| 1.2675 | B.49 | C.13 |
| 1.2676 | B.50 | C.13 |
| 1.2677 | B.51 | C.13 |
| 1.2678 | B.52 | C.13 |
| 1.2679 | B.53 | C.13 |
| 1.2680 | B.54 | C.13 |
| 1.2681 | B.55 | C.13 |
| 1.2682 | B.56 | C.13 |
| 1.2683 | B.57 | C.13 |
| 1.2684 | B.58 | C.13 |
| 1.2685 | B.59 | C.13 |
| 1.2686 | B.60 | C.13 |
| 1.2687 | B.61 | C.13 |
| 1.2688 | B.62 | C.13 |
| 1.2689 | B.63 | C.13 |
| 1.2690 | B.64 | C.13 |
| 1.2691 | B.65 | C.13 |
| 1.2692 | B.66 | C.13 |
| 1.2693 | B.67 | C.13 |
| 1.2694 | B.68 | C.13 |
| 1.2695 | B.69 | C.13 |
| 1.2696 | B.70 | C.13 |
| 1.2697 | B.71 | C.13 |
| 1.2698 | B.72 | C.13 |
| 1.2699 | B.73 | C.13 |
| 1.2700 | B.74 | C.13 |
| 1.2701 | B.75 | C.13 |
| 1.2702 | B.76 | C.13 |
| 1.2703 | B.77 | C.13 |
| 1.2704 | B.78 | C.13 |
| 1.2705 | B.79 | C.13 |
| 1.2706 | B.80 | C.13 |
| 1.2707 | B.81 | C.13 |
| 1.2708 | B.82 | C.13 |
| 1.2709 | B.83 | C.13 |
| 1.2710 | B.84 | C.13 |
| 1.2711 | B.85 | C.13 |
| 1.2712 | B.86 | C.13 |
| 1.2713 | B.87 | C.13 |
| 1.2714 | B.88 | C.13 |
| 1.2715 | B.89 | C.13 |
| 1.2716 | B.90 | C.13 |
| 1.2717 | B.91 | C.13 |
| 1.2718 | B.92 | C.13 |
| 1.2719 | B.93 | C.13 |
| 1.2720 | B.94 | C.13 |
| 1.2721 | B.95 | C.13 |
| 1.2722 | B.96 | C.13 |
| 1.2723 | B.97 | C.13 |
| 1.2724 | B.98 | C.13 |
| 1.2725 | B.99 | C.13 |
| 1.2726 | B.100 | C.13 |
| 1.2727 | B.101 | C.13 |
| 1.2728 | B.102 | C.13 |
| 1.2729 | B.103 | C.13 |
| 1.2730 | B.104 | C.13 |
| 1.2731 | B.105 | C.13 |
| 1.2732 | B.106 | C.13 |
| 1.2733 | B.107 | C.13 |
| 1.2734 | B.108 | C.13 |
| 1.2735 | B.109 | C.13 |
| 1.2736 | B.110 | C.13 |
| 1.2737 | B.111 | C.13 |
| 1.2738 | B.112 | C.13 |
| 1.2739 | B.113 | C.13 |
| 1.2740 | B.114 | C.13 |
| 1.2741 | B.115 | C.13 |
| 1.2742 | B.116 | C.13 |
| 1.2743 | B.117 | C.13 |
| 1.2744 | B.118 | C.13 |
| 1.2745 | B.119 | C.13 |
| 1.2746 | B.120 | C.13 |
| 1.2747 | B.121 | C.13 |
| 1.2748 | B.122 | C.13 |
| 1.2749 | B.123 | C.13 |
| 1.2750 | B.124 | C.13 |
| 1.2751 | B.125 | C.13 |
| 1.2752 | B.126 | C.13 |
| 1.2753 | B.127 | C.13 |
| 1.2754 | B.128 | C.13 |
| 1.2755 | B.129 | C.13 |
| 1.2756 | B.130 | C.13 |
| 1.2757 | B.131 | C.13 |
| 1.2758 | B.132 | C.13 |
| 1.2759 | B.133 | C.13 |
| 1.2760 | B.134 | C.13 |
| 1.2761 | B.135 | C.13 |
| 1.2762 | B.136 | C.13 |
| 1.2763 | B.137 | C.13 |
| 1.2764 | B.138 | C.13 |
| 1.2765 | B.139 | C.13 |
| 1.2766 | B.140 | C.13 |
| 1.2767 | B.141 | C.13 |
| 1.2768 | B.142 | C.13 |
| 1.2769 | B.143 | C.13 |
| 1.2770 | B.144 | C.13 |
| 1.2771 | B.145 | C.13 |
| 1.2772 | B.146 | C.13 |
| 1.2773 | B.147 | C.13 |
| 1.2774 | B.148 | C.13 |
| 1.2775 | B.149 | C.13 |
| 1.2776 | B.150 | C.13 |
| 1.2777 | B.151 | C.13 |
| 1.2778 | B.152 | C.13 |
| 1.2779 | B.153 | C.13 |
| 1.2780 | B.154 | C.13 |
| 1.2781 | B.155 | C.13 |
| 1.2782 | B.156 | C.13 |
| 1.2783 | B.157 | C.13 |
| 1.2784 | B.158 | C.13 |
| 1.2785 | B.159 | C.13 |
| 1.2786 | B.160 | C.13 |
| 1.2787 | B.161 | C.13 |
| 1.2788 | B.162 | C.13 |
| 1.2789 | B.163 | C.13 |
| 1.2790 | B.164 | C.13 |
| 1.2791 | B.165 | C.13 |
| 1.2792 | B.166 | C.13 |
| 1.2793 | B.167 | C.13 |
| 1.2794 | B.168 | C.13 |
| 1.2795 | B.169 | C.13 |
| 1.2796 | B.170 | C.13 |
| 1.2797 | B.171 | C.13 |
| 1.2798 | B.172 | C.13 |
| 1.2799 | B.173 | C.13 |
| 1.2800 | B.174 | C.13 |
| 1.2801 | B.175 | C.13 |
| 1.2802 | B.176 | C.13 |
| 1.2803 | B.177 | C.13 |
| 1.2804 | B.178 | C.13 |
| 1.2805 | B.179 | C.13 |
| 1.2806 | B.180 | C.13 |
| 1.2807 | B.181 | C.13 |
| 1.2808 | B.182 | C.13 |
| 1.2809 | B.183 | C.13 |
| 1.2810 | B.184 | C.13 |
| 1.2811 | B.185 | C.13 |
| 1.2812 | B.186 | C.13 |
| 1.2813 | B.187 | C.13 |
| 1.2814 | B.188 | C.13 |
| 1.2815 | B.189 | C.13 |
| 1.2816 | B.190 | C.13 |
| 1.2817 | B.191 | C.13 |
| 1.2818 | B.192 | C.13 |
| 1.2819 | B.193 | C.13 |
| 1.2820 | B.194 | C.13 |
| 1.2821 | B.195 | C.13 |
| 1.2822 | B.196 | C.13 |
| 1.2823 | B.197 | C.13 |
| 1.2824 | B.198 | C.13 |
| 1.2825 | B.199 | C.13 |
| 1.2826 | B.200 | C.13 |
| 1.2827 | B.201 | C.13 |
| 1.2828 | B.202 | C.13 |
| 1.2829 | B.1 | C.14 |
| 1.2830 | B.2 | C.14 |
| 1.2831 | B.3 | C.14 |
| 1.2832 | B.4 | C.14 |
| 1.2833 | B.5 | C.14 |
| 1.2834 | B.6 | C.14 |
| 1.2835 | B.7 | C.14 |
| 1.2836 | B.8 | C.14 |
| 1.2837 | B.9 | C.14 |
| 1.2838 | B.10 | C.14 |
| 1.2839 | B.11 | C.14 |
| 1.2840 | B.12 | C.14 |
| 1.2841 | B.13 | C.14 |
| 1.2842 | B.14 | C.14 |
| 1.2843 | B.15 | C.14 |
| 1.2844 | B.16 | C.14 |
| 1.2845 | B.17 | C.14 |
| 1.2846 | B.18 | C.14 |
| 1.2847 | B.19 | C.14 |
| 1.2848 | B.20 | C.14 |
| 1.2849 | B.21 | C.14 |
| 1.2850 | B.22 | C.14 |
| 1.2851 | B.23 | C.14 |
| 1.2852 | B.24 | C.14 |
| 1.2853 | B.25 | C.14 |
| 1.2854 | B.26 | C.14 |
| 1.2855 | B.27 | C.14 |
| 1.2856 | B.28 | C.14 |
| 1.2857 | B.29 | C.14 |
| 1.2858 | B.30 | C.14 |
| 1.2859 | B.31 | C.14 |
| 1.2860 | B.32 | C.14 |
| 1.2861 | B.33 | C.14 |
| 1.2862 | B.34 | C.14 |
| 1.2863 | B.35 | C.14 |
| 1.2864 | B.36 | C.14 |
| 1.2865 | B.37 | C.14 |
| 1.2866 | B.38 | C.14 |
| 1.2867 | B.39 | C.14 |
| 1.2868 | B.40 | C.14 |
| 1.2869 | B.41 | C.14 |
| 1.2870 | B.42 | C.14 |
| 1.2871 | B.43 | C.14 |
| 1.2872 | B.44 | C.14 |
| 1.2873 | B.45 | C.14 |
| 1.2874 | B.46 | C.14 |
| 1.2875 | B.47 | C.14 |
| 1.2876 | B.48 | C.14 |
| 1.2877 | B.49 | C.14 |
| 1.2878 | B.50 | C.14 |
| 1.2879 | B.51 | C.14 |
| 1.2880 | B.52 | C.14 |
| 1.2881 | B.53 | C.14 |
| 1.2882 | B.54 | C.14 |
| 1.2883 | B.55 | C.14 |
| 1.2884 | B.56 | C.14 |
| 1.2885 | B.57 | C.14 |
| 1.2886 | B.58 | C.14 |
| 1.2887 | B.59 | C.14 |
| 1.2888 | B.60 | C.14 |
| 1.2889 | B.61 | C.14 |
| 1.2890 | B.62 | C.14 |
| 1.2891 | B.63 | C.14 |
| 1.2892 | B.64 | C.14 |
| 1.2893 | B.65 | C.14 |
| 1.2894 | B.66 | C.14 |
| 1.2895 | B.67 | C.14 |
| 1.2896 | B.68 | C.14 |
| 1.2897 | B.69 | C.14 |
| 1.2898 | B.70 | C.14 |
| 1.2899 | B.71 | C.14 |
| 1.2900 | B.72 | C.14 |
| 1.2901 | B.73 | C.14 |
| 1.2902 | B.74 | C.14 |
| 1.2903 | B.75 | C.14 |
| 1.2904 | B.76 | C.14 |
| 1.2905 | B.77 | C.14 |
| 1.2906 | B.78 | C.14 |
| 1.2907 | B.79 | C.14 |
| 1.2908 | B.80 | C.14 |
| 1.2909 | B.81 | C.14 |
| 1.2910 | B.82 | C.14 |
| 1.2911 | B.83 | C.14 |
| 1.2912 | B.84 | C.14 |
| 1.2913 | B.85 | C.14 |
| 1.2914 | B.86 | C.14 |
| 1.2915 | B.87 | C.14 |
| 1.2916 | B.88 | C.14 |
| 1.2917 | B.89 | C.14 |
| 1.2918 | B.90 | C.14 |
| 1.2919 | B.91 | C.14 |
| 1.2920 | B.92 | C.14 |
| 1.2921 | B.93 | C.14 |
| 1.2922 | B.94 | C.14 |
| 1.2923 | B.95 | C.14 |
| 1.2924 | B.96 | C.14 |
| 1.2925 | B.97 | C.14 |
| 1.2926 | B.98 | C.14 |
| 1.2927 | B.99 | C.14 |
| 1.2928 | B.100 | C.14 |
| 1.2929 | B.101 | C.14 |
| 1.2930 | B.102 | C.14 |
| 1.2931 | B.103 | C.14 |
| 1.2932 | B.104 | C.14 |
| 1.2933 | B.105 | C.14 |
| 1.2934 | B.106 | C.14 |
| 1.2935 | B.107 | C.14 |
| 1.2936 | B.108 | C.14 |
| 1.2937 | B.109 | C.14 |
| 1.2938 | B.110 | C.14 |
| 1.2939 | B.111 | C.14 |
| 1.2940 | B.112 | C.14 |
| 1.2941 | B.113 | C.14 |
| 1.2942 | B.114 | C.14 |
| 1.2943 | B.115 | C.14 |
| 1.2944 | B.116 | C.14 |
| 1.2945 | B.117 | C.14 |
| 1.2946 | B.118 | C.14 |
| 1.2947 | B.119 | C.14 |
| 1.2948 | B.120 | C.14 |
| 1.2949 | B.121 | C.14 |
| 1.2950 | B.122 | C.14 |
| 1.2951 | B.123 | C.14 |
| 1.2952 | B.124 | C.14 |
| 1.2953 | B.125 | C.14 |
| 1.2954 | B.126 | C.14 |
| 1.2955 | B.127 | C.14 |
| 1.2956 | B.128 | C.14 |
| 1.2957 | B.129 | C.14 |
| 1.2958 | B.130 | C.14 |
| 1.2959 | B.131 | C.14 |
| 1.2960 | B.132 | C.14 |
| 1.2961 | B.133 | C.14 |
| 1.2962 | B.134 | C.14 |
| 1.2963 | B.135 | C.14 |
| 1.2964 | B.136 | C.14 |
| 1.2965 | B.137 | C.14 |
| 1.2966 | B.138 | C.14 |
| 1.2967 | B.139 | C.14 |
| 1.2968 | B.140 | C.14 |
| 1.2969 | B.141 | C.14 |
| 1.2970 | B.142 | C.14 |
| 1.2971 | B.143 | C.14 |
| 1.2972 | B.144 | C.14 |
| 1.2973 | B.145 | C.14 |
| 1.2974 | B.146 | C.14 |
| 1.2975 | B.147 | C.14 |
| 1.2976 | B.148 | C.14 |
| 1.2977 | B.149 | C.14 |
| 1.2978 | B.150 | C.14 |
| 1.2979 | B.151 | C.14 |
| 1.2980 | B.152 | C.14 |
| 1.2981 | B.153 | C.14 |
| 1.2982 | B.154 | C.14 |
| 1.2983 | B.155 | C.14 |
| 1.2984 | B.156 | C.14 |
| 1.2985 | B.157 | C.14 |
| 1.2986 | B.158 | C.14 |
| 1.2987 | B.159 | C.14 |
| 1.2988 | B.160 | C.14 |
| 1.2989 | B.161 | C.14 |
| 1.2990 | B.162 | C.14 |
| 1.2991 | B.163 | C.14 |
| 1.2992 | B.164 | C.14 |
| 1.2993 | B.165 | C.14 |
| 1.2994 | B.166 | C.14 |
| 1.2995 | B.167 | C.14 |
| 1.2996 | B.168 | C.14 |
| 1.2997 | B.169 | C.14 |
| 1.2998 | B.170 | C.14 |
| 1.2999 | B.171 | C.14 |
| 1.3000 | B.172 | C.14 |
| 1.3001 | B.173 | C.14 |
| 1.3002 | B.174 | C.14 |
| 1.3003 | B.175 | C.14 |
| 1.3004 | B.176 | C.14 |
| 1.3005 | B.177 | C.14 |
| 1.3006 | B.178 | C.14 |
| 1.3007 | B.179 | C.14 |
| 1.3008 | B.180 | C.14 |
| 1.3009 | B.181 | C.14 |
| 1.3010 | B.182 | C.14 |
| 1.3011 | B.183 | C.14 |
| 1.3012 | B.184 | C.14 |
| 1.3013 | B.185 | C.14 |
| 1.3014 | B.186 | C.14 |
| 1.3015 | B.187 | C.14 |
| 1.3016 | B.188 | C.14 |
| 1.3017 | B.189 | C.14 |
| 1.3018 | B.190 | C.14 |
| 1.3019 | B.191 | C.14 |
| 1.3020 | B.192 | C.14 |
| 1.3021 | B.193 | C.14 |
| 1.3022 | B.194 | C.14 |
| 1.3023 | B.195 | C.14 |
| 1.3024 | B.196 | C.14 |
| 1.3025 | B.197 | C.14 |
| 1.3026 | B.198 | C.14 |
| 1.3027 | B.199 | C.14 |
| 1.3028 | B.200 | C.14 |
| 1.3029 | B.201 | C.14 |
| 1.3030 | B.202 | C.14 |
| 1.3031 | B.1 | C.15 |
| 1.3032 | B.2 | C.15 |
| 1.3033 | B.3 | C.15 |
| 1.3034 | B.4 | C.15 |
| 1.3035 | B.5 | C.15 |
| 1.3036 | B.6 | C.15 |
| 1.3037 | B.7 | C.15 |
| 1.3038 | B.8 | C.15 |
| 1.3039 | B.9 | C.15 |
| 1.3040 | B.10 | C.15 |
| 1.3041 | B.11 | C.15 |
| 1.3042 | B.12 | C.15 |
| 1.3043 | B.13 | C.15 |
| 1.3044 | B.14 | C.15 |
| 1.3045 | B.15 | C.15 |
| 1.3046 | B.16 | C.15 |
| 1.3047 | B.17 | C.15 |
| 1.3048 | B.18 | C.15 |
| 1.3049 | B.19 | C.15 |
| 1.3050 | B.20 | C.15 |
| 1.3051 | B.21 | C.15 |
| 1.3052 | B.22 | C.15 |
| 1.3053 | B.23 | C.15 |
| 1.3054 | B.24 | C.15 |
| 1.3055 | B.25 | C.15 |
| 1.3056 | B.26 | C.15 |
| 1.3057 | B.27 | C.15 |
| 1.3058 | B.28 | C.15 |
| 1.3059 | B.29 | C.15 |
| 1.3060 | B.30 | C.15 |
| 1.3061 | B.31 | C.15 |
| 1.3062 | B.32 | C.15 |
| 1.3063 | B.33 | C.15 |
| 1.3064 | B.34 | C.15 |
| 1.3065 | B.35 | C.15 |
| 1.3066 | B.36 | C.15 |
| 1.3067 | B.37 | C.15 |
| 1.3068 | B.38 | C.15 |
| 1.3069 | B.39 | C.15 |
| 1.3070 | B.40 | C.15 |
| 1.3071 | B.41 | C.15 |
| 1.3072 | B.42 | C.15 |
| 1.3073 | B.43 | C.15 |
| 1.3074 | B.44 | C.15 |
| 1.3075 | B.45 | C.15 |
| 1.3076 | B.46 | C.15 |
| 1.3077 | B.47 | C.15 |
| 1.3078 | B.48 | C.15 |
| 1.3079 | B.49 | C.15 |
| 1.3080 | B.50 | C.15 |
| 1.3081 | B.51 | C.15 |
| 1.3082 | B.52 | C.15 |
| 1.3083 | B.53 | C.15 |
| 1.3084 | B.54 | C.15 |
| 1.3085 | B.55 | C.15 |
| 1.3086 | B.56 | C.15 |
| 1.3087 | B.57 | C.15 |
| 1.3088 | B.58 | C.15 |
| 1.3089 | B.59 | C.15 |
| 1.3090 | B.60 | C.15 |
| 1.3091 | B.61 | C.15 |
| 1.3092 | B.62 | C.15 |
| 1.3093 | B.63 | C.15 |
| 1.3094 | B.64 | C.15 |
| 1.3095 | B.65 | C.15 |
| 1.3096 | B.66 | C.15 |
| 1.3097 | B.67 | C.15 |
| 1.3098 | B.68 | C.15 |
| 1.3099 | B.69 | C.15 |
| 1.3100 | B.70 | C.15 |
| 1.3101 | B.71 | C.15 |
| 1.3102 | B.72 | C.15 |
| 1.3103 | B.73 | C.15 |
| 1.3104 | B.74 | C.15 |
| 1.3105 | B.75 | C.15 |
| 1.3106 | B.76 | C.15 |
| 1.3107 | B.77 | C.15 |
| 1.3108 | B.78 | C.15 |
| 1.3109 | B.79 | C.15 |
| 1.3110 | B.80 | C.15 |
| 1.3111 | B.81 | C.15 |
| 1.3112 | B.82 | C.15 |
| 1.3113 | B.83 | C.15 |
| 1.3114 | B.84 | C.15 |
| 1.3115 | B.85 | C.15 |
| 1.3116 | B.86 | C.15 |
| 1.3117 | B.87 | C.15 |
| 1.3118 | B.88 | C.15 |
| 1.3119 | B.89 | C.15 |
| 1.3120 | B.90 | C.15 |
| 1.3121 | B.91 | C.15 |
| 1.3122 | B.92 | C.15 |
| 1.3123 | B.93 | C.15 |
| 1.3124 | B.94 | C.15 |
| 1.3125 | B.95 | C.15 |
| 1.3126 | B.96 | C.15 |
| 1.3127 | B.97 | C.15 |
| 1.3128 | B.98 | C.15 |
| 1.3129 | B.99 | C.15 |
| 1.3130 | B.100 | C.15 |
| 1.3131 | B.101 | C.15 |
| 1.3132 | B.102 | C.15 |
| 1.3133 | B.103 | C.15 |
| 1.3134 | B.104 | C.15 |
| 1.3135 | B.105 | C.15 |
| 1.3136 | B.106 | C.15 |
| 1.3137 | B.107 | C.15 |
| 1.3138 | B.108 | C.15 |
| 1.3139 | B.109 | C.15 |
| 1.3140 | B.110 | C.15 |
| 1.3141 | B.111 | C.15 |
| 1.3142 | B.112 | C.15 |
| 1.3143 | B.113 | C.15 |
| 1.3144 | B.114 | C.15 |
| 1.3145 | B.115 | C.15 |
| 1.3146 | B.116 | C.15 |
| 1.3147 | B.117 | C.15 |
| 1.3148 | B.118 | C.15 |
| 1.3149 | B.119 | C.15 |
| 1.3150 | B.120 | C.15 |
| 1.3151 | B.121 | C.15 |
| 1.3152 | B.122 | C.15 |
| 1.3153 | B.123 | C.15 |
| 1.3154 | B.124 | C.15 |
| 1.3155 | B.125 | C.15 |
| 1.3156 | B.126 | C.15 |
| 1.3157 | B.127 | C.15 |
| 1.3158 | B.128 | C.15 |
| 1.3159 | B.129 | C.15 |
| 1.3160 | B.130 | C.15 |
| 1.3161 | B.131 | C.15 |
| 1.3162 | B.132 | C.15 |
| 1.3163 | B.133 | C.15 |
| 1.3164 | B.134 | C.15 |
| 1.3165 | B.135 | C.15 |
| 1.3166 | B.136 | C.15 |
| 1.3167 | B.137 | C.15 |
| 1.3168 | B.138 | C.15 |
| 1.3169 | B.139 | C.15 |
| 1.3170 | B.140 | C.15 |
| 1.3171 | B.141 | C.15 |
| 1.3172 | B.142 | C.15 |
| 1.3173 | B.143 | C.15 |
| 1.3174 | B.144 | C.15 |
| 1.3175 | B.145 | C.15 |
| 1.3176 | B.146 | C.15 |
| 1.3177 | B.147 | C.15 |
| 1.3178 | B.148 | C.15 |
| 1.3179 | B.149 | C.15 |
| 1.3180 | B.150 | C.15 |
| 1.3181 | B.151 | C.15 |
| 1.3182 | B.152 | C.15 |
| 1.3183 | B.153 | C.15 |
| 1.3184 | B.154 | C.15 |
| 1.3185 | B.155 | C.15 |
| 1.3186 | B.156 | C.15 |
| 1.3187 | B.157 | C.15 |
| 1.3188 | B.158 | C.15 |
| 1.3189 | B.159 | C.15 |
| 1.3190 | B.160 | C.15 |
| 1.3191 | B.161 | C.15 |
| 1.3192 | B.162 | C.15 |
| 1.3193 | B.163 | C.15 |
| 1.3194 | B.164 | C.15 |
| 1.3195 | B.165 | C.15 |
| 1.3196 | B.166 | C.15 |
| 1.3197 | B.167 | C.15 |
| 1.3198 | B.168 | C.15 |
| 1.3199 | B.169 | C.15 |
| 1.3200 | B.170 | C.15 |
| 1.3201 | B.171 | C.15 |
| 1.3202 | B.172 | C.15 |
| 1.3203 | B.173 | C.15 |
| 1.3204 | B.174 | C.15 |
| 1.3205 | B.175 | C.15 |
| 1.3206 | B.176 | C.15 |
| 1.3207 | B.177 | C.15 |
| 1.3208 | B.178 | C.15 |
| 1.3209 | B.179 | C.15 |
| 1.3210 | B.180 | C.15 |
| 1.3211 | B.181 | C.15 |
| 1.3212 | B.182 | C.15 |
| 1.3213 | B.183 | C.15 |
| 1.3214 | B.184 | C.15 |
| 1.3215 | B.185 | C.15 |
| 1.3216 | B.186 | C.15 |
| 1.3217 | B.187 | C.15 |
| 1.3218 | B.188 | C.15 |
| 1.3219 | B.189 | C.15 |
| 1.3220 | B.190 | C.15 |
| 1.3221 | B.191 | C.15 |
| 1.3222 | B.192 | C.15 |
| 1.3223 | B.193 | C.15 |
| 1.3224 | B.194 | C.15 |
| 1.3225 | B.195 | C.15 |
| 1.3226 | B.196 | C.15 |
| 1.3227 | B.197 | C.15 |
| 1.3228 | B.198 | C.15 |
| 1.3229 | B.199 | C.15 |
| 1.3230 | B.200 | C.15 |
| 1.3231 | B.201 | C.15 |
| 1.3232 | B.202 | C.15 |
| 1.3233 | B.1 | C.16 |
| 1.3234 | B.2 | C.16 |
| 1.3235 | B.3 | C.16 |
| 1.3236 | B.4 | C.16 |
| 1.3237 | B.5 | C.16 |
| 1.3238 | B.6 | C.16 |
| 1.3239 | B.7 | C.16 |
| 1.3240 | B.8 | C.16 |
| 1.3241 | B.9 | C.16 |
| 1.3242 | B.10 | C.16 |
| 1.3243 | B.11 | C.16 |
| 1.3244 | B.12 | C.16 |
| 1.3245 | B.13 | C.16 |
| 1.3246 | B.14 | C.16 |
| 1.3247 | B.15 | C.16 |
| 1.3248 | B.16 | C.16 |
| 1.3249 | B.17 | C.16 |
| 1.3250 | B.18 | C.16 |
| 1.3251 | B.19 | C.16 |
| 1.3252 | B.20 | C.16 |
| 1.3253 | B.21 | C.16 |
| 1.3254 | B.22 | C.16 |
| 1.3255 | B.23 | C.16 |
| 1.3256 | B.24 | C.16 |
| 1.3257 | B.25 | C.16 |
| 1.3258 | B.26 | C.16 |
| 1.3259 | B.27 | C.16 |
| 1.3260 | B.28 | C.16 |
| 1.3261 | B.29 | C.16 |
| 1.3262 | B.30 | C.16 |
| 1.3263 | B.31 | C.16 |
| 1.3264 | B.32 | C.16 |
| 1.3265 | B.33 | C.16 |
| 1.3266 | B.34 | C.16 |
| 1.3267 | B.35 | C.16 |
| 1.3268 | B.36 | C.16 |
| 1.3269 | B.37 | C.16 |
| 1.3270 | B.38 | C.16 |
| 1.3271 | B.39 | C.16 |
| 1.3272 | B.40 | C.16 |
| 1.3273 | B.41 | C.16 |
| 1.3274 | B.42 | C.16 |
| 1.3275 | B.43 | C.16 |
| 1.3276 | B.44 | C.16 |
| 1.3277 | B.45 | C.16 |
| 1.3278 | B.46 | C.16 |
| 1.3279 | B.47 | C.16 |
| 1.3280 | B.48 | C.16 |
| 1.3281 | B.49 | C.16 |
| 1.3282 | B.50 | C.16 |
| 1.3283 | B.51 | C.16 |
| 1.3284 | B.52 | C.16 |
| 1.3285 | B.53 | C.16 |
| 1.3286 | B.54 | C.16 |
| 1.3287 | B.55 | C.16 |
| 1.3288 | B.56 | C.16 |
| 1.3289 | B.57 | C.16 |
| 1.3290 | B.58 | C.16 |
| 1.3291 | B.59 | C.16 |
| 1.3292 | B.60 | C.16 |
| 1.3293 | B.61 | C.16 |
| 1.3294 | B.62 | C.16 |
| 1.3295 | B.63 | C.16 |
| 1.3296 | B.64 | C.16 |
| 1.3297 | B.65 | C.16 |
| 1.3298 | B.66 | C.16 |
| 1.3299 | B.67 | C.16 |
| 1.3300 | B.68 | C.16 |
| 1.3301 | B.69 | C.16 |
| 1.3302 | B.70 | C.16 |
| 1.3303 | B.71 | C.16 |
| 1.3304 | B.72 | C.16 |
| 1.3305 | B.73 | C.16 |
| 1.3306 | B.74 | C.16 |
| 1.3307 | B.75 | C.16 |
| 1.3308 | B.76 | C.16 |
| 1.3309 | B.77 | C.16 |
| 1.3310 | B.78 | C.16 |
| 1.3311 | B.79 | C.16 |
| 1.3312 | B.80 | C.16 |
| 1.3313 | B.81 | C.16 |
| 1.3314 | B.82 | C.16 |
| 1.3315 | B.83 | C.16 |
| 1.3316 | B.84 | C.16 |
| 1.3317 | B.85 | C.16 |
| 1.3318 | B.86 | C.16 |
| 1.3319 | B.87 | C.16 |
| 1.3320 | B.88 | C.16 |
| 1.3321 | B.89 | C.16 |
| 1.3322 | B.90 | C.16 |
| 1.3323 | B.91 | C.16 |
| 1.3324 | B.92 | C.16 |
| 1.3325 | B.93 | C.16 |
| 1.3326 | B.94 | C.16 |
| 1.3327 | B.95 | C.16 |
| 1.3328 | B.96 | C.16 |
| 1.3329 | B.97 | C.16 |
| 1.3330 | B.98 | C.16 |
| 1.3331 | B.99 | C.16 |
| 1.3332 | B.100 | C.16 |
| 1.3333 | B.101 | C.16 |
| 1.3334 | B.102 | C.16 |
| 1.3335 | B.103 | C.16 |
| 1.3336 | B.104 | C.16 |
| 1.3337 | B.105 | C.16 |
| 1.3338 | B.106 | C.16 |
| 1.3339 | B.107 | C.16 |
| 1.3340 | B.108 | C.16 |
| 1.3341 | B.109 | C.16 |
| 1.3342 | B.110 | C.16 |
| 1.3343 | B.111 | C.16 |
| 1.3344 | B.112 | C.16 |
| 1.3345 | B.113 | C.16 |
| 1.3346 | B.114 | C.16 |
| 1.3347 | B.115 | C.16 |
| 1.3348 | B.116 | C.16 |
| 1.3349 | B.117 | C.16 |
| 1.3350 | B.118 | C.16 |
| 1.3351 | B.119 | C.16 |
| 1.3352 | B.120 | C.16 |
| 1.3353 | B.121 | C.16 |
| 1.3354 | B.122 | C.16 |
| 1.3355 | B.123 | C.16 |
| 1.3356 | B.124 | C.16 |
| 1.3357 | B.125 | C.16 |
| 1.3358 | B.126 | C.16 |
| 1.3359 | B.127 | C.16 |
| 1.3360 | B.128 | C.16 |
| 1.3361 | B.129 | C.16 |
| 1.3362 | B.130 | C.16 |
| 1.3363 | B.131 | C.16 |
| 1.3364 | B.132 | C.16 |
| 1.3365 | B.133 | C.16 |
| 1.3366 | B.134 | C.16 |
| 1.3367 | B.135 | C.16 |
| 1.3368 | B.136 | C.16 |
| 1.3369 | B.137 | C.16 |
| 1.3370 | B.138 | C.16 |
| 1.3371 | B.139 | C.16 |
| 1.3372 | B.140 | C.16 |
| 1.3373 | B.141 | C.16 |
| 1.3374 | B.142 | C.16 |
| 1.3375 | B.143 | C.16 |
| 1.3376 | B.144 | C.16 |
| 1.3377 | B.145 | C.16 |
| 1.3378 | B.146 | C.16 |
| 1.3379 | B.147 | C.16 |
| 1.3380 | B.148 | C.16 |
| 1.3381 | B.149 | C.16 |
| 1.3382 | B.150 | C.16 |
| 1.3383 | B.151 | C.16 |
| 1.3384 | B.152 | C.16 |
| 1.3385 | B.153 | C.16 |
| 1.3386 | B.154 | C.16 |
| 1.3387 | B.155 | C.16 |
| 1.3388 | B.156 | C.16 |
| 1.3389 | B.157 | C.16 |
| 1.3390 | B.158 | C.16 |
| 1.3391 | B.159 | C.16 |
| 1.3392 | B.160 | C.16 |
| 1.3393 | B.161 | C.16 |
| 1.3394 | B.162 | C.16 |
| 1.3395 | B.163 | C.16 |
| 1.3396 | B.164 | C.16 |
| 1.3397 | B.165 | C.16 |
| 1.3398 | B.166 | C.16 |
| 1.3399 | B.167 | C.16 |
| 1.3400 | B.168 | C.16 |
| 1.3401 | B.169 | C.16 |
| 1.3402 | B.170 | C.16 |
| 1.3403 | B.171 | C.16 |
| 1.3404 | B.172 | C.16 |
| 1.3405 | B.173 | C.16 |
| 1.3406 | B.174 | C.16 |
| 1.3407 | B.175 | C.16 |
| 1.3408 | B.176 | C.16 |
| 1.3409 | B.177 | C.16 |
| 1.3410 | B.178 | C.16 |
| 1.3411 | B.179 | C.16 |
| 1.3412 | B.180 | C.16 |
| 1.3413 | B.181 | C.16 |
| 1.3414 | B.182 | C.16 |
| 1.3415 | B.183 | C.16 |
| 1.3416 | B.184 | C.16 |
| 1.3417 | B.185 | C.16 |
| 1.3418 | B.186 | C.16 |
| 1.3419 | B.187 | C.16 |
| 1.3420 | B.188 | C.16 |
| 1.3421 | B.189 | C.16 |
| 1.3422 | B.190 | C.16 |
| 1.3423 | B.191 | C.16 |
| 1.3424 | B.192 | C.16 |
| 1.3425 | B.193 | C.16 |
| 1.3426 | B.194 | C.16 |
| 1.3427 | B.195 | C.16 |
| 1.3428 | B.196 | C.16 |
| 1.3429 | B.197 | C.16 |
| 1.3430 | B.198 | C.16 |
| 1.3431 | B.199 | C.16 |
| 1.3432 | B.200 | C.16 |
| 1.3433 | B.201 | C.16 |
| 1.3434 | B.202 | C.16 |
| 1.3435 | B.1 | C.17 |
| 1.3436 | B.2 | C.17 |
| 1.3437 | B.3 | C.17 |
| 1.3438 | B.4 | C.17 |
| 1.3439 | B.5 | C.17 |
| 1.3440 | B.6 | C.17 |
| 1.3441 | B.7 | C.17 |
| 1.3442 | B.8 | C.17 |
| 1.3443 | B.9 | C.17 |
| 1.3444 | B.10 | C.17 |
| 1.3445 | B.11 | C.17 |
| 1.3446 | B.12 | C.17 |
| 1.3447 | B.13 | C.17 |
| 1.3448 | B.14 | C.17 |
| 1.3449 | B.15 | C.17 |
| 1.3450 | B.16 | C.17 |
| 1.3451 | B.17 | C.17 |
| 1.3452 | B.18 | C.17 |
| 1.3453 | B.19 | C.17 |
| 1.3454 | B.20 | C.17 |
| 1.3455 | B.21 | C.17 |
| 1.3456 | B.22 | C.17 |
| 1.3457 | B.23 | C.17 |
| 1.3458 | B.24 | C.17 |
| 1.3459 | B.25 | C.17 |
| 1.3460 | B.26 | C.17 |
| 1.3461 | B.27 | C.17 |
| 1.3462 | B.28 | C.17 |
| 1.3463 | B.29 | C.17 |
| 1.3464 | B.30 | C.17 |
| 1.3465 | B.31 | C.17 |
| 1.3466 | B.32 | C.17 |
| 1.3467 | B.33 | C.17 |
| 1.3468 | B.34 | C.17 |
| 1.3469 | B.35 | C.17 |
| 1.3470 | B.36 | C.17 |
| 1.3471 | B.37 | C.17 |
| 1.3472 | B.38 | C.17 |
| 1.3473 | B.39 | C.17 |
| 1.3474 | B.40 | C.17 |
| 1.3475 | B.41 | C.17 |
| 1.3476 | B.42 | C.17 |
| 1.3477 | B.43 | C.17 |
| 1.3478 | B.44 | C.17 |
| 1.3479 | B.45 | C.17 |
| 1.3480 | B.46 | C.17 |
| 1.3481 | B.47 | C.17 |
| 1.3482 | B.48 | C.17 |
| 1.3483 | B.49 | C.17 |
| 1.3484 | B.50 | C.17 |
| 1.3485 | B.51 | C.17 |
| 1.3486 | B.52 | C.17 |
| 1.3487 | B.53 | C.17 |
| 1.3488 | B.54 | C.17 |
| 1.3489 | B.55 | C.17 |
| 1.3490 | B.56 | C.17 |
| 1.3491 | B.57 | C.17 |
| 1.3492 | B.58 | C.17 |
| 1.3493 | B.59 | C.17 |
| 1.3494 | B.60 | C.17 |
| 1.3495 | B.61 | C.17 |
| 1.3496 | B.62 | C.17 |
| 1.3497 | B.63 | C.17 |
| 1.3498 | B.64 | C.17 |
| 1.3499 | B.65 | C.17 |
| 1.3500 | B.66 | C.17 |
| 1.3501 | B.67 | C.17 |
| 1.3502 | B.68 | C.17 |
| 1.3503 | B.69 | C.17 |
| 1.3504 | B.70 | C.17 |
| 1.3505 | B.71 | C.17 |
| 1.3506 | B.72 | C.17 |
| 1.3507 | B.73 | C.17 |
| 1.3508 | B.74 | C.17 |
| 1.3509 | B.75 | C.17 |
| 1.3510 | B.76 | C.17 |
| 1.3511 | B.77 | C.17 |
| 1.3512 | B.78 | C.17 |
| 1.3513 | B.79 | C.17 |
| 1.3514 | B.80 | C.17 |
| 1.3515 | B.81 | C.17 |
| 1.3516 | B.82 | C.17 |
| 1.3517 | B.83 | C.17 |
| 1.3518 | B.84 | C.17 |
| 1.3519 | B.85 | C.17 |
| 1.3520 | B.86 | C.17 |
| 1.3521 | B.87 | C.17 |
| 1.3522 | B.88 | C.17 |
| 1.3523 | B.89 | C.17 |
| 1.3524 | B.90 | C.17 |
| 1.3525 | B.91 | C.17 |
| 1.3526 | B.92 | C.17 |
| 1.3527 | B.93 | C.17 |
| 1.3528 | B.94 | C.17 |
| 1.3529 | B.95 | C.17 |
| 1.3530 | B.96 | C.17 |
| 1.3531 | B.97 | C.17 |
| 1.3532 | B.98 | C.17 |
| 1.3533 | B.99 | C.17 |
| 1.3534 | B.100 | C.17 |
| 1.3535 | B.101 | C.17 |
| 1.3536 | B.102 | C.17 |
| 1.3537 | B.103 | C.17 |
| 1.3538 | B.104 | C.17 |
| 1.3539 | B.105 | C.17 |
| 1.3540 | B.106 | C.17 |
| 1.3541 | B.107 | C.17 |
| 1.3542 | B.108 | C.17 |
| 1.3543 | B.109 | C.17 |
| 1.3544 | B.110 | C.17 |
| 1.3545 | B.111 | C.17 |
| 1.3546 | B.112 | C.17 |
| 1.3547 | B.113 | C.17 |
| 1.3548 | B.114 | C.17 |
| 1.3549 | B.115 | C.17 |
| 1.3550 | B.116 | C.17 |
| 1.3551 | B.117 | C.17 |
| 1.3552 | B.118 | C.17 |
| 1.3553 | B.119 | C.17 |
| 1.3554 | B.120 | C.17 |
| 1.3555 | B.121 | C.17 |
| 1.3556 | B.122 | C.17 |
| 1.3557 | B.123 | C.17 |
| 1.3558 | B.124 | C.17 |
| 1.3559 | B.125 | C.17 |
| 1.3560 | B.126 | C.17 |
| 1.3561 | B.127 | C.17 |
| 1.3562 | B.128 | C.17 |
| 1.3563 | B.129 | C.17 |
| 1.3564 | B.130 | C.17 |
| 1.3565 | B.131 | C.17 |
| 1.3566 | B.132 | C.17 |
| 1.3567 | B.133 | C.17 |
| 1.3568 | B.134 | C.17 |
| 1.3569 | B.135 | C.17 |
| 1.3570 | B.136 | C.17 |
| 1.3571 | B.137 | C.17 |
| 1.3572 | B.138 | C.17 |
| 1.3573 | B.139 | C.17 |
| 1.3574 | B.140 | C.17 |
| 1.3575 | B.141 | C.17 |
| 1.3576 | B.142 | C.17 |
| 1.3577 | B.143 | C.17 |
| 1.3578 | B.144 | C.17 |
| 1.3579 | B.145 | C.17 |
| 1.3580 | B.146 | C.17 |
| 1.3581 | B.147 | C.17 |
| 1.3582 | B.148 | C.17 |
| 1.3583 | B.149 | C.17 |
| 1.3584 | B.150 | C.17 |
| 1.3585 | B.151 | C.17 |
| 1.3586 | B.152 | C.17 |
| 1.3587 | B.153 | C.17 |
| 1.3588 | B.154 | C.17 |
| 1.3589 | B.155 | C.17 |
| 1.3590 | B.156 | C.17 |
| 1.3591 | B.157 | C.17 |
| 1.3592 | B.158 | C.17 |
| 1.3593 | B.159 | C.17 |
| 1.3594 | B.160 | C.17 |
| 1.3595 | B.161 | C.17 |
| 1.3596 | B.162 | C.17 |
| 1.3597 | B.163 | C.17 |
| 1.3598 | B.164 | C.17 |
| 1.3599 | B.165 | C.17 |
| 1.3600 | B.166 | C.17 |
| 1.3601 | B.167 | C.17 |
| 1.3602 | B.168 | C.17 |
| 1.3603 | B.169 | C.17 |
| 1.3604 | B.170 | C.17 |
| 1.3605 | B.171 | C.17 |
| 1.3606 | B.172 | C.17 |
| 1.3607 | B.173 | C.17 |
| 1.3608 | B.174 | C.17 |
| 1.3609 | B.175 | C.17 |
| 1.3610 | B.176 | C.17 |
| 1.3611 | B.177 | C.17 |
| 1.3612 | B.178 | C.17 |
| 1.3613 | B.179 | C.17 |
| 1.3614 | B.180 | C.17 |
| 1.3615 | B.181 | C.17 |
| 1.3616 | B.182 | C.17 |
| 1.3617 | B.183 | C.17 |
| 1.3618 | B.184 | C.17 |
| 1.3619 | B.185 | C.17 |
| 1.3620 | B.186 | C.17 |
| 1.3621 | B.187 | C.17 |
| 1.3622 | B.188 | C.17 |
| 1.3623 | B.189 | C.17 |
| 1.3624 | B.190 | C.17 |
| 1.3625 | B.191 | C.17 |
| 1.3626 | B.192 | C.17 |
| 1.3627 | B.193 | C.17 |
| 1.3628 | B.194 | C.17 |
| 1.3629 | B.195 | C.17 |
| 1.3630 | B.196 | C.17 |
| 1.3631 | B.197 | C.17 |
| 1.3632 | B.198 | C.17 |
| 1.3633 | B.199 | C.17 |
| 1.3634 | B.200 | C.17 |
| 1.3635 | B.201 | C.17 |
| 1.3636 | B.202 | C.17 |
| 1.3637 | — | C.1 |
| 1.3638 | — | C.2 |
| 1.3639 | — | C.3 |
| 1.3640 | — | C.4 |
| 1.3641 | — | C.5 |
| 1.3642 | — | C.6 |
| 1.3643 | — | C.7 |
| 1.3644 | — | C.8 |
| 1.3645 | — | C.9 |
| 1.3646 | — | C.10 |
| 1.3647 | — | C.11 |
| 1.3648 | — | C.12 |
| 1.3649 | — | C.13 |
| 1.3650 | — | C.14 |
| 1.3651 | — | C.15 |
| 1.3652 | — | C.16 |
| 1.3653 | — | C.17 |

The specific number for each single composition is deductible as follows:

Composition 1.203 e.g. comprises the compound (1.1.I-25), clethodim (B.1) and benoxacor (C.1) (see table B, entry B.1 and table C, entry C.1).

Also especially preferred are compositions 2.1 to 2.3653 which differ from the corresponding compositions 1.3 to 1.3653 only in that they comprise the compound (1.3.I-4) 1.3 in place of the compound (1.3.I-3).

Also especially preferred are compositions 3.1 to 3.3653 which differ from the corresponding compositions 1.1 to 1.3653 only in that they additionally comprise B.2 as further herbicide B.

Also especially preferred are compositions 4.1 to 4.3653 which differ from the corresponding compositions 1.1 to 1.3653 only in that they additionally comprise B.8 as further herbicide B.

Also especially preferred are compositions 5.1 to 5.3653 which differ from the corresponding compositions 1.1 to 1.3653 only in that they additionally comprise B.30 as further herbicide B.

Also especially preferred are compositions 6.1 to 6.3653 which differ from the corresponding compositions 1.1 to 1.3653 only in that they additionally comprise B.32 as further herbicide B.

Also especially preferred are compositions 7.1 to 7.3653 which differ from the corresponding compositions 1.1 to 1.3653 only in that they additionally comprise B.35 as further herbicide B.

Also especially preferred are compositions 8.1 to 8.3653 which differ from the corresponding compositions 1.1 to 1.3653 only in that they additionally comprise B.38 as further herbicide B.

Also especially preferred are compositions 9.1 to 9.3653 which differ from the corresponding compositions 1.1 to 1.3653 only in that they additionally comprise B.40 as further herbicide B.

Also especially preferred are compositions 10.1 to 10.3653 which differ from the corresponding compositions 1.1 to 1.3653 only in that they additionally comprise B.51 as further herbicide B.

Also especially preferred are compositions 11.1 to 11.3653 which differ from the corresponding compositions 1.1 to 1.3653 only in that they additionally comprise B.55 as further herbicide B.

Also especially preferred are compositions 12.1 to 12.3653 which differ from the corresponding compositions 1.1 to 1.3653 only in that they additionally comprise B.56 as further herbicide B.

Also especially preferred are compositions 13.1 to 13.3653 which differ from the corresponding compositions 1.1 to 1.3653 only in that they additionally comprise B.64 as further herbicide B.

Also especially preferred are compositions 14.1 to 14.3653 which differ from the corresponding compositions 1.1 to 1.3653 only in that they additionally comprise B.66 as further herbicide B.

Also especially preferred are compositions 15.1 to 15.3653 which differ from the corresponding compositions 1.1 to 1.3653 only in that they additionally comprise B.67 as further herbicide B.

Also especially preferred are compositions 16.1 to 16.3653 which differ from the corresponding compositions 1.1 to 1.3653 only in that they additionally comprise B.68 as further herbicide B.

Also especially preferred are compositions 17.1 to 17.3653 which differ from the corresponding compositions 1.1 to 1.3653 only in that they additionally comprise B.69 as further herbicide B.

Also especially preferred are compositions 18.1 to 18.3653 which differ from the corresponding compositions 1.1 to 1.3653 only in that they additionally comprise B.73 as further herbicide B.

Also especially preferred are compositions 19.1 to 19.3653 which differ from the corresponding compositions 1.1 to 1.3653 only in that they additionally comprise B.76 as further herbicide B.

Also especially preferred are compositions 20.1 to 20.3653 which differ from the corresponding compositions 1.1 to 1.3653 only in that they additionally comprise B.81 as further herbicide B.

Also especially preferred are compositions 21.1 to 21.3653 which differ from the corresponding compositions 1.1 to 1.3653 only in that they additionally comprise B.82 as further herbicide B.

Also especially preferred are compositions 22.1 to 22.3653 which differ from the corresponding compositions 1.1 to 1.3653 only in that they additionally comprise B.85 as further herbicide B.

Also especially preferred are compositions 23.1 to 23.3653 which differ from the corresponding compositions 1.1 to 1.3653 only in that they additionally comprise B.88 as further herbicide B.

Also especially preferred are compositions 24.1 to 24.3653 which differ from the corresponding compositions 1.1 to 1.3653 only in that they additionally comprise B.89 as further herbicide B.

Also especially preferred are compositions 25.1 to 25.3653 which differ from the corresponding compositions 1.1 to 1.3653 only in that they additionally comprise B.94 as further herbicide B.

Also especially preferred are compositions 26.1 to 26.3653 which differ from the corresponding compositions 1.1 to 1.3653 only in that they additionally comprise B.95 as further herbicide B.

Also especially preferred are compositions 27.1 to 27.3653 which differ from the corresponding compositions 1.1 to 1.3653 only in that they additionally comprise B.98 as further herbicide B.

Also especially preferred are compositions 28.1 to 28.3653 which differ from the corresponding compositions 1.1 to 1.3653 only in that they additionally comprise B.100 as further herbicide B.

Also especially preferred are compositions 29.1 to 29.3653 which differ from the corresponding compositions 1.1 to 1.3653 only in that they additionally comprise B.103 as further herbicide B.

Also especially preferred are compositions 30.1 to 30.3653 which differ from the corresponding compositions 1.1 to 1.3653 only in that they additionally comprise B.103 and B.67 as further herbicides B.

Also especially preferred are compositions 31.1 to 31.3653 which differ from the corresponding compositions 1.1 to 1.3653 only in that they additionally comprise B.103 and B.76 as further herbicides B.

Also especially preferred are compositions 32.1 to 32.3653 which differ from the corresponding compositions 1.1 to 1.3653 only in that they additionally comprise B.103 and B.82 as further herbicides B.

Also especially preferred are compositions 33.1 to 33.3653 which differ from the corresponding compositions 1.1 to 1.3653 only in that they additionally comprise B.104 as further herbicide B.

Also especially preferred are compositions 34.1 to 34.3653 which differ from the corresponding compositions 1.1 to 1.3653 only in that they additionally comprise B.104 and B.67 as further herbicides B.

Also especially preferred are compositions 35.1 to 35.3653 which differ from the corresponding compositions 1.1 to 1.3653 only in that they additionally comprise B.104 and B.76 as further herbicides B.

Also especially preferred are compositions 36.1 to 36.3653 which differ from the corresponding compositions 1.1 to 1.3653 only in that they additionally comprise B.104 and B.82 as further herbicides B.

Also especially preferred are compositions 37.1 to 37.3653 which differ from the corresponding compositions 1.1 to 1.3653 only in that they additionally comprise B.106 as further herbicide B.

Also especially preferred are compositions 38.1 to 38.3653 which differ from the corresponding compositions 1.1 to 1.3653 only in that they additionally comprise B.107 as further herbicide B.

Also especially preferred are compositions 39.1 to 39.3653 which differ from the corresponding compositions 1.1 to 1.3653 only in that they additionally comprise B. 107 and B.67 as further herbicides B.

Also especially preferred are compositions 40.1 to 40.3653 which differ from the corresponding compositions 1.1 to 1.3653 only in that they additionally comprise B. 107 and B.76 as further herbicides B.

Also especially preferred are compositions 41.1 to 41.3653 which differ from the corresponding compositions 1.1 to 1.3653 only in that they additionally comprise B. 107 and B.82 as further herbicides B.

Also especially preferred are compositions 42.1 to 42.3653 which differ from the corresponding compositions 1.1 to 1.3653 only in that they additionally comprise B.109 as further herbicide B.

Also especially preferred are compositions 43.1 to 43.3653 which differ from the corresponding compositions 1.1 to 1.3653 only in that they additionally comprise B.111 as further herbicide B.

Also especially preferred are compositions 44.1 to 44.3653 which differ from the corresponding compositions 1.1 to 1.3653 only in that they additionally comprise B.111 and B.67 as further herbicides B.

Also especially preferred are compositions 45.1 to 45.3653 which differ from the corresponding compositions 1.1 to 1.3653 only in that they additionally comprise B.111 and B.76 as further herbicides B.

Also especially preferred are compositions 46.1 to 46.3653 which differ from the corresponding compositions 1.1 to 1.3653 only in that they additionally comprise B.111 and B.82 as further herbicides B.

Also especially preferred are compositions 47.1 to 47.3653 which differ from the corresponding compositions 1.1 to 1.3653 only in that they additionally comprise B. 116 as further herbicide B.

Also especially preferred are compositions 48.1 to 48.3653 which differ from the corresponding compositions 1.1 to 1.3653 only in that they additionally comprise B.116 and B.67 as further herbicides B.

Also especially preferred are compositions 49.1 to 49.3653 which differ from the corresponding compositions 1.1 to 1.3653 only in that they additionally comprise B.116 and B.94 as further herbicides B.

Also especially preferred are compositions 50.1 to 50.3653 which differ from the corresponding compositions 1.1 to 1.3653 only in that they additionally comprise B.116 and B.103 as further herbicides B.

Also especially preferred are compositions 51.1 to 51.3653 which differ from the corresponding compositions 1.1 to 1.3653 only in that they additionally comprise B.116 and B.128 as further herbicides B.

Also especially preferred are compositions 52.1 to 52.3653 which differ from the corresponding compositions 1.1 to 1.3653 only in that they additionally comprise B.116 and B.104 as further herbicides B.

Also especially preferred are compositions 53.1 to 53.3653 which differ from the corresponding compositions 1.1 to 1.3653 only in that they additionally comprise B.116 and B.107 as further herbicides B.

Also especially preferred are compositions 54.1 to 54.3653 which differ from the corresponding compositions 1.1 to 1.3653 only in that they additionally comprise B.116 and B.111 as further herbicides B.

Also especially preferred are compositions 55.1 to 55.3653 which differ from the corresponding compositions 1.1 to 1.3653 only in that they additionally comprise B.122 as further herbicide B.

Also especially preferred are compositions 56.1 to 56.3653 which differ from the corresponding compositions 1.1 to 1.3653 only in that they additionally comprise B.126 as further herbicide B.

Also especially preferred are compositions 57.1 to 57.3653 which differ from the corresponding compositions 1.1 to 1.3653 only in that they additionally comprise B.128 as further herbicide B.

Also especially preferred are compositions 58.1 to 58.3653 which differ from the corresponding compositions 1.1 to 1.3653 only in that they additionally comprise B.131 as further herbicide B.

Also especially preferred are compositions 59.1 to 59.3653 which differ from the corresponding compositions 1.1 to 1.3653 only in that they additionally comprise B.132 as further herbicide B.

Also especially preferred are compositions 60.1 to 60.3653 which differ from the corresponding compositions 1.1 to 1.3653 only in that they additionally comprise B.133 as further herbicide B.

Also especially preferred are compositions 61.1 to 61.3653 which differ from the corresponding compositions 1.1 to 1.3653 only in that they additionally comprise B.135 as further herbicide B.

Also especially preferred are compositions 62.1 to 62.3653 which differ from the corresponding compositions 1.1 to 1.3653 only in that they additionally comprise B.137 as further herbicide B.

Also especially preferred are compositions 63.1 to 63.3653 which differ from the corresponding compositions 11.1 to 1.3653 only in that they additionally comprise B.138 as further herbicide B.

Also especially preferred are compositions 64.1 to 64.3653 which differ from the corresponding compositions 1.1 to 1.3653 only in that they additionally comprise B.140 as further herbicide B.

Also especially preferred are compositions 65.1 to 65.3653 which differ from the corresponding compositions 1.1 to 1.3653 only in that they additionally comprise B.145 as further herbicide B.

Also especially preferred are compositions 66.1 to 66.3653 which differ from the corresponding compositions 1.1 to 1.3653 only in that they additionally comprise B.153 as further herbicide B.

Also especially preferred are compositions 67.1 to 67.3653 which differ from the corresponding compositions 1.1 to 1.3653 only in that they additionally comprise B.156 as further herbicide B.

Also especially preferred are compositions 68.1 to 68.3653 which differ from the corresponding compositions 1.1 to 1.3653 only in that they additionally comprise B.171 as further herbicide B.

Also especially preferred are compositions 69.1 to 69.3653 which differ from the corresponding compositions 1.1 to 1.3653 only in that they additionally comprise B.174 as further herbicide B.

Also especially preferred are compositions 70.1 to 70.3653 which differ from the corresponding compositions 1.1 to 1.3653 only in that they comprise the compound (1.3.I-10) in place of the compound (1.3.I-3).

Also especially preferred are compositions 71.1 to 71.3653 which differ from the corresponding compositions 1.1 to 1.3653 only in that they comprise the compound (1.3.I-11) in place of the compound (1.3.I-3).

Also especially preferred are compositions 72.1 to 72.3653 which differ from the corresponding compositions 1.1 to 1.3653 only in that they comprise the compound (1.3.I-17) in place of the compound (1.3.I-3).

Also especially preferred are compositions 73.1 to 73.3653 which differ from the corresponding compositions 1.1 to 1.3653 only in that they comprise the compound (1.3.I-18) in place of the compound (1.3.I-3).

Also especially preferred are compositions 74.1 to 74.3653 which differ from the corresponding compositions 1.1 to 1.3653 only in that they comprise the compound (1.3.I-24) in place of the compound (1.3.I-3).

Also especially preferred are compositions 75.1 to 75.3653 which differ from the corresponding compositions 1.1 to 1.3653 only in that they comprise the compound (1.3.I-25) in place of the compound (1.3.I-3).

Also especially preferred are compositions 76.1 to 76.3653 which differ from the corresponding compositions 1.1 to 1.3653 only in that they comprise the compound (1.3.I-31) in place of the compound (1.3.I-3).

Also especially preferred are compositions 77.1 to 77.3653 which differ from the corresponding compositions 1.1 to 1.3653 only in that they comprise the compound (1.3.I-32) in place of the compound (1.3.I-3).

Also especially preferred are compositions 78.1 to 78.3653 which differ from the corresponding compositions 1.1 to 1.3653 only in that they comprise the compound (1.3.I-38) in place of the compound (1.3.I-3).

Also especially preferred are compositions 79.1 to 79.3653 which differ from the corresponding compositions 1.1 to 1.3653 only in that they comprise the compound (1.3.I-39) in place of the compound (1.3.I-3).

Also especially preferred are compositions 80.1 to 80.3653 which differ from the corresponding compositions 1.1 to 1.3653 only in that they comprise the compound (1.3.I-45) in place of the compound (1.3.I-3).

Also especially preferred are compositions 81.1 to 81.3653 which differ from the corresponding compositions 1.1 to 1.3653 only in that they comprise the compound (1.3.I-46) in place of the compound (1.3.I-3).

Also especially preferred are compositions 82.1 to 82.3653 which differ from the corresponding compositions 1.1 to 1.3653 only in that they comprise the compound (1.3.I-52) in place of the compound (1.3.I-3).

Also especially preferred are compositions 83.1 to 83.3653 which differ from the corresponding compositions 1.1 to 1.3653 only in that they comprise the compound (1.3.I-53) in place of the compound (1.3.I-3).

Also especially preferred are compositions 84.1 to 84.3653 which differ from the corresponding compositions 1.1 to 1.3653 only in that they comprise the compound (1.3.I-59) in place of the compound (1.3.I-3).

Also especially preferred are compositions 85.1 to 85.3653 which differ from the corresponding compositions 1.1 to 1.3653 only in that they comprise the compound (1.3.I-60) in place of the compound (1.3.I-3).

Also especially preferred are compositions 86.1 to 86.3653 which differ from the corresponding compositions 1.1 to 1.3653 only in that they comprise the compound (1.3.I-66) in place of the compound (1.3.I-3).

Also especially preferred are compositions 87.1 to 87.3653 which differ from the corresponding compositions 1.1 to 1.3653 only in that they comprise the compound (1.3.I-67) in place of the compound (1.3.I-3).

Also especially preferred are compositions 88.1 to 88.3653 which differ from the corresponding compositions 1.1 to 1.3653 only in that they comprise the compound (1.15.I-3) in place of the compound (1.3.I-3).

Also especially preferred are compositions 89.1 to 89.3653 which differ from the corresponding compositions 1.1 to 1.3653 only in that they comprise the compound (1.15.I-4) in place of the compound (1.3.I-3).

Also especially preferred are compositions 90.1 to 90.3653 which differ from the corresponding compositions 1.1 to 1.3653 only in that they comprise the compound (1.15.I-10) in place of the compound (1.3.I-3).

Also especially preferred are compositions 91.1 to 91.3653 which differ from the corresponding compositions 1.1 to 1.3653 only in that they comprise the compound (1.15.I-11) in place of the compound (1.3.I-3).

Also especially preferred are compositions 92.1 to 92.3653 which differ from the corresponding compositions 1.1 to 1.3653 only in that they comprise the compound (1.15.I-17) in place of the compound (1.3.I-3).

Also especially preferred are compositions 93.1 to 93.3653 which differ from the corresponding compositions 1.1 to 1.3653 only in that they comprise the compound (1.15.I-24) in place of the compound (1.3.I-3).

Also especially preferred are compositions 94.1 to 94.3653 which differ from the corresponding compositions 1.1 to 1.3653 only in that they comprise the compound (1.15.I-25) in place of the compound (1.3.I-3).

Also especially preferred are compositions 95.1 to 95.3653 which differ from the corresponding compositions 1.1 to 1.3653 only in that they comprise the compound (1.15.I-31) in place of the compound (1.3.I-3).

Also especially preferred are compositions 96.1 to 96.3653 which differ from the corresponding compositions 1.1 to 1.3653 only in that they comprise the compound (1.15.I-32) in place of the compound (1.3.I-3).

Also especially preferred are compositions 97.1 to 97.3653 which differ from the corresponding compositions 1.1 to 1.3653 only in that they comprise the compound (1.15.I-38) in place of the compound (1.3.I-3).

Also especially preferred are compositions 98.1 to 98.3653 which differ from the corresponding compositions 1.1 to 1.3653 only in that they comprise the compound (1.15.I-39) in place of the compound (1.3.I-3).

Also especially preferred are compositions 99.1 to 99.3653 which differ from the corresponding compositions 1.1 to 1.3653 only in that they comprise the compound (1.15.I-45) in place of the compound (1.3.I-3).

Also especially preferred are compositions 100.1 to 100.3653 which differ from the corresponding compositions 1.1 to 1.3653 only in that they comprise the compound (1.15.I-46) in place of the compound (1.3.I-3).

Also especially preferred are compositions 101.1 to 101.3653 which differ from the corresponding compositions 1.1 to 1.3653 only in that they comprise the compound (1.15.I-52) in place of the compound (1.3.I-3).

Also especially preferred are compositions 102.1 to 102.3653 which differ from the corresponding compositions 1.1 to 1.3653 only in that they comprise the compound (1.15.I-53) in place of the compound (1.3.I-3).

Also especially preferred are compositions 103.1 to 103.3653 which differ from the corresponding compositions 1.1 to 1.3653 only in that they comprise the compound (1.15.I-59) in place of the compound (1.3.I-3).

Also especially preferred are compositions 104.1 to 104.3653 which differ from the corresponding compositions 1.1 to 1.3653 only in that they comprise the compound (1.15.I-60) in place of the compound (1.3.I-3).

Also especially preferred are compositions 105.1 to 105.3653 which differ from the corresponding compositions 1.1 to 1.3653 only in that they comprise the compound (1.15.I-66) in place of the compound (1.3.I-3).

Also especially preferred are compositions 106.1 to 106.3653 which differ from the corresponding compositions 1.1 to 1.3653 only in that they comprise the compound (1.15.I-67) in place of the compound (1.3.I-3).

Also especially preferred are compositions 107.1 to 107.3653 which differ from the corresponding compositions 1.1 to 1.3653 only in that they comprise the compound (1.16.I-3) in place of the compound (1.3.I-3).

Also especially preferred are compositions 108.1 to 108.3653 which differ from the corresponding compositions 1.1 to 1.3653 only in that they comprise the compound (1.16.I-4) in place of the compound (1.3.I-3).

Also especially preferred are compositions 109.1 to 109.3653 which differ from the corresponding compositions 1.1 to 1.3653 only in that they comprise the compound (1.16.I-10) in place of the compound (1.3.I-3).

Also especially preferred are compositions 110.1 to 110.3653 which differ from the corresponding compositions 1.1 to 1.3653 only in that they comprise the compound (1.16.I-11) in place of the compound (1.3.I-3).

Also especially preferred are compositions 111.1 to 111.3653 which differ from the corresponding compositions 1.1 to 1.3653 only in that they comprise the compound (1.16.I-17) in place of the compound (1.3.I-3).

Also especially preferred are compositions 112.1 to 112.3653 which differ from the corresponding compositions 1.1 to 1.3653 only in that they comprise the compound (1.16.I-18) in place of the compound (1.3.I-3).

Also especially preferred are compositions 113.1 to 113.3653 which differ from the corresponding compositions 1.1 to 1.3653 only in that they comprise the compound (1.16.I-24) in place of the compound (1.3.I-3).

Also especially preferred are compositions 114.1 to 114.3653 which differ from the corresponding compositions 1.1 to 1.3653 only in that they comprise the compound (1.16.I-25) in place of the compound (1.3.I-3).

Also especially preferred are compositions 115.1 to 115.3653 which differ from the corresponding compositions 1.1 to 1.3653 only in that they comprise the compound (1.16.I-31) in place of the compound (1.3.I-3).

Also especially preferred are compositions 116.1 to 116.3653 which differ from the corresponding compositions 1.1 to 1.3653 only in that they comprise the compound (1.16.I-32) in place of the compound (1.3.I-3).

Also especially preferred are compositions 117.1 to 117.3653 which differ from the corresponding compositions 1.1 to 1.3653 only in that they comprise the compound (1.16.I-38) in place of the compound (1.3.I-3).

Also especially preferred are compositions 118.1 to 118.3653 which differ from the corresponding compositions 1.1 to 1.3653 only in that they comprise the compound (1.16.I-39) in place of the compound (1.3.I-3).

Also especially preferred are compositions 119.1 to 119.3653 which differ from the corresponding compositions 1.1 to 1.3653 only in that they comprise the compound (1.16.I-45) in place of the compound (1.3.I-3).

Also especially preferred are compositions 120.1 to 120.3653 which differ from the corresponding compositions 1.1 to 1.3653 only in that they comprise the compound (1.16.I-46) in place of the compound (1.3.I-3).

Also especially preferred are compositions 121.1 to 121.3653 which differ from the corresponding compositions 1.1 to 1.3653 only in that they comprise the compound (1.16.I-52) in place of the compound (1.3.I-3).

Also especially preferred are compositions 122.1 to 122.3653 which differ from the corresponding compositions 1.1 to 1.3653 only in that they comprise the compound (1.16.I-53) in place of the compound (1.3.I-3).

Also especially preferred are compositions 123.1 to 123.3653 which differ from the corresponding compositions 1.1 to 1.3653 only in that they comprise the compound (1.16.I-59) in place of the compound (1.3.I-3).

Also especially preferred are compositions 124.1 to 124.3653 which differ from the corresponding compositions 1.1 to 1.3653 only in that they comprise the compound (1.16.I-60) in place of the compound (1.3.I-3).

Also especially preferred are compositions 125.1 to 125.3653 which differ from the corresponding compositions 1.1 to 1.3653 only in that they comprise the compound (1.16.I-66) in place of the compound (1.3.I-3).

Also especially preferred are compositions 126.1 to 126.3653 which differ from the corresponding compositions 1.1 to 1.3653 only in that they comprise the compound (1.16.I-67) in place of the compound (1.3.I-3).

Also especially preferred are compositions 127.1 to 127.3653 which differ from the corresponding compositions 1.1 to 1.3653 only in that they comprise the compound (1.15.I-18) in place of the compound (1.3.I-3).

The invention also relates to agrochemical compositions comprising at least an auxiliary and at least one pyrimidine compound of formula (I) according to the invention.

An agrochemical composition comprises a pesticidal effective amount of a pyrimidine compound of formula (I). The term “effective amount” denotes an amount of the composition or of the compounds I, which is sufficient for controlling unwanted plants, especially for controlling unwanted plants in cultivated plants and which does not result in a substantial damage to the treated plants. Such an amount can vary in a broad range and is dependent on various factors, such as the plants to be controlled, the treated cultivated plant or material, the climatic conditions and the specific pyrimidine compound of formula (I) used.

The pyrimidine compounds of formula (I), their N-oxides, salts or derivatives can be converted into customary types of agrochemical compositions, e. g. solutions, emulsions, suspensions, dusts, powders, pastes, granules, pressings, capsules, and mixtures thereof. Examples for agrochemical composition types are suspensions (e.g. SC, OD, FS), emulsifiable concentrates (e.g. EC), emulsions (e.g. EW, EO, ES, ME), capsules (e.g. CS, ZC), pastes, pastilles, wettable powders or dusts (e.g. WP, SP, WS, DP, DS), pressings (e.g. BR, TB, DT), granules (e.g. WG, SG, GR, FG, GG, MG), insecticidal articles (e.g. LN), as well as gel formulations for the treatment of plant propagation materials such as seeds (e.g. GF). These and further agrochemical compositions types are defined in the “Catalogue of pesticide formulation types and international coding system”, Technical Monograph No. 2, 6^{th} Ed. May 2008, CropLife International.

The agrochemical compositions are prepared in a known manner, such as described by Mollet and Grubemann, Formulation technology, Wiley VCH, Weinheim, 2001; or Knowles, New developments in crop protection product formulation, Agrow Reports DS243, T&F Informa, London, 2005.

Suitable auxiliaries are solvents, liquid carriers, solid carriers or fillers, surfactants, dispersants, emulsifiers, wetters, adjuvants, solubilizers, penetration enhancers, protective colloids, adhesion agents, thickeners, humectants, repellents, attractants, feeding stimulants, compatibilizers, bactericides, anti-freezing agents, anti-foaming agents, colorants, tackifiers and binders.

Suitable solvents and liquid carriers are water and organic solvents, such as mineral oil fractions of medium to high boiling point, e.g. kerosene, diesel oil; oils of vegetable or animal origin; aliphatic, cyclic, and aromatic hydrocarbons, e. g. toluene, paraffin, tetrahydronaphthalene, alkylated naphthalenes; alcohols, e.g. ethanol, propanol, butanol, benzylalcohol, cyclohexanol; glycols; DMSO; ketones, e.g. cyclohexanone; esters, e.g. lactates, carbonates, fatty acid esters, gamma-butyrolactone; fatty acids; phosphonates; amines; amides, e.g. N-methylpyrrolidone, fatty acid dimethylamides; and mixtures thereof.

Suitable solid carriers or fillers are mineral earths, e.g. silicates, silica gels, talc, kaolins, limestone, lime, chalk, clays, dolomite, diatomaceous earth, bentonite, calcium sulfate, magnesium sulfate, magnesium oxide; polysaccharides, e.g. cellulose, starch; fertilizers, e.g. ammonium sulfate, ammonium phosphate, ammonium nitrate, ureas; products of vegetable origin, e.g. cereal meal, tree bark meal, wood meal, nutshell meal, and mixtures thereof.

Suitable surfactants are surface-active compounds, such as anionic, cationic, non-ionic, and amphoteric surfactants, block polymers, polyelectrolytes, and mixtures thereof. Such surfactants can be used as emulsifier, dispersant, solubilizer, wetter, penetration enhancer, protective colloid, or adjuvant. Examples of surfactants are listed in McCutcheon's, Vol. 1: Emulsifiers & Detergents, McCutcheon's Directories, Glen Rock, USA, 2008 (International Ed. or North American Ed.).

Suitable anionic surfactants are alkali, alkaline earth or ammonium salts of sulfonates, sulfates, phosphates, carboxylates, and mixtures thereof. Examples of sulfonates are alkylarylsulfonates, diphenylsulfonates, alpha-olefin sulfonates, lignine sulfonates, sulfonates of fatty acids and oils, sulfonates of ethoxylated alkylphenols, sulfonates of alkoxylated arylphenols, sulfonates of condensed naphthalenes, sulfonates of dodecyl- and tridecylbenzenes, sulfonates of naphthalenes and alkylnaphthalenes, sulfosuccinates or sulfosuccinamates. Examples of sulfates are sulfates of fatty acids and oils, of ethoxylated alkylphenols, of alcohols, of ethoxylated alcohols, or of fatty acid esters. Examples of phosphates are phosphate esters. Examples of carboxylates are alkyl carboxylates, and carboxylated alcohol or alkylphenol ethoxylates.

Suitable nonionic surfactants are alkoxylates, N-substituted fatty acid amides, amine oxides, esters, sugar-based surfactants, polymeric surfactants, and mixtures thereof. Examples of alkoxylates are compounds such as alcohols, alkylphenols, amines, amides, arylphenols, fatty acids, or fatty acid esters which have been alkoxylated with 1 to 50 equivalents. Ethylene oxide and/or propylene oxide may be employed for the alkoxylation, preferably ethylene oxide. Examples of N-substituted fatty acid amides are fatty acid glucamides or fatty acid alkanolamides. Examples of esters are fatty acid esters, glycerol esters, or monoglycerides. Examples of sugar-based surfactants are sorbitans, ethoxylated sorbitans, sucrose, and glucose esters, or alkylpoly-glucosides. Examples of polymeric surfactants are home- or copolymers of vinylpyrrolidone, vinylalcohols, or vinylacetate.

Suitable cationic surfactants are quaternary surfactants, e.g. quaternary ammonium compounds with one or two hydrophobic groups, or salts of long-chain primary amines. Suitable amphoteric surfactants are alkylbetains and imidazolines. Suitable block polymers are block polymers of the A-B or A-B-A type comprising blocks of polyethylene oxide and polypropylene oxide, or of the A-B—C type comprising alkanol, polyethylene oxide and polypropylene oxide. Suitable polyelectrolytes are polyacids or polybases. Examples of polyacids are alkali salts of polyacrylic acid or polyacid comb polymers. Examples of polybases are polyvinylamines or polyethyleneamines.

Suitable adjuvants are compounds, which have a neglectable or even no pesticidal activity themselves, and which improve the biological performance of the pyrimidine compounds of formula (I) on the target. Examples are surfactants, mineral or vegetable oils, and other auxiliaries. Further examples are listed by Knowles, Adjuvants, and additives, Agrow Reports DS256, T&F Informa UK, 2006, chapter 5.

Suitable thickeners are polysaccharides (e.g. xanthan gum, carboxymethylcellulose), inorganic clays (organically modified or unmodified), polycarboxylates, and silicates.

Suitable bactericides are bronopol and isothiazolinone derivatives such as alkylisothiazolinones and benzisothiazolinones.

Suitable anti-freezing agents are ethylene glycol, propylene glycol, urea, and glycerin.

Suitable anti-foaming agents are silicones, long chain alcohols, and salts of fatty acids.

Suitable colorants (e.g. in red, blue, or green) are pigments of low water solubility and water-soluble dyes. Examples are inorganic colorants (e.g. iron oxide, titan oxide, iron hexacyanoferrate) and organic colorants (e.g. alizarin-, azo-, and phthalocyanine colorants).

Suitable tackifiers or binders are polyvinylpyrrolidons, polyvinylacetates, polyvinyl alcohols, polyacrylates, biological or synthetic waxes, and cellulose ethers.

Examples for agrochemical composition types and their preparation are:

i) Water-Soluble Concentrates (SL, LS)

10-60 wt % of a pyrimidine compound of formula (I) according to the invention and 5-15 wt % wetting agent (e.g. alcohol alkoxylates) are dissolved in water and/or in a water-soluble solvent (e.g. alcohols) ad 100 wt %. The active substance dissolves upon dilution with water.

ii) Dispersible Concentrates (DC)

5-25 wt % of a pyrimidine compound of formula (I) according to the invention and 1-10 wt % dispersant (e. g. polyvinylpyrrolidone) are dissolved in organic solvent (e.g. cyclohexanone) ad 100 wt %. Dilution with water gives a dispersion.

iii) Emulsifiable Concentrates (EC)

15-70 wt % of a pyrimidine compound of formula (I) according to the invention and 5-10 wt % emulsifiers (e.g. calcium dodecylbenzenesulfonate and castor oil ethoxylate) are dissolved in water-insoluble organic solvent (e.g. aromatic hydrocarbon) ad 100 wt %. Dilution with water gives an emulsion.

iv) Emulsions (EW, EO, ES)

5-40 wt % of a pyrimidine compound of formula (I) according to the invention and 1-10 wt % emulsifiers (e.g. calcium dodecylbenzenesulfonate and castor oil ethoxylate) are dissolved in 20-40 wt % water-insoluble organic solvent (e.g. aromatic hydrocarbon). This mixture is introduced into water ad 100 wt % by means of an emulsifying machine and made into a homogeneous emulsion. Dilution with water gives an emulsion.

v) Suspensions (SC, OD, FS)

In an agitated ball mill, 20-60 wt % of a pyrimidine compound of formula (I) according to the invention are comminuted with addition of 2-10 wt % dispersants and wetting agents (e.g. sodium lignosulfonate and alcohol ethoxylate), 0, 1-2 wt % thickener (e.g. xanthan gum) and water ad 100 wt % to give a fine active substance suspension. Dilution with water gives a stable suspension of the active substance. For FS type composition up to 40 wt % binder (e.g. polyvinylalcohol) is added.

vi) Water-Dispersible Granules and Water-Soluble Granules (WG, SG)

50-80 wt % of a pyrimidine compound of formula (I) according to the invention are ground finely with addition of dispersants and wetting agents (e.g. sodium lignosulfonate and alcohol ethoxylate) ad 100 wt % and prepared as water-dispersible or water-soluble granules by means of technical appliances (e. g. extrusion, spray tower, fluidized bed). Dilution with water gives a stable dispersion or solution of the active substance.

vii) Water-Dispersible Powders and Water-Soluble Powders (WP, SP, WS)

50-80 wt % of a pyrimidine compound of formula (I) according to the invention are ground in a rotor-stator mill with addition of 1-5 wt % dispersants (e.g. sodium lignosulfonate), 1-3 wt % wetting agents (e.g. alcohol ethoxylate) and solid carrier (e.g. silica gel) ad 100 wt %. Dilution with water gives a stable dispersion or solution of the active substance.

viii) Gel (GW, GF)

In an agitated ball mill, 5-25 wt % of a pyrimidine compound of formula (I) according to the invention are comminuted with addition of 3-10 wt % dispersants (e.g. sodium lignosulfonate), 1-5 wt % thickener (e.g. carboxymethylcellulose) and water ad 100 wt % to give a fine suspension of the active substance. Dilution with water gives a stable suspension of the active substance.

iv) Microemulsion (ME)

5-20 wt % of a pyrimidine compound of formula (I) according to the invention are added to 5-30 wt % organic solvent blend (e.g. fatty acid dimethylamide and cyclohexanone), 10-25 wt % surfactant blend (e.g. alcohol ethoxylate and arylphenol ethoxylate), and water ad 100%. This mixture is stirred for 1 h to produce spontaneously a thermodynamically stable microemulsion.

iv) Microcapsules (CS)

An oil phase comprising 5-50 wt % of a pyrimidine compound of formula (I) according to the invention, 0-40 wt % water insoluble organic solvent (e.g. aromatic hydrocarbon), 2-15 wt % acrylic monomers (e.g. methylmethacrylate, methacrylic acid, and a di- or triacrylate) are dispersed into an aqueous solution of a protective colloid (e.g. polyvinyl alcohol). Radical polymerization initiated by a radical initiator results in the formation of poly(meth)acrylate microcapsules. Alternatively, an oil phase comprising 5-50 wt % of a pyrimidine compound of formula (I) according to the invention, 0-40 wt % water insoluble organic solvent (e.g. aromatic hydrocarbon), and an isocyanate monomer (e.g. diphenylmethene-4,4′-diisocyanate) are dispersed into an aqueous solution of a protective colloid (e.g. polyvinyl alcohol). The addition of a polyamine (e.g. hexamethylenediamine) results in the formation of polyurea microcapsules. The monomers amount to 1-10 wt %. The wt % relate to the total CS composition.

ix) Dustable Powders (DP, DS)

1-10 wt % of a pyrimidine compound of formula (I) according to the invention are ground finely and mixed intimately with solid carrier (e.g. finely divided kaolin) ad 100 wt %.

x) Granules (GR, FG)

0.5-30 wt % of a pyrimidine compound of formula (I) according to the invention is ground finely and associated with solid carrier (e.g. silicate) ad 100 wt %. Granulation is achieved by extrusion, spray-drying or the fluidized bed.

xi) Ultra-Low Volume Liquids (UL)

1-50 wt % of a pyrimidine compound of formula (I) according to the invention are dissolved in organic solvent (e.g. aromatic hydrocarbon) ad 100 wt %.

The agrochemical compositions types i) to xi) may optionally comprise further auxiliaries, such as 0.1-1 wt % bactericides, 5-15 wt % anti-freezing agents, 0.1-1 wt % anti-foaming agents, and 0.1-1 wt % colorants.

The agrochemical compositions comprising generally comprise between 0.01 and 95%, preferably between 0.1 and 90%, and in particular between 0.5 and 75%, by weight of the pyrimidine compound of formula (I). The pyrimidine compounds of formula (I) are employed in a purity of from 90% to 100%, preferably from 95% to 100% (according to NMR spectrum).

Solutions for seed treatment (LS), suspoemulsions (SE), flowable concentrates (FS), powders for dry treatment (DS), water-dispersible powders for slurry treatment (WS), water-soluble powders (SS), emulsions (ES), emulsifiable concentrates (EC) and gels (GF) are usually employed for the purposes of treatment of plant propagation materials, particularly seeds. The agrochemical compositions in question give, after two-to-tenfold dilution, active substance concentrations of from 0.01 to 60% by weight, preferably from 0.1 to 40% by weight, in the ready-to-use preparations. Application can be carried out before or during sowing.

Methods for applying pyrimidine compounds of formula (I) and agrochemical compositions thereof, on to plant propagation material, especially seeds, include dressing, coating, pelleting, dusting, soaking and in-furrow application methods of the propagation material. Preferably, pyrimidine compounds of formula (I) and agrochemical compositions thereof, are applied on to the plant propagation material by a method such that germination is not induced, e. g. by seed dressing, pelleting, coating and dusting.

Various types of oils, wetters, adjuvants, fertilizer, or micronutrients, and further pesticides (e.g. herbicides, insecticides, fungicides, growth regulators, safeners) may be added to the pyrimidine compounds of formula (I) and the agrochemical compositions comprising them as premix or, if appropriate not until immediately prior to use (tank mix). These agents can be admixed with the agrochemical compositions according to the invention in a weight ratio of 1:100 to 100:1, preferably 1:10 to 10:1.

The user applies the pyrimidine compound of formula (I) according to the invention and the agrochemical compositions comprising them usually from a pre-dosage device, a knapsack sprayer, a spray tank, a spray plane, or an irrigation system. Usually, the agrochemical composition is made up with water, buffer, and/or further auxiliaries to the desired application concentration and the ready-to-use spray liquor or the agrochemical composition according to the invention is thus obtained. Usually, 20 to 2000 liters, preferably 50 to 400 liters, of the ready-to-use spray liquor are applied per hectare of agricultural useful area.

According to one embodiment, either individual components of the agrochemical composition according to the invention or partially premixed components, e. g. components comprising pyrimidine compounds of formula (I) may be mixed by the user in a spray tank and further auxiliaries and additives may be added, if appropriate.

In a further embodiment, individual components of the agrochemical composition according to the invention such as parts of a kit or parts of a binary or ternary mixture may be mixed by the user himself in a spray tank and further auxiliaries may be added, if appropriate.

In a further embodiment, either individual components of the agrochemical composition according to the invention or partially premixed components, e. g components comprising pyrimidine compounds of formula (I) can be applied jointly (e.g. after tank mix) or consecutively.

The pyrimidine compounds of formula (I), are suitable as herbicides. They are suitable as such or as an appropriately formulated composition (agrochemical composition).

The pyrimidine compounds of formula (I), or the agrochemical compositions comprising the pyrimidine compounds of formula (I), control vegetation on non-crop areas very efficiently, especially at high rates of application. They act against broad-leaved weeds and grass weeds in crops such as wheat, rice, maize, soya, and cotton without causing any significant damage to the crop plants. This effect is mainly observed at low rates of application.

The pyrimidine compounds of formula (I), or the agrochemical compositions comprising them, are applied to the plants mainly by spraying the leaves. Here, the application can be carried out using, e.g., water as carrier by customary spraying techniques using spray liquor amounts of from about 100 to 1000 l/ha (e.g. from 300 to 400 l/ha). The pyrimidine compounds of formula (I), or the agrochemical compositions comprising them, may also be applied by the low-volume or the ultra-low-volume method, or in the form of micro granules.

Application of the pyrimidine compounds of formula (I), or the agrochemical compositions comprising them, can be done before, during, and/or after, preferably during and/or after, the emergence of the undesirable plants.

The pyrimidine compounds of formula (I), or the agrochemical compositions comprising them, can be applied pre-, post-emergence or pre-plant, or together with the seed of a crop plant. It is also possible to apply the pyrimidine compounds of formula (I), or the agrochemical compositions comprising them, by applying seed, pretreated with the pyrimidine compounds of formula (I), or the agrochemical compositions comprising them, of a crop plant. If the active ingredients are less well tolerated by certain crop plants, application techniques may be used in which the herbicidal compositions are sprayed, with the aid of the spraying equipment, in such a way that as far as possible they do not come into contact with the leaves of the sensitive crop plants, while the active ingredients reach the leaves of undesirable plants growing underneath, or the bare soil surface (post-directed, lay-by).

In a further embodiment, the pyrimidine compounds of formula (I), or the agrochemical compositions comprising them, can be applied by treating seed. The treatment of seeds comprises essentially all procedures familiar to the person skilled in the art (seed dressing, seed coating, seed dusting, seed soaking, seed film coating, seed multilayer coating, seed encrusting, seed dripping and seed pelleting) based on the pyrimidine compounds of formula (I), or the agrochemical compositions prepared therefrom. Here, the herbicidal compositions can be applied diluted or undiluted.

The term “seed” comprises seed of all types, such as, e.g., corns, seeds, fruits, tubers, seedlings and similar forms. Here, preferably, the term seed describes corns and seeds. The seed used can be seed of the useful plants mentioned above, but also the seed of transgenic plants or plants obtained by customary breeding methods.

When employed in plant protection, the amounts of active substances applied, i.e. the pyrimidine compounds of formula (I) without formulation auxiliaries, are, depending on the kind of effect desired, from 0.001 to 2 kg per ha, preferably from 0.005 to 2 kg per ha, more preferably from 0.05 to 0.9 kg per ha and in particular from 0.1 to 0.75 kg per ha.

In another embodiment of the invention, the application rate of the pyrimidine compounds of formula (I) is from 0.001 to 3 kg/ha, preferably from 0.005 to 2.5 kg/ha and in particular from 0.01 to 2 kg/ha of active substance (a.s.).

In another preferred embodiment of the invention, the rates of application of the pyrimidine compounds of formula (I) according to the present invention (total amount of pyrimidine compounds of formula (I)) are from 0.1 g/ha to 3000 g/ha, preferably 10 g/ha to 1000 g/ha, depending on the control target, the season, the target plants and the growth stage.

In another preferred embodiment of the invention, the application rates of the pyrimidine compounds of formula (I) are in the range from 0.1 g/ha to 5000 g/ha and preferably in the range from 1 g/ha to 2500 g/ha or from 5 g/ha to 2000 g/ha.

In another preferred embodiment of the invention, the application rate of the pyrimidine compounds of formula (I) is 0.1 to 1000 g/ha, preferably 1 to 750 g/ha, more preferably 5 to 500 g/ha.

In treatment of plant propagation materials such as seeds, e. g. by dusting, coating or drenching seed, amounts of active substance of from 0.1 to 1000 g, preferably from 1 to 1000 g, more preferably from 1 to 100 g, and most preferably from 5 to 100 g, per 100 kilogram of plant propagation material (preferably seeds) are generally required.

In another embodiment of the invention, to treat the seed, the amounts of active substances applied, i.e. the pyrimidine compounds of formula (I) are generally employed in amounts of from 0.001 to 10 kg per 100 kg of seed.

When used in the protection of materials or stored products, the amount of active substance applied depends on the kind of application area and on the desired effect. Amounts customarily applied in the protection of materials are 0.001 g to 2 kg, preferably 0.005 g to 1 kg, of active substance per cubic meter of treated material.

Depending on the application method in question, the pyrimidine compounds of formula (I), or the agrochemical compositions comprising them, can additionally be employed in a further number of crop plants for eliminating undesirable plants. Examples of suitable crops are the following: Allium cepa, Ananas comosus, Arachis hypogaea, Asparagus officinalis, Avena sativa, Beta vulgaris spec. altissima, Beta vulgaris spec. rapa, Brassica napus var. napus, Brassica napus var. napobrassica, Brassica rapa var. silvestris, Brassica oleracea, Brassica nigra, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N.rustica), Olea europaea, Oryza sativa, Phaseolus lunatus, Phaseolus vulgaris, Picea abies, Pinus spec., Pistacia vera, Pisum sativum, Prunus avium, Prunus persica, Pyrus communis, Prunus armeniaca, Prunus cerasus, Prunus dulcis and Prunus domestica, Ribes sylvestre, Ricinus communis, Saccharum officinarum, Secale cereale, Sinapis alba, Solanum tuberosum, Sorghum bicolor (s. vulgare), Theobroma cacao, Trifolium pratense, Triticum aestivum, Triticale, Triticum durum, Vicia faba, Vitis vinifera, and Zea mays.

Preferred crops are Arachis hypogaea, Beta vulgaris spec. altissima, Brassica napus var. napus, Brassica oleracea, Citrus limon, Citrus sinensis, Coffea arabica (Coffea canephora, Coffea liberica), Cynodon dactylon, Glycine max, Gossypium hirsutum, (Gossypium arboreum, Gossypium herbaceum, Gossypium vitifolium), Helianthus annuus, Hordeum vulgare, Juglans regia, Lens culinaris, Linum usitatissimum, Lycopersicon lycopersicum, Malus spec., Medicago sativa, Nicotiana tabacum (N. rustica), Olea europaea, Oryza sativa, Phaseolus lunatus, Phaseolus vulgaris, Pistacia vera, Pisum sativum, Prunus dulcis, Saccharum officinarum, Secale cereale, Solanum tuberosum, Sorghum bicolor (s. vulgare), Triticale, Triticum aestivum, Triticum durum, Vicia faba, Vitis vinifera, and Zea mays.

Especially preferred crops are crops of cereals, corn, soybeans, rice, oilseed rape, cotton, potatoes, peanuts, or permanent crops.

The pyrimidine compounds of formula (I) according to the invention, or the agrochemical compositions comprising them, can also be used in genetically modified plants. The term “genetically modified plants” is to be understood as plants whose genetic material has been modified by the use of recombinant DNA techniques to include an inserted sequence of DNA that is not native to that plant species' genome or to exhibit a deletion of DNA that was native to that species' genome, wherein the modification(s) cannot readily be obtained by cross breeding, mutagenesis or natural recombination alone. Often, a particular genetically modified plant will be one that has obtained its genetic modification(s) by inheritance through a natural breeding or propagation process from an ancestral plant whose genome was the one directly treated by use of a recombinant DNA technique. Typically, one or more genes have been integrated into the genetic material of a genetically modified plant in order to improve certain properties of the plant. Such genetic modifications also include but are not limited to targeted post-translational modification of protein(s), oligo- or polypeptides. e. g., by inclusion therein of amino acid mutation(s) that permit, decrease, or promote glycosylation or polymer additions such as prenylation, acetylation farnesylation, or PEG moiety attachment.

Plants that have been modified by breeding, mutagenesis or genetic engineering, e.g. have been rendered tolerant to applications of specific classes of herbicides, such as auxin herbicides such as dicamba or 2,4-D; bleacher herbicides such as hydroxyphenylpyruvate dioxygenase (HPPD) inhibitors or phytoene desaturase (PDS) inhibitors; acetolactate synthase (ALS) inhibitors such as sulfonyl ureas or imidazolinones; enolpyruvyl shikimate 3-phosphate synthase (EPSP) inhibitors such as glyphosate; glutamine synthetase (GS) inhibitors such as glufosinate; protoporphyrinogen-IX oxidase inhibitors; lipid biosynthesis inhibitors such as acetyl CoA carboxylase (ACCase) inhibitors; or oxynil (i. e. bromoxynil or ioxynil) herbicides as a result of conventional methods of breeding or genetic engineering; furthermore, plants have been made resistant to multiple classes of herbicides through multiple genetic modifications, such as resistance to both glyphosate and glufosinate or to both glyphosate and a herbicide from another class such as ALS inhibitors, HPPD inhibitors, auxin herbicides, or ACCase inhibitors. These herbicide resistance technologies are, e.g., described in Pest Management Science 61, 2005, 246; 61, 2005, 258; 61, 2005, 277; 61, 2005, 269; 61, 2005, 286; 64, 2008, 326; 64, 2008, 332; Weed Science 57, 2009, 108; Australian Journal of Agricultural Research 58, 2007, 708; Science 316, 2007, 1185; and references quoted therein. Several cultivated plants have been rendered tolerant to herbicides by mutagenesis and conventional methods of breeding, e. g., Clearfield® summer rape (Canola, BASF SE, Germany) being tolerant to imidazolinones, e. g., imazamox, or ExpressSun® sunflowers (DuPont, USA) being tolerant to sulfonyl ureas, e. g., tribenuron. Genetic engineering methods have been used to render cultivated plants such as soybean, cotton, corn, beets and rape, tolerant to herbicides such as glyphosate, imidazolinones, and glufosinate, some of which are under development or commercially available under the brands or trade names RoundupReady® (glyphosate tolerant, Monsanto, USA), Cultivance® (imidazolinone tolerant, BASF SE, Germany), and LibertyLink® (glufosinate tolerant, Bayer CropScience, Germany).

Furthermore, plants are also covered that are by the use of recombinant DNA techniques capable to synthesize one or more insecticidal proteins, especially those known from the bacterial genus Bacillus, particularly from Bacillus thuringiensis, such as delta-endotoxins, e. g., CryIA(b), CryIA(c), CryIF, CryIF(a2), CryIIA(b), CryIIIA, CryIIIB(b1) or Cry9c; vegetative insecticidal proteins (VIP), e. g., VIP1, VIP2, VIP3, or VIP3A; insecticidal proteins of bacteria colonizing nematodes, e. g., Photorhabdus spp. or Xenorhabdus spp.; toxins produced by animals, such as scorpion toxins, arachnid toxins, wasp toxins, or other insect-specific neurotoxins; toxins produced by fungi, such as Streptomycetes toxins, plant lectins, such as pea or barley lectins; agglutinins; proteinase inhibitors, such as trypsin inhibitors, serine protease inhibitors, patatin, cystatin or papain inhibitors; ribosome-inactivating proteins (RIP), such as ricin, maize-RIP, abrin, luffin, saporin or bryodin; steroid metabolism enzymes, such as 3-hydroxy-steroid oxidase, ecdysteroid-IDP-glycosyl-transferase, cholesterol oxidases, ecdysone inhibitors or HMG-CoA-reductase; ion channel blockers, such as blockers of sodium or calcium channels; juvenile hormone esterase; diuretic hormone receptors (helicokinin receptors); stilbene synthase, bibenzyl synthase, chitinases or glucanases. In the context of the present invention these insecticidal proteins or toxins are to be understood expressly also as including pre-toxins, hybrid proteins, truncated or otherwise modified proteins. Hybrid proteins are characterized by a new combination of protein domains, (see, e. g., WO 02/015701). Further examples of such toxins or genetically modified plants capable of synthesizing such toxins are disclosed, e. g., in EP-A 374 753, WO 93/007278, WO 95/34656, EP-A 427 529, EP-A 451 878, WO 03/18810, and WO 03/52073. The methods for producing such genetically modified plants are generally known to the person skilled in the art and are described, e. g., in the publications mentioned above. These insecticidal proteins contained in the genetically modified plants impart to the plants producing these proteins tolerance to harmful pests from all taxonomic groups of arthropods, especially to beetles (Coeloptera), two-winged insects (Diptera), and moths (Lepidoptera) and to nematodes (Nematoda). Genetically modified plants capable to synthesize one or more insecticidal proteins are, e. g., described in the publications mentioned above, and some of which are commercially available such as YieldGard® (corn cultivars producing the Cry1Ab toxin), YieldGard® Plus (corn cultivars producing Cry1Ab and Cry3Bb1 toxins), Starlink® (corn cultivars producing the Cry9c toxin), Herculex® RW (corn cultivars producing Cry34Ab1, Cry35Ab1 and the enzyme Phosphinothricin-N-Acetyltransferase [PAT]); NuCOTN® 33B (cotton cultivars producing the Cry1Ac toxin), Bollgard® I (cotton cultivars producing the Cry1Ac toxin), Bollgard® II (cotton cultivars producing Cry1Ac and Cry2Ab2 toxins); VIPCOT® (cotton cultivars producing a VIP-toxin); NewLeaf® (potato cultivars producing the Cry3A toxin); Bt-Xtra®, NatureGard®, KnockOut®, BiteGard®, Protecta®, Bt11 (e. g., Agrisure® CB) and Bt176 from Syngenta Seeds SAS, France, (corn cultivars producing the Cry1Ab toxin and PAT enzyme), MIR604 from Syngenta Seeds SAS, France (corn cultivars producing a modified version of the Cry3A toxin, c.f. WO 03/018810), MON 863 from Monsanto Europe S.A., Belgium (corn cultivars producing the Cry3Bb1 toxin), IPC 531 from Monsanto Europe S.A., Belgium (cotton cultivars producing a modified version of the Cry1Ac toxin) and 1507 from Pioneer Overseas Corporation, Belgium (corn cultivars producing the Cry1F toxin and PAT enzyme).

Furthermore, plants are also covered that are by the use of recombinant DNA techniques capable to synthesize one or more proteins to increase the resistance or tolerance of those plants to bacterial, viral or fungal pathogens. Examples of such proteins are the so-called “pathogenesis-related proteins” (PR proteins, see, e.g., EP-A 392 225), plant disease resistance genes (e. g., potato culti-vars, which express resistance genes acting against Phytophthora infestans derived from the Mexican wild potato, Solanum bulbocastanum) or T4-lyso-zym (e.g., potato cultivars capable of synthesizing these proteins with increased resistance against bacteria such as Erwinia amylovora). The methods for producing such genetically modi-fied plants are generally known to the person skilled in the art and are described, e.g., in the publications mentioned above.

Furthermore, plants are also covered that are by the use of recombinant DNA techniques capable to synthesize one or more proteins to increase the productivity (e.g., bio-mass production, grain yield, starch content, oil content or protein content), tolerance to drought, salinity or other growth-limiting environmental factors or tolerance to pests and fungal, bacterial or viral pathogens of those plants.

Furthermore, plants are also covered that contain by the use of recombinant DNA techniques a modified amount of ingredients or new ingredients, specifically to improve human or animal nutrition, e. g., oil crops that produce health-promoting long-chain omega-3 fatty acids or unsaturated omega-9 fatty acids (e. g., Nexera® rape, Dow AgroSciences, Canada).

Furthermore, plants are also covered that contain by the use of recombinant DNA techniques a modified amount of ingredients or new ingredients, specifically to improve raw material production, e.g., potatoes that produce increased amounts of amylopectin (e.g. Amflora® potato, BASF SE, Germany).

The preparation of the pyrimidine compounds of formula (I) is illustrated by the following examples.

### A PREPARATION EXAMPLES

### Example 1

### 1.1 (Z)-3-(1,3-benzodioxol-5-yl)-4-(dimethylamino)but-3-en-2-one

A solution of 1-(1,3-benzodioxol-5-yl)propan-2-one (CAS 4676-39-5, 36.7 g) in dimethylformamidedimethylacetal (240 mL) was stirred for 16 h at 60° C. TLC showed that the SM was consumed. The mixture was concentrated to give (Z)-3-(1,3-benzodioxol-5-yl)-4-(dimethylamino)but-3-en-2-one (45.0 g, 93.7%), which was directly used in the next step.

HPLC/MS Rₜ: 0.677 min; m/z: 234.0 [M+H+]

### 1.2 5-(1,3-benzodioxol-5-yl)-2-cyclopropyl-4-methyl-pyrimidine

To a solution of (Z)-3-(1,3-benzodioxol-5-yl)-4-(dimethylamino)but-3-en-2-one (45.0 g) and cyclopropanecarboxamidine hydrochloride (46.5 g) in EtOH (1300 mL) was added slowly NaOEt (32.8 g). The resulting mixture was stirred for 16 h at reflux. The mixture was concentrated, the residue was taken up in EtOAc, washed with water, separated and concentrated to give 5-(1,3-benzodioxol-5-yl)-2-cyclopropyl-4-methyl-pyrimidine (32.0 g, 65.2%), which was directly used in the next step.

HPLC/MS Rₜ: 1.010 min; m/z: 255.0 [M+H+].

### 1.3 5-(6-chloro-1,3-benzodioxol-5-yl)-2-cyclopropyl-4-methyl-pyrimidine

To a solution of 5-(1,3-benzodioxol-5-yl)-2-cyclopropyl-4-methyl-pyrimidine (34.0 g) in DMF (250 mL) was added N-Chlorosuccinimide (21.4 g) at room temperature. The reaction mixture was stirred at 50° C. for 18 h and then water (500 mL) was added. The aqueous phase was extracted with ethyl acetate (3×200 mL). The combined organic layers were dried (MgSO₄), filtered and concentrated in vacuo. The crude product was purified by reverse phase chromatography using a gradient of acetonitrile in water yielding the product (20.4 g, 52.8%).

¹H NMR (400 MHz, Chloroform-d) δ 8.25 (s, 1H), 6.96 (s, 1H), 6.64 (s, 1H), 6.05 (d, J=1.7 Hz, 2H), 2.31 (s, 3H), 2.30-2.22 (m, 1H), 1.21-1.04 (m, 4H). HPLC/MS Rₜ: 1.120 min; m/z: 288.9 [M+H+].

### Example 8: 1.4 1-[[5-(6-chloro-1,3-benzodioxol-5-yl)-2-cyclopropyl-pyrimidin-4-yl]methyl]cyclohexanol

To a solution of 5-(6-chloro-1,3-benzodioxol-5-yl)-2-cyclopropyl-4-methyl-pyrimidine (3.5 g) in THF (60 mL) was added LHMDS (24.2 mL, 1M in THF) at 0° C. The reaction was stirred at 0° C. for 30 min and then cyclohexanone (2.5 mL) was added. The reaction mixture was allowed to warm to room temperature and stirred for 18 h. Hydrochloric acid (1M in water, 25 mL) was added and resulting mixture was extracted with ethyl acetate (3×100 mL). The combined organic layers were dried (MgSO₄), filtered and concentrated in vacuo. The crude product was purified by reverse phase chromatography using a gradient of acetonitrile in water, yielding the final product (1.1 g, 23.5%).

¹H NMR (400 MHz, Chloroform-d) δ 8.32 (s, 1H), 6.97 (s, 1H), 6.61 (s, 1H), 6.12-6.03 (m, 2H), 5.93 (s, 1H), 2.79-2.57 (m, 2H), 2.36-2.18 (m, 1H), 1.74-1.10 (m, 14H). HPLC/MS Rₜ: 1.304 min; m/z: 387.1 [M+H+].

### Examples 9 and 10: 5-(2-chlorophenyl)-4-(cyclohexen-1-ylmethyl)-2-cyclopropyl-pyrimidine and 5-(2-chlorophenyl)-4-(cyclohexylidenemethyl)-2-cyclopropyl-pyrimidine

To a solution of 1-[[5-(6-chloro-1,3-benzodioxol-5-yl)-2-cyclopropyl-pyrimidin-4-yl]methyl]cyclohexanol (1.1 g) in toluene (100 mL) was added p-toluene sulfonic acid (661 mg). The reaction mixture was heated under reflux for 1 h, allowed to cool to room temperature and diluted with ethyl acetate (200 mL). The organic layer was extracted with saturated sodium hydrogen carbonate solution (100 mL). The organic layer was dried (MgSO₄), filtered and concentrated in vacuo. The crude mixture was purified by flash chromatography using a gradient of ethyl acetate in cyclohexane, yielding two isomeric products. 5-(2-chlorophenyl)-4-(cyclohexen-1-ylmethyl)-2-cyclopropyl-pyrimidine (Example 9, 0.107 g, Yield 10%): ¹H NMR (500 MHz, Chloroform-d) δ 8.24 (s, 1H), 6.93 (s, 1H), 6.63 (s, 1H), 6.07-6.01 (m, 2H), 5.03 (s, 1H), 3.38-3.08 (m, 2H), 2.32-2.23 (m, 1H), 1.97-1.42 (m, 8H), 1.22-1.15 (m, 2H), 1.11-1.04 (m, 2H). HPLC/MS Rₜ: 1.412 min; m/z: 369.1 [M+H+]. 5-(2-chlorophenyl)-4-(cyclohexylidenemethyl)-2-cyclopropyl-pyrimidine (Example 10, 0.093 g, Yield 8.5%) HPLC/MS Rₜ: 1.345 min; m/z: 369.1 [M+H+].

With appropriate modification of the starting materials, the procedures given in the synthesis examples below were used to obtain further compounds of formula I. The compounds obtained in this manner are listed in the Table Z that follows, together with physical data.

The products shown below were characterized by melting point determination, by the masses ([m/z]) or retention time (RT; [min.]) determined by HPLC-MS or HPLC spectrometry.

HPLC-MS=high performance liquid chromatography-coupled mass spectrometry; HPLC methods:

Method: Column: Phenomenex Kinetex 1.7 μm XB-C18 100A; 50×2.1 mm; mobile phase: A: water+0.1% trifluoroacetic acid (TFA); B: acetonitrile+0.1% TFA; gradient: 5-100% B in 1.50 minutes; 100% B 0.25 min; flow: 0.8-1.0 ml/min in 1.51 minutes at 60° C. MS: quadrupole electrospray ionization, 80 V (positive mode).

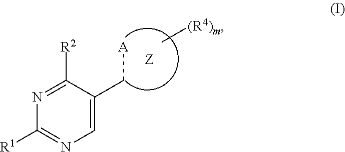

TABLE Z

| Exam- ple. | R¹ | R² | 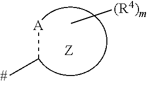 | HPLC/ MS, Rₜ [min] | HPLC/ MS, m/z |
|---|---|---|---|---|---|
| Ex.1 | c-C₃H₅ | 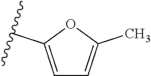 [SMILES: *c1ccc(C)o1] | 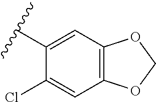 [SMILES: *c1cc2c(cc1Cl)OCO2] | 1.244 | 354.8 |
| Ex.2 | c-C₃H₅ | 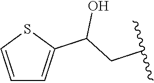 [SMILES: *CC(O)c1cccs1] | 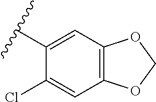 [SMILES: *c1cc2c(cc1Cl)OCO2] | 1.205 | 401.1 |
| Ex.3 | c-C₃H₅ | 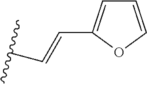 [SMILES: */C=C/c1ccco1] | 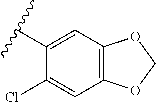 [SMILES: *c1cc2c(cc1Cl)OCO2] | 1.287 | 366.9 |
| Ex.4 | c-C₃H₅ | 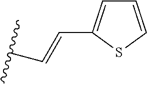 [SMILES: */C=C/c1cccs1] | 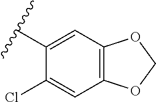 [SMILES: *c1cc2c(cc1Cl)OCO2] | 1.324 | 383.0 |
| Ex.5 | c-C₃H₅ | 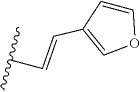 [SMILES: */C=C/c1ccoc1] | 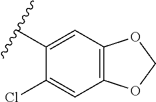 [SMILES: *c1cc2c(cc1Cl)OCO2] | 1.242 | 367.1 |
| Ex.6 | c-C₃H₅ | 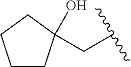 [SMILES: *CC1(O)CCCC1] | 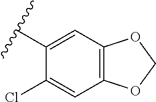 [SMILES: *c1cc2c(cc1Cl)OCO2] | 1.252 | 373.1 |
| Ex.7 | c-C₃H₅ | 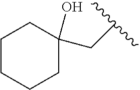 [SMILES: *CC1(O)CCCCC1] | 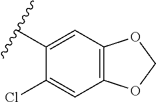 [SMILES: *c1cc2c(cc1Cl)OCO2] | 1.304 | 387.1 |
| Ex.8 | c-C₃H₅ | 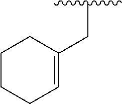 [SMILES: *CC1=CCCCC1] | 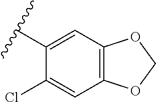 [SMILES: *c1cc2c(cc1Cl)OCO2] | 1.412 | 369.1 |
| Ex.9 | c-C₃H₅ | 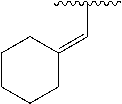 [SMILES: *C=C1CCCCC1] | 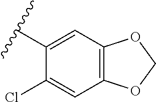 [SMILES: *c1cc2c(cc1Cl)OCO2] | 1.345 | 369.1 |
| Ex.10 | c-C₃H₅ | CH₂CO₂C₂H₅ | 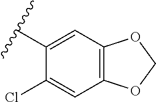 [SMILES: *c1cc2c(cc1Cl)OCO2] | 1.190 | 361.0 |
| Ex.11 | c-C₃H₅ | CHCH₃CO₂C₂H₅ | 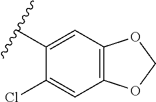 [SMILES: *c1cc2c(cc1Cl)OCO2] | 1.307 | 374.9 |

### B USE EXAMPLES

The herbicidal activity of the pyrimidine compounds of formula (I) was demonstrated by the following greenhouse experiments:

The culture containers used were plastic flowerpots containing loamy sand with approximately 3.0% of humus as the substrate. The seeds of the test plants were sown separately for each species.

For the pre-emergence treatment, the active ingredients, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this had been impaired by the active ingredients.

For the post-emergence treatment, the test plants were first grown to a height of 3 to 15 cm, depending on the plant habit, and only then treated with the active ingredients which had been suspended or emulsified in water. For this purpose, the test plants were either sown directly and grown in the same containers, or they were first grown separately as seedlings and transplanted into the test containers a few days prior to treatment.

Depending on the species, the plants were kept at 10-25° C. or 20-35° C., respectively.

The test period extended over 2 to 4 weeks. During this time, the plants were tended, and their response to the individual treatments was evaluated.

Evaluation was carried out using a scale from 0 to 100. 100 means no emergence of the plants, or complete destruction of at least the aerial moieties, and 0 means no damage, or normal course of growth. A good herbicidal activity is given at values of at least 70 and a very good herbicidal activity is given at values of at least 85.

The plants used in the greenhouse experiments were of the following species:

| Bayer code | Scientific name |
|---|---|
| AMARE | Amaranthus retroflexus |
| ECHCG | Echinocloa crus-galli |

At an application rate of 1000 g/ha, Ex. 1 applied by the post-emergence method showed very good herbicidal activity against AMARE.

At an application rate of 500 g/ha, Ex. 2 applied by the post-emergence method showed very good herbicidal activity against AMARE.

At an application rate of 2000 g/ha, Ex. 3 applied by the post-emergence method showed very good herbicidal activity against AMARE.

At an application rate of 2000 g/ha, Ex. 4 applied by the post-emergence method showed good herbicidal activity against AMARE and ECHCG.

At an application rate of 2000 g/ha, Ex. 8 applied by the post-emergence method showed very good herbicidal activity against ECHCG.

1-15. (canceled)

16. A pyrimidine compound of formula (I) 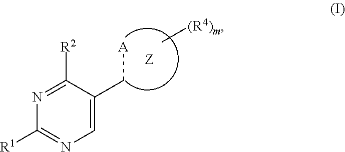
wherein
the dotted line (------) is a single bond or a double bond;
R¹ is C₁-C₆-alkyl, C₁-C₆-haloalkyl, HO—C₁-C₆-alkyl, C₂-C₆-alkenyl, C₂-C₆-haloalkenyl, C₂-C₆-alkynyl, C₃-C₆-haloalkynyl, C₁-C₆-alkoxy-C₁-C₆-alkyl, C₁-C₆-alkoxy, C₃-C₆-alkenyloxy, C₃-C₆-haloalkenyloxy, C₃-C₆-alkynyloxy, C₃-C₆-haloalkynyloxy, C₁-C₆-haloalkoxy, C₃-C₆-cycloalkoxy, C₃-C₆-halocycloalkoxy, C₃-C₆-cycloalkenyloxy, C₃-C₆-halocycloalkenyloxy, C₁-C₆-alkylthio, C₁-C₆-haloalkylthio, (C₁-C₆-alkyl)amino, di(C₁-C₆-alkyl)amino, C₁-C₆-alkylsulfinyl, C₁-C₆-alkylsulfonyl, C₃-C₆-cycloalkyl, C₃-C₆-cycloalkenyl, C₃-C₆-halocycloalkyl, C₃-C₆-halocycloalkenyl, [1-(C₁-C₆-alkyl)]-C₃-C₆-cycloalkyl, [1-(C₂-C₆-alkenyl)]-C₃-C₆-cycloalkyl, [1-(C₂-C₆-alkynyl)]-C₃-C₆-cycloalkyl, [1-(C₁-C₆-haloalkyl)]-C₃-C₆-cycloalkyl, [1-(C₂-C₆-haloalkenyl)]-C₃-C₆-cycloalkyl, [1-(C₃-C₆-haloalkynyl)]-C₃-C₆-cycloalkyl, C₃-C₆-cycloalkyl-C₁-C₆-alkyl, C₃-C₆-cycloalkyl-C₁-C₆-haloalkyl, C₃-C₆-cycloalkyl-C₁-C₆-alkoxy, C₃-C₆-cycloalkyl-C₁-C₆-haloalkoxy, phenyl, 5- or 6-membered heteroaryl, or 3- to 6-membered heterocyclyl;
wherein the cyclic groups of R¹ are unsubstituted or substituted by R^{a};
R² is C₁-C₆-alkyl, C₁-C₆-haloalkyl, C₂-C₆-alkenyl, C₂-C₆-haloalkenyl, C₁-C₆-alkoxy-C₂-C₆-alkenyl, C₁-C₆-alkoxy-C₂-C₆-haloalkenyl, C₁-C₆-haloalkoxy-C₂-C₆-alkenyl, C₁-C₆-haloalkoxy-C₂-C₆-haloalkenyl, C₂-C₆-alkynyl, C₂-C₆-haloalkynyl, C₁-C₆-alkoxy-C₂-C₆-alkynyl, C₁-C₆-alkoxy-C₃-C₆-haloalkynyl, C₁-C₆-haloalkoxy-C₂-C₆-alkynyl, C₁-C₆-haloalkoxy-C₃-C₆-haloalkynyl, C₃-C₆-cycloalkyl, C₃-C₆-halocycloalkyl, C₃-C₆-cycloalkenyl, C₃-C₆-halocycloalkenyl, C₃-C₆-cycloalkyl-C₁-C₆-alkyl, C₃-C₆-cycloalkyl-C₁-C₆-haloalkyl, C₃-C₆-halocycloalkyl-C₁-C₆-alkyl, C₃-C₆-halocycloalkyl-C₁-C₆-haloalkyl, C₃-C₆-cycloalkenyl-C₁-C₆-alkyl, C₃-C₆-cycloalkenyl-C₁-C₆-haloalkyl, C₃-C₆-halocycloalkenyl-C₁-C₆-alkyl, C₃-C₆-halocycloalkenyl-C₁-C₆-haloalkyl, C₃-C₆-cycloalkyl-C₂-C₆-alkenyl, C₃-C₆-cycloalkyl-C₂-C₆-haloalkenyl, C₃-C₆-halocycloalkyl-C₂-C₆-alkenyl, C₃-C₆-halocycloalkyl-C₂-C₆-haloalkenyl, C₃-C₆-cycloalkenyl-C₂-C₆-alkenyl, C₃-C₆-cycloalkenyl-C₂-C₆-haloalkenyl, C₃-C₆-halocycloalkenyl-C₂-C₆-alkenyl, C₃-C₆-halocycloalkenyl-C₂-C₆-haloalkenyl, C₃-C₆-cycloalkyl-C₂-C₆-alkynyl, C₃-C₆-cycloalkyl-C₃-C₆-haloalkynyl, C₃-C₆-halocycloalkyl-C₂-C₆-alkynyl, C₃-C₆-halocycloalkyl-C₃-C₆-haloalkynyl, C₃-C₆-cycloalkenyl-C₂-C₆-alkynyl, C₃-C₆-cycloalkenyl-C₃-C₆-haloalkynyl, C₃-C₆-halocycloalkenyl-C₂-C₆-alkynyl, C₃-C₆-halocycloalkenyl-C₃-C₆-haloalkynyl, C₃-C₆-cycloalkyl-C₁-C₆-alkylidenyl, C₃-C₆-cycloalkyl-C₂-C₆-haloalkylidenyl, C₃-C₆-halocycloalkyl-C₁-C₆-alkylidenyl, C₃-C₆-halocycloalkyl-C₂-C₆-haloalkylidenyl, C₃-C₆-cycloalkenyl-C₁-C₆-alkylidenyl, C₃-C₆-cycloalkenyl-C₂-C₆-haloalkylidenyl, C₃-C₆-halocycloalkenyl-C₁-C₆-alkylidenyl, C₃-C₆-halocycloalkenyl-C₂-C₆-haloalkylidenyl, heterocyclyl-C₁-C₆-alkylidenyl, heterocyclyl-C₁-C₆-haloalkylidenyl, C₃-C₆-hydroxycycloalkyl-C₁-C₆-alkyl, C₃-C₆-hydroxycycloalkyl-C₁-C₆-haloalkykylC₃-C₆-hydroxycycloalkenyl-C₁-C₆-alkyl, C₃-C₆-hydroxycycloalkenyl-C₁-C₆-haloalkyl, C₁-C₆-hydroxyalkyl, C₂-C₆-hydroxyhaloalkyl, C₃-C₆-hydroxyalkenyl, C₃-C₆-hydroxyhaloalkenyl, C₃-C₆-hydroxyalkynyl, C₄-C₆-hydroxyhaloalkynyl, C₃-C₆-hydroxycycloalkyl, C₃-C₆-hydroxyhalocycloalkyl, C₃-C₆-hydroxycycloalkenyl, C₃-C₆-hydroxyhalocycloalkenyl, C₃-C₆-cycloalkyl-C₁-C₆-hydroxyalkyl, C₃-C₆-cycloalkyl-C₂-C₆-hydroxyhaloalkyl, C₃-C₆-halocycloalkyl-C₁-C₆-hydroxyalkyl, C₃-C₆-halocycloalkyl-C₂-C₆-hydroxyhaloalkyl, C₃-C₆-cycloalkenyl-C₁-C₆-hydroxyalkyl, C₃-C₆-cycloalkenyl-C₂-C₆-hydroxyhaloalkyl, C₃-C₆-halocycloalkenyl-C₁-C₆-hydroxyalkylC₃-C₆-halocycloalkenyl-C₂-C₆-hydroxyhaloalkyl, C₃-C₆-cycloalkyl-C₃-C₆-hydroxyalkenyl, C₃-C₆-cycloalkyl-C₃-C₆-hydroxyhaloalkenyl, C₃-C₆-halocycloalkyl-C₃-C₆-hydroxyalkenyl, C₃-C₆-halocycloalkyl-C₃-C₆-hydroxyhaloalkenyl, C₃-C₆-cycloalkenyl-C₃-C₆-hydroxyalkenyl, C₃-C₆-cycloalkenyl-C₃-C₆-hydroxyhaloalkenyl, C₃-C₆-halocycloalkenyl-C₃-C₆-hydroxyalkenyl, C₃-C₆-halocycloalkenyl-C₃-C₆-hydroxyhaloalkenyl, C₃-C₆-cycloalkyl-C₃-C₆-hydroxyalkynyl, C₃-C₆-halocycloalkyl-C₃-C₆-hydroxyalkynyl, C₃-C₆-cycloalkenyl-C₃-C₆-hydroxyalkynyl, C₃-C₆-halocycloalkenyl-C₃-C₆-hydroxyalkynyl, C₃-C₆-cycloalkyl-C₂-C₆-hydroxyalkylidenyl, C₃-C₆-halocycloalkyl-C₂-C₆-hydroxyalkylidenyl, C₃-C₆-cycloalkenyl-C₂-C₆-hydroxyalkylidenyl, C₃-C₆-halocycloalkyl-C₂-C₆-hydroxyalkylidenyl, heterocyclyl-C₂-C₆-hydroxyalkylidenyl, hydroxycarbonyl-C₁-C₆-hydroxyalkyl, hydroxycarbonyl-C₁-C₆-alkyl, hydroxycarbonyl-C₁-C₆-haloalkyl, C₁-C₆-alkoxycarbonyl-C₁-C₆-hydroxyalkyl, C₁-C₆-haloalkoxycarbonyl-C₁-C₆-hydroxyalkyl, C₁-C₆-alkoxycarbonyl-C₁-C₆-haloalkyl, C₁-C₆-haloalkoxycarbonyl-C₁-C₆-haloalkyl, C₁-C₆-alkoxycarbonyl-C₁-C₆-alkyl, C₁-C₆-haloalkoxycarbonyl-C₁-C₆-alkyl, C₃-C₆-hydroxycycloalkyl-C₁-C₆-hydroxyalkyl, C₃-C₆-hydroxycycloalkenyl-C₁-C₆-hydroxyalkyl, C₃-C₆-hydroxycycloalkyl-C₃-C₆-hydroxyalkenyl, C₃-C₆-hydroxycycloalkenyl-C₃-C₆-hydroxyalkenyl, C₃-C₆-hydroxycycloalkyl-C₃-C₆-hydroxyalkynyl, C₃-C₆-hydroxycycloalkenyl-C₃-C₆-hydroxyalkenyl, C₂-C₆-dihydroxyalkyl, C₃-C₆-dihydroxyhaloalkyl, C₄-C₆-dihydroxyalkenyl, C₄-C₆-dihydroxyhaloalkenyl, C₄-C₆-dihydroxyalkynyl, C₅-C₆-dihydroxyhaloalkynyl, C₄-C₆-dihydroxycycloalkyl, C₄-C₆-dihydroxyhalocycloalkyl, C₄-C₆-dihydroxycycloalkenyl, C₄-C₆-dihydroxyhalocycloalkenyl, C₃-C₆-cycloalkyl-C₂-C₆-dihydroxyalkyl, C₃-C₆-halocycloalkyl-C₂-C₆-dihydroxyalkyl, C₃-C₆-cycloalkenyl-C₂-C₆-dihydroxyalkyl, C₃-C₆-halocycloalkenyl-C₂-C₆-dihydroxyalkyl, C₃-C₆-cycloalkyl-C₃-C₆-dihydroxyalkenyl, C₃-C₆-halocycloalkyl-C₃-C₆-dihydroxyalkenyl, C₃-C₆-cycloalkenyl-C₃-C₆-dihydroxyalkenyl, C₃-C₆-halocycloalkenyl-C₃-C₆-dihydroxyalkenyl, C₃-C₆-cycloalkyl-C₄-C₆-dihydroxyalkynyl, C₃-C₆-halocycloalkyl-C₄-C₆-dihydroxyalkynyl, C₃-C₆-cycloalkenyl-C₄-C₆-dihydroxyalkynyl, C₃-C₆-halocycloalkyl-C₄-C₆-dihydroxyalkynyl, C₃-C₆-cycloalkyl-C₃-C₆-dihydroxyalkylidenyl, C₃-C₆-halocycloalkyl-C₃-C₆-dihydroxyalkylidenyl, heterocyclyl-C₃-C₆-dihydroxyalkylidenyl, hydroxycarbonyl-C₂-C₆-dihydroxyalkyl, hydroxycarbonyl-C₃-C₆-dihydroxyhaloalkyl, C₁-C₆-alkoxycarbonyl-C₂-C₆-dihydroxyalkyl, C₁-C₆-haloalkoxycarbonyl-C₂-C₆-dihydroxyalkyl, C₁-C₆-haloalkoxycarbonyl-C₃-C₆-dihydroxyhaloalkyl, C₃-C₆-dihydroxycycloalkyl-C₃-C₆-dihydroxycycloalkyl-C₁-C₆-haloalkylC₃-C₆-dihydroxycycloalkyl-C₂-C₆-alkenyl, C₃-C₆-dihydroxycycloalkyl-C₂-C₆-haloalkenyl, C₃-C₆-dihydroxycycloalkyl-C₂-C₆-alkynyl, C₃-C₆-dihydroxycycloalkyl-C₃-C₆-haloalkynyl, C₁-C₆-haloalkylcarbonyl-C₁-C₆-alkyl, C₁-C₆-alkylcarbonyl-C₁-C₆-haloalkyl, C₁-C₆-haloalkylcarbonyl-C₁-C₆-haloalkyl, hydroxycarbonyl-C₂-C₆-alkenyl, hydroxycarbonyl-C₂-C₆-haloalkenyl, C₁-C₆-alkoxycarbonyl-C₂-C₆-alkenyl, C₁-C₆-haloalkoxycarbonyl-C₂-C₆-alkenyl, C₁-C₆-alkoxycarbonyl-C₂-C₆-haloalkenyl, C₁-C₆-haloalkoxycarbonyl-C₂-C₆-haloalkenyl, hydroxycarbonyl-C₂-C₆-alkynyl, hydroxycarbonyl-C₃-C₆-haloalkynyl, C₁-C₆-alkoxycarbonyl-C₂-C₆-alkynyl, C₁-C₆-haloalkoxycarbonyl-C₂-C₆-alkynyl, C₁-C₆-alkoxycarbonyl-C₃-C₆-haloalkynyl, C₁-C₆-haloalkoxycarbonyl-C₃-C₆-haloalkynyl, C₁-C₆-cyanoalkyl, C₂-C₆-cyanohaloalkyl, C₁-C₆-dicyanoalkyl, C₂-C₆-dicyanohaloalkyl, di(hydroxycarbonyl)-C₁-C₆-alkyl, di(hydroxycarbonyl)-C₁-C₆-haloalkyl, di(C₁-C₆-alkoxycarbonyl)-C₁-C₆-alkyl, di(C₁-C₆-haloalkoxycarbonyl)-C₁-C₆-alkyl, di(C₁-C₆-alkoxycarbonyl)-C₁-C₆-haloalkyl, di(C₁-C₆-haloalkoxycarbonyl)-C₁-C₆-haloalkyl, di(C₁-C₆-alkoxyl)phosphoryl-C₁-C₆-alkyl, di(C₁-C₆-haloalkoxyl)phosphoryl-C₁-C₆-alkyl, di(C₁-C₆-alkoxyl)phosphoryl-C₁-C₆-haloalkyl, di(C₁-C₆-haloalkoxyl)phosphoryl-C₁-C₆-haloalkyl, phosphoryl-C₁-C₆-alkyl, phosphoryl-C₁-C₆-haloalkyl, di[di(C₁-C₆-alkoxyl)phosphoryl-]C₁-C₆-alkyl, di[di(C₁-C₆-haloalkoxyl)phosphoryl-)]-C₆-alkyl, di[di(C₁-C₆-alkoxyl)phosphoryl-)]C₁-C₆-haloalkyl, di[di(C₁-C₆-haloalkoxyl)phosphoryl-]C₁-C₆-haloalkyl, diphosphoryl-C₁-C₆-alkyl, diphosphoryl-C₁-C₆-haloalkyl, alkylthio-C₁-C₆-alkyl, C₁-C₆-haloalkylthio-C₁-C₆-alkyl, C₁-C₆-alkylthio-C₁-C₆-C₁-C₆-haloalkylsulfonyl-C₁-C₆-haloalkyl, phenyl, 5- or 6-membered heteroaryl, or 3- to 6-membered heterocyclyl;
wherein hydroxy groups of R² are unsubstituted or substituted by R^{b};
cyclic groups of R² are unsubstituted or substituted by R^{c}; and
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl; wherein the substituent R^{d} is unsubstituted or substituted by R^{e};
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylsulfonyl;
Z is a 9 or 10 membered bicyclic ring comprising A, wherein the ring comprising A is a 5- or 6-membered aromatic ring comprising 0, 1, 2, or 3 heteroatoms selected from O, N, and S, and which is fused with another 5- or 6-membered partially or fully unsaturated carbocycle comprising 0, 1, 2, or 3 heteroatoms selected from O, N, and S;
A is C*, CR³, NR^{3A}, N, O, or S;
C* is a bridge carbon of the bicyclic ring Z;
R³ is halogen, CN, CHO, NO₂, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R³ are unsubstituted or substituted by substituents R^{a};
R^{3A} is H, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, C₁-C₆-alkoxycarbonyl, C₁-C₆-alkylthio, C₁-C₆-haloalkylthio, NH₂, (C₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R^{3A} are unsubstituted or substituted by R^{a};
R⁴ is halogen, CN, CHO, NO₂, C₁-C₆-alkyl, C₁-C₆-haloalkyl, C₁-C₆-alkylcarbonyl, C₂-C₆-alkenyl, C₂-C₆-haloalkenyl, C₂-C₆-alkenyl, C₂-C₆-haloalkenyl, C₁-C₆-alkoxy, C₁-C₆-haloalkoxy, C₃-C₆-alkenyloxy, C₃-C₆-haloalkenyloxy, C₃-C₆-alkenyloxy, C₃-C₆-haloalkenyloxy, C₁-C₆-alkoxy-C₁-C₆-alkoxy, hydroxycarbonyl, C₁-C₆-alkoxycarbonyl, C₁-C₆-alkylthio, C₁-C₆-haloalkylthio, NH₂, (C ₁-C₆-alkyl)amino, di(C₁-C₆-alkyl)amino, (C₁-C₆-alkyl)sulfinyl, (C₁-C₆-alkyl)sulfonyl, C₃-C₆-cycloalkyl, (C₃-C₆-cycloalkyl)oxy, or phenyl;
wherein the cyclic groups of R⁴ are unsubstituted or substituted by R^{a};
R^{a} is halogen, CN, NO₂, C₁-C₆-alkyl, C₁-C₆-haloalkyl, C₁-C₆-alkoxy, or C₁-C₆-haloalkoxy;
m is 0, 1, 2, or 3;
including agriculturally acceptable salts or derivatives of the pyrimidine compounds of formula (I) having an acidic functionality.

17. The pyrimidine compound of formula (I) of claim 16, wherein
R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₄-C₆-haloalkynyloxy, C₁-C₆-alkylthio, or C₃-C₆-cycloalkyl;
wherein the cycloalkyl substituent is unsubstituted.

18. The pyrimidine compound of formula (I) of claim 16, wherein
R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, hydroxycycloalkenyl-C₁-C₆-alkyl, C₁-C₆-hydroxyalkyl, C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;
wherein hydroxy groups of R² are unsubstituted or substituted by R^{b};
cyclic groups of R² are unsubstituted or substituted by R^{e};
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylsulfonyl.

19. The pyrimidine compound of formula (I) of claim 16, wherein Z is selected from below groups A to O, 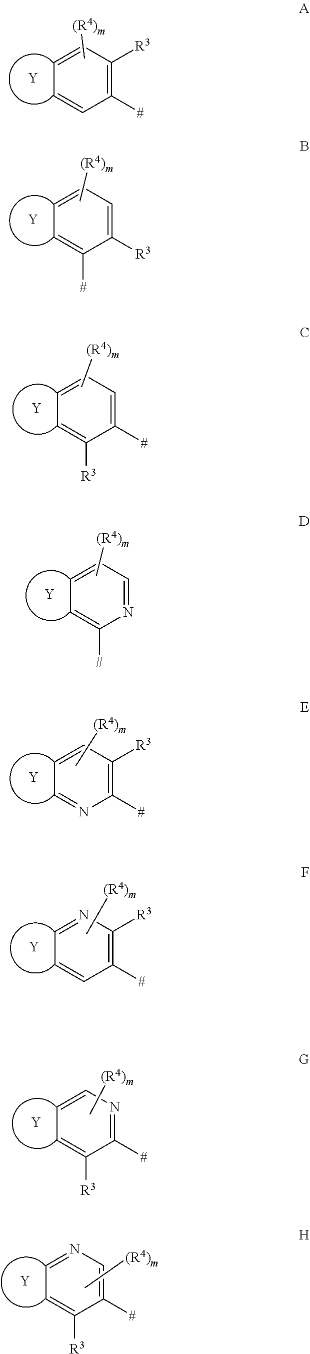 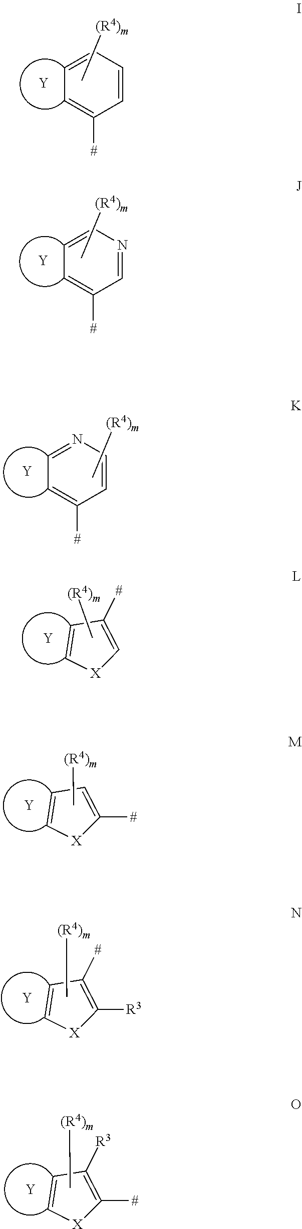
wherein
Y is 5- or 6-membered partially or fully unsaturated carbocycle comprising 0, 1, 2, or 3 heteroatoms selected from O, N, and S;
R³ is halogen, CN, C₁-C₆-alkyl, C₁-C₆-haloalkyl, or C₁-C₆-alkoxy;
m is 0, 1 or 2;
R⁴ is halogen, CN, C₁-C₆-alkyl, C₁-C₆-haloalkyl, or C₁-C₆-alkoxy;
X is O, S, or NR^{3A};
R^{3A} is H, C₁-C₆-alkyl, C₁-C₆-haloalkyl, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl; and
# denotes the point of attachment to the pyrimidine ring.

20. The pyrimidine compound of formula (I) of claim 16, wherein
R¹ is c-C₃H₅;
R² is C₁-C₆-alkyl, C₁-C₆-haloalkyl, C₂-C₆-alkenyl, C₃-C₆-cycloalkyl-C₁-C₆-alkyl, C₃-C₆-cycloalkenyl-C₁-C₆-alkyl, C₃-C₆-cycloalkyl-C₁-C₆-alkylidenyl, C₁-C₆-hydroxyalkyl, C₁-C₆-alkoxycarbonyl-C₁-C₆-alkyl, or 5-membered heteroaryl;
wherein OH groups of R² are unsubstituted or substituted by R^{b},
cyclic groups of R² are unsubstituted or substituted by R^{e}, and
acyclic aliphatic groups of R² are unsubstituted or substituted by R^{d};
R^{b} is C₁-C₆-alkyl;
R^{e} is C₁-C₆-alkyl or OH;
R^{d} is 5- or 6-membered heteroaryl or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy, C₁-C₆-haloalkoxy, or C₁-C₆-alkylsulfonyl;
Z is A;
Y is 5- or 6-membered partially unsaturated carbocycle comprising 1 or 2 oxygen atoms;
R³ is R³ is halogen, C₁-C₆-alkyl, C₁-C₆-haloalkyl, C₁-C₆-alkoxy, or C₁-C₆-haloalkoxy; m is 0 or 1; R⁴ is F, Br, Cl, CHF₂, CH₃, CF₃, or C₂H₅.

21. A herbicidal composition comprising:
A) at least one pyrimidine compound of formula (I), including agriculturally acceptable salts or derivatives of the compound of formula (I) having an acidic functionality, as defined in claim 16;
and
B) herbicides of class b1) to b15):
b1) lipid biosynthesis inhibitors;
b2) acetolactate synthase inhibitors (ALS inhibitors);
b3) photosynthesis inhibitors;
b4) protoporphyrinogen-IX oxidase inhibitors,
b5) bleacher herbicides;
b6) enolpyruvyl shikimate 3-phosphate synthase inhibitors (EPSP inhibitors);
b7) glutamine synthetase inhibitors;
b8) 7,8-dihydropteroate synthase inhibitors (DHP inhibitors);
b9) mitosis inhibitors;
b10) inhibitors of the synthesis of very long chain fatty acids (VLCFA inhibitors);
b11) cellulose biosynthesis inhibitors;
b12) decoupler herbicides;
b13) auxinic herbicides;
b14) auxin transport inhibitors; and
b15) other herbicides selected from the group consisting of bromobutide, chlorflurenol, chlorflurenol-methyl, cinmethylin, cumyluron, dalapon, dazomet, difenzoquat, difenzoquat-metilsulfate, dimethipin, DSMA, dymron, endothal and its salts, etobenzanid, flamprop, flamprop-isopropyl, flamprop-methyl, flamprop-M-isopropyl, flamprop-M-methyl, flurenol, flurenol-butyl, flurprimidol, fosamine, fosamine-ammonium, indanofan, indaziflam, maleic hydrazide, mefluidide, metam, methiozolin (CAS 403640-27-7), methyl azide, methyl bromide, methyl-dymron, methyl iodide, MSMA, oleic acid, oxaziclomefone, pelargonic acid, pyributicarb, quinoclamine, triaziflam, tridiphane and 6-chloro-3-(2-cyclopropyl-6-methylphenoxy)-4-pyridazinol, and its salts and esters;
including their agriculturally acceptable salts or derivatives.

22. A herbicidal composition comprising the herbicidal composition of claim 21, and safeners.

23. The herbicidal composition of claim 21, wherein the herbicidal composition comprises at least one herbicide B selected from herbicides of class b1, b2, b3, b4, b5, b6, b9, b10, b13 and b14.

24. The herbicidal composition of claim 21, wherein the herbicidal composition comprises at least one herbicide B selected from herbicides of class b1, b2, b4, b5, b9, b10, b13 and b14.

25. The herbicidal composition of claim 21, wherein the weight ratio of component A to component B is in the range of from 1:500 to 500:1.

26. A herbicidal composition comprising a herbicidally active amount of at least one pyrimidine compound of formula (I including agriculturally acceptable salts or derivatives of the compound of formula (I) having an acidic functionality, as defined in claim 16, and at least one inert liquid and/or solid carrier and, if appropriate, at least one surface-active substance.

27. A herbicidal composition comprising the herbicidal composition of claim 21, and at least one inert liquid and/or solid carrier and, if appropriate, at least one surface-active substance.

28. A method of controlling undesired vegetation, which comprises allowing a herbicidally active amount of at least one pyrimidine compound of formula (I) of claim 16, including agriculturally acceptable salts or derivatives of the compound of formula (I) having an acidic functionality, to act on plants, their environment or on seed.

29. A method of controlling undesired vegetation, which comprises allowing a herbicidally active amount of the composition of claim 21 to act on plants, their environment or on seed.

30. The method of claim 28, wherein
R¹ is C₁-C₆-alkyl, C₁-C₆-alkoxy, C₁-C₆-haloalkoxy, C₃-C₆-alkenyloxy, C₃-C₆-haloalkenyloxy C₃-C₆-alkynyloxy, C₄-C₆-haloalkynyloxy, C₁-C₆-alkylthio, or C₃-C₆-cycloalkyl;
wherein the cycloalkyl substituent is unsubstituted.

31. The p method of claim 28, wherein
R² is C₂-C₆-alkenyl, C₂-C₆-haloalkenyl, C₁-C₆-alkoxy-C₂-C₆-alkenyl, C₂-C₆-alkynyl, C₃-C₆-cycloalkenyl, C₃-C₆-cycloalkenyl-C₁-C₆-alkyl, C₃-C₆-cycloalkyl-C₁-C₆-alkylidenyl, C₃-C₆-halocycloalkyl-C₁-C₆-alkylidenyl, C₃-C₆-cycloalkenyl-C₁-C₆-alkylidenyl, C₃-C₆-hydroxycycloalkyl-C₁-C₆-alkyl, hydroxycycloalkenyl-C₁-C₆-alkyl C₃-C₆-cycloalkyl-C₂-C₆-hydroxyalkylidenyl, hydroxycarbonyl-C₁-C₆-alkyl, hydroxycarbonyl-C₁-C₆-haloalkyl, C₁-C₆-alkoxycarbonyl-C₁-C₆-alkyl, C₃-C₆-hydroxycycloalkyl-C₁-C₆-hydroxyalkyl, C₂-C₆-dihydroxyalkyl, C₃-C₆-cycloalkyl-C₃-C₆-dihydroxyalkylidenyl, hydroxycarbonyl-C₂-C₆-dihydroxyalkyl, C₁-C₆-alkoxycarbonyl-C₂-C₆-dihydroxyalkyl, C₁-C₆-dicyanoalkyl, or 5- or 6-membered heteroaryl;
wherein hydroxy groups of R² are unsubstituted or substituted by R^{b};
cyclic groups of R² are unsubstituted or substituted by R^{c};
acyclic aliphatic groups of R² are unsubstituted or substituted by R^{d};
R^{b} is C₁-C₆-alkyl, C₁-C₆-haloalkyl, C₃-C₆-alkenyl, C₃-C₆-haloalkenyl, C₃-C₆-alkynyl, C₃-C₆-haloalkynyl, C₃-C₆-cycloalkyl, C₃-C₆-halocycloalkyl, C₄-C₆-cycloalkenyl, C₃-C₆-halocycloalkenyl, C₁-C₆-alkoxycarbonyl-C₁-C₆-alkyl, C₁-C₆-haloalkoxycarbonyl-C₁-C₆-alkyl, C₁-C₆-alkoxycarbonyl-C₁-C₆-haloalkyl, C₁-C₆-haloalkoxycarbonyl-C₁-C₆-haloalkyl, C₁-C₆-alkylcarbonyl, C₁-C₆-haloalkylcarbonyl, hydroxycarbonyl-C₁-C₆-alkyl, hydroxycarbonyl-C₁-C₆-haloalkyl, C₁-C₆-alkyloxycarbonyl, C₁-C₆-haloalkyloxycarbonyl, C₁-C₆-alkylthiocarbonyl, C₁-C₆-haloalkylthiocarbonyl, C₁-C₆-alkylaminocarbonyl, C₁-C₆-haloalkylaminocarbonyl, C₁-C₆-dialkylaminocarbonyl, C₁-C₆-dihaloalkylaminocarbonyl, C₁-C₆-alkylsulfonyl, C₁-C₆-haloalkylsulfonyl, C₁-C₆-alkoxy-C₁-C₆-alkyl, C₁-C₆-haloalkoxy-C₁-C₆-alkyl, C₁-C₆-alkoxy-C₁-C₆-haloalkyl, C₁-C₆-haloalkoxy-C₁-C₆-haloalkyl, phenyl-C₁-C₆-alkyl, or phenyl-C₁-C₆-haloalkyl;
R^{c} is halogen, CN, NO₂, C₁-C₆-alkyl, C₁-C₆-haloalkyl, hydroxy, C₁-C₆-alkoxy or C₁-C₆-haloalkoxy, C₁-C₆-alkylthio, C₁-C₆-alkylsulfinyl, or C₁-C₆-alkylsulfonyl;
R^{d} is phenyl, 5- or 6-membered heteroaryl, or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, hydroxy, C₁-C₆-alkoxy or C₁-C₆-haloalkoxy, C₁-C₆-alkylsulfonyl.

32. The py method of claim 28, wherein Z is selected from below groups A to O, 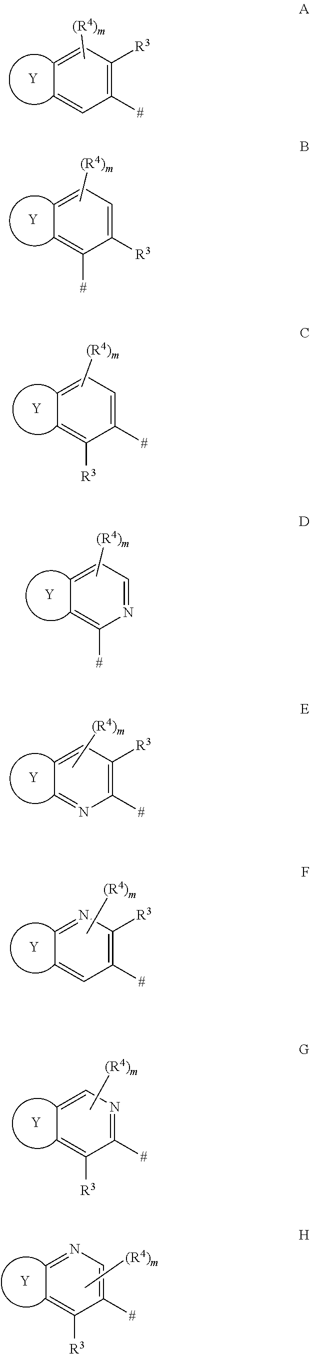 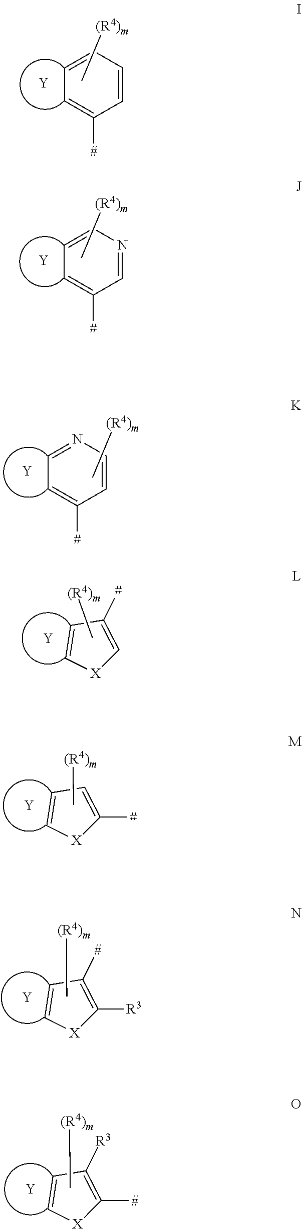
wherein
Y is 5- or 6-membered partially or fully unsaturated carbocycle comprising 0, 1, 2, or 3 heteroatoms selected from O, N, and S;
R³ is halogen, CN, C₁-C₆-haloalkyl, or C₁-C₆-alkoxy;
m is 0, 1 or 2;
R⁴ is halogen, CN, C₁-C₆-haloalkyl, or C₁-C₆-alkoxy;
X is O, S, or NR^{3A};
R^{3A} is H, C₁-C₆-alkylcarbonyl, C₃-C₆-alkenyl, C₃-C₆-haloalkenyl, C₃-C₆-alkenyl, C₃-C₆-haloalkenyl, or C₃-C₆-cycloalkyl; and
# denotes the point of attachment to the pyrimidine ring.

33. The method of claim 28, wherein
R¹ is c-C₃H₅;
R² is C₁-C₆-alkyl, C₂-C₆-alkenyl, C₃-C₆-cycloalkenyl-C₁-C₆-alkyl, C₃-C₆-cycloalkyl-C₁-C₆-alkylidenyl, C₁-C₆-hydroxyalkyl, C₁-C₆-alkoxycarbonyl-C₁-C₆-alkyl, or 5-membered heteroaryl;
wherein OH groups of R² are unsubstituted or substituted by R^{b},
cyclic groups of R² are unsubstituted or substituted by R^{e}, and
acyclic aliphatic groups of R² are unsubstituted or substituted by R^{d};
R^{b} is C₁-C₆-alkyl;
R^{c} is C₁-C₆-alkyl or OH;
R^{d} is 5- or 6-membered heteroaryl or 3- to 6-membered heterocyclyl;
wherein the substituent R^{d} is unsubstituted or substituted by R^{e};
R^{e} is halogen, CN, NO₂, C₁-C₆-alkyl, C₁-C₆-haloalkyl, OH, C₁-C₆-alkoxy, C₁-C₆-haloalkoxy, or C₁-C₆-alkylsulfonyl;
Z is A;
Y is 5- or 6-membered partially unsaturated carbocycle comprising 1 or 2 oxygen atoms;
R³ is R³ is halogen, C₁-C₆-alkyl, C₁-C₆-haloalkyl, C₁-C₆-alkoxy, or C₁-C₆-haloalkoxy; m is 0 or 1; R⁴ is F, Br, Cl, CHF₂, CH₃, CF₃, or C₂H₅.
